(19) United States
(12) Patent Application Publication
(10) Pub. No.: US 2021/0300912 A1
(43) Pub. Date: Sep. 30, 2021

(54) ARYL HYDROCARBON RECEPTOR (AHR) AGONISTS AND USES THEREOF

(71) Applicant: Ikena Oncology, Inc., Boston, MA (US)

(72) Inventors: Alfredo C. Castro, Sommerville, MA (US); Karen J. McGovern, Groton, MA (US); Michael Burke, Melrose, MA (US)

(21) Appl. No.: 17/126,514

(22) Filed: Dec. 18, 2020

Related U.S. Application Data
(60) Provisional application No. 62/951,386, filed on Dec. 20, 2019.
(60) Provisional application No. 63/122,107, filed on Dec. 7, 2020.

(51) Int. Cl. C07D 417/12; C07D 413/12; C07D 403/12; C07D 409/12; C07D 401/14; C07D 417/14; A61P 29/00

(52) U.S. Cl. CPC C07D 417/12; C07D 413/12; C07D 403/12; A61P 29/00; C07D 401/14; C07D 417/14; C07D 409/12

(57) ABSTRACT

The present invention provides AHR agonists, compositions thereof, and methods of using the same.

### CROSS-REFERENCE TO RELATED APPLICATIONS

This application claims the benefit under 35 U.S.C. § 119(e) of U.S. Provisional Patent Application No. 62/951,386, filed Dec. 20, 2019, and U.S. Provisional Patent Application No. 63/122,107, filed Dec. 7, 2020, the contents of each of which are herein incorporated by reference in their entireties.

### TECHNICAL FIELD OF THE INVENTION

The present invention relates to compounds and methods useful for activating aryl hydrocarbon receptor (AHR). The invention also provides pharmaceutically acceptable compositions comprising compounds of the present invention and methods of using said compositions in the treatment of various disorders.

### BACKGROUND

The aryl hydrocarbon receptor (AHR) is a ligand-inducible transcription factor that mediates a number of important biological and pharmacological processes. AHR agonists have been shown to be potentially useful for treating disorders such as cancer (U.S. Pat. No. 8,604,067, Wang et al., 2013, Cheng et al., 2015), obesity (U.S. Pat. No. 7,419,992), and conditions related to imbalanced actions of the immune system (Quintana et al., 2010, Nugent et al., 2013). AHR has also been shown to be involved in immune regulation, hematopoiesis, cell cycle, carcinogenesis and in the maintenance of intestinal barrier integrity and homeostasis.

### SUMMARY OF THE INVENTION

It has now been found that compounds of the present invention, and pharmaceutically acceptable compositions thereof, are effective as AHR agonists. In one aspect, the instant invention provides a compound of formula (I):

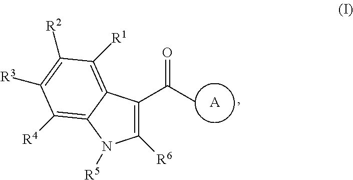

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with AHR. Such diseases, disorders, or conditions include, for example, cancer, obesity, and inflammatory disorders as described herein.

### DETAILED DESCRIPTION OF CERTAIN EMBODIMENTS

### 1. General Description of Certain Embodiments of the Invention

Compounds of the present invention, and pharmaceutical compositions thereof, are useful as AHR agonists. Without wishing to be bound by any particular theory, it is believed that compounds of the present invention, and pharmaceutical compositions thereof, may activate AHR and thus treat certain diseases, disorders, or conditions associated with AHR, such as those described herein.

It has now been found that compounds of this invention, and pharmaceutically acceptable compositions thereof, are effective as AHR agonists. In one aspect, the present invention provides a compound of Formula (I):

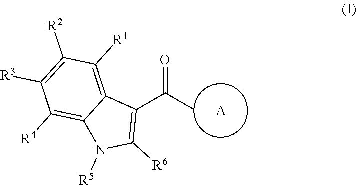

or a pharmaceutically acceptable salt thereof, wherein:
Ring A is an optionally substituted 5-membered heteroaromatic ring having 1-3 heteroatoms independently selected from N, O, or S;
each of R¹, R², R³, R⁴, and R⁶ is independently halogen, —CN, —NO₂, R^{W}, —C(O)—R^{W}, —C(═NR^{W})—R^{W}, —N(R^{W})—C(O)—R^{W}, —N(R^{W})—C(═NR^{W})—R^{W}, —OC(O)—R^{W}, —OC(═NR^{W})—R^{W}, —S(O)₂—R^{W}, —N(R^{W})—S(O)₂—R^{W}, —OS(O)₂—R^{W}, —S(O)—R^{W}, —N(R^{W})—S(O)—R^{W}, or —OS(O)—R^{W};
R⁵ is —R, —C(O)—R^{W}, —C(═NR^{W})—R^{W}, —S(O)₂—R^{W}, or —S(O)—R^{W}; R^{W} is —R, —N(R)₂, —NR—OR, —N(R)—N(R)₂, —N(OR)—N(R)₂, —N(R)—N(OR)R, —OR, —O—N(R)₂, or —SR; and
R is hydrogen, optionally substituted C₁₋₆ aliphatic, an optionally substituted 3-7 membered carbocyclic ring, or an optionally substituted 3-7 membered heterocyclic ring having 1-3 heteroatoms independently selected from N, O, or S, or two R's together with the nitrogen to which they attach form an optionally substituted 5-7 membered heterocyclic ring having 0-2 heteroatoms independently selected from N, O, or S in addition to the nitrogen to which the two R's attach.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

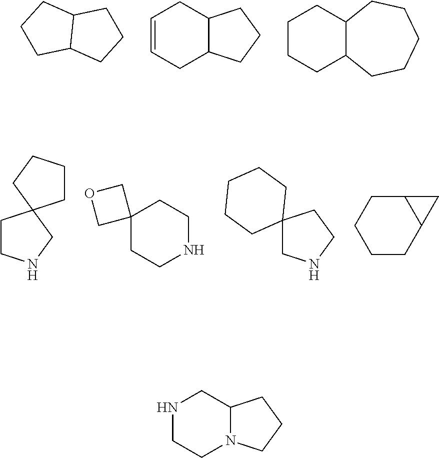

Exemplary bridged bicyclics include:

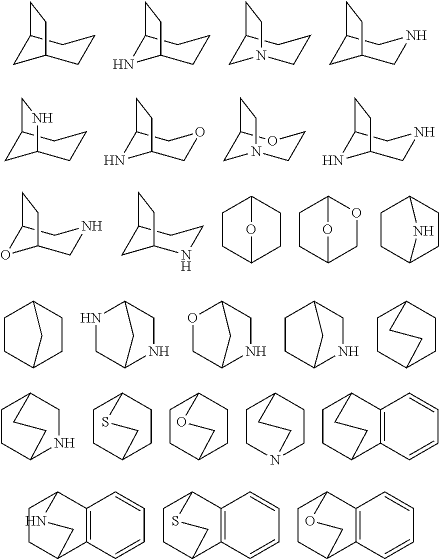

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

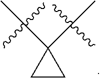 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 n electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, AH quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H pyrrolyl), NH (as in pyrrolidinyl), or ⁺NR (as in N substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R* is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of RJ, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R* is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphor sulfonate, citrate, cyclopentanepropionate, digluconate, dodecyl sulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the term “agonist” is defined as a compound that binds to and/or activates AHR with measurable affinity. In certain embodiments, an agonist has an IC₅₀ and/or binding constant of less than about 100 μM, less than about 50 μM, less than about 1 μM, less than about 500 nM, less than about 100 nM, less than about 10 nM, or less than about 1 nM.

The terms “measurable affinity” and “measurably activate,” as used herein, means a measurable change in AHR activity between a sample comprising a compound of the present invention, or composition thereof, and AHR, and an equivalent sample comprising AHR, in the absence of said compound, or composition thereof.

### 3. Description of Exemplary Embodiments

In one aspect, the present invention provides a compound of Formula (I):

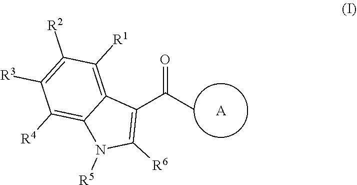

or a pharmaceutically acceptable salt thereof, wherein:
Ring A is an optionally substituted 5-membered heteroaromatic ring having 1-3 heteroatoms independently selected from N, O, or S;
each of R¹, R², R³, R⁴, and R⁶ is independently halogen, —CN, —NO₂, R^{W}, —C(O)—R^{W}, —C(═NR^{W})—R^{W}, —N(R^{W})—C(O)—R^{W}, —N(R^{W})—C(═NR^{W})—R^{W}, —OC(O)—R^{W}, —OC(═NR^{W})—R^{W}, —S(O)₂—R^{W}, —N(R^{W})—S(O)₂—R^{W}, —OS(O)₂—R^{W}, —S(O)—R^{W}, —N(R^{W})—S(O)—R^{W}, or —OS(O)—R^{W};
R⁵ is —R, —C(O)—R^{W}, —C(═NR^{W})—R^{W}, —S(O)₂—R^{W}, or —S(O)—R^{W};
R^{W} is —R, —N(R)₂, —NR—OR, —N(R)—N(R)₂, —N(OR)—N(R)₂, —N(R)—N(OR)R, —OR, —O—N(R)₂, or —SR; and
R is hydrogen, optionally substituted C₁₋₆ aliphatic, an optionally substituted 3-7 membered carbocyclic ring, or an optionally substituted 3-7 membered heterocyclic ring having 1-3 heteroatoms independently selected from N, O, or S, or two R's together with the nitrogen to which they attach form an optionally substituted 5-7 membered heterocyclic ring having 0-2 heteroatoms independently selected from N, O, or S in addition to the nitrogen to which the two R's attach.

As defined generally above, Ring A is an optionally substituted 5-membered heteroaromatic ring having 1-3 heteroatoms independently selected from N, O, or S.

In some embodiments, Ring A is an unsubstituted 5-membered heteroaromatic ring having 1-3 heteroatoms independently selected from N, O, or S. In some embodiments, Ring A is a 5-membered heteroaromatic ring having 1-3 heteroatoms independently selected from N, O, or S, which is substituted 1 or 2 times by R¹², wherein each R¹² is independently an optional substituent as defined above and described in embodiments herein.

In some embodiments, Ring A is an unsubstituted 5-membered heteroaromatic ring having 1, 2, or 3 heteroatoms independently selected from N or S. In some embodiments, Ring A is a 5-membered heteroaromatic ring having 1, 2, or 3 heteroatoms independently selected from N or S, which is substituted 1 or 2 times by R¹², wherein each R¹² is independently an optional substituent as defined above and described in embodiments herein.

In some embodiments, Ring A is an unsubstituted 5-membered heteroaromatic ring having 1, 2, or 3 heteroatoms independently selected from N or O. In some embodiments, Ring A is a 5-membered heteroaromatic ring having 1, 2, or 3 heteroatoms independently selected from N or O, which is substituted 1 or 2 times by R¹², wherein each R¹² is independently an optional substituent as defined above and described in embodiments herein.

In some embodiments, Ring A is optionally substituted

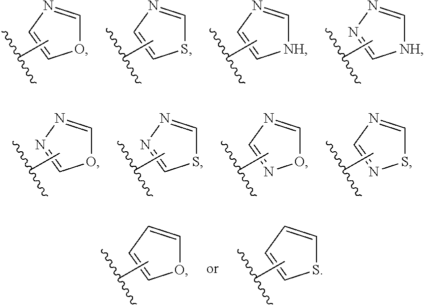

In some embodiments, Ring A is unsubstituted

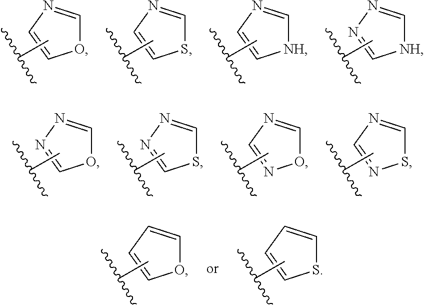

In some embodiments, Ring A is

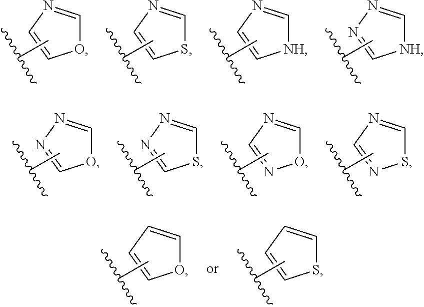

each of which is substituted 1 or 2 times by R¹², wherein each R¹² is independently an optional substituent as defined above and described in embodiments herein.

In some embodiments, Ring A is

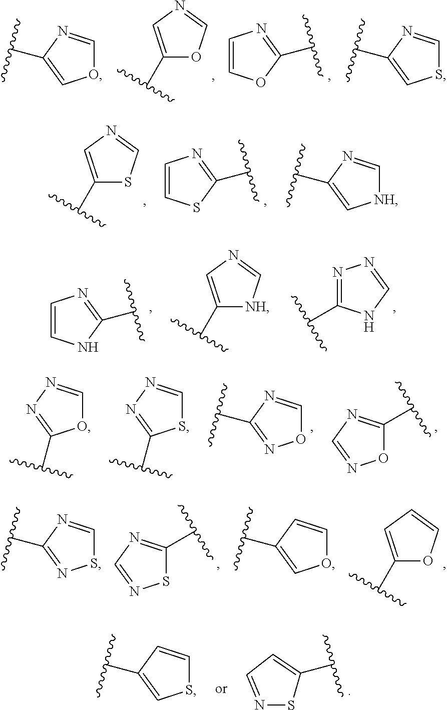

In some embodiments, Ring A is

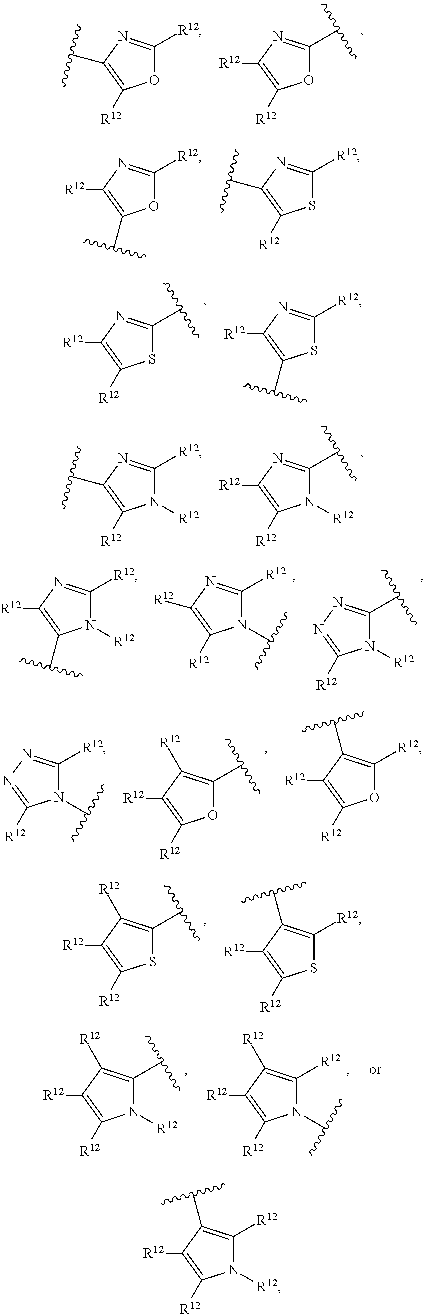

wherein each R¹² is independently an optional substituent as defined above and described in embodiments herein.

In some embodiments, Ring A is

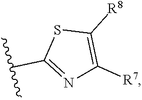

wherein each of R⁷ and R⁸ is independently an optional substituent as defined above and described in embodiments herein.

In some embodiments, each of R⁷, R⁸, and R¹² is halogen, —CN, —NO₂, R^{W}, —C(O)—R^{W}, —C(═NR^{W})—R^{W}, —N(R^{W})—C(O)—R^{W}, —N(R^{W})—C(═NR^{W})—R^{W}, —OC(O)—R^{W}, —OC(═NR^{W})—R^{W}, —S(O)₂—R^{W}, —N(R^{W})—S(O)₂—R^{W}, —OS(O)₂—R^{W}, —S(O)—R^{W}, —N(R^{W})—S(O)—R^{W}, or —OS(O)—R^{W}, wherein each R^{W} is independently as defined below and described in embodiments herein.

In some embodiments, R⁷ is halogen. In some embodiments, R⁷ is —CN. In some embodiments, R⁷ is —NO₂. In some embodiments, R⁷ is R^{W} as defined below and described in embodiments herein. In some embodiments, R⁷ is —C(O)—R^{W}, wherein R^{W} is as defined below and described in embodiments herein. In some embodiments, R⁷ is —C(═NR^{W})—R^{W}, wherein each R^{W} is independently as defined below and described in embodiments herein. In some embodiments, R⁷ is —N(R^{W})—C(O)—R^{W}, wherein each R^{W} is independently as defined below and described in embodiments herein. In some embodiments, R⁷ is —N(R^{W})—C(═NR^{W})—R^{W}, wherein each R^{W} is independently as defined below and described in embodiments herein. In some embodiments, R⁷ is —OC(O)—R^{W}, wherein each R^{W} is independently as defined below and described in embodiments herein. In some embodiments, R⁷ is —OC(═NR^{W})—R^{W}, wherein each R^{W} is independently as defined below and described in embodiments herein. In some embodiments, R⁷ is —S(O)₂—R^{W}, wherein R^{W} is as defined below and described in embodiments herein. In some embodiments, R⁷ is —N(R^{W})—S(O)₂—R^{W}, wherein each R^{W} is independently as defined below and described in embodiments herein. In some embodiments, R⁷ is —OS(O)₂—R^{W}, wherein R^{W} is as defined below and described in embodiments herein. In some embodiments, R⁷ is —S(O)—R^{W}, wherein R^{W} is as defined below and described in embodiments herein. In some embodiments, R⁷ is —N(R^{W})—S(O)—R^{W}, wherein each R^{W} is independently as defined below and described in embodiments herein. In some embodiments, R⁷ is —OS(O)—R^{W}, wherein R^{W} is as defined below and described in embodiments herein.

In some embodiments, R⁷ is F. In some embodiments, R⁷ is Cl. In some embodiments, R⁷ is Br. In some embodiments, R⁷ is optionally substituted —C₁₋₆ aliphatic. In some embodiments, R⁷ is unsubstituted —C₁₋₆ aliphatic. In some embodiments, R⁷ is unsubstituted —C₁₋₆ alkyl. In some embodiments, R⁷ is —C₁₋₆ aliphatic substituted 1-6 times by halogen. In some embodiments, R⁷ is —C₁₋₆ alkyl substituted 1-6 times by halogen. In some embodiments, R⁷ is —C₁₋₆ alkyl substituted 1-6 times by F. In some embodiments, R⁷ is —CF₃.

In some embodiments, R⁷ is

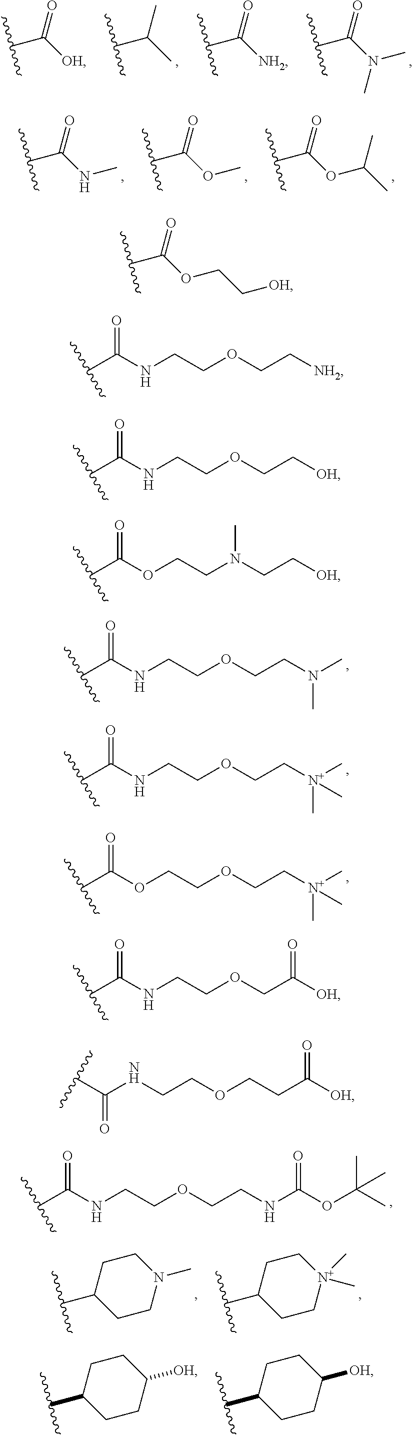

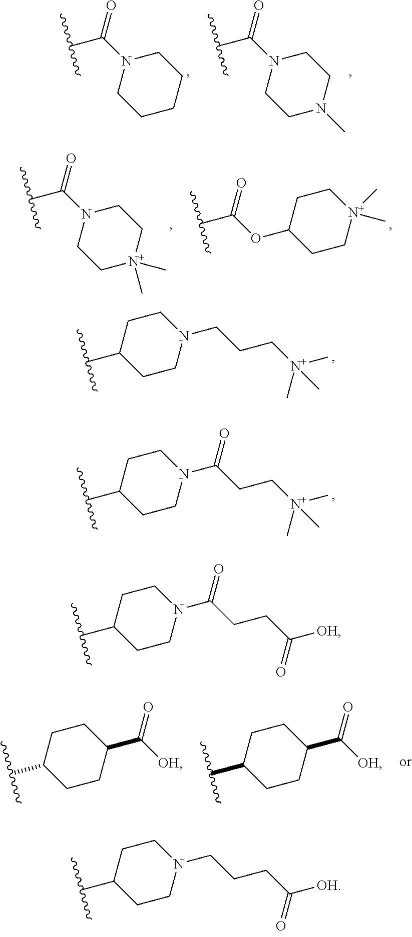

In some embodiments, R⁷ is —NH₂, —CH₂CH₃,

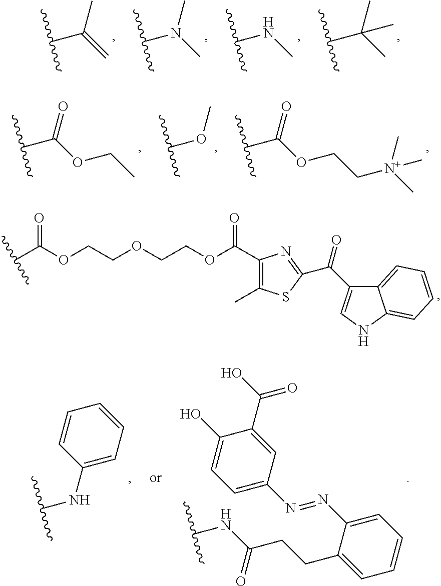

In some embodiments, R⁸ is halogen. In some embodiments, R⁸ is —CN. In some embodiments, R⁸ is —NO₂. In some embodiments, R⁸ is R^{W} as defined below and described in embodiments herein. In some embodiments, R⁸ is —C(O)—R^{W}, wherein R^{W} is as defined below and described in embodiments herein. In some embodiments, R⁸ is —C(═NR^{W})—R^{W}, wherein each R^{W} is independently as defined below and described in embodiments herein. In some embodiments, R⁸ is —N(R^{W})—C(O)—R^{W}, wherein each R^{W} is independently as defined below and described in embodiments herein. In some embodiments, R⁸ is —N(R^{W})—C(═NR^{W})—R^{W}, wherein each R^{W} is independently as defined below and described in embodiments herein. In some embodiments, R⁸ is —OC(O)—R^{W}, wherein each R^{W} is independently as defined below and described in embodiments herein. In some embodiments, R⁸ is —OC(═NR^{W})—R^{W}, wherein each R^{W} is independently as defined below and described in embodiments herein. In some embodiments, R⁸ is —S(O)₂—R^{W}, wherein R^{W} is as defined below and described in embodiments herein. In some embodiments, R⁸ is —N(R^{W})—S(O)₂—R^{W}, wherein each R^{W} is independently as defined below and described in embodiments herein. In some embodiments, R⁸ is —OS(O)₂—R^{W}, wherein R^{W} is as defined below and described in embodiments herein. In some embodiments, R⁸ is —S(O)—R^{W}, wherein R^{W} is as defined below and described in embodiments herein. In some embodiments, R⁸ is —N(R^{W})—S(O)—R^{W}, wherein each R^{W} is independently as defined below and described in embodiments herein. In some embodiments, R⁸ is —OS(O)—R^{W}, wherein R^{W} is as defined below and described in embodiments herein.

In some embodiments, R⁸ is F. In some embodiments, R⁸ is Cl. In some embodiments, R⁸ is Br. In some embodiments, R⁸ is optionally substituted —C₁₋₆ aliphatic. In some embodiments, R⁸ is unsubstituted —C₁₋₆ aliphatic. In some embodiments, R⁸ is unsubstituted —C₁₋₆ alkyl. In some embodiments, R⁸ is —C₁₋₆ aliphatic substituted 1-6 times by halogen. In some embodiments, R⁸ is —C₁₋₆ alkyl substituted 1-6 times by halogen. In some embodiments, R⁸ is —C₁₋₆ alkyl substituted 1-6 times by F. In some embodiments, R⁸ is —CF₃.

In some embodiments, R⁸ is —CH₃,

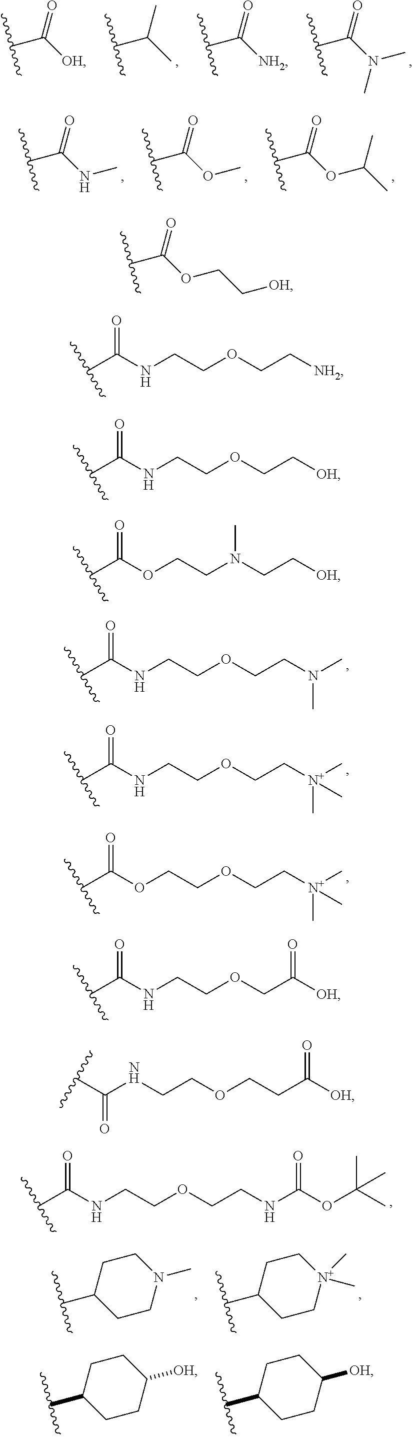

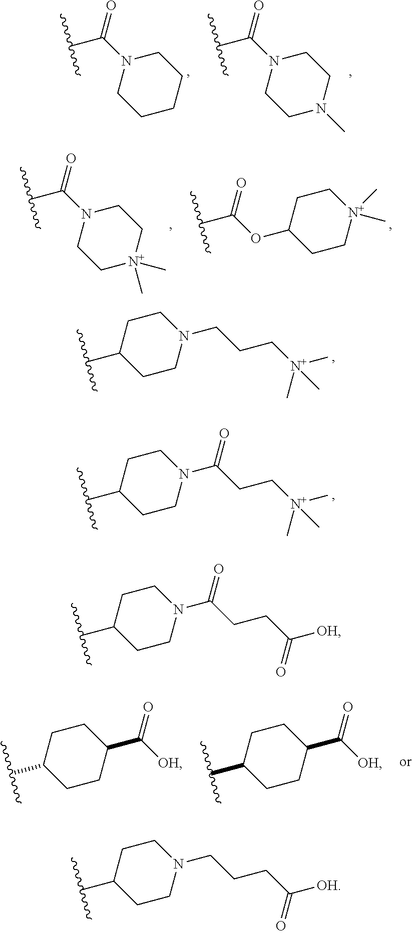

In some embodiments, R⁸ is —NH₂, —CH₂CH₃,

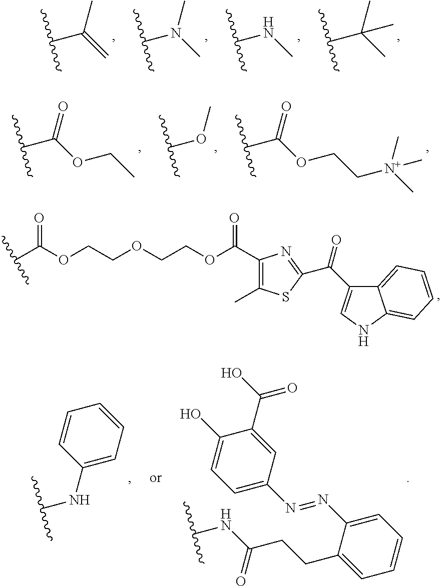

In some embodiments, R¹² is halogen. In some embodiments, R¹² is —CN. In some embodiments, R¹² is —NO₂. In some embodiments, R¹² is R^{W} as defined below and described in embodiments herein. In some embodiments, R¹² is —C(O)—R^{W}, wherein R^{W} is as defined below and described in embodiments herein. In some embodiments, R¹² is —C(═NR^{W})—R^{W}, wherein each R^{W} is independently as defined below and described in embodiments herein. In some embodiments, R¹² is —N(R^{W})—C(O)—R^{W}, wherein each R^{W} is independently as defined below and described in embodiments herein. In some embodiments, R¹² is —N(R^{W})—C(═NR^{W})—R^{W}, wherein each R^{W} is independently as defined below and described in embodiments herein. In some embodiments, R¹² is —OC(O)—R^{W}, wherein each R^{W} is independently as defined below and described in embodiments herein. In some embodiments, R¹² is —OC(═NR^{W})—R^{W}, wherein each R^{W} is independently as defined below and described in embodiments herein. In some embodiments, R¹² is —S(O)₂—R^{W}, wherein R^{W} is as defined below and described in embodiments herein. In some embodiments, R¹² is —N(R^{W})—S(O)₂—R^{W}, wherein each R^{W} is independently as defined below and described in embodiments herein. In some embodiments, R¹² is —OS(O)₂—R^{W}, wherein R^{W} is as defined below and described in embodiments herein. In some embodiments, R¹² is —S(O)—R^{W}, wherein R^{W} is as defined below and described in embodiments herein. In some embodiments, R¹² is —N(R^{W})—S(O)—R^{W}, wherein each R^{W} is independently as defined below and described in embodiments herein. In some embodiments, R¹² is —OS(O)—R^{W}, wherein R^{W} is as defined below and described in embodiments herein.

In some embodiments, R¹² is F. In some embodiments, R¹² is Cl. In some embodiments, R¹² is Br. In some embodiments, R¹² is optionally substituted —C₁₋₆ aliphatic. In some embodiments, R¹² is unsubstituted —C₁₋₆ aliphatic. In some embodiments, R¹² is unsubstituted —C₁₋₆ alkyl. In some embodiments, R¹² is —C₁₋₆ aliphatic substituted 1-6 times by halogen. In some embodiments, R¹² is —C₁₋₆ alkyl substituted 1-6 times by halogen. In some embodiments, R¹² is —C₁₋₆ alkyl substituted 1-6 times by F. In some embodiments, R¹² is —CF₃.

In some embodiments, R¹² is-CH₃,

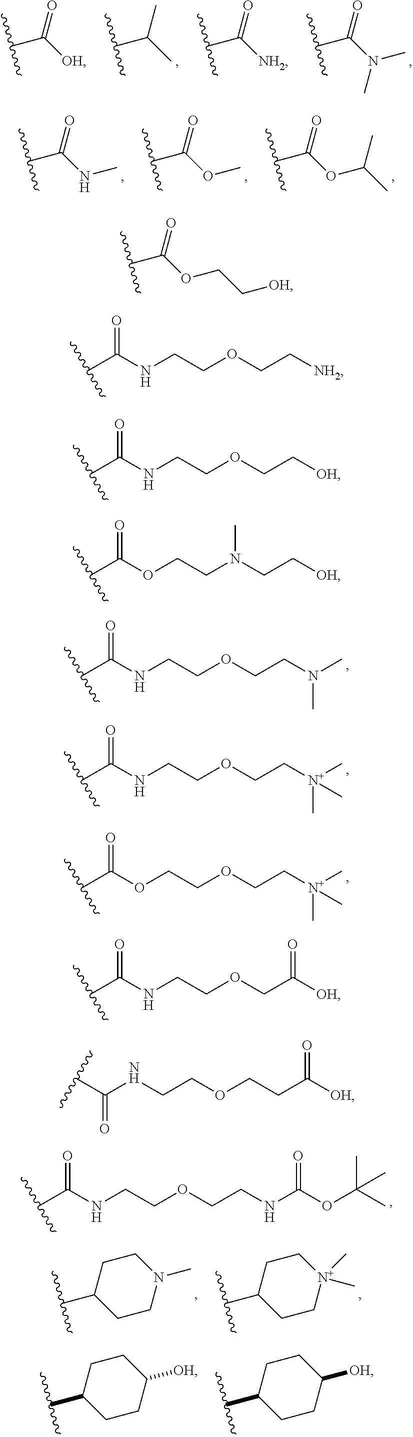

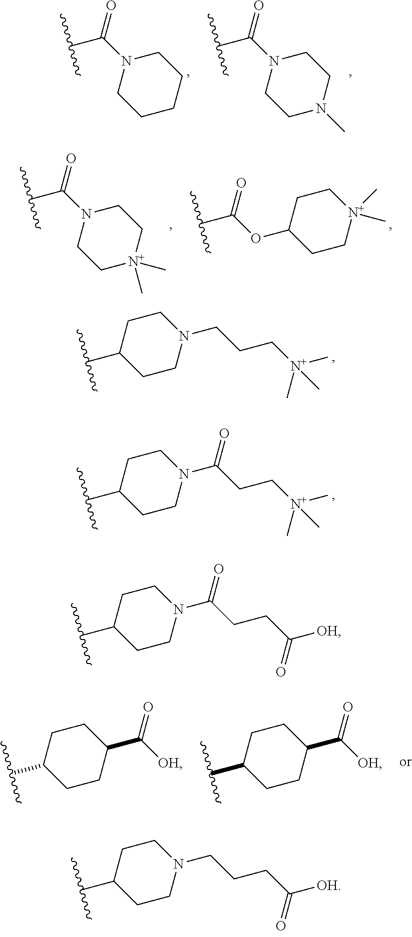

In some embodiments, Ring A is selected from those depicted in Table 1-a, below.

As defined generally above, each of R¹, R², R³, R⁴, and R⁶ is independently halogen, —CN, —NO₂, R^{W}, —C(O)—R^{W}, —C(═NR^{W})—R^{W}, —N(R^{W})—C(O)—R^{W}, —N(R^{W})—C(═NR^{W})—R^{W}, —OC(O)—R^{W}, —OC(═NR^{W})—R^{W}, —S(O)₂—R^{W}, —N(R^{W})—S(O)₂—R^{W}, —OS(O)₂—R^{W}, —S(O)—R^{W}, —N(R^{W})—S(O)—R^{W}, or —OS(O)—R^{W}, wherein each R^{W} is independently as defined below and described in embodiments herein.

In some embodiments, R¹ is halogen. In some embodiments, R¹ is —CN. In some embodiments, R¹ is —NO₂. In some embodiments, R¹ is R^{W} as defined below and described in embodiments herein. In some embodiments, R¹ is —C(O)—R^{W}, wherein R^{W} is as defined below and described in embodiments herein. In some embodiments, R¹ is —C(═NR^{W})—R^{W}, wherein each R^{W} is independently as defined below and described in embodiments herein. In some embodiments, R¹ is —N(R^{W})—C(O)—R^{W}, wherein each R^{W} is independently as defined below and described in embodiments herein. In some embodiments, R¹ is —N(R^{W})—C(═NR^{W})—R^{W}, wherein each R^{W} is independently as defined below and described in embodiments herein. In some embodiments, R¹ is —OC(O)—R^{W}, wherein each R^{W} is independently as defined below and described in embodiments herein. In some embodiments, R¹ is —OC(═NR^{W})—R^{W}, wherein each R^{W} is independently as defined below and described in embodiments herein. In some embodiments, R¹ is —S(O)₂—R^{W}, wherein R^{W} is as defined below and described in embodiments herein. In some embodiments, R¹ is —N(R^{W})—S(O)₂—R^{W}, wherein each R^{W} is independently as defined below and described in embodiments herein. In some embodiments, R¹ is —OS(O)₂—R^{W}, wherein R^{W} is as defined below and described in embodiments herein. In some embodiments, R¹ is —S(O)—R^{W}, wherein R^{W} is as defined below and described in embodiments herein. In some embodiments, R¹ is —N(R^{W})—S(O)—R^{W}, wherein each R^{W} is independently as defined below and described in embodiments herein. In some embodiments, R¹ is —OS(O)—R^{W}, wherein R^{W} is as defined below and described in embodiments herein.

In some embodiments, R² is halogen. In some embodiments, R² is —CN. In some embodiments, R² is —NO₂. In some embodiments, R² is R^{W} as defined below and described in embodiments herein. In some embodiments, R² is —C(O)—R^{W}, wherein R^{W} is as defined below and described in embodiments herein. In some embodiments, R² is —C(═NR^{W})—R^{W}, wherein each R^{W} is independently as defined below and described in embodiments herein. In some embodiments, R² is —N(R^{W})—C(O)—R^{W}, wherein each R^{W} is independently as defined below and described in embodiments herein. In some embodiments, R² is —N(R^{W})—C(═NR^{W})—R^{W}, wherein each R^{W} is independently as defined below and described in embodiments herein. In some embodiments, R² is —OC(O)—R^{W}, wherein each R^{W} is independently as defined below and described in embodiments herein. In some embodiments, R² is —OC(═NR^{W})—R^{W}, wherein each R^{W} is independently as defined below and described in embodiments herein. In some embodiments, R² is —S(O)₂—R^{W}, wherein R^{W} is as defined below and described in embodiments herein. In some embodiments, R² is —N(R^{W})—S(O)₂—R^{W}, wherein each R^{W} is independently as defined below and described in embodiments herein. In some embodiments, R² is —OS(O)₂—R^{W}, wherein R^{W} is as defined below and described in embodiments herein. In some embodiments, R² is —S(O)—R^{W}, wherein R^{W} is as defined below and described in embodiments herein. In some embodiments, R² is —N(R^{W})—S(O)—R^{W}, wherein each R^{W} is independently as defined below and described in embodiments herein. In some embodiments, R² is —OS(O)—R^{W}, wherein R^{W} is as defined below and described in embodiments herein.

In some embodiments, R³ is halogen. In some embodiments, R³ is —CN. In some embodiments, R³ is —NO₂. In some embodiments, R³ is R^{W} as defined below and described in embodiments herein. In some embodiments, R³ is —C(O)—R^{W}, wherein R^{W} is as defined below and described in embodiments herein. In some embodiments, R³ is —C(═NR^{W})—R^{W}, wherein each R^{W} is independently as defined below and described in embodiments herein. In some embodiments, R³ is —N(R^{W})—C(O)—R^{W}, wherein each R^{W} is independently as defined below and described in embodiments herein. In some embodiments, R³ is —N(R^{W})—C(═NR^{W})—R^{W}, wherein each R^{W} is independently as defined below and described in embodiments herein. In some embodiments, R³ is —OC(O)—R^{W}, wherein each R^{W} is independently as defined below and described in embodiments herein. In some embodiments, R³ is —OC(═NR^{W})—R^{W}, wherein each R^{W} is independently as defined below and described in embodiments herein. In some embodiments, R³ is —S(O)₂—R^{W}, wherein R^{W} is as defined below and described in embodiments herein. In some embodiments, R³ is —N(R^{W})—S(O)₂—R^{W}, wherein each R^{W} is independently as defined below and described in embodiments herein. In some embodiments, R³ is —OS(O)₂—R^{W}, wherein R^{W} is as defined below and described in embodiments herein. In some embodiments, R³ is —S(O)—R^{W}, wherein R^{W} is as defined below and described in embodiments herein. In some embodiments, R³ is —N(R^{W})—S(O)—R^{W}, wherein each R^{W} is independently as defined below and described in embodiments herein. In some embodiments, R³ is —OS(O)—R^{W}, wherein R^{W} is as defined below and described in embodiments herein.

In some embodiments, R⁴ is halogen. In some embodiments, R⁴ is —CN. In some embodiments, R⁴ is —NO₂. In some embodiments, R⁴ is R^{W} as defined below and described in embodiments herein. In some embodiments, R⁴ is —C(O)—R^{W}, wherein R^{W} is as defined below and described in embodiments herein. In some embodiments, R⁴ is —C(═NR^{W})—R^{W}, wherein each R^{W} is independently as defined below and described in embodiments herein. In some embodiments, R⁴ is —N(R^{W})—C(O)—R^{W}, wherein each R^{W} is independently as defined below and described in embodiments herein. In some embodiments, R⁴ is —N(R^{W})—C(═NR^{W})—R^{W}, wherein each R^{W} is independently as defined below and described in embodiments herein. In some embodiments, R⁴ is —OC(O)—R^{W}, wherein each R^{W} is independently as defined below and described in embodiments herein. In some embodiments, R⁴ is —OC(═NR^{W})—R^{W}, wherein each R^{W} is independently as defined below and described in embodiments herein. In some embodiments, R⁴ is —S(O)₂—R^{W}, wherein R^{W} is as defined below and described in embodiments herein. In some embodiments, R⁴ is —N(R^{W})—S(O)₂—R^{W}, wherein each R^{W} is independently as defined below and described in embodiments herein. In some embodiments, R⁴ is —OS(O)₂—R^{W}, wherein R^{W} is as defined below and described in embodiments herein. In some embodiments, R⁴ is —S(O)—R^{W}, wherein R^{W} is as defined below and described in embodiments herein. In some embodiments, R⁴ is —N(R^{W})—S(O)—R^{W}, wherein each R^{W} is independently as defined below and described in embodiments herein. In some embodiments, R⁴ is —OS(O)—R^{W}, wherein R^{W} is as defined below and described in embodiments herein.

In some embodiments, R⁶ is halogen. In some embodiments, R⁶ is —CN. In some embodiments, R⁶ is —NO₂. In some embodiments, R⁶ is R^{W} as defined below and described in embodiments herein. In some embodiments, R⁶ is —C(O)—R^{W}, wherein R^{W} is as defined below and described in embodiments herein. In some embodiments, R⁶ is —C(═NR^{W})—R^{W}, wherein each R^{W} is independently as defined below and described in embodiments herein. In some embodiments, R⁶ is —N(R^{W})—C(O)—R^{W}, wherein each R^{W} is independently as defined below and described in embodiments herein. In some embodiments, R⁶ is —N(R^{W})—C(═NR^{W})—R^{W}, wherein each R^{W} is independently as defined below and described in embodiments herein. In some embodiments, R⁶ is —OC(O)—R^{W}, wherein each R^{W} is independently as defined below and described in embodiments herein. In some embodiments, R⁶ is —OC(═NR^{W})—R^{W}, wherein each R^{W} is independently as defined below and described in embodiments herein. In some embodiments, R⁶ is —S(O)₂—R^{W}, wherein R^{W} is as defined below and described in embodiments herein. In some embodiments, R⁶ is —N(R^{W})—S(O)₂—R^{W}, wherein each R^{W} is independently as defined below and described in embodiments herein. In some embodiments, R⁶ is —OS(O)₂—R^{W}, wherein R^{W} is as defined below and described in embodiments herein. In some embodiments, R⁶ is —S(O)—R^{W}, wherein R^{W} is as defined below and described in embodiments herein. In some embodiments, R⁶ is —N(R^{W})—S(O)—R^{W}, wherein each R^{W} is independently as defined below and described in embodiments herein. In some embodiments, R⁶ is —OS(O)—R^{W}, wherein R^{W} is as defined below and described in embodiments herein.

In some embodiments, each of R¹, R², R³, R⁴, and R⁶ is independently hydrogen, Cl, Br, F, —OH, —OCH₃, —CH₃, —C(O)OC(CH₃)₃, or —S(O)₂OH.

In some embodiments, each of R¹, R², R³, R⁴, and R⁶ is independently —NH₂, —OCH₂CH₃, —COOH, —C(O)OCH₃, —C(O)OCH(CH₃)₂, —C(O)OCH₂CH₃, or

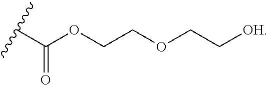 [SMILES: *C(=O)OCCOCCO]

In some embodiments, each of R¹, R², R³, R⁴, and R⁶ is independently selected from those depicted in Table 1-a, below.

As defined generally above, R⁵ is —R, —C(O)—R^{W}, —C(═NR^{W})—R^{W}, —S(O)₂—R^{W}, or —S(O)—R^{W}, wherein each R^{W} is independently as defined below and described in embodiments herein.

In some embodiments, R⁵ is —R, wherein R is as defined below and described in embodiments herein. In some embodiments, R⁵ is —C(O)—R^{W}, wherein R^{W} is as defined below and described in embodiments herein. In some embodiments, R⁵ is —C(═NR^{W})—R^{W}, wherein each R^{W} is independently as defined below and described in embodiments herein. In some embodiments, R⁵ is —S(O)₂—R^{W}, wherein R^{W} is as defined below and described in embodiments herein. In some embodiments, R⁵ is —S(O)—R^{W}, wherein R^{W} is as defined below and described in embodiments herein.

In some embodiments, R⁵ is H. In some embodiments, R⁵ is optionally substituted C₁₋₆ aliphatic. In some embodiments, R⁵ is optionally substituted C₁₋₆ alkyl.

In some embodiments, R⁵ is selected from those depicted in Table 1-a, below.

As defined generally above, R^{W} is —R, —N(R)₂, —NR—OR, —N(R)—N(R)₂, —N(OR)—N(R)₂, —N(R)—N(OR)R, —OR, —O—N(R)₂, or —SR.

In some embodiments, R^{W} is —R, wherein R is as defined below and described in embodiments herein. In some embodiments, R^{W} is —N(R)₂, wherein each R is independently as defined below and described in embodiments herein. In some embodiments, R^{W} is —NR—OR, wherein each R is independently as defined below and described in embodiments herein. In some embodiments, R^{W} is —N(R)—N(R)₂, wherein each R is independently as defined below and described in embodiments herein. In some embodiments, R^{W} is —N(OR)—N(R)₂, wherein each R is independently as defined below and described in embodiments herein. In some embodiments, R^{W} is —N(R)—N(OR)R, wherein each R is independently as defined below and described in embodiments herein. In some embodiments, R^{W} is —OR, wherein R is as defined below and described in embodiments herein. In some embodiments, R^{W} is —O—N(R)₂, wherein each R is independently as defined below and described in embodiments herein. In some embodiments, R^{W} is —SR, wherein R is as defined below and described in embodiments herein.

In some embodiments, R^{W} is selected from those depicted in Table 1-a, below.

As defined generally above, R is hydrogen, optionally substituted C₁₋₆ aliphatic, an optionally substituted 3-7 membered carbocyclic ring, or an optionally substituted 3-7 membered heterocyclic ring having 1-3 heteroatoms independently selected from N, O, or S, or two R's together with the nitrogen to which they attach form an optionally substituted 5-7 membered heterocyclic ring having 0-2 heteroatoms independently selected from N, O, or S in addition to the nitrogen to which the two R's attach.

In some embodiments, R is hydrogen. In some embodiments, R is optionally substituted C₁₋₆ aliphatic. In some embodiments, R is optionally substituted C₁₋₆ alkyl. In some embodiments, R is unsubstituted —C₁₋₆ aliphatic. In some embodiments, R is unsubstituted —C₁₋₆ alkyl. In some embodiments, R is —C₁₋₆ aliphatic which is substituted by —CH₃, —CF₃, —OH, —OCH₃, —OCF₃, —N(CH₃)₂, —N⁺(CH₃)₃,

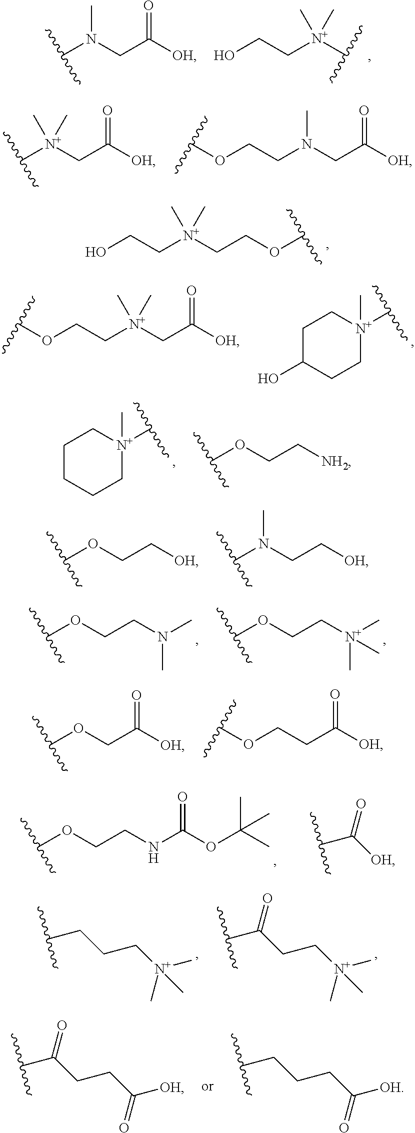

In some embodiments, R is —C₁₋₆ aliphatic substituted 1-6 times by halogen. In some embodiments, R is —C₁₋₆ alkyl substituted 1-6 times by halogen. In some embodiments, R is —C₁₋₆ alkyl substituted 1-6 times by F. In some embodiments, R is —CH₃. In some embodiments, R is —CH₂CH₃. In some embodiments, R is —CH₂CH₂CH₃. In some embodiments, R is —CH(CH₃)₂. In some embodiments, R is —CH₂CH₂CH₂CH₃. In some embodiments, R is —CH₂CH(CH₃)₂. In some embodiments, R is —C(CH₃)₃. In some embodiments, R is —CF₃.

In some embodiments, R is an optionally substituted 3, 4, 5, 6, or 7 membered carbocyclic ring. In some embodiments, R is a 3, 4, 5, 6, or 7 membered carbocyclic ring, which is substituted 1-5 times by —CH₃—CF₃, —OH, —OCH₃, —OCF₃, —N(CH₃)₂, —N⁺(CH₃)₃,

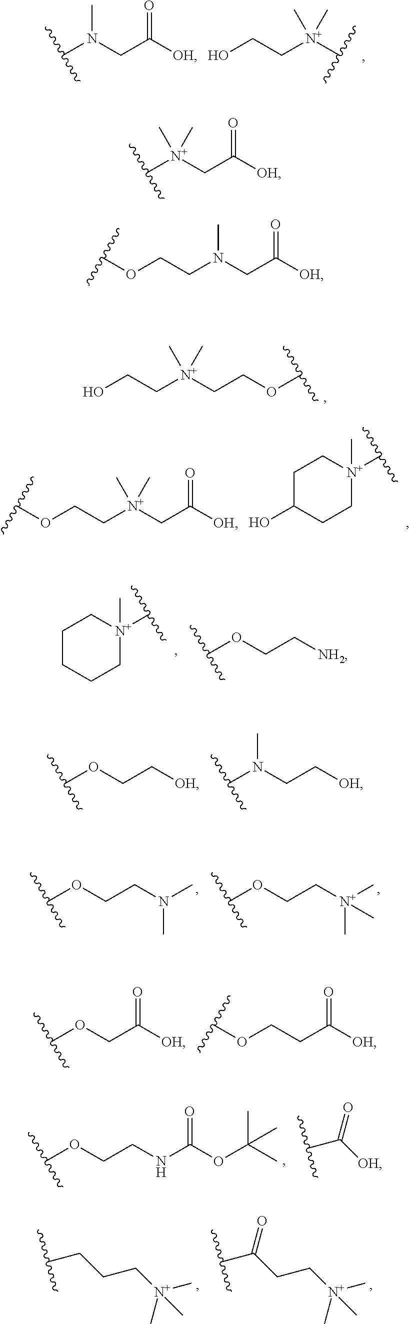

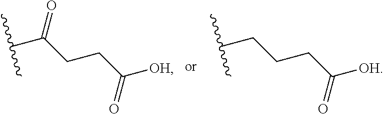 [SMILES: *C(=O)CCC(=O)O ; "or" ; *CCCC(=O)O]

some embodiments, R is an optionally substituted

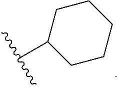 [SMILES: *C1CCCCC1]

In some embodiments, R is an optionally substituted 3, 4, 5, 6, or 7 membered heterocyclic ring having 1, 2, or 3 heteroatoms independently selected from N, O, or S. In some embodiments, R is a 3, 4, 5, 6, or 7 membered heterocyclic ring having 1, 2, or 3 heteroatoms independently selected from N, O, or S, which is substituted 1-5 times by —CH₃, —CF₃, —OH, —OCH₃, —OCF₃, —N(CH₃)₂, —N⁺(CH₃)₃,

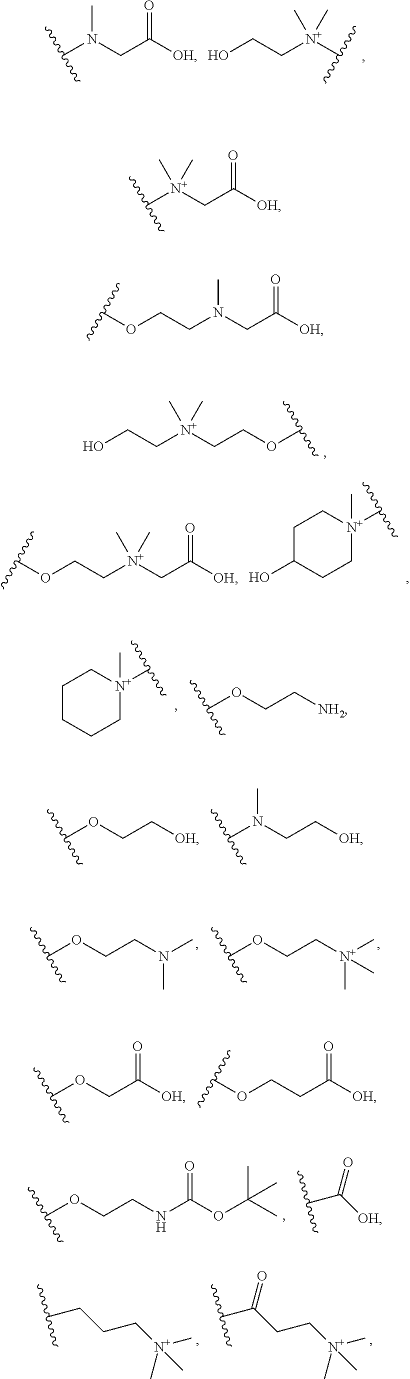

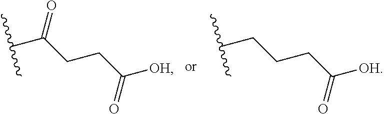 [SMILES: *C(=O)CCC(=O)O ; "or" ; *CCCC(=O)O]

In some embodiments, R is an optionally substituted 6-membered heterocyclic ring having 1 or 2 heteroatoms independently selected from N, O, or S. In some embodiments, R is optionally substituted or

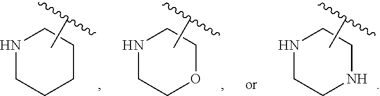

In some embodiments, R is optionally substituted

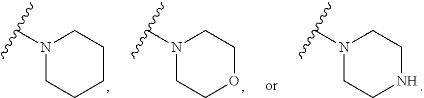 [SMILES: *N1CCCCC1 ; *N1CCOCC1 ; *N1CCNCC1]

In some embodiments, R is

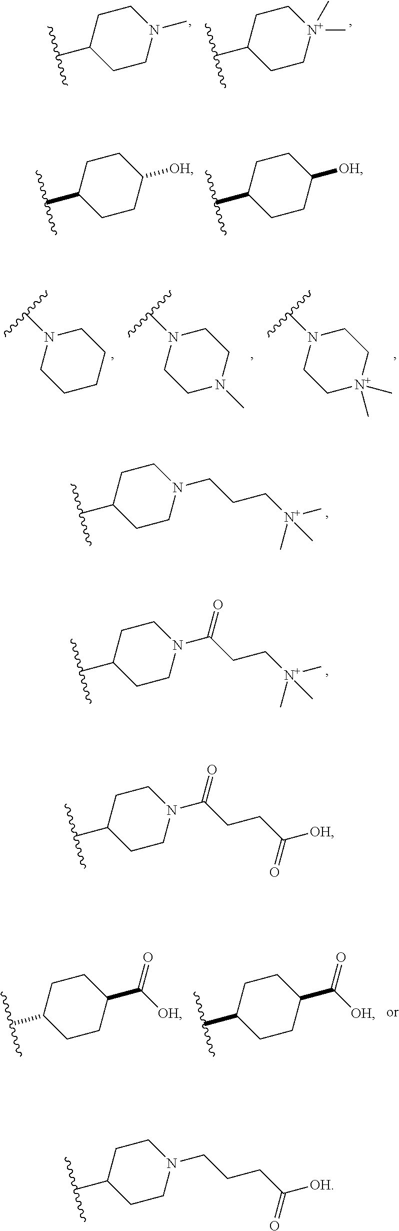

In some embodiments, two R's together with the nitrogen to which they attach form an optionally substituted 5-7 membered heterocyclic ring having 0-2 heteroatoms independently selected from N, O, or S in addition to the nitrogen to which the two R's attach. In some embodiments, two R's together with the nitrogen to which they attach form an optionally substituted 5-7 membered heterocyclic ring having 0 or 1 heteroatom independently selected from N, O, or S in addition to the nitrogen to which the two R's attach. In some embodiments, —N(R)₂ is optionally substituted

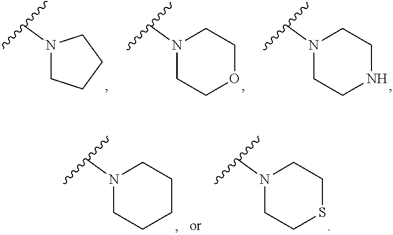 [SMILES: *N1CCCC1 ; *N1CCOCC1 ; *N1CCNCC1 ; *N1CCCCC1 ; *N1CCSCC1]

In some embodiments, R is selected from those depicted in Table 1-a, below.

In some embodiments, the present invention provides a compound of Formula (II):

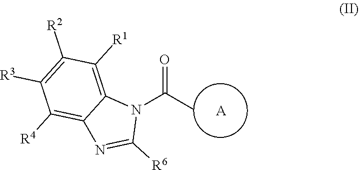

or a pharmaceutically acceptable salt thereof, wherein each variable is as defined above and described in embodiments herein, both singly and in combination.

In some embodiments, the present invention provides a compound of Formula (I-a):

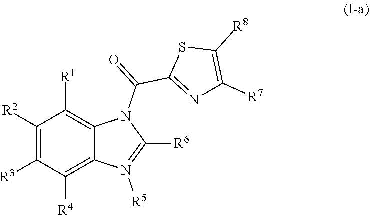

or a pharmaceutically acceptable salt thereof, wherein each variable is as defined above and described in embodiments herein, both singly and in combination.

In some embodiments, the present invention provides a compound of Formula (I-a), or a pharmaceutically acceptable salt thereof, wherein R⁷ is —C(O)—R^{W}, each of R^{W}, R¹, R², R³, R⁴, R⁵, R⁶, and R⁸ is independently as defined above and described in embodiments herein, both singly and in combination.

In some embodiments, the present invention provides a compound of Formula (I-a), or a pharmaceutically acceptable salt thereof, wherein R⁷ is —C(O)—OR or —C(O)O—N(R)₂, each of R, R¹, R², R³, R⁴, R⁵, R⁶, and R⁸ is independently as defined above and described in embodiments herein, both singly and in combination.

In some embodiments, the present invention provides a compound of Formulas (I-a), or a pharmaceutically acceptable salt thereof, wherein R⁷ is —C(O)—N(R)₂, —C(O)—NR—OR, —C(O)—N(R)—N(R)₂, —C(O)—N(OR)—N(R)₂, or —C(O)—N(R)—N(OR)R, each of R, R¹, R², R³, R⁴, R⁵, R⁶, and R⁸ is independently as defined above and described in embodiments herein, both singly and in combination.

In some embodiments, the present invention provides a compound selected from Formulas (I-b) to (I-h):

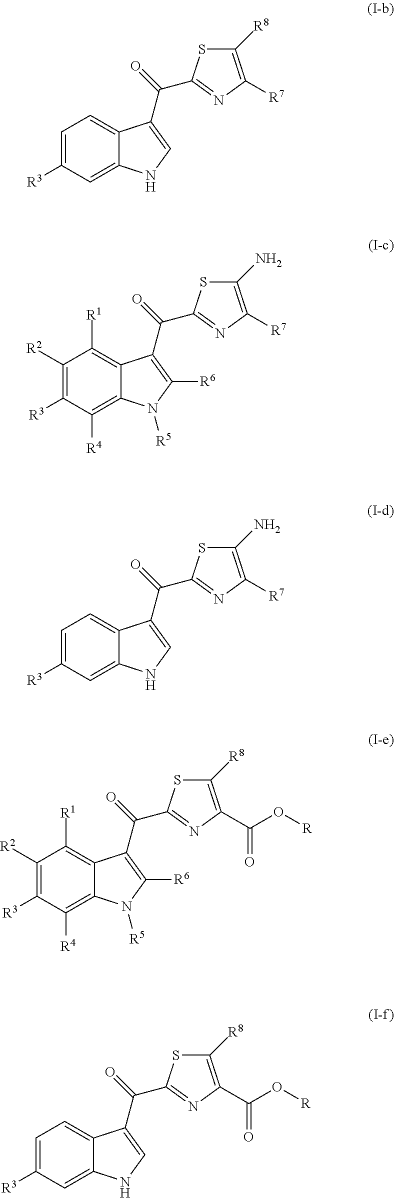

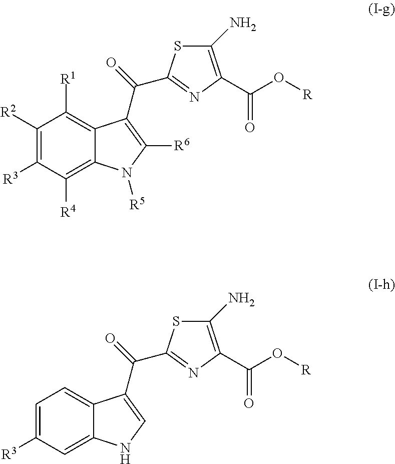

or a pharmaceutically acceptable salt thereof, wherein each variable is as defined above and described in embodiments herein, both singly and in combination.

Exemplary compounds of the invention are set forth in Table 1-a, below.

TABLE I-a

| | |
|---|---|
| 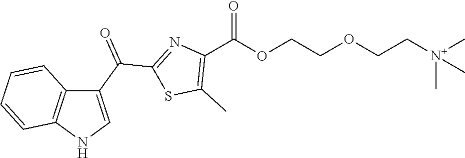 [SMILES: Cc1sc(C(=O)c2c[nH]c3ccccc23)nc1C(=O)OCCOCC[N+](C)(C)C] | I-1 |
| 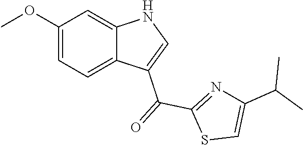 [SMILES: COc1ccc2c(C(=O)c3nc(C(C)C)cs3)c[nH]c2c1] | I-2 |
| 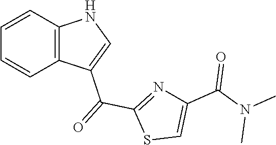 [SMILES: CN(C)C(=O)c1csc(C(=O)c2c[nH]c3ccccc23)n1] | I-3 |
| 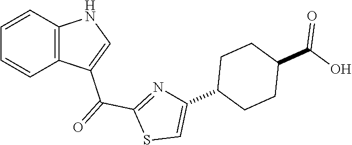 | I-4 |
| 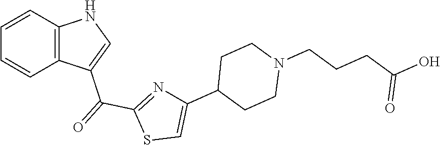 [SMILES: O=C(O)CCCN1CCC(c2csc(C(=O)c3c[nH]c4ccccc34)n2)CC1] | I-5 |
| 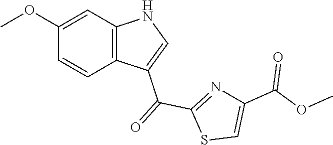 | I-6 |
| 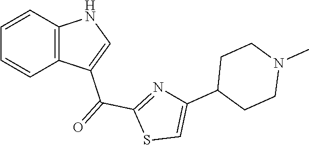 [SMILES: CN1CCC(c2csc(C(=O)c3c[nH]c4ccccc34)n2)CC1] | I-7 |
| 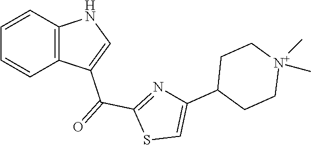 [SMILES: C[N+]1(C)CCC(c2csc(C(=O)c3c[nH]c4ccccc34)n2)CC1] | I-8 |
| 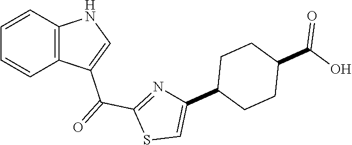 | I-9 |
| 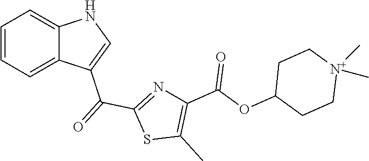 [SMILES: Cc1sc(C(=O)c2c[nH]c3ccccc23)nc1C(=O)OC1CC[N+](C)(C)CC1] | I-10 |
| 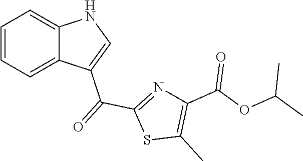 [SMILES: Cc1sc(C(=O)c2c[nH]c3ccccc23)nc1C(=O)OC(C)C] | I-11 |
| 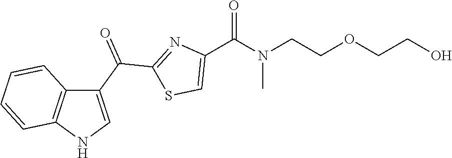 [SMILES: CN(CCOCCO)C(=O)c1csc(C(=O)c2c[nH]c3ccccc23)n1] | I-12 |
| 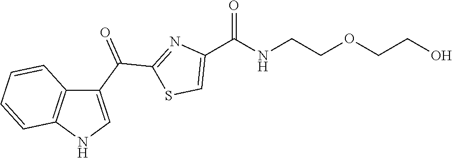 [SMILES: O=C(NCCOCCO)c1csc(C(=O)c2c[nH]c3ccccc23)n1] | I-13 |
| 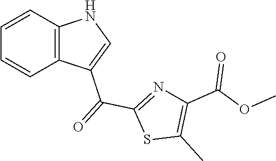 [SMILES: COC(=O)c1nc(C(=O)c2c[nH]c3ccccc23)sc1C] | I-14 |
| 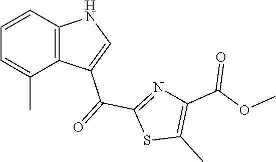 [SMILES: COC(=O)c1nc(C(=O)c2c[nH]c3cccc(C)c23)sc1C] | I-15 |
| 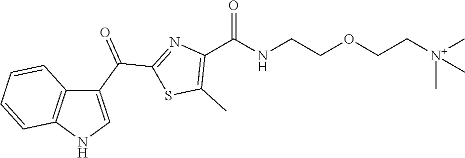 [SMILES: Cc1sc(C(=O)c2c[nH]c3ccccc23)nc1C(=O)NCCOCC[N+](C)(C)C] | I-16 |
| 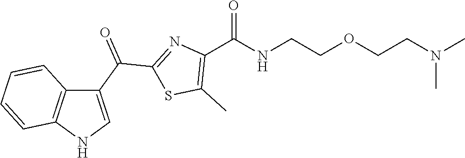 [SMILES: Cc1sc(C(=O)c2c[nH]c3ccccc23)nc1C(=O)NCCOCCN(C)C] | I-17 |
| 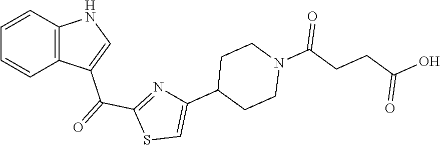 [SMILES: O=C(O)CCC(=O)N1CCC(c2csc(C(=O)c3c[nH]c4ccccc34)n2)CC1] | I-18 |
| 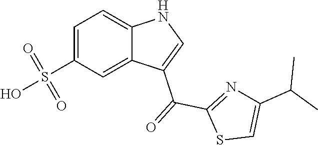 [SMILES: CC(C)c1csc(C(=O)c2c[nH]c3ccc(S(=O)(=O)O)cc23)n1] | I-19 |
| 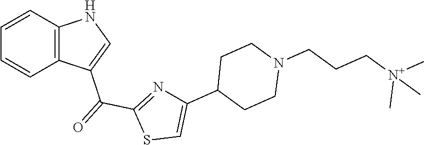 [SMILES: C[N+](C)(C)CCCN1CCC(c2csc(C(=O)c3c[nH]c4ccccc34)n2)CC1] | I-20 |
| 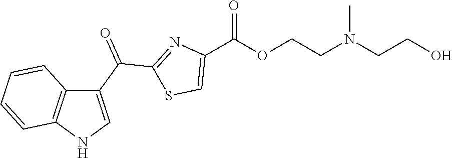 [SMILES: CN(CCO)CCOC(=O)c1csc(C(=O)c2c[nH]c3ccccc23)n1] | I-21 |
| 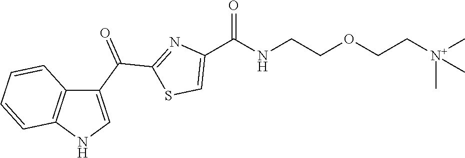 [SMILES: C[N+](C)(C)CCOCCNC(=O)c1csc(C(=O)c2c[nH]c3ccccc23)n1] | I-22 |
| 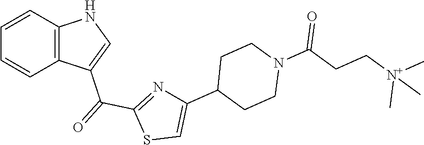 [SMILES: C[N+](C)(C)CCC(=O)N1CCC(c2csc(C(=O)c3c[nH]c4ccccc34)n2)CC1] | I-23 |
| 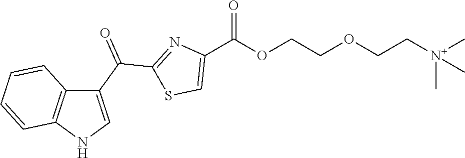 [SMILES: C[N+](C)(C)CCOCCOC(=O)c1csc(C(=O)c2c[nH]c3ccccc23)n1] | I-24 |
| 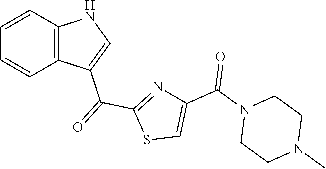 [SMILES: CN1CCN(C(=O)c2csc(C(=O)c3c[nH]c4ccccc34)n2)CC1] | I-25 |
| 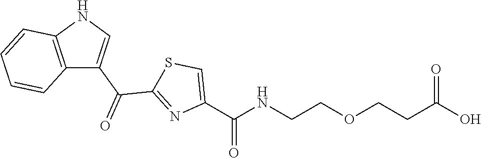 [SMILES: O=C(O)CCOCCNC(=O)c1csc(C(=O)c2c[nH]c3ccccc23)n1] | I-26 |
| 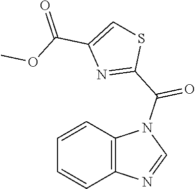 [SMILES: COC(=O)c1csc(C(=O)n2cnc3ccccc32)n1] | I-27 |
| 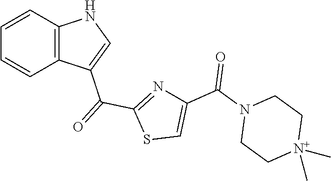 [SMILES: C[N+]1(C)CCN(C(=O)c2csc(C(=O)c3c[nH]c4ccccc34)n2)CC1] | I-28 |
| 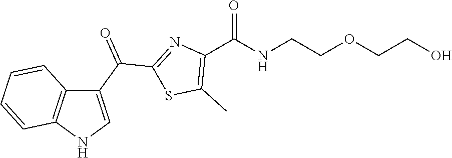 [SMILES: Cc1sc(C(=O)c2c[nH]c3ccccc23)nc1C(=O)NCCOCCO] | I-29 |
| 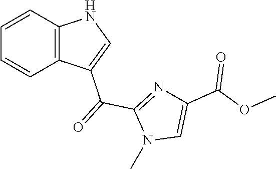 [SMILES: COC(=O)c1cn(C)c(C(=O)c2c[nH]c3ccccc23)n1] | I-30 |
| 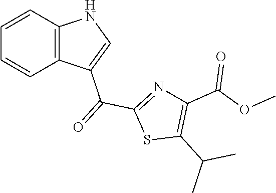 [SMILES: COC(=O)c1nc(C(=O)c2c[nH]c3ccccc23)sc1C(C)C] | I-31 |
| 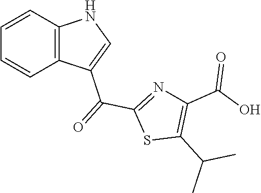 [SMILES: CC(C)c1sc(C(=O)c2c[nH]c3ccccc23)nc1C(=O)O] | I-32 |
| 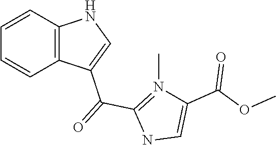 | I-33 |
| 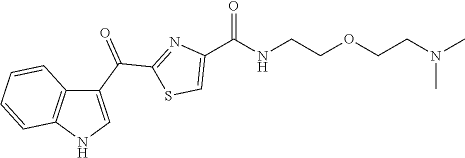 [SMILES: CN(C)CCOCCNC(=O)c1csc(C(=O)c2c[nH]c3ccccc23)n1] | I-34 |
| 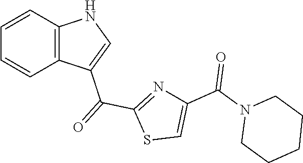 [SMILES: O=C(c1nc(C(=O)N2CCCCC2)cs1)c1c[nH]c2ccccc12] | I-35 |
| 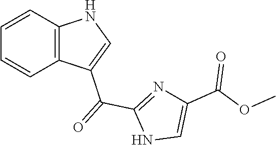 [SMILES: COC(=O)c1c[nH]c(C(=O)c2c[nH]c3ccccc23)n1] | I-36 |
| 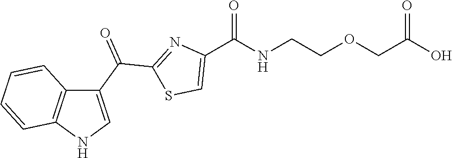 [SMILES: O=C(O)COCCNC(=O)c1csc(C(=O)c2c[nH]c3ccccc23)n1] | I-37 |
| 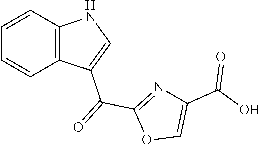 [SMILES: O=C(O)c1coc(C(=O)c2c[nH]c3ccccc23)n1] | I-38 |
| 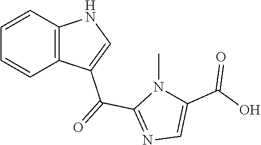 [SMILES: Cn1c(C(=O)O)cnc1C(=O)c1c[nH]c2ccccc12] | I-39 |
| 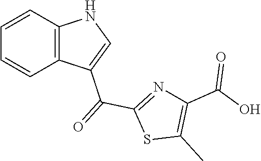 [SMILES: Cc1sc(C(=O)c2c[nH]c3ccccc23)nc1C(=O)O] | I-40 |
| 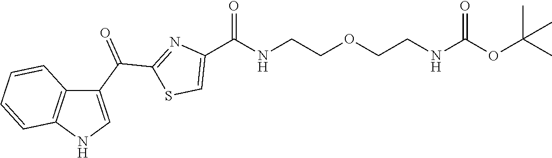 [SMILES: CC(C)(C)OC(=O)NCCOCCNC(=O)c1csc(C(=O)c2c[nH]c3ccccc23)n1] | I-41 |
| 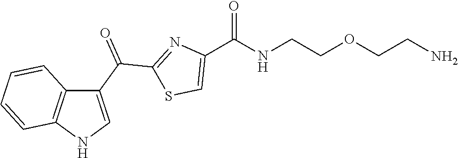 [SMILES: NCCOCCNC(=O)c1csc(C(=O)c2c[nH]c3ccccc23)n1] | I-42 |
| 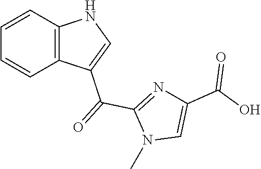 [SMILES: Cn1cc(C(=O)O)nc1C(=O)c1c[nH]c2ccccc12] | I-43 |
| 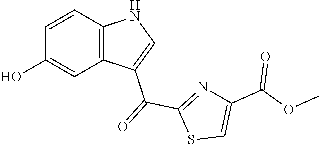 [SMILES: COC(=O)c1csc(C(=O)c2c[nH]c3ccc(O)cc23)n1] | I-44 |
| 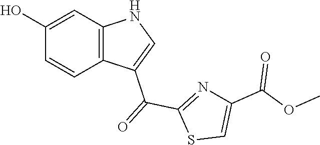 [SMILES: COC(=O)c1csc(C(=O)c2c[nH]c3cc(O)ccc23)n1] | I-45 |
| 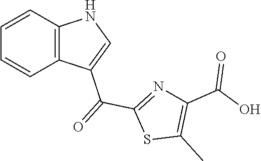 [SMILES: Cc1sc(C(=O)c2c[nH]c3ccccc23)nc1C(=O)O] | I-46 |
| 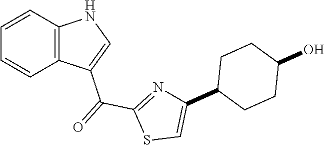 | I-47 |
| 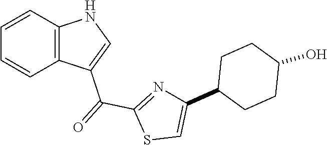 | I-48 |
| 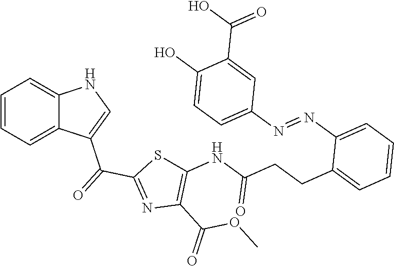 [SMILES: COC(=O)c1nc(C(=O)c2c[nH]c3ccccc23)sc1NC(=O)CCc1ccccc1/N=N/c1ccc(O)c(C(=O)O)c1] | I-49 |
| 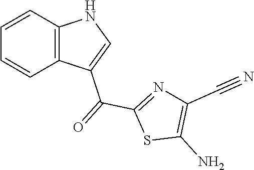 [SMILES: N#Cc1nc(C(=O)c2c[nH]c3ccccc23)sc1N] | I-50 |
| 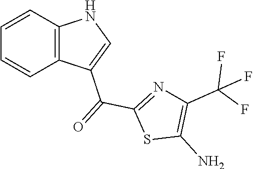 [SMILES: Nc1sc(C(=O)c2c[nH]c3ccccc23)nc1C(F)(F)F] | I-51 |
| 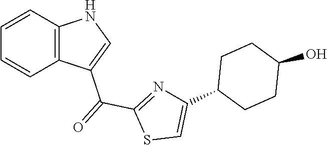 [SMILES: O=C(c1nc([C@H]2CC[C@H](O)CC2)cs1)c1c[nH]c2ccccc12] | I-52 |
| 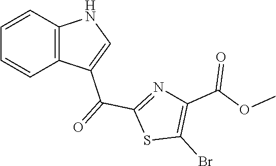 [SMILES: COC(=O)c1nc(C(=O)c2c[nH]c3ccccc23)sc1Br] | I-53 |
| 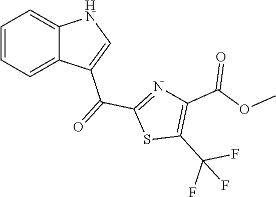 [SMILES: COC(=O)c1nc(C(=O)c2c[nH]c3ccccc23)sc1C(F)(F)F] | I-54 |
| 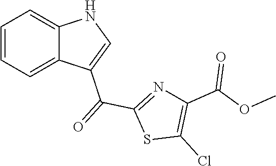 [SMILES: COC(=O)c1nc(C(=O)c2c[nH]c3ccccc23)sc1Cl] | I-55 |
| 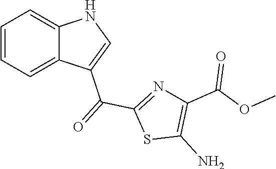 [SMILES: COC(=O)c1nc(C(=O)c2c[nH]c3ccccc23)sc1N] | I-56 |
| 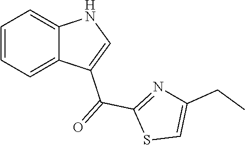 [SMILES: CCc1csc(C(=O)c2c[nH]c3ccccc23)n1] | I-57 |
| 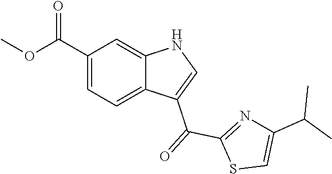 [SMILES: COC(=O)c1ccc2c(C(=O)c3nc(C(C)C)cs3)c[nH]c2c1] | I-58 |
| 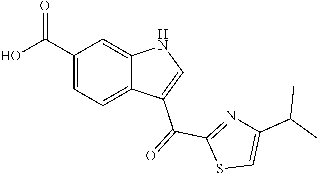 [SMILES: CC(C)c1csc(C(=O)c2c[nH]c3cc(C(=O)O)ccc23)n1] | I-59 |
| 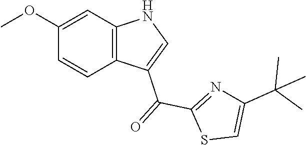 [SMILES: COc1ccc2c(C(=O)c3nc(C(C)(C)C)cs3)c[nH]c2c1] | I-60 |
| 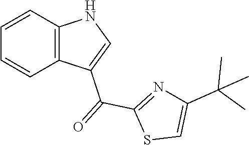 [SMILES: CC(C)(C)c1csc(C(=O)c2c[nH]c3ccccc23)n1] | I-61 |
| 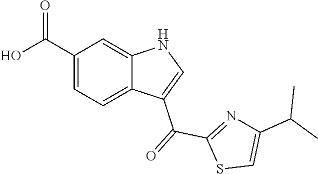 [SMILES: CC(C)c1csc(C(=O)c2c[nH]c3cc(C(=O)O)ccc23)n1] | I-62 |
| 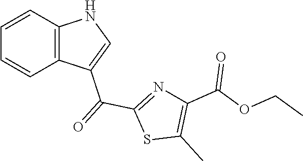 [SMILES: CCOC(=O)c1nc(C(=O)c2c[nH]c3ccccc23)sc1C] | I-63 |
| 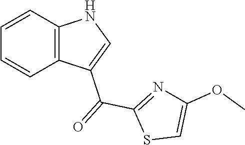 [SMILES: COc1csc(C(=O)c2c[nH]c3ccccc23)n1] | I-64 |
| 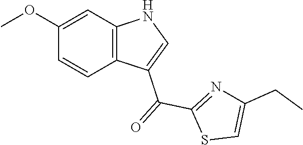 [SMILES: CCc1csc(C(=O)c2c[nH]c3cc(OC)ccc23)n1] | I-65 |
| 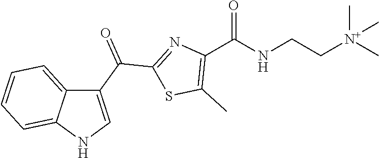 [SMILES: Cc1sc(C(=O)c2c[nH]c3ccccc23)nc1C(=O)NCC[N+](C)(C)C] | I-66 |
| 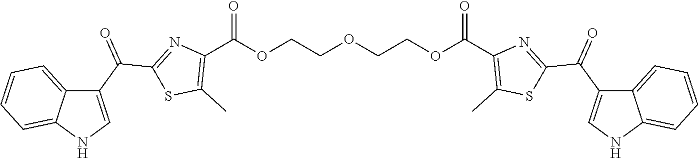 [SMILES: Cc1sc(C(=O)c2c[nH]c3ccccc23)nc1C(=O)OCCOCCOC(=O)c1nc(C(=O)c2c[nH]c3ccccc23)sc1C] | I-67 |
| 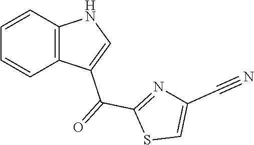 [SMILES: N#Cc1csc(C(=O)c2c[nH]c3ccccc23)n1] | I-68 |
| 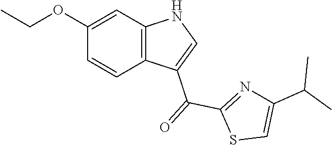 [SMILES: CCOc1ccc2c(C(=O)c3nc(C(C)C)cs3)c[nH]c2c1] | I-69 |
| 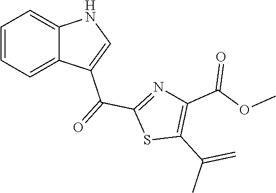 [SMILES: C=C(C)c1sc(C(=O)c2c[nH]c3ccccc23)nc1C(=O)OC] | I-70 |
| 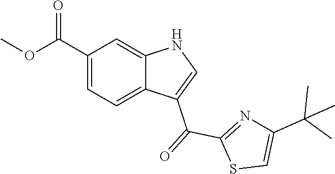 [SMILES: COC(=O)c1ccc2c(C(=O)c3nc(C(C)(C)C)cs3)c[nH]c2c1] | I-71 |
| 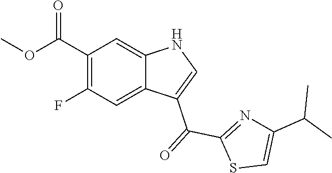 [SMILES: COC(=O)c1cc2[nH]cc(C(=O)c3nc(C(C)C)cs3)c2cc1F] | I-72 |
| 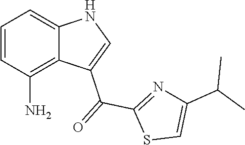 [SMILES: CC(C)c1csc(C(=O)c2c[nH]c3cccc(N)c23)n1] | I-73 |
| 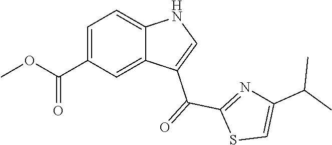 [SMILES: COC(=O)c1ccc2[nH]cc(C(=O)c3nc(C(C)C)cs3)c2c1] | I-74 |
| 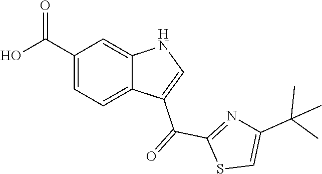 [SMILES: CC(C)(C)c1csc(C(=O)c2c[nH]c3cc(C(=O)O)ccc23)n1] | I-75 |
| 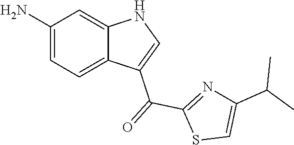 [SMILES: CC(C)c1csc(C(=O)c2c[nH]c3cc(N)ccc23)n1] | I-76 |
| 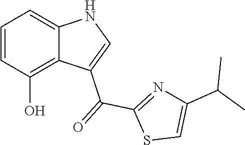 [SMILES: CC(C)c1csc(C(=O)c2c[nH]c3cccc(O)c23)n1] | I-77 |
| 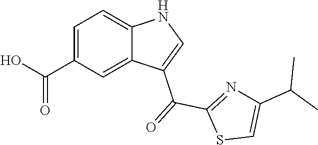 [SMILES: CC(C)c1csc(C(=O)c2c[nH]c3ccc(C(=O)O)cc23)n1] | I-78 |
| 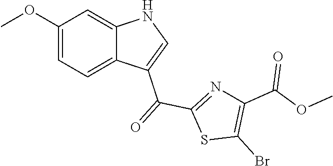 | I-79 |
| 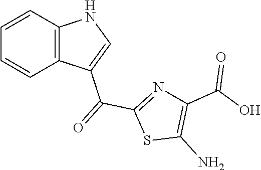 [SMILES: Nc1sc(C(=O)c2c[nH]c3ccccc23)nc1C(=O)O] | I-80 |
| 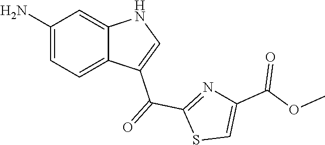 [SMILES: COC(=O)c1csc(C(=O)c2c[nH]c3cc(N)ccc23)n1] | I-81 |
| 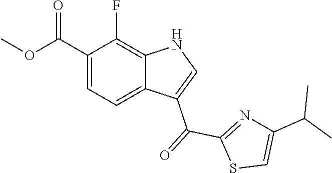 [SMILES: COC(=O)c1ccc2c(C(=O)c3nc(C(C)C)cs3)c[nH]c2c1F] | I-82 |
| 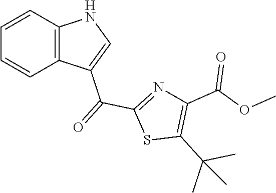 [SMILES: COC(=O)c1nc(C(=O)c2c[nH]c3ccccc23)sc1C(C)(C)C] | I-83 |
| 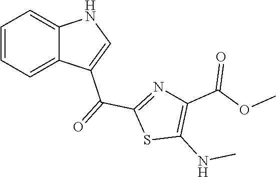 [SMILES: CNc1sc(C(=O)c2c[nH]c3ccccc23)nc1C(=O)OC] | I-84 |
| 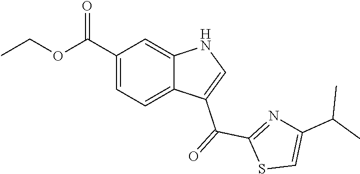 [SMILES: CCOC(=O)c1ccc2c(C(=O)c3nc(C(C)C)cs3)c[nH]c2c1] | I-85 |
| 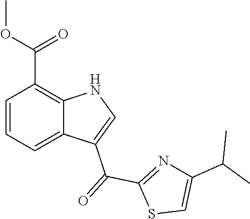 [SMILES: COC(=O)c1cccc2c(C(=O)c3nc(C(C)C)cs3)c[nH]c12] | I-86 |
| 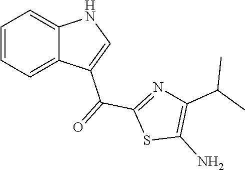 [SMILES: CC(C)c1nc(C(=O)c2c[nH]c3ccccc23)sc1N] | I-87 |
| 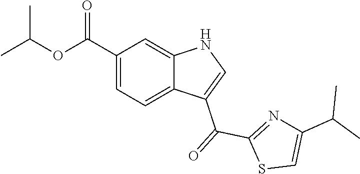 [SMILES: CC(C)OC(=O)c1ccc2c(C(=O)c3nc(C(C)C)cs3)c[nH]c2c1] | I-88 |
| 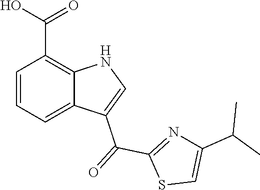 [SMILES: CC(C)c1csc(C(=O)c2c[nH]c3c(C(=O)O)cccc23)n1] | I-89 |
| 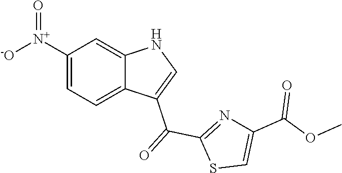 | I-90 |
| 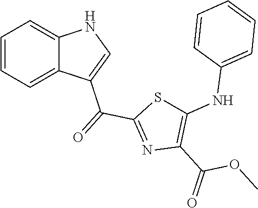 [SMILES: COC(=O)c1nc(C(=O)c2c[nH]c3ccccc23)sc1Nc1ccccc1] | I-91 |
| 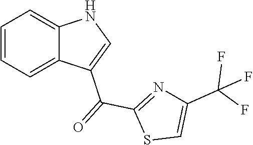 [SMILES: O=C(c1nc(C(F)(F)F)cs1)c1c[nH]c2ccccc12] | I-92 |
| 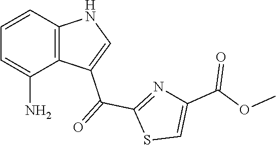 [SMILES: COC(=O)c1csc(C(=O)c2c[nH]c3cccc(N)c23)n1] | I-93 |
| 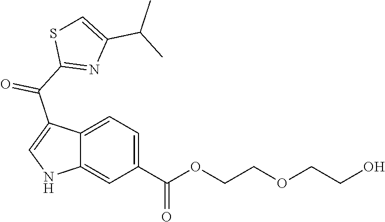 [SMILES: CC(C)c1csc(C(=O)c2c[nH]c3cc(C(=O)OCCOCCO)ccc23)n1] | I-94 |
| 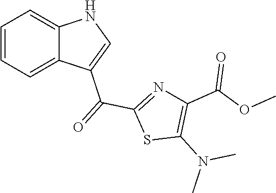 [SMILES: COC(=O)c1nc(C(=O)c2c[nH]c3ccccc23)sc1N(C)C] | I-95 |
| 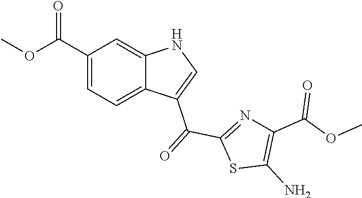 | I-96 |
| 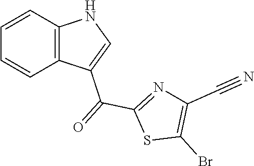 [SMILES: N#Cc1nc(C(=O)c2c[nH]c3ccccc23)sc1Br] | I-97 |

In some embodiments, the present invention provides a compound set forth in Table 1-a above, or a pharmaceutically acceptable salt thereof.

In some embodiments, a compound of the present invention is not

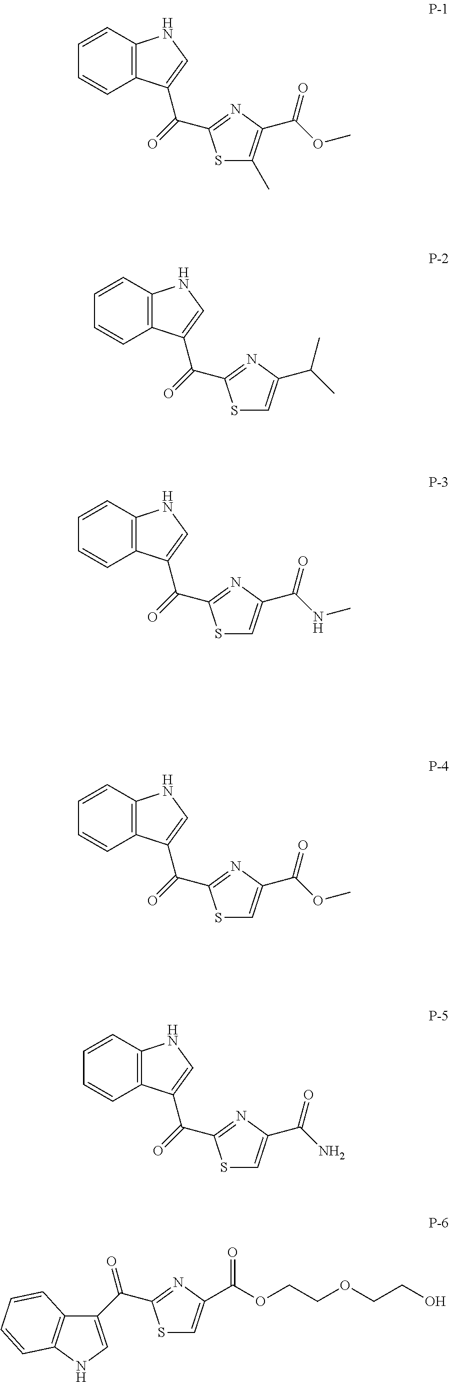 [SMILES: COC(=O)c1nc(C(=O)c2c[nH]c3ccccc23)sc1C ; CC(C)c1csc(C(=O)c2c[nH]c3ccccc23)n1 ; CNC(=O)c1csc(C(=O)c2c[nH]c3ccccc23)n1 ; COC(=O)c1csc(C(=O)c2c[nH]c3ccccc23)n1 ; NC(=O)c1csc(C(=O)c2c[nH]c3ccccc23)n1 ; O=C(OCCOCCO)c1csc(C(=O)c2c[nH]c3ccccc23)n1]

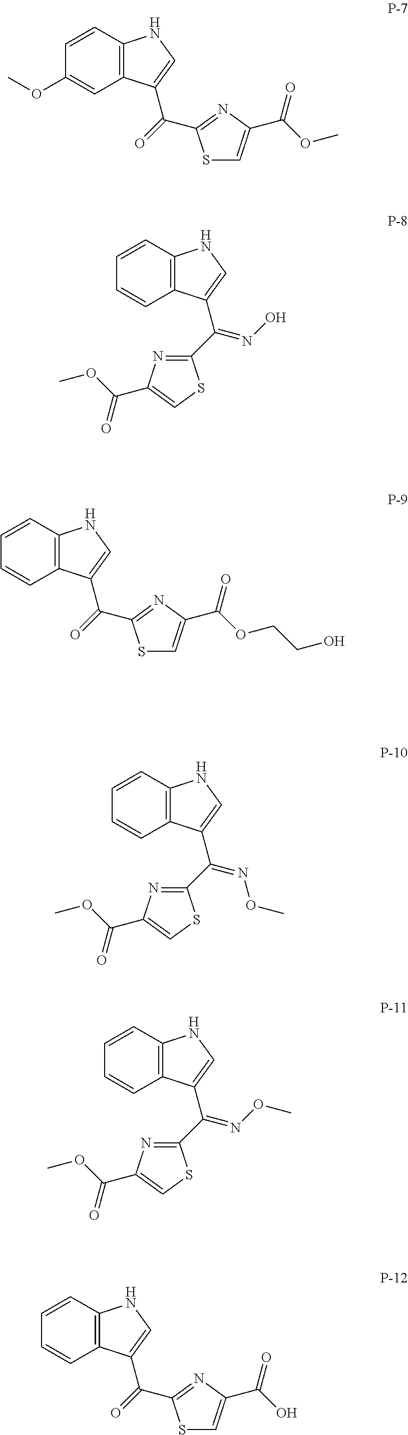

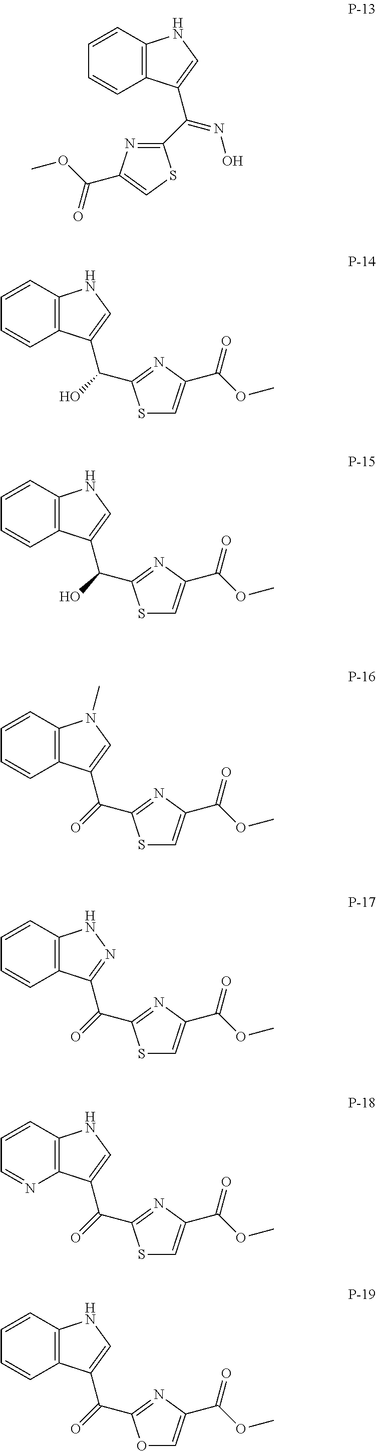

In some embodiments, a compound of the present invention is not

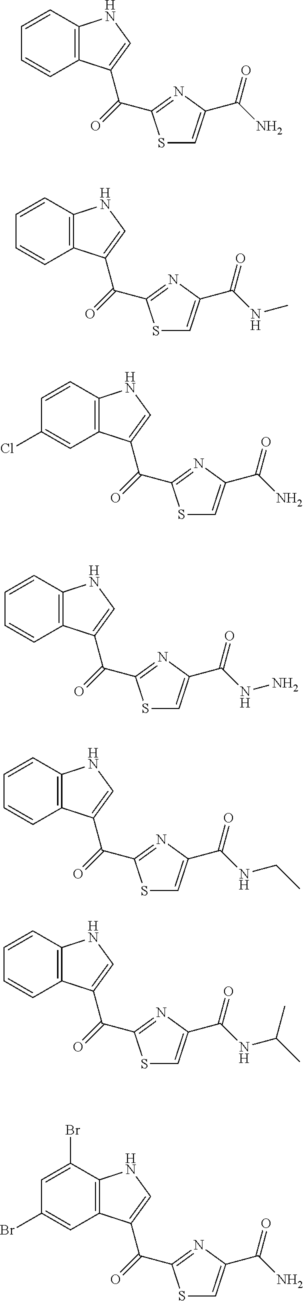

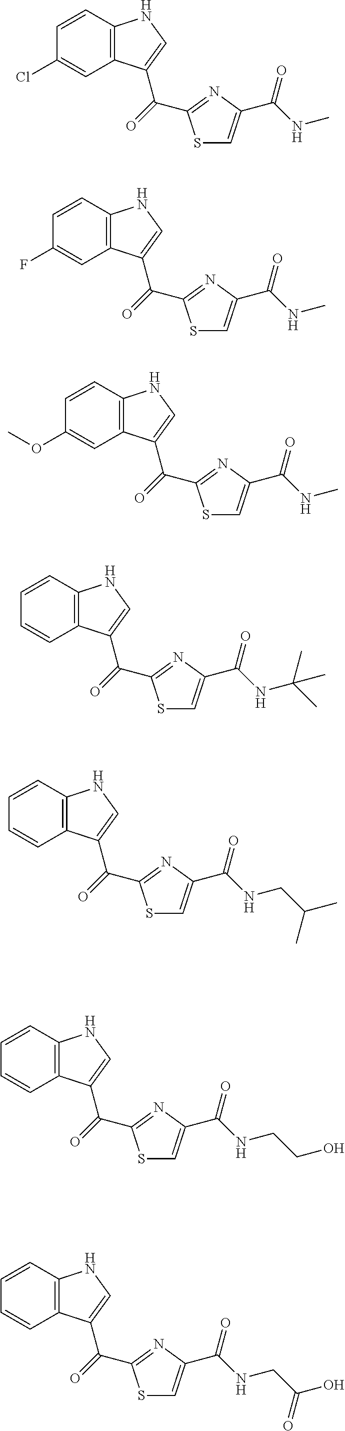

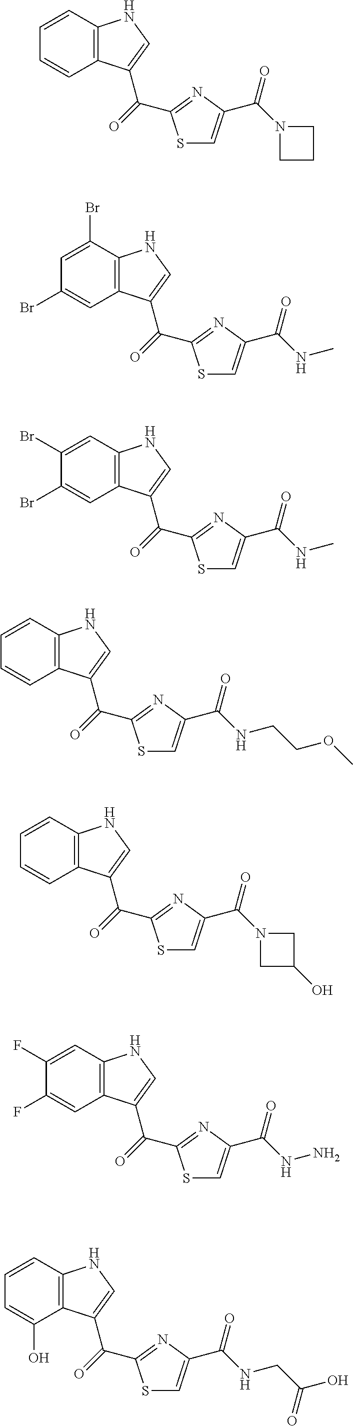

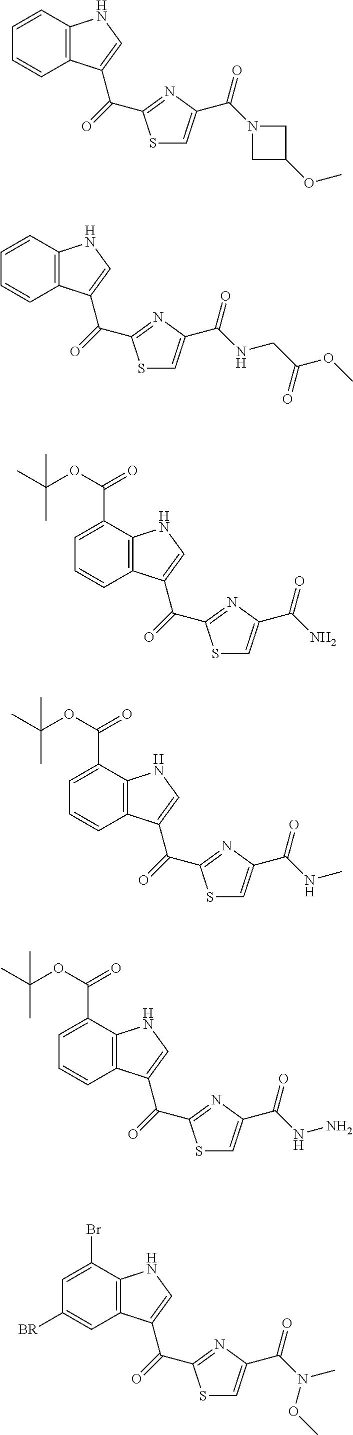

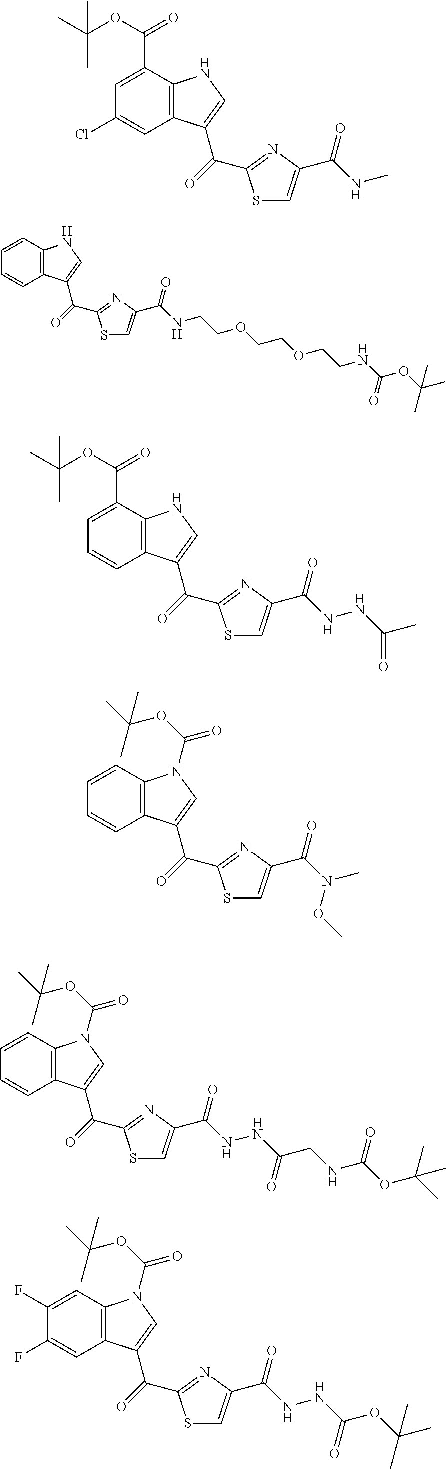

The compounds of this invention may be prepared or isolated in general by synthetic and/or semi-synthetic methods known to those skilled in the art for analogous compounds and by methods described in detail in the Examples, herein. In some embodiments, the present invention provides a compound or an intermediate compound as described in the Examples, or a salt thereof.

### 4. Uses, Formulation and Administration

Pharmaceutically Acceptable Compositions

According to another embodiment, the invention provides a pharmaceutical composition comprising a compound of this invention or a pharmaceutically acceptable derivative thereof and a pharmaceutically acceptable carrier, adjuvant, or vehicle. The amount of compound in compositions of this invention is such that is effective to measurably activate AHR, or a mutant thereof, in a biological sample or in a patient. The amount of compound in compositions of this invention is such that is effective to measurably activate AHR, or a variant or mutant thereof, in a biological sample or in a patient. In certain embodiments, the amount of compound in compositions of this invention is such that is effective to measurably activate AHR, or a mutant thereof, in a biological sample or in a patient. In certain embodiments, the amount of compound in compositions of this invention is such that is effective to measurably activate AHR, or a variant or mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an active metabolite or residue thereof.

As used herein, the term “active metabolite or residue thereof” means that a metabolite or residue thereof also activates AHR, or a mutant thereof. The term “active metabolite or residue thereof” also means that a metabolite or residue thereof activates AHR, or a variant or mutant thereof.

Compositions of the present invention can be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions can be formulated according to techniques known in the art using suitable dispersing or wetting agents and suspending agents. The sterile injectable preparation can also be a sterile injectable solution or suspension in a non-toxic parenterally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium.

For this purpose, any bland fixed oil can be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention can be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention can be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that can be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient depends upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition also depends upon the particular compound in the composition.

### Uses of Compounds and Pharmaceutically Acceptable Compositions

In some embodiments, the present invention provides a method of using a compound as described herein for treating a disease or disorder associated with AHR. In some embodiments, a disease or disorder associated with AHR is an angiogenesis implicated disorder as described herein. In some embodiments, a disease or disorder associated with AHR is a cancer as described herein. In some embodiments, a disease or disorder associated with AHR is an inflammatory disorder as described herein. In some embodiments, a disease or disorder associated with AHR is a disease or disorder as described in Gutiérrez-Vázquez C. et al. Immunity 2018, 48(1): 19-33, and Rothhammer V., et al., Nat Rev Immunol. 2019; 19(3): 184-197, each of which is incorporated herein by reference in its entirety.

### Angiogenesis Implicated Disorders

In one aspect, the present invention provides a method for treating or preventing or reducing the risk of an angiogenesis implicated disorder in a patient comprising administering to the patient a compound of the invention, or a pharmaceutically acceptable salt thereof, or a pharmaceutical composition thereof. In some embodiments, an angiogenesis implicated disorder is associated with a reduced expression or activation of an AHR.

In some embodiments, an angiogenesis implicated disorder is a retinopathy, psoriasis, rheumatoid arthritis, obesity, or cancer (for example, as described below).

### Cancer

In some embodiments, the present invention provides a method for treating or preventing or reducing the risk of cancer in patient comprising administering to the patient a compound of the invention, or a pharmaceutically acceptable salt thereof, or a pharmaceutical composition thereof. In some embodiments, a cancer is associated with a reduced expression or activation of an aryl hydrocarbon receptor (AHR).

The cancer or proliferative disorder or tumor to be treated using the compounds and methods and uses described herein include, but are not limited to, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.

In some embodiments, a cancer includes, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, a cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, a cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, a cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, a cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, a cancer is a viral-associated cancer, including human immunodeficiency virus (HIV) associated solid tumors, human papillomavirus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, a cancer is melanoma cancer. In some embodiments, a cancer is breast cancer. In some embodiments, a cancer is lung cancer. In some embodiments, a cancer is small cell lung cancer (SCLC). In some embodiments, a cancer is non-small cell lung cancer (NSCLC). In some embodiments, a cancer is selected from prostate cancer, liver cancer, and ovarian cancer.

### Inflammatory Disorders

In some embodiments, the present invention provides a method for treating or preventing or reducing the risk of an inflammatory disorder in patient comprising administering to the patient a compound of the invention, or a pharmaceutically acceptable salt thereof, or a pharmaceutical composition thereof. In some embodiments, an inflammatory disorder is associated with a reduced expression or activation of an aryl hydrocarbon receptor (AHR). In some embodiments, an inflammatory disorder is associated with a reduced expression or reduced activation of an aryl hydrocarbon receptor (AHR).

Inflammatory disorders include a large number of disorders or conditions that are involved in a variety of diseases, including those involving the immune system, including those demonstrated in allergic reactions and myopathies, or non-immune diseases with causal origins in inflammatory processes including, but not limited to cancer, atherosclerosis, and ischemic heart disease. Non-limiting examples of disorders associated with inflammation include, but are not limited to, acne vulgaris, asthma, autoimmune diseases, autoinflammatory diseases, celiac disease, chronic prostatitis, diverticulitis, glomerulonephritis, hidradenitis suppurativa, hypersensitivities, inflammatory bowel diseases, interstitial cystitis, otitis, pelvic inflammatory disease, reperfusion injury, rheumatic fever, rheumatoid arthritis, sarcoidosis, transplant rejection, and vasculitis.

In some embodiments, an inflammatory disorder is necrotizing enterocolitis, inflammatory bowel disease (IBD), autoimmune diseases, Crohn's disease, celiac disease, ulcerative colitis, cardiovascular disease, ocular Behcet's disease, breast cancer, and others.

Other non-limiting examples of inflammatory disease include, without limitation, acne, acid-induced lung injury, Addison's disease, adrenal hyperplasia, adrenocortical insufficiency, adult-onset Still's disease, adult respiratory distress syndrome (ARDS), age-related macular degeneration, aging, alcoholic hepatitis, alcoholic liver disease, allergen-induced asthma, allergic bronchopulmonary, allergic conjunctivitis, allergic contact dermatitis, allergies, allergic encephalomyelitis, allergic neuritis, allograft rejection, alopecia, alopecia areata, Alzheimer's disease, amyloidosis, amyotrophic lateral sclerosis, angina pectoris, angioedema, angiofibroma, anhidrotic ectodermal dysplasia-ill, anti-glomerular basement membrane disease, antigen-antibody complex mediated diseases, ankylosing spondylitis, antiphospholipid syndrome, aphthous stomatitis, appendicitis, arthritis, ascites, aspergillosis, asthma, atherosclerosis, atherosclerotic plaques, atopic dermatitis, atrophic thyroiditis, autoimmune diseases, autoimmune hemolytic anemia (immune pancytopenia, paroxysmal nocturnal hemoglobinuria), autoimmune polyendocrinopathies, autoimmune thrombocytopenia (idiopathic thrombocytopenic purpura, immune-mediated thrombocytopenia), autoimmune hepatitis, autoimmune thyroid disorders, autoinflammatory diseases, back pain, Bacillus anthracis infection, Bechet's disease, bee sting-induced inflammation, Behget's syndrome, Bell's palsy, berylliosis, Blau syndrome, bone pain, bronchiolitis, bullous pemphigoid (BP) asthma, burns, bursitis, cardiac hypertrophy, carpal tunnel syndrome, Castleman's disease, catabolic disorders, cataracts, Celiac disease, cerebral aneurysm, chemical irritant-induced inflammation, chorioretinitis, chronic atypical neutrophilic dermatosis with lipodystrophy and elevated temperature (CANDLE) syndrome, chronic heart failure, chronic lung disease of prematurity, chronic obstructive pulmonary disease (COPD), chronic pancreatitis, chronic prostatitis, chronic recurrent multifocal osteomyelitis, cicatricial alopecia, colitis, complex regional pain syndrome, complications of organ transplantation, conjunctivitis, connective tissue disease, contact dermatitis, corneal graft neovascularization, corneal ulcer, Crohn's disease, cryopyrin-associated periodic syndromes, cutaneous lupus erythematosus (CLE), cryptococcosis, cystic fibrosis, deficiency of the interleukin-1 receptor antagonist (DIRA), dermatitis, dermatitis endotoxemia, dermatomyositis, diabetic macular edema, diverticulitis, eczema, encephalitis, endometriosis, endotoxemia, eosinophilic pneumonias, epicondylitis, epidermolysis bullosa, erythema multiforme, erythroblastopenia, esophagitis, familial amyloidotic polyneuropathy, familial cold urticarial, familial Mediterranean fever, fetal growth retardation, fibromyalgia, fistulizing Crohn's disease, food allergies, giant cell arteritis, glaucoma, glioblastoma, glomerular disease, glomerular nephritis, glomerulonephritis, gluten-sensitive enteropathy, gout, gouty arthritis, graft-versus-host disease (GVHD), granulomatous hepatitis, Graves' disease, growth plate injuries, Guillain-Barre syndrome, gut diseases, hair loss, Hashimoto's thyroiditis, head injury, headache, hearing loss, heart disease, hemangioma, hemolytic anemia, hemophilic joints, Henoch-Scholein purpura, hepatitis, hereditary periodic fever syndrome, heritable disorders of connective tissue, herpes zoster and simplex, hidradenitis suppurativa (HS), hip replacement, Hodgkin's disease, Huntington's disease, hyaline membrane disease, hyperactive inflammatory response, hyperammonemia, hypercalcemia, hypercholesterolemia, hypereosinophilic syndrome (HES), hyperimmunoglobulinemia D with recurrent fever (HIDS), hypersensitivity pneumonitis, hypertropic bone formation, hypoplastic and other anemias, hypoplastic anemia, ichthyosis, idiopathic demyelinating polyneuropathy, Idiopathic inflammatory myopathies (dermatomyositis, polymyositis), idiopathic pulmonary fibrosis, idiopathic thrombocytopenic purpura, immunoglobulin nephropathies, immune complex nephritis, immune thrombocytopenic purpura (ITP), incontinentia pigmenti (IP, Bloch-Siemens syndrome), infectious mononucleosis, infectious diseases including viral diseases such as AIDS (HIV infection), hepatitis A, B, C, D, and E, herpes; inflammation, inflammation of the CNS, inflammatory bowel disease (IBD), inflammatory disease of the lower respiratory tract including bronchitis or chronic obstructive pulmonary diseases, inflammatory disease of the upper respiratory tract including the nose and sinuses such as rhinitis or sinusitis, inflammatory diseases of the respiratory tract, inflammatory ischemic event such as stroke or cardiac arrest, inflammatory lung disease, inflammatory myopathy such as myocarditis, inflammatory liver disease, inflammatory neuropathy, inflammatory pain, insect bite-induced inflammation, interstitial cystitis, interstitial lung disease, iritis, irritant-induced inflammation, ischemia/reperfusion, joint replacement, juvenile arthritis, juvenile rheumatoid arthritis, keratitis, kidney injury caused by parasitic infections, kidney transplant rejection, leptospirosis, leukocyte adhesion deficiency, lichen sclerosus (LS), Lambert-Eaton myasthenic syndrome, Loeffler's syndrome, lupus, lupus nephritis, Lyme disease, Marfan syndrome (MFS), mast cell activation syndrome, mastocytosis, meningitis, meningioma, mesothelioma, mixed connective tissue disease, Muckle-Wells syndrome (urticaria deafness amyloidosis), mucositis, multiple organ injury syndrome, multiple sclerosis, muscle wasting, muscular dystrophy, myasthenia gravis (MG), myelodysplastic syndrome, myocarditis, myositis, nasal sinusitis, necrotizing enterocolitis, neonatal onset multisystem inflammatory disease (NOMID), neovascular glaucoma, nephrotic syndrome, neuritis, neuropathological diseases, non-allergen induced asthma, obesity, ocular allergy, optic neuritis, organ transplant rejection, Osier-Weber syndrome, osteoarthritis, osteogenesis imperfecta, osteonecrosis, osteoporosis, osterarthritis, otitis, pachyonychia congenita, Paget's disease, Paget's disease of bone, pancreatitis, Parkinson's disease, pediatric rheumatology, pelvic inflammatory disease, pemphigus, pemphigus vulgaris (PV), bullous pemphigoid (BP), pericarditis, periodic fever, periodontitis, peritoneal endometriosis, pernicious anemia (Addison's disease), pertussis, PFAPA (periodic fever aphthous pharyngitis and cervical adenopathy), pharyngitis and adenitis (PFAPA syndrome), plant irritant-induced inflammation, pneumocystis infection, pneumonia, pneumonitis, poison ivy/urushiol oil-induced inflammation, polyarthritis nodosa, polychondritis, polycystic kidney disease, polymyalgia rheumatic, giant cell arteritis, polymyositis, pouchitis, reperfusion injury and transplant rejection, primary biliary cirrhosis, primary pulmonary hypertension, primary sclerosing cholangitis (PSC), proctitis, psoriasis, psoriasis vulgaris, psoriatic arthritis, psoriatic epidermis, psychosocial stress diseases, pulmonary disease, pulmonary fibrosis, pulmonary hypertension, pyoderma gangrenosum, pyogenic granuloma retrolental fibroplasias, pyogenic sterile arthritis, Raynaud's syndrome, Reiter's disease, reactive arthritis, renal disease, renal graft rejection, reperfusion injury, respiratory distress syndrome, retinal disease, retrolental fibroplasia, Reynaud's syndrome, rheumatic carditis, rheumatic diseases, rheumatic fever, rheumatoid arthritis, rhinitis, rhinitis psoriasis, rosacea, sarcoidosis, Schnitzler syndrome, scleritis, sclerosis, scleroderma, scoliosis, seborrhea, sepsis, septic shock, severe pain, Sezary syndrome, sickle cell anemia, silica-induced disease (Silicosis), Sjogren's syndrome, skin diseases, skin irritation, skin rash, skin sensitization (contact dermatitis or allergic contact dermatitis), sleep apnea, spinal cord injury, spinal stenosis, spondyloarthropathies, sports injuries, sprains and strains, Stevens-Johnson syndrome (SJS), stroke, subarachnoid hemorrhage, sunburn, synovial inflammation, systemic inflammatory response syndrome (SIRS), systemic lupus erythematosus, systemic mast cell disease (SMCD), systemic vasculitis, systemic-onset juvenile idiopathic arthritis, temporal arteritis, tendinitis, tenosynovitis, thrombocytopenia, thyroditis, thyroiditis, tissue transplant, toxoplasmosis, trachoma, transplantation rejection, traumatic brain injury, tuberculosis, tubulointerstitial nephritis, tumor necrosis factor (TNF) receptor associated periodic syndrome (TRAPS), type 1 diabetes, type 2 diabetes, complications from type 1 or type 2 diabetes, ulcerative colitis, urticaria, uterine fibroids, uveitis, uveoretinitis, vascular restenosis, vasculitis, vasculitis (NHLBI), vitiligo, Wegener's granulomatosis, and Whipple's disease.

The term “inflammatory bowel disease” or “IBD” as used herein is a collective term describing inflammatory disorders of the gastrointestinal tract, the most common forms of which are ulcerative colitis and Crohn's disease. Other forms of IBD that can be treated with the presently disclosed compounds, compositions and methods include diversion colitis, ischemic colitis, infectious colitis, chemical colitis, microscopic colitis (including collagenous colitis and lymphocytic colitis), atypical colitis, pseudomembranous colitis, fulminant colitis, autistic enterocolitis, indeterminate colitis, Behget's disease, gastroduodenal CD, jejunoileitis, ileitis, ileocolitis, Crohn's (granulomatous) colitis, irritable bowel syndrome, mucositis, radiation induced enteritis, short bowel syndrome, celiac disease, stomach ulcers, diverticulitis, pouchitis, proctitis, and chronic diarrhea.

As used herein, treating or preventing an inflammatory disease also includes ameliorating or reducing one or more symptoms of the inflammatory disease. Where the inflammatory disease or disorder is IBD, the term “symptoms of IBD” can refer to detected symptoms such as abdominal pain, diarrhea, rectal bleeding, weight loss, fever, loss of appetite, and other more serious complications, such as dehydration, anemia and malnutrition. A number of such symptoms are subject to quantitative analysis (e.g., weight loss, fever, anemia, etc.). Some symptoms are readily determined from a blood test (e.g., anemia) or a test that detects the presence of blood (e.g., rectal bleeding). The term “wherein said symptoms are reduced” refers to a qualitative or quantitative reduction in detectable symptoms, including but not limited to, a detectable impact on the rate of recovery from disease (e.g, rate of weight gain). The diagnosis is typically determined by way of an endoscopic observation of the mucosa, and pathologic examination of endoscopic biopsy specimens. The course of IBD varies, and is often associated with intermittent periods of disease remission and disease exacerbation. Various methods have been described for characterizing disease activity and severity of IBD as well as response to treatment in subjects having IBD. Treatment according to the present methods is generally applicable to a subject having IBD of any level or degree of disease activity.

In some embodiments, the present invention provides a method for treating or preventing or reducing the risk of an angiogenesis implicated disorder, cancer, or an inflammatory disorder, such as those described above, comprising administering to the patient a compound selected from:

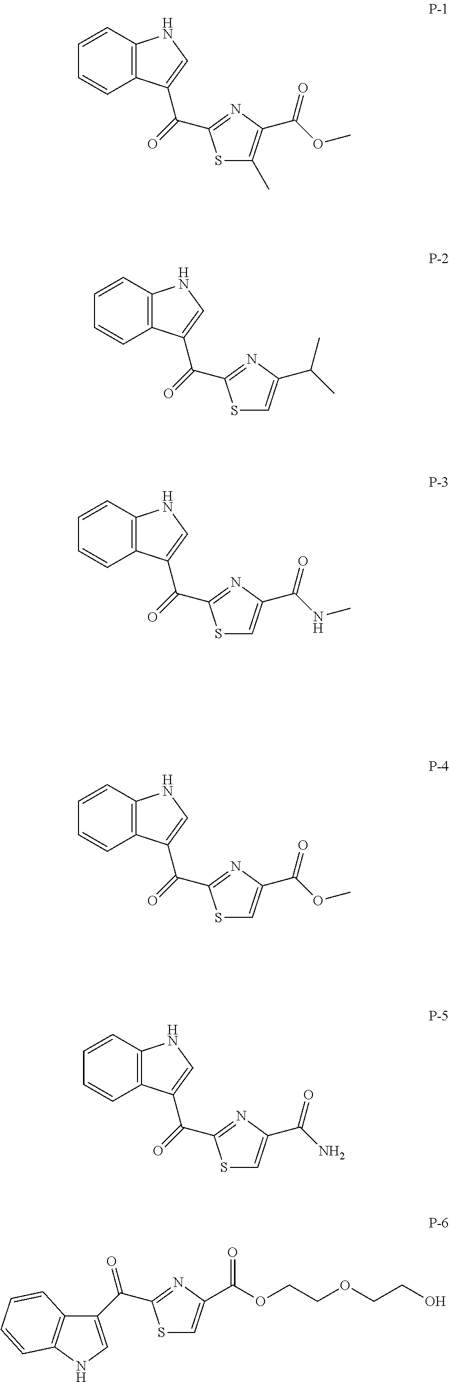 [SMILES: COC(=O)c1nc(C(=O)c2c[nH]c3ccccc23)sc1C ; CC(C)c1csc(C(=O)c2c[nH]c3ccccc23)n1 ; CNC(=O)c1csc(C(=O)c2c[nH]c3ccccc23)n1 ; COC(=O)c1csc(C(=O)c2c[nH]c3ccccc23)n1 ; NC(=O)c1csc(C(=O)c2c[nH]c3ccccc23)n1 ; O=C(OCCOCCO)c1csc(C(=O)c2c[nH]c3ccccc23)n1]

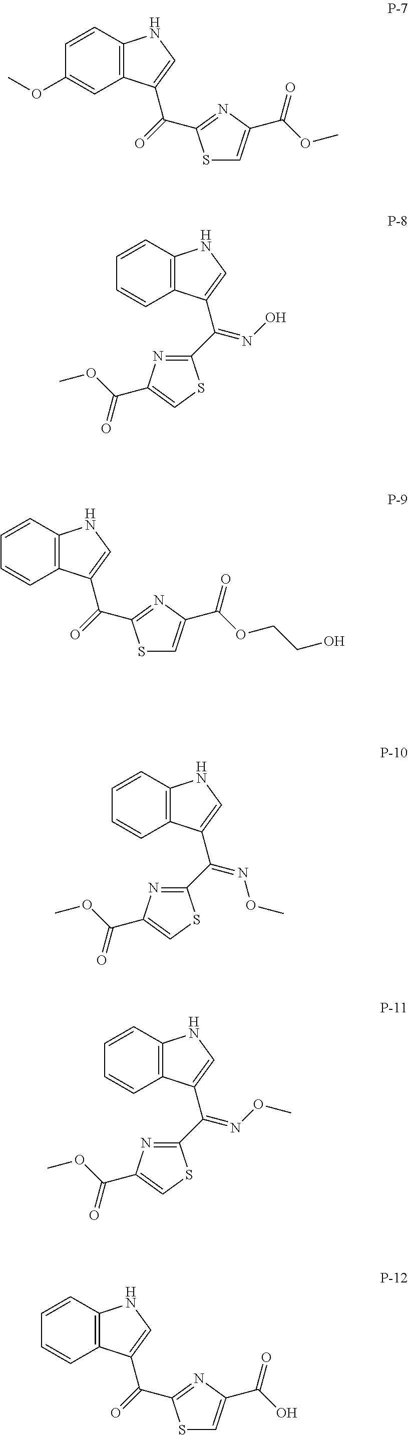

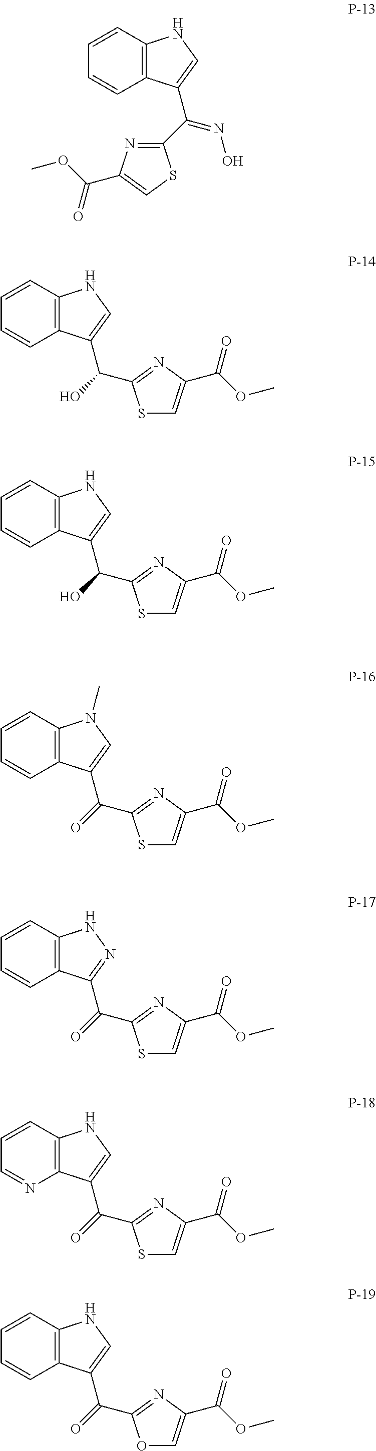

or a pharmaceutically acceptable salt thereof, or a pharmaceutical composition thereof.

The compounds and compositions, according to the method of the present invention, can be administered using any amount and any route of administration effective for activating AHR and treating or lessening the severity of a disease, for example, as those described herein. The exact amount required will vary from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Co-Administration with One or More Other Therapeutic Agent(s)

Depending upon the particular condition, or disease, to be treated, additional therapeutic agents that are normally administered to treat that condition, can also be present in the compositions of this invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds.

One or more other therapeutic agent(s) can be administered separately from a compound or composition of the invention, as part of a multiple dosage regimen. Alternatively, one or more other therapeutic agent(s) may be part of a single dosage form, mixed together with a compound of this invention in a single composition. If administered as a multiple dosage regime, one or more other therapeutic agent(s) and a compound or composition of the invention can be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent(s) and a compound or composition of the invention are administered as a multiple dosage regimen within greater than 24 hours apart.

As used herein, the term “combination,” “combined,” and related terms refers to the simultaneous or sequential administration of therapeutic agents in accordance with this invention. For example, a compound of the present invention can be administered with one or more other therapeutic agent(s) simultaneously or sequentially in separate unit dosage forms or together in a single unit dosage form. Accordingly, the present invention provides a single unit dosage form comprising a compound of the current invention, one or more other therapeutic agent(s), and a pharmaceutically acceptable carrier, adjuvant, or vehicle.

The amount of a compound of the invention and one or more other therapeutic agent(s) (in those compositions which comprise an additional therapeutic agent as described above) that can be combined with the carrier materials to produce a single dosage form varies depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent(s), the one or more other therapeutic agent(s) and a compound of the invention can act synergistically. Therefore, the amount of the one or more other therapeutic agent(s) in such compositions may be less than that required in a monotherapy utilizing only that therapeutic agent. In such compositions a dosage of between 0.01-1,000 g/kg body weight/day of the one or more other therapeutic agent(s) can be administered.

The amount of one or more other therapeutic agent(s) present in the compositions of this invention may be no more than the amount that would normally be administered in a composition comprising that therapeutic agent as the only active agent. Preferably the amount of one or more other therapeutic agent(s) in the presently disclosed compositions ranges from about 50% to 100% of the amount normally present in a composition comprising that agent as the only therapeutically active agent. In some embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approved for dosing per the FDA label insert.

The compounds of this invention, or pharmaceutical compositions thereof, can also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Other Therapeutic Agents

In some embodiments, provided herein are methods of treatment in which an AHR agonist compound described herein is administered in combination with an agent for treatment of an inflammatory disease or condition. Examples of agents for treatment of an inflammatory disease or condition that can be used in combination with compounds described herein, include alpha-fetoprotein modulators; adenosine A3 receptor antagonist; adrenomedullin ligands; AKT1 gene inhibitors; antibiotics; antifungals; ASK1 inhibitors; ATPase inhibitors; beta adrenoceptor antagonists; BTK inhibitors; calcineurin inhibitors; carbohydrate metabolism modulators; cathepsin S inhibitors; CCR9 chemokine antagonists; CD233 modulators; CD29 modulators; CD3 antagonists; CD40 ligand inhibitors; CD40 ligand receptor antagonists; chemokine CXC ligand inhibitors; CHST15 gene inhibitors; collagen modulators; CSF-1 antagonists; CX3CR1 chemokine modulators; ecobiotics; eotaxin ligand inhibitors; EP4 prostanoid receptor agonists; FI FO ATP synthase modulators; farnesoid X receptor (FXR and NR1 H4) agonists or modulators; fecal microbiota transplantation (FMT); fractalkine ligand inhibitors; free fatty acid receptor 2 antagonists; GATA 3 transcription factor inhibitors; glucagon-like peptide 2 agonists; glucocorticoid agonists; Glucocorticoid receptor modulators; guanylate cyclase receptor agonists; HIF prolyl hydroxylase inhibitors; histone deacetylase inhibitors; HLA class II antigen modulators; hypoxia inducible factor-1 stimulator; ICAM1 gene inhibitors; IL-1 beta ligand modulators; IL-12 antagonists; IL-13 antagonists; IL-18 antagonists; IL-22 agonists; IL-23 antagonists; IL-23A inhibitors; IL-6 antagonists; IL-7 receptor antagonists; IL-8 receptor antagonists; integrin alpha-4/beta-1 antagonists; integrin alpha-4/beta-7 antagonists; integrin antagonists; interleukin ligand inhibitors; interleukin receptor 17A antagonists; interleukin-1 beta ligands; interleukin 1 like receptor 2 inhibitors; IL-6 receptor modulators; JAK tyrosine kinase inhibitors; Jak1 tyrosine kinase inhibitors; Jak3 tyrosine kinase inhibitors; lactoferrin stimulators; LanC like protein 2 modulators; leukocyte elastate inhibitors; leukocyte proteinase-3 inhibitors; MAdCAM inhibitors; melanin concentrating hormone (MCH-1) antagonist; melanocortin agonists; metalloprotease-9 inhibitors; microbiome-targeting therapeutics; natriuretic peptide receptor C agonists; neuregulin-4 ligands; NLPR3 inhibitors; NKG2 D activating NK receptor antagonists; nuclear factor kappa B inhibitors; opioid receptor antagonists; 0X40 ligand inhibitors; oxidoreductase inhibitors; P2X7 purinoceptor modulators; PDE 4 inhibitors; Pellino homolog 1 inhibitors; PPAR alpha/delta agonists; PPAR gamma agonists; protein fimH inhibitors; P-selectin glycoprotein ligand-1 inhibitors; Ret tyrosine kinase receptor inhibitors; RIP-1 kinase inhibitors; RIP-2 kinase inhibitors; RNA polymerase inhibitors; sphingosine 1 phosphate phosphatase 1 stimulators; sphingosine-1-phosphate receptor-1 agonists; sphingosine-1-phosphate receptor-5 agonists; sphingosine-1-phosphate receptor-1 antagonists; sphingosine-1-phosphate receptor-1 modulators; stem cell antigen-1 inhibitors; superoxide dismutase modulators; SYK inhibitors; tissue transglutaminase inhibitor; TLR-3 antagonists; TLR-4 antagonists; Toll-like receptor 8 (TLR8) inhibitors; TLR-9 agonists; TNF alpha ligand inhibitors; TNF ligand inhibitors; TNF alpha ligand modulators; TNF antagonists; TPL-2 inhibitors; tumor necrosis factor 14 ligand modulators; tumor necrosis factor 15 ligand inhibitors; Tyk2 tyrosine kinase inhibitors; type IIL-1 receptor antagonists; vanilloid VR1 agonists; and zonulin inhibitors, and combinations thereof.

In some embodiments, the one or more other therapeutic agents is an anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate. Non-limiting examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors (i.e., a compound that inhibits COX-2 with an IC50 that is at least 50-fold lower than the IC50 for COX-1) such as celecoxib, valdecoxib, lumiracoxib, etoricoxib and/or rofecoxib.

In a further embodiment, the anti-inflammatory agent is a salicylate. Salicylates include, but are not limited to, acetylsalicylic acid or aspirin, sodium salicylate, and choline and magnesium salicylates.

The anti-inflammatory agent can also be a corticosteroid. For example, the corticosteroid can be chosen from cortisone, dexamethasone, methylprednisolone, prednisolone, prednisolone sodium phosphate, and prednisone. In some embodiments, the anti-inflammatory therapeutic agent is a gold compound such as gold sodium thiomalate or auranofin.

In some embodiments, the anti-inflammatory agent is a metabolic inhibitor such as a dihydrofolate reductase inhibitor, such as methotrexate or a dihydroo rotate dehydrogenase inhibitor, such as leflunomide.

In some embodiments, the anti-inflammatory compound is an anti-C5 monoclonal antibody (such as eculizumab or pexelizumab), a TNF antagonist, such as entanercept, or infliximab, which is an anti-TNF alpha monoclonal antibody.

Included herein are methods of treatment in which a compound described herein, is administered in combination with an immunosuppressant. In some embodiments, the immunosuppressant is methotrexate, leflunomide, cyclosporine, tacrolimus, azathioprine, or mycophenolate mofetil.

Included herein are methods of treatment in which an AHR agonist compound described herein, is administered in combination with a class of agent for treatment of IBD. Examples of classes of agents for treatment of IBD that can be used in combination with a compound described herein include ASK1 inhibitors, beta adrenoceptor antagonists, BTK inhibitors, beta-glucuronidase inhibitors, bradykinin receptor modulators, calcineurin inhibitors, calcium channel inhibitors, cathepsin S inhibitors, CCR3 chemokine antagonists, CD40 ligand receptor antagonists, chemokine CXC ligand inhibitors, CHST15 gene inhibitors, collagen modulators, CSF-1 antagonists, cyclooxygenase inhibitors, cytochrome P450 3A4 inhibitors, eotaxin ligand inhibitors, EP4 prostanoid receptor agonists, erythropoietin receptor agonists, fractalkine ligand inhibitors, free fatty acid receptor 2 antagonists, GATA 3 transcription factor inhibitors, glucagon-like peptide 2 agonists, glucocorticoid agonists, guanylate cyclase receptor agonists, histone deacetylase inhibitors, HLA class II antigen modulators, IL-12 antagonists, IL-13 antagonists, IL-23 antagonists, IL-6 antagonists, IL-6 receptor modulators, interleukin-7 receptor modulators, IL-7 antagonists, IL-8 antagonists, integrin alpha-4/beta-1 antagonists, integrin alpha-4/beta-7 antagonists, integrin alpha-E antagonists, integrin antagonists, integrin beta-7 antagonists, interleukin ligand inhibitors, interleukin-2 ligand, interleukin receptor 17A antagonists, interleukin-1 beta ligands, interleukin-1 beta ligand modulators, IRAK4 inhibitors, JAK tyrosine kinase inhibitors, Jak1 tyrosine kinase inhibitors, Jak3 tyrosine kinase inhibitors, LanC like protein 2 modulators, lipoxygenase modulators, MAdCAM inhibitors, matrix metalloprotease inhibitors, melanocortin agonists, metalloprotease-9 inhibitors, natriuretic peptide receptor C agonists, neuregulin-4 ligands, NKG2 D activating NK receptor antagonists, opioid receptor antagonists, opioid receptor delta antagonists, oxidoreductase inhibitors, P2X7 purinoceptor agonists, PDE 4 inhibitors, phagocytosis stimulating peptide modulators, potassium channel inhibitors, PPAR alpha agonists, PPAR delta agonists, PPAR gamma agonists, protein fimH inhibitors, P-selectin glycoprotein ligand-1 inhibitors, RNA polymerase inhibitors, sphingosine 1 phosphate phosphatase 1 stimulators, sphingosine 1 phosphate phosphatase modulators, sphingosine-1-phosphate receptor-1 agonists, sphingosine-1-phosphate receptor-1 antagonists, sphingosine-1-phosphate receptor-1 modulators, sphingosine-1-phosphate receptor-5 modulators, STAT3 gene inhibitors, stem cell antigen-1 inhibitors, superoxide dismutase modulators, superoxide dismutase stimulators, SYK inhibitors, TGF beta 1 ligand inhibitors, thymulin agonists, TLR antagonists, TLR agonists, TNF alpha ligand inhibitors, TNF antagonists, tumor necrosis factor 14 ligand modulators, type II TNF receptor modulators, Tpl 2 inhibitors, and Zonulin inhibitors.

Included herein are methods of treatment in which a compound described herein is administered in combination with an agent for treatment of IBD. Examples of agents for treatment of IBD that can be used in combination with a compound described herein, or a pharmaceutically acceptable salt, stereoisomer, mixture of stereoisomers, tautomer, or deuterated analog thereof, include those provided herein for the treatment of an inflammatory disease or condition, and ABX-464, adalimumab; alicaforsen, ALLO-ASC-CD, AMG-966, anakinra, apremilast; Alequel; AMG-139; amiselimod, ASD-003, ASP-3291, AX-1505, BBT-401, balsalazide; beclomethasone dipropionate; BI-655130, BMS-986184; budesonide; CEQ-508; certolizumab; ChAdOx2-HAV, dexamethasone sodium phosphate, DNVX-078, etanercept; cibinetide; Clostridium butyricum; ETX-201, golimumab; GS-4997, GS-9876, GS-4875, GS-4059, infliximab; mesalazine, HLD-400, LYC-30937 EC; IONIS-JBI1-2.5Rx, JNJ-64304500, JNJ-4447, naltrexone; natalizumab; neihulizumab, olsalazine; PH-46-A, propionyl-L-carnitine; PTG-100; remestemcel-L; tacrolimus; teduglutide; tofacitinib; ASP-1002; ustekinumab; vedolizumab; AVX-470; INN-108; SGM-1019; PF-06480605; PF-06651600; PF-06687234; RBX-8225, SER-287; Thetanix; TOP-1288; VBY-129; 99mTc-annexin V-128; bertilimumab; DLX-105; dolcanatide; FFP-104; filgotinib; foralumab; GED-0507-34-Levo; givinostat; GLPG-0974; iberogast; JNJ-40346527; K(D)PT; KAG-308; KHK-4083; KRP-203; larazotide acetate; LY-3074828, midismase; olokizumab; OvaSave; P-28-GST; PF-547659; prednisolone; QBECO; RBX-2660, RG-7835; JKB-122; SB-012; STNM-01; Debio-0512; TRK-170; zucapsaicin; ABT-494; Ampion; BI-655066; carotegast methyl; cobitolimod; elafibranor; etrolizumab; GS-5745; HMPL-004; LP-02, ozanimod; peficitinib; quetmolimab (E-6011); RHB-104; rifaximin; tildrakizumab; tralokinumab; brodalumab; laquinimod; plecanatide; vidofludimus; and AZD-058.

### EXEMPLIFICATION

The following examples are intended to illustrate the invention and are not to be construed as being limitations thereon. Unless otherwise stated, one or more tautomeric forms of compounds of the examples described hereinafter may be prepared in situ and/or isolated. All tautomeric forms of compounds of the examples described hereafter should be considered to be disclosed. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art. Further, the compounds of the present invention can be produced by organic synthesis methods known to one of ordinary skill in the art as shown in the following examples.

### Example 1: Synthesis of Exemplary Compounds

Certain exemplary compounds are prepared following the following schemes.

### I-3

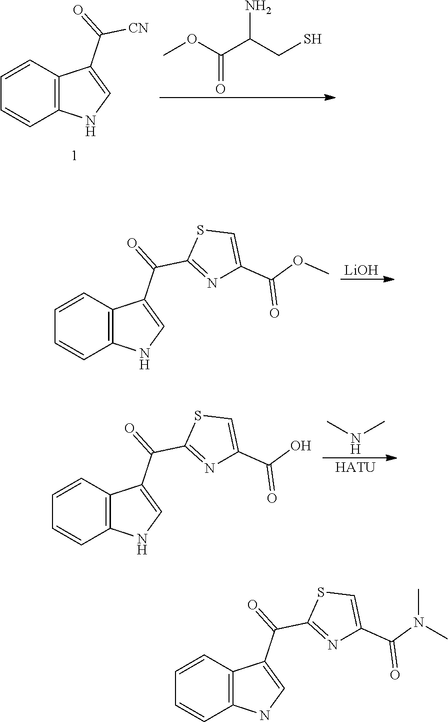

### Step 1: Methyl 2-(1H-indole-3-carbonyl)thiazole-4-carboxylate

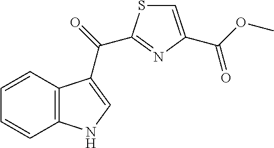 [SMILES: COC(=O)c1csc(C(=O)c2c[nH]c3ccccc23)n1]

To a solution of 1H-indole-3-carbonyl cyanide (3 g, 15.87 mmol, 1 eq) in pyridine (20 mL) was added DBU (241.55 mg, 1.59 mmol, 239.16 μL, 0.1 eq) and methyl 2-amino-3-sulfanyl-propanoate (2.72 g, 15.87 mmol, 1 eq, HCl). After stirring at 40° C. for 2 h, the reaction mixture was diluted with DCM (400 mL) and cooled to 0° C., added DBU (4.83 g, 31.73 mmol, 4.78 mL, 2 eq), followed by NBS (3.11 g, 17.45 mmol, 1.1 eq) portion-wise. The mixture was stirred at 0° C. for 1 h. The mixture was quenched with 1 N HCl solution (200 mL) and extracted with DCM (200 mL×2). The combined DCM layers were washed with 1 N HCl solution (50 mL) and brine (50 mL), dried over anhydrous Na₂SO₄, filtered, concentrated to yield a residue which was added DCM/MeOH (10/1, 50 mL), and stirred at 10° C. for 0.5 h. The slurry was filtered, and the cake was rinsed with DCM (2×10 mL). The solid was collected and dried in vacuo to yield methyl 2-(1H-indole-3-carbonyl)thiazole-4-carboxylate (3 g, 10.48 mmol, 66.0% yield, 100% purity) as a brown solid. ¹H NMR (400 MHz, DMSO-d₆) δ ppm 12.39 (s, 1H), 9.09 (d, J=3.1 Hz, 1H), 8.88 (s, 1H), 8.35-8.26 (m, 1H), 7.63-7.53 (m, 1H), 7.37-7.23 (m, 2H), 3.92 (s, 3H); ES-LCMS m/z 287.0 [M+H]⁺.

### Step 2: 2-(1H-Indole-3-carbonyl)thiazole-4-carboxylic acid

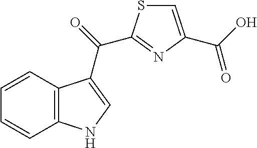 [SMILES: O=C(O)c1csc(C(=O)c2c[nH]c3ccccc23)n1]

To a solution of methyl 2-(1H-indole-3-carbonyl)thiazole-4-carboxylate (850 mg, 2.97 mmol, 1 eq) in EtOH (20 mL) and H₂O (20 mL) was added LiOH.H₂O (622.92 mg, 14.84 mmol, 5 eq). The mixture was stirred at 25° C. for 5 h. The mixture was adjusted pH to 4 with 1 N aq. HCl, then filtered and the solid was dried under reduce pressure to yield 2-(1H-indole-3-carbonyl)thiazole-4-carboxylic acid (800 mg, 2.91 mmol, 97.9% yield, 99% purity) as a yellow solid which was used in the next step without further purification. ¹H NMR (500 MHz, DMSO-d₆) δ ppm 13.41 (s, 1H), 12.38 (s, 1H), 9.15 (d, J=3.2 Hz, 1H), 8.80 (s, 1H), 8.31 (dd, J=2.7, 5.8 Hz, 1H), 7.62-7.57 (m, 1H), 7.31-7.28 (m, 2H); ES-LCMS m/z 273.0 [M+H]⁺.

### Step 3: 2-(1H-Indole-3-carbonyl)-N,N-dimethyl-thiazole-4-carboxamide

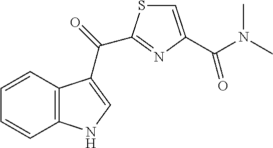 [SMILES: CN(C)C(=O)c1csc(C(=O)c2c[nH]c3ccccc23)n1]

To a solution of 2-(1H-indole-3-carbonyl)thiazole-4-carboxylic acid (800 mg, 2.91 mmol, 1 eq) in DMF (30 mL) was added HATU (1.99 g, 5.24 mmol, 1.8 eq), DIEA (1.13 g, 8.73 mmol, 1.52 mL, 3 eq) and A-methylmethanamine (711.58 mg, 8.73 mmol, 799.53 μL, 3 eq, HCl). The mixture was stirred at 25° C. for 3 h. The resulting product was filtered and concentrated under reduced pressure to yield a residue which was purified by preparative HPLC (column: Agela DuraShell C18 250*25 mm*10 μm; mobile phase: [water (0.04% NH₃.H₂O+10 mM NH₄HCO₃)-ACN]; B %: 27%-57%, 10 min), followed by lyophilization to yield 2-(1H-indole-3-carbonyl)-N,N-dimethyl-thiazole-4-carboxamide (30.99 mg, 103.53 μmol, 3.5% yield, 100% purity) as a yellow solid. ¹H NMR (500 MHz, CD₃OD) δ ppm 9.13-9.05 (m, 1H), 8.40-8.36 (m, 1H), 8.31 (s, 1H), 7.54-7.49 (m, 1H), 7.32-7.26 (m, 2H), 3.35 (s, 3H), 3.19 (s, 3H); ES-LCMS m/z 300.1 [M+H]⁺.

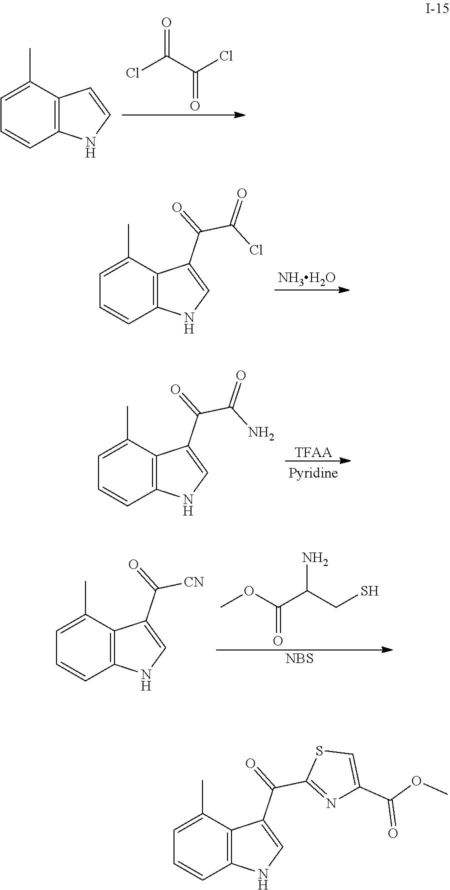

### Step 1: 2-(4-Methyl-1H-indol-3-yl)-2-oxo-acetyl chloride

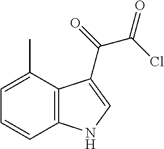

To a solution of 4-methyl-1H-indole (2 g, 15.25 mmol, 1 eq) in THF (20 mL) was added drop-wise (COCl)₂ (1.97 g, 15.55 mmol, 1.36 mL, 1.02 eq) at 0-5° C. under N₂. The mixture was stirred at 0-5° C. for 3 h. The reaction mixture was concentrated to yield crude 2-(4-methyl-1H-indol-3-yl)-2-oxo-acetyl chloride (3.38 g, crude) as a yellow solid which was used in the next step without further purification.

### Step 2: 2-(4-Methyl-1H-indol-3-yl)-2-oxo-acetamide

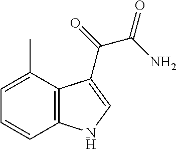

To a solution of THF (100 mL) and NH₃.H₂O (45.50 g, 363.52 mmol, 50 mL, 28%, 23.84 eq) was added 2-(4-methyl-1H-indol-3-yl)-2-oxo-acetyl chloride (3.38 g, 15.25 mmol, 1 eq). The mixture was stirred at 0° C. for 2 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 1/2, TLC:PE/EtOAc=1/1, R_{f}=0.16) to yield 2-(4-methyl-1H-indol-3-yl)-2-oxo-acetamide (900 mg, 3.91 mmol, 25.6% yield, 87.9% purity) as a white solid. ¹H NMR (500 MHz, DMSO-d₆) δ ppm 12.15 (brs, 1H), 8.43 (d, J=3.1 Hz, 1H), 8.05 (br s, 1H), 7.65 (br s, 1H), 7.32 (d, J=7.9 Hz, 1H), 7.14 (t, J=7.6 Hz, 1H), 6.98 (d, J=7.2 Hz, 1H), 2.77 (s, 3H); ES-LCMS m/z 203.0 [M+H]⁺.

### Step 3: 4-Methyl-1H-indole-3-carbonyl cyanide

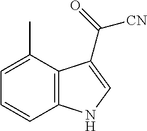

To a solution of 2-(4-methyl-1H-indol-3-yl)-2-oxo-acetamide (0.9 g, 3.91 mmol, 1 eq), pyridine (1.86 g, 23.47 mmol, 1.89 mL, 6 eq) in EtOAc (40 mL) was added TFAA (2.47 g, 11.74 mmol, 1.63 mL, 3 eq) under N₂ while the solution turned clean. The mixture was stirred at 25° C. for 12 h. The reaction mixture was quenched by addition of NaHCO₃ solution (100 mL), extracted with EtOAc (80 mL×3). The combined organic layers were washed with 0.5 N aq. HCl solution (20 mL), brine (20 mL), dried over Na₂SO₄, filtered and concentrated under reduced pressure to yield a residue which was purified by flash silica gel chromatography (from PE/EtOAc=1/0 to 3/1, TLC:PE/EtOAc=3/1, R_{f}=0.46) to yield 4-methyl-1H-indole-3-carbonyl cyanide (600 mg, 2.83 mmol, 72.2% yield, 86.8% purity) as a gray solid. ¹H NMR (500 MHz, DMSO-d₆) δ ppm 12.89 (br s, 1H), 8.68-8.55 (m, 1H), 7.37 (d, J=8.1 Hz, 1H), 7.24 (t, J=7.7 Hz, 1H), 7.06 (d, J=13 Hz, 1H), 2.78-2.69 (m, 3H); ES-LCMS m/z 185.0 [M+H]⁺.

### Step 4: Methyl 2-(4-methyl-1H-indole-3-carbonyl)thiazole-4-carboxylate

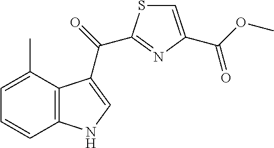 [SMILES: COC(=O)c1csc(C(=O)c2c[nH]c3cccc(C)c23)n1]

To a solution of 4-methyl-1H-indole-3-carbonyl cyanide (600 mg, 2.83 mmol, 1 eq) in pyridine (10 mL) was added DBU (43.05 mg, 282.75 μmol, 42.62 μL, 0.1 eq) and methyl 2-amino-3-sulfanyl-propanoate (485.32 mg, 2.83 mmol, 1 eq, HCl salt). After being stirred at 40° C. for 2 h, the reaction mixture was diluted with DCM (200 mL), cooled to 0° C., added DBU (860.90 mg, 5.65 mmol, 852.38 μL, 2.0 eq), followed by NBS (553.55 mg, 3.11 mmol, 1.1 eq) portion-wise. The mixture was stirred at 0° C. for 1 h. The mixture was quenched with 1 N HCl solution (200 mL) and extracted with DCM (200 mL) twice. The combined DCM layers were washed with 1 N HCl solution (50 mL) and brine (50 mL), dried over anhydrous Na₂SO₄, filtered, concentrated to yield a residue which was purified by flash silica gel chromatography (from PE/EtOAc=1/0 to 3/1, TLC:PE/EtOAc=3/1, Rf=0.39) to yield 2-(4-methyl-1H-indole-3-carbonyl)thiazole-4-carboxylate (250 mg, 832.41 μmol, 29.4% yield, 100% purity) as a white solid. Note: 31 mg of product was delivered. ¹H NMR (500 MHz, CDCl₃) δ ppm 9.10 (br s, 1H), 9.01 (d, J=3.4 Hz, 1H), 8.45 (s, 1H), 7.26-7.17 (m, 2H), 7.08 (d, J=7.0 Hz, 1H), 4.08-3.88 (m, 3H), 2.86 (s, 3H); ES-LCMS m/z 301.0 [M+H]⁺.

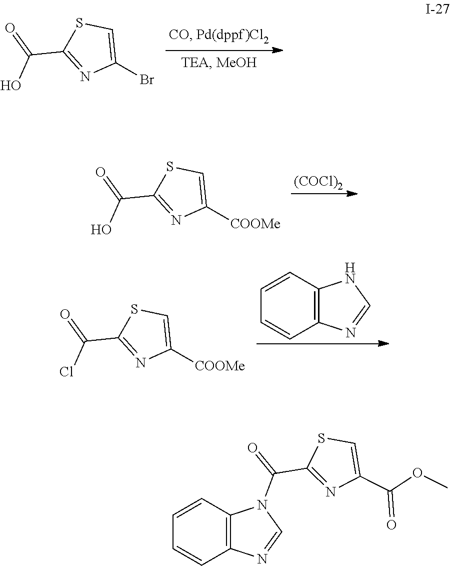 [SMILES: O=C(O)c1nc(Br)cs1 ; COC(=O)c1csc(C(=O)O)n1 ; "(COCl)2" ; COC(=O)c1csc(C(=O)Cl)n1 ; c1ccc2[nH]cnc2c1 ; COC(=O)c1csc(C(=O)n2cnc3ccccc32)n1]

### Step 1: 4-Methoxycarbonylthiazole-2-carboxylic acid

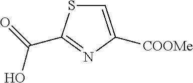 [SMILES: COC(=O)c1csc(C(=O)O)n1]

A solution of 4-bromothiazole-2-carboxylic acid (400 mg, 1.92 mmol, 1 eq), Pd(dppf)Cl₂ (140.69 mg, 192.28 μmol, 0.1 eq) and TEA (583.70 mg, 5.77 mmol, 802.88 μL, 3 eq) in MeOH (20 mL) saturated with CO (gas) was stirred under 50 Psi at 80° C. for 60 h in a 50 mL of sealed tube. The reaction mixture was concentrated under reduced pressure to yield a residue which was purified by flash silica gel chromatography (from DCM/MeOH=100/1 to 10/1, TLC:DCM/MeOH=10/1, R_{f}=0.39) to yield 4-methoxycarbonylthiazole-2-carboxylic acid (350 mg, 1.83 mmol, 95.3% yield, 98% purity) as a brown solid. ¹H NMR (500 MHz, CD₃OD) δ ppm 8.94-8.52 (m, 1H), 3.92 (s, 3H); ES-LCMS m/z 188.2 [M+H]⁺.

### Step 2: Methyl 2-chlorocarbonylthiazole-4-carboxylate

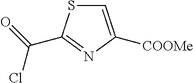 [SMILES: COC(=O)c1csc(C(=O)Cl)n1]

To a solution of 4-methoxycarbonylthiazole-2-carboxylic acid (300 mg, 1.57 mmol, 1 eq) in DCM (6 mL) was added (COCl)₂ (797.50 mg, 6.28 mmol, 550.00 μL, 4 eq) dropwise at 0° C. And DMF (114.81 mg, 1.57 mmol, 120.85 μL, 1 eq) was added the mixture. The mixture was stirred at 20° C. for 1 h. The reaction mixture was concentrated under reduced pressure to yield methyl 2-chlorocarbonylthiazole-4-carboxylate (300 mg, crude) as a yellow solid which was used in the next step without further purification.

### Step 3: Methyl 2-(benzimidazole-1-carbonyl)thiazole-4-carboxylate

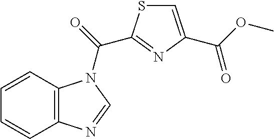 [SMILES: COC(=O)c1csc(C(=O)n2cnc3ccccc32)n1]

To a solution of benzimidazole (344.72 mg, 2.92 mmol, 2 eq) in pyridine (2.5 mL) was added the mixture solution of methyl 2-chlorocarbonylthiazole-4-carboxylate (300 mg, 1.46 mmol, 1 eq) in DCM (8 mL). The mixture was stirred at 20° C. for 1 h. The reaction mixture was concentrated under reduced pressure to yield a residue which was purified by flash silica gel chromatography (from PE/EtOAc=200/1 to 3/1, TLC:PE/EtOAc=1/1, R_{f}=0.45) to yield methyl 2-(benzimidazole-1-carbonyl)thiazole-4-carboxylate (24.99 mg, 86.98 μmol, 5.9% yield, 100% purity) as a white solid. ¹H NMR (500 MHz, DMSO-d₆) δ ppm 9.65 (s, 1H), 9.07 (s, 1H), 8.30-8.25 (m, 1H), 7.88-7.82 (m, 1H), 7.50-7.46 (m, 2H), 3.94 (s, 3H); ES-LCMS m/z 288.0 [M+H]⁺.

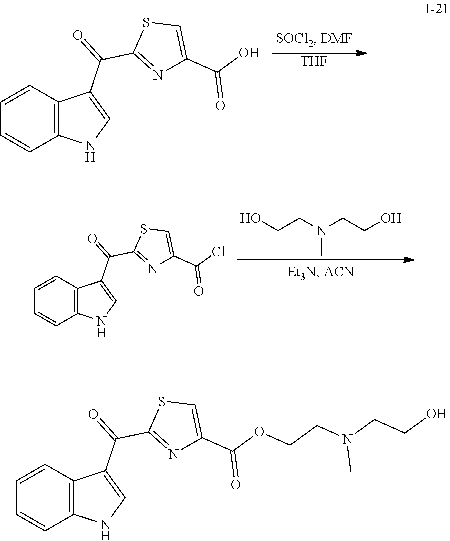

Step 1: 2-(1H-Indole-3-carbonyl)thiazole-4-carbonyl chloride

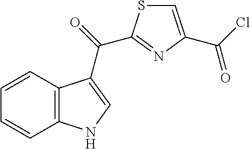 [SMILES: O=C(Cl)c1csc(C(=O)c2c[nH]c3ccccc23)n1]

To a stirred solution of 2-(1H-indole-3-carbonyl)thiazole-4-carboxylic acid (200 mg, 734.54 μmol, 1 eq) in THF (30 mL) was added SOCl₂ (6.56 g, 55.14 mmol, 4 mL, 75.07 eq) and DMF (53.69 mg, 734.54 μmol, 56.52 μL, 1 eq). The reaction mixture was stirred at 25° C. for 2 h. TLC (PE/EtOAc=3/1, R_{f}=0.50) showed starting material was almost consumed and one new spot was detected. The reaction mixture was concentrated to yield 2-(1H-indole-3-carbonyl)thiazole-4-carbonyl chloride (210 mg, crude) as a yellow solid which was used in the next step without further purification.

### Step 2: 2-[2-Hydroxyethyl(methyl)amino]ethyl 2-(1H-indole-3-carbonyl)thiazole-4-carboxylate

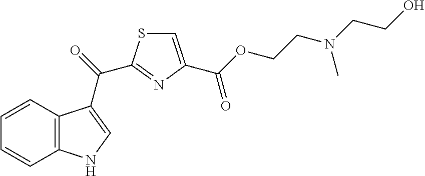 [SMILES: CN(CCO)CCOC(=O)c1csc(C(=O)c2c[nH]c3ccccc23)n1]

To a stirred solution of 2-(1H-indole-3-carbonyl)thiazole-4-carbonyl chloride (210 mg, 722.33 μmol, 1 eq) in ACN (20 mL) was added TEA (219.28 mg, 2.17 mmol, 301.62 μL, 3 eq) and 2-[2-hydroxyethyl(methyl)amino]ethanol (1.04 g, 8.73 mmol, 1 mL, 12.08 eq). The reaction mixture was stirred at 20° C. for 12 h. The reaction mixture was concentrated to yield a residue which was purified by preparative HPLC (Basic condition; column: Phenomenex Synergi C18 150*30 mm*4 um; mobile phase: [water (0.05% HCl)-ACN]; B %: 18%-38%, 10 min). The desired fraction was lyophilized to yield 2-[2-hydroxyethyl(methyl)amino]ethyl 2-(1H-indole-3-carbonyl)thiazole-4-carboxylate (48.79 mg, 130.66 μmol, 18.1% yield, 100.0% purity) as a white solid. ¹H NMR (400 MHz, CDCl₃) δ ppm 9.37 (d, J=3.2 Hz, 1H), 9.28 (s, 1H), 8.55-8.46 (m, 2H), 7.51-7.44 (m, 1H), 7.39-7.30 (m, 2H), 4.51 (t, J=5.1 Hz, 2H), 3.72 (t, J=5.3 Hz, 2H), 2.93 (t, J=5.1 Hz, 2H), 2.76 (t, J=5.4 Hz, 2H), 2.43 (s, 3H); ES-LCMS m/z 374.1 [M+H]⁺.

### I-35

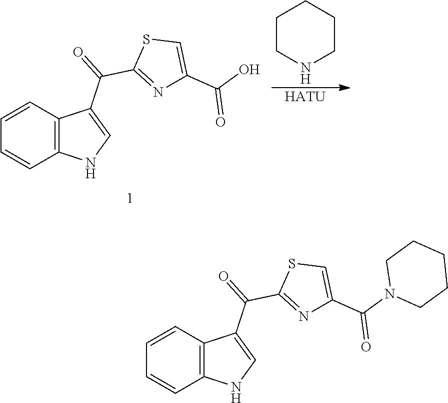

### Step 1: 1H-indol-3-yl-[4-(piperidine-1-carbonyl)thiazol-2-yl]methanone

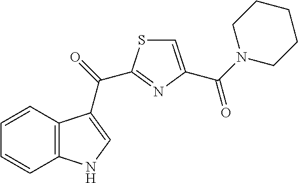 [SMILES: O=C(c1nc(C(=O)N2CCCCC2)cs1)c1c[nH]c2ccccc12]

To a solution of 2-(1H-indole-3-carbonyl)thiazole-4-carboxylic acid (300 mg, 1.10 mmol, 1 eq) and piperidine (469.08 mg, 5.51 mmol, 544.05 μL, 5 eq) in DMF (10 mL) was added HATU (628.41 mg, 1.65 mmol, 1.5 eq) and DIEA (427.19 mg, 3.31 mmol, 575.73 μL, 3 eq). The mixture was stirred at 25° C. for 3 h. The resulting product was concentrated under reduced pressure to yield a residue which was purified by preparative HPLC (column: Agela DuraShell C18 150*25 mm*5 μm; mobile phase: [water (0.04% NH₃.H₂O+10 mM NH₄HCO₃)-ACN]; B %: 37%-67%, 10 min), followed by lyophilization to yield 1H-indol-3-yl-[4-(piperidine-1-carbonyl)thiazol-2-yl]methanone (63.95 mg, 188.41 μmol, 17.1% yield, 100% purity) as a yellow solid. ¹H NMR (500 MHz, MeOD) δ ppm 9.08 (s, 1H), 8.40-8.36 (m, 1H), 8.27 (s, 1H), 7.53-7.50 (m, 1H), 7.32-7.26 (m, 2H), 3.79 (d, J=4.7 Hz, 4H), 1.81-1.72 (m, 6H); ES-LCMS m/z 340.0 [M+H]⁺.

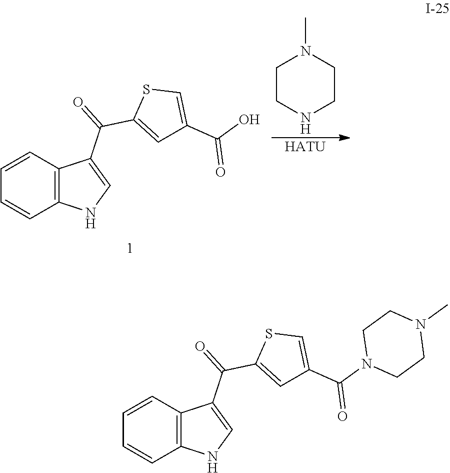

### Step 1: 1H-Indol-3-yl-[4-(4-methylpiperazine-1-carbonyl)thiazol-2-yl]methanone

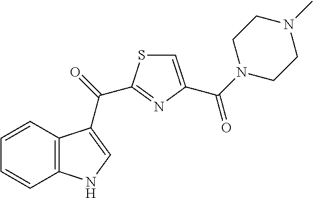 [SMILES: CN1CCN(C(=O)c2csc(C(=O)c3c[nH]c4ccccc34)n2)CC1]

To a solution of 2-(1H-indole-3-carbonyl)thiazole-4-carboxylic acid (300 mg, 1.10 mmol, 1 eq) and 1-methylpiperazine (551.80 mg, 5.51 mmol, 611.07 μL, 5 eq) in DMF (10 mL) was added HATU (628.41 mg, 1.65 mmol, 1.5 eq) and DIEA (427.19 mg, 3.31 mmol, 575.73 μL, 3 eq). The mixture was stirred at 25° C. for 3 h. The resulting product was concentrated under reduced pressure to yield a residue which was purified by preparative HPLC (column: Agela DuraShell C18 150*25 mm*5 μm; mobile phase: [water (0.04% NH₃.H₂O+10 mM NH₄HCO₃)-ACN]; B %: 29%-59%, 10 min), followed by lyophilization to yield 1H-indol-3-yl-[4-(4-methylpiperazine-1-carbonyl)thiazol-2-yl]methanone (80 mg, 225.72 μmol, 20.5% yield, 100% purity) as a yellow solid. (23.12 mg of product was delivered and 56.88 mg of product was used in the next step.) ¹H NMR (500 MHz, CD₃OD) δ ppm 9.04 (s, 1H), 8.37 (dd, J=2.8, 6.0 Hz, 1H), 8.33 (s, 1H), 7.53-7.49 (m, 1H), 7.32-7.25 (m, 2H), 3.95-3.84 (m, 4H), 2.58 (s, 4H), 2.37 (s, 3H); ES-LCMS m/z 355.1 [M+H]⁺.

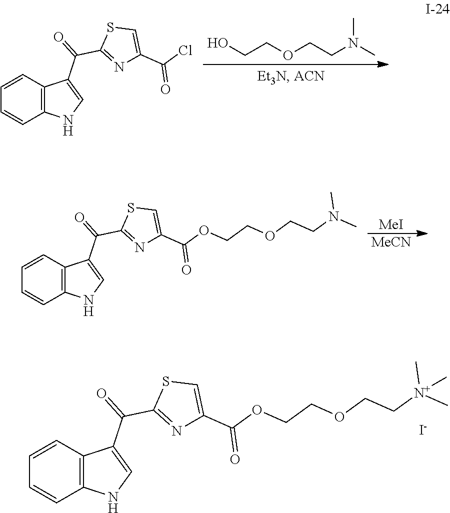

### Step 1: 2-[2-(Dimethylamino)ethoxy]ethyl 2-(1H-indole-3-carbonyl)thiazole-4-carboxylate

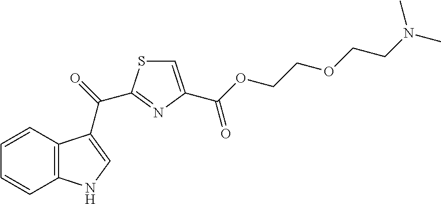 [SMILES: CN(C)CCOCCOC(=O)c1csc(C(=O)c2c[nH]c3ccccc23)n1]

To a stirred solution of 2-[2-(dimethylamino)ethoxy]ethanol (1.83 g, 13.76 mmol, 1.92 mL, 10 eq) in ACN (20 mL) was added TEA (417.67 mg, 4.13 mmol, 574.52 μL, 3 eq). 2-(1H-indole-3-carbonyl)thiazole-4-carbonyl chloride (400 mg, 1.38 mmol, 1 eq) was dissolved in ACN (10 mL) then added to the above mixture dropwise. The reaction mixture was stirred at 20° C. for 30 min. The reaction mixture was concentrated to yield a residue which was purified by preparative HPLC (Basic condition; column: Agela DuraShell C18 150*25 mm*5 um; mobile phase: [water (0.04% NH₃.H₂O+10 mM NH₄HCO₃)-ACN]; B %: 41%-71%, 10 min). The desired fraction was lyophilized to yield 2-[2-(dimethylamino)ethoxy]ethyl 2-(1H-indole-3-carbonyl)thiazole-4-carboxylate (220 mg, 567.81 μmol, 41.3% yield, 100.0% purity) as a white solid. ¹H NMR (400 MHz, DMSO-d₆) δ ppm 9.10 (s, 1H), 8.86 (s, 1H), 8.34-8.29 (m, 1H), 7.62-7.57 (m, 1H), 7.34-7.27 (m, 2H), 4.50-4.43 (m, 2H), 3.80-3.73 (m, 2H), 3.58 (t, J=5.9 Hz, 2H), 2.43 (t, J=5.9 Hz, 2H), 2.14 (s, 6H); ES-LCMS m/z 388.0 [M+H]⁺.

### Step 2: 2-[2-[BLAH(Trimethyl)-azanyl]ethoxy]ethyl 2-(1H-indole-3-carbonyl)thiazole-4-carboxylate

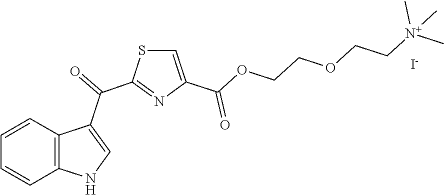 [SMILES: C[N+](C)(C)CCOCCOC(=O)c1csc(C(=O)c2c[nH]c3ccccc23)n1 ; [I-]]

To a stirred solution of 2-[2-(dimethylamino)ethoxy]ethyl 2-(1H-indole-3-carbonyl)thiazole-4-carboxylate (120 mg, 309.72 μmol, 1 eq) in ACN (20 mL) was added CH₃I (1.32 g, 9.29 mmol, 578.43 μL, 30 eq). The reaction mixture was stirred at 20° C. for 1 h. The reaction mixture was concentrated to yield 2-[2-[BLAH(trimethyl)-azanyl]ethoxy]ethyl 2-(1H-indole-3-carbonyl)thiazole-4-carboxylate (143.06 mg, 258.62 μmol, 83.5% yield, 95.7% purity) as a yellow solid which was lyophilized for delivery without further purification. ¹H NMR (400 MHz, DMSO-d₆) δ ppm 12.41 (s, 1H), 9.08 (s, 1H), 8.86 (s, 1H), 8.35-8.28 (m, 1H), 7.64-7.58 (m, 1H), 7.35-7.26 (m, 2H), 4.56-4.48 (m, 2H), 3.93 (s, 2H), 3.88-3.82 (m, 2H), 3.59-3.52 (m, 2H), 3.08 (s, 9H); ES-LCMS m/z 402.1 [M-I]⁺.

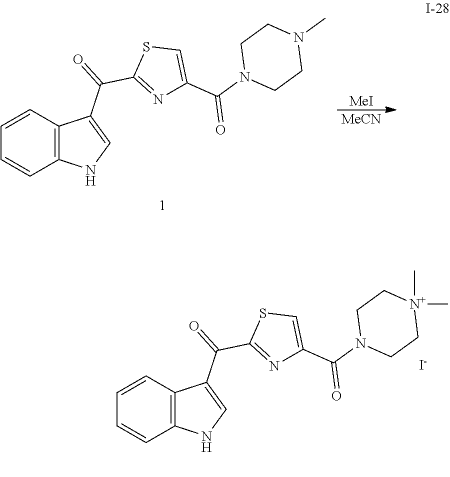 [SMILES: "MeI" ; "MeCN" ; CN1CCN(C(=O)c2csc(C(=O)c3c[nH]c4ccccc34)n2)CC1 ; C[N+]1(C)CCN(C(=O)c2csc(C(=O)c3c[nH]c4ccccc34)n2)CC1 ; [I-]]

### Step 1: [4-(4-BLAH-4,4-dimethyl-1,4diazinane-1-carbonyl)thiazol-2-yl]-(1H-indol-3-yl)methanone

To a solution of 1H-indol-3-yl-[4-(4-methylpiperazine-1-carbonyl)thiazol-2-yl]methanone (30 mg, 84.64 μmol, 1 eq) in acetonitrile (5 mL) was added CH₃I (1.98 g, 13.95 mmol, 868.42 μL, 164.80 eq). The mixture was stirred at 25° C. for 1 h. To the mixture was added water (5 mL) and lyophilized to yield[4-(4-BLAH-4,4-dimethyl-1,4diazinane-1-carbonyl)thiazol-2-yl]-(1H-indol-3-yl)methanone (32.59 mg, 65.66 μmol, 77.6% yield, 100% purity) as a yellow solid. ¹H NMR (500 MHz, DMSO-d₆) δ ppm 12.40 (s, 1H), 8.98 (s, 1H), 8.58 (s, 1H), 8.33-8.29 (m, 1H), 7.61-7.58 (m, 1H), 7.33-7.27 (m, 2H), 4.24-4.00 (m, 4H), 3.54 (d, J=13.4 Hz, 4H), 3.23 (s, 6H); ES-LCMS m/z 369.1 [M-I⁻]⁺.

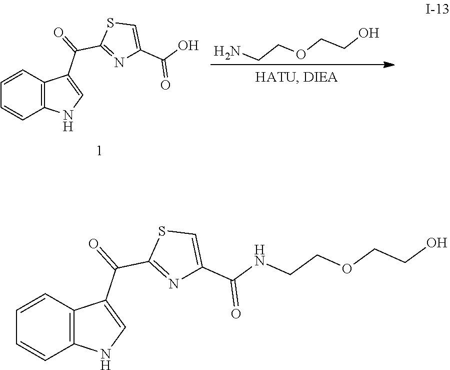 [SMILES: O=C(O)c1csc(C(=O)c2c[nH]c3ccccc23)n1 ; NCCOCCO ; O=C(NCCOCCO)c1csc(C(=O)c2c[nH]c3ccccc23)n1]

### Step 1: 4-Chloro-2-iodo-pyrimidine

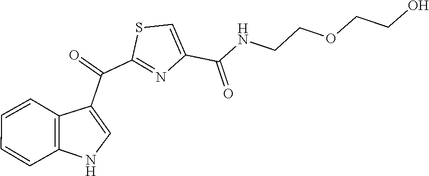 [SMILES: O=C(NCCOCCO)c1csc(C(=O)c2c[nH]c3ccccc23)n1]

To a stirred solution of 2-(1H-indole-3-carbonyl)thiazole-4-carboxylic acid (100 mg, 348.91 μmol, 1 eq) and 2-(2-aminoethoxy)ethanol (55.02 mg, 523.36 μmol, 52.40 μL, 1.5 eq) in DMF (5 mL) was added HATU (199.00 mg, 523.36 μmol, 1.5 eq) and DIEA (90.19 mg, 697.81 μmol, 121.55 μL, 2 eq). The reaction mixture was stirred at 25° C. for 12 h. The reaction mixture was filtered and the filtrate was purified by preparative HPLC (column: Agela DuraShell C18 150*25 mm*5 um; mobile phase: [water (0.04% NH₃.H₂O+10 mM NH₄HCO₃)-ACN]; B %: 20%-50%, 10 min). The desired fraction was lyophilized to yield N-[2-(2-hydroxyethoxy)ethyl]-2-(1H-indole-3-carbonyl)thiazole-4-carboxamide (55.91 mg, 154.29 μmol, 44.2% yield, 99.2% purity) as a yellow solid. ¹H NMR (500 MHz, DMSO-d₆) δ ppm 9.40 (s, 1H), 8.76 (t, J=5.6 Hz, 1H), 8.62 (s, 1H), 8.36-8.29 (m, 1H), 7.58 (d, J=7.0 Hz, 1H), 7.34-7.24 (m, 2H), 3.64-3.58 (m, 2H), 3.55-3.47 (m, 6H), 3.33-3.29 (m, 2H); ES-LCMS m/z 360.0 [M+H]⁺.

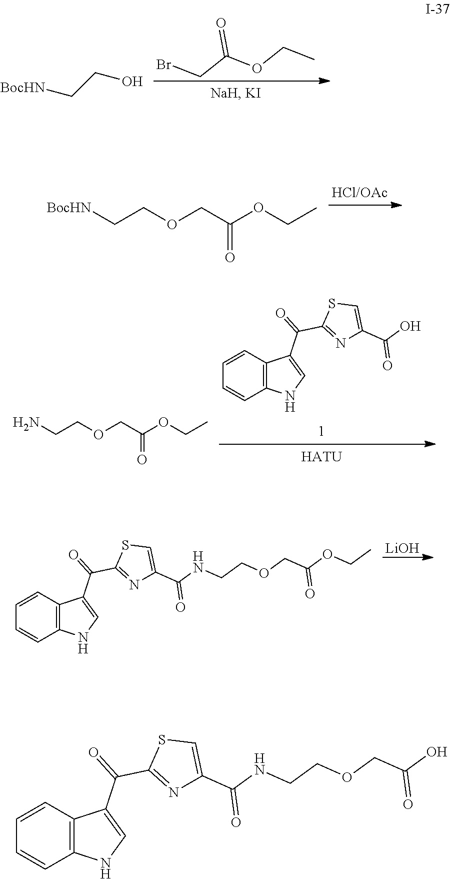

### Step 1: Ethyl 2-[2-(tert-butoxycarbonylamino)ethoxy]acetate

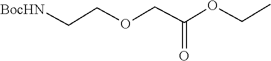 [SMILES: CCOC(=O)COCCNC(=O)OC(C)(C)C]

To a mixture of tert-butyl N-(2-hydroxyethyl)carbamate (2 g, 12.41 mmol, 1.92 mL, 1 eq), NaI (297.56 mg, 1.99 mmol, 0.16 eq) in THF (40 mL) was added NaH (793.97 mg, 19.85 mmol, 60% purity, 1.6 eq) at 0° C. Then the mixture was stirred at 0° C. for 0.5 h. Ethyl 2-bromoacetate (4.14 g, 24.81 mmol, 2.74 mL, 2 eq) was added to above mixture dropwise at 0° C. The mixture was stirred for 2.5 h at 20° C. under N₂ atmosphere. TLC (PE/EA=3/1, R_{f}=0.3) showed starting material disappeared and a new spot was formed. The reaction mixture was filtered and the filtrate was concentrated under reduced pressure to yield a residue which was purified by flash silica gel chromatography (ISCO®; 20 g SEPAFLASH® Silica Flash Column, Eluent of 0-30% Ethylacetate/Petroleum ethergradient @ 30 mL/min) to yield the product of ethyl 2-[2-(tert-butoxycarbonylamino)ethoxy]acetate (2.3 g, 8.84 mmol, 71.22% yield, 95.0% purity) as light yellow oil. ¹H NMR (500 MHz, CDCl₃) δ ppm 5.14 (s, 1H), 4.20 (q, J=7.1 Hz, 2H), 4.07-4.03 (m, 2H), 3.58 (t, J=4.9 Hz, 2H), 3.32 (d, J=4.9 Hz, 2H), 1.42 (s, 9H), 1.27 (t, J=7.2 Hz, 3H).

### Step 2: Ethyl 2-(2-aminoethoxy)acetate

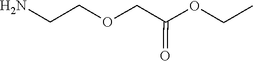

A solution of ethyl 2-[2-(tert-butoxycarbonylamino)ethoxy]acetate (300 mg, 1.15 mmol, 1 eq) in HCl/EtOAc (4 M, 9.50 mL, 32.97 eq) was stirred for 0.5 h at 20° C. TLC (PE/EA=3/1, R_{f}=0.3) showed starting material disappeared. The reaction mixture was concentrated under reduced pressure to yield the product of ethyl 2-(2-aminoethoxy)acetate (210 mg, 1.09 mmol, 94.3% yield, 95.0% purity, HCl) as light yellow oil which was used for the next step without further purification. ¹H NMR (400 MHz, CD₃OD) δ ppm 4.25-4.20 (m, 2H), 4.19 (s, 2H), 3.79-3.74 (m, 2H), 3.14 (t, J=4.9 Hz, 2H), 1.27 (t, J=7.2 Hz, 3H).

### Step 3: Ethyl 2-[2-[[2-(1H-indole-3-carbonyl)thiazole-4-carbonyl]amino]ethoxy]acetate

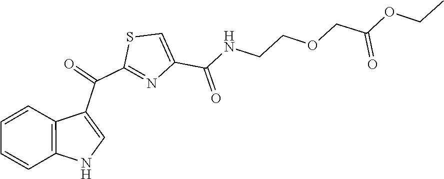 [SMILES: CCOC(=O)COCCNC(=O)c1csc(C(=O)c2c[nH]c3ccccc23)n1]

To a stirred solution of 2-(1H-indole-3-carbonyl)thiazole-4-carboxylic acid (160 mg, 558.25 μmol, 1 eq) in DMF (5 mL) was added HATU (318.40 mg, 837.38 μmol, 1.5 eq), DIEA (288.60 mg, 2.23 mmol, 388.95 μL, 4 eq) and ethyl 2-(2-aminoethoxy)acetate (120 mg, 620.80 μmol, 1.11 eq, HCl). The reaction mixture was stirred at 25° C. for 4 h. The reaction mixture was diluted with water (100 mL) and extracted with EtOAc (50 mL×3). The combined organic layers were dried over Na₂SO₄, filtered and the filtrate was concentrated to yield ethyl 2-[2-[[2-(1H-indole-3-carbonyl)thiazole-4-carbonyl]amino]ethoxy]acetate (160 mg, 358.71 μmol, 64.3% yield, 90.0% purity) as a yellow solid which was used in the next step without further purification. ¹H NMR (500 MHz, DMSO-d₆) δ ppm 9.39 (d, J=3.1 Hz, 1H), 8.75 (t, J=5.6 Hz, 1H), 8.62 (s, 1H), 8.36-8.30 (m, 1H), 7.88 (s, 1H), 7.58 (dd, J=2.0, 6.4 Hz, 1H), 7.34-7.26 (m, 2H), 4.18 (s, 2H), 3.69 (t, J=6.0 Hz, 2H), 3.54 (q, J=5.8 Hz, 2H), 3.20 (q, J=5.7 Hz, 2H), 1.29-1.22 (m, 3H); ES-LCMS m/z 402.0 [M+H]⁺.

### Step 4: 2-[2-[BLAH(Trimethyl)-azanyl]ethoxy]ethyl 2-(1H-indole-3-carbonyl)thiazole-4-carboxylate

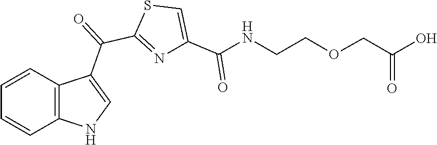 [SMILES: O=C(O)COCCNC(=O)c1csc(C(=O)c2c[nH]c3ccccc23)n1]

To a stirred solution of ethyl 2-[2-[[2-(1H-indole-3-carbonyl)thiazole-4-carbonyl]amino]ethoxy]acetate (160 mg, 358.71 μmol, 1 eq) in a solution of MeOH (5 mL) and water (1 mL) was added LiOH (68.72 mg, 2.87 mmol, 8 eq). The reaction mixture was stirred at 25° C. for 1 h. The reaction mixture was concentrated to yield a residue which was purified by preparative HPLC (column: Agela DuraShell C18 150*25 mm*5 um; mobile phase: [water (0.04% NH₃.H₂O+10 mM NH₄HCO₃)-ACN]; B %: 8%-38%, 10 min). The desired fraction was lyophilized to yield 2-[2-[[2-(1H-indole-3-carbonyl)thiazole-4-carbonyl]amino]ethoxy]acetic acid (50.59 mg, 135.49 μmol, 37.8% yield, 100.0% purity) as a yellow solid. ¹H NMR (400 MHz, DMSO-d₆) δ ppm 10.18-10.00 (m, 2H), 8.59 (s, 1H), 8.31-8.27 (m, 1H), 7.60-7.55 (m, 1H), 7.28-7.22 (m, 2H), 3.78 (s, 2H), 3.73 (t, J=5.1 Hz, 2H), 3.43 (s, 2H); ES-LCMS m/z 374.0 [M+H]⁺.

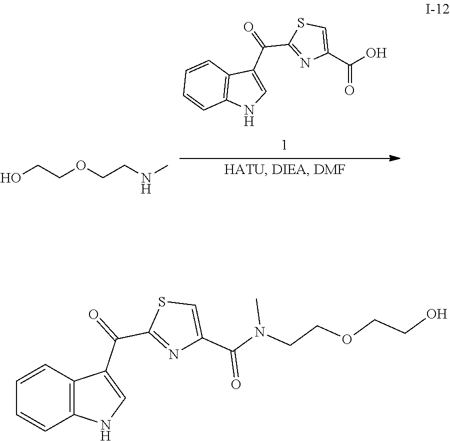

### Step 1: N-[2-(2-Hydroxyethoxy)ethyl]-2-(1H-indole-3-carbonyl)-N-methyl-thiazole-4-carboxamide

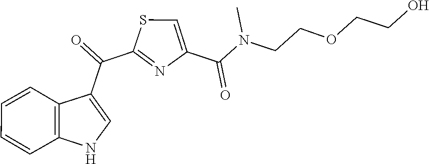 [SMILES: CN(CCOCCO)C(=O)c1csc(C(=O)c2c[nH]c3ccccc23)n1]

To a stirred solution of 2-(1H-indole-3-carbonyl)thiazole-4-carboxylic acid (100 mg, 348.91 μmol, 1 eq) and 2-[2-(methylamino)ethoxy]ethanol (50 mg, 419.60 μmol, 52.40 μL, 1.2 eq) in DMF (5 mL) was added HATU (199.00 mg, 523.36 μmol, 1.5 eq) and DIEA (90.19 mg, 697.81 μmol, 121.54 μL, 2 eq). The reaction mixture was stirred at 25° C. for 12 h. The reaction mixture was filtered and the filtrate was purified by preparative HPLC (column: Agela DuraShell C18 150*25 mm*5 um; mobile phase: [water (0.04% NH₃.H₂O+10 mM NH₄HCO₃)-ACN]; B %: 27%-57%, 10 min). The desired fraction was lyophilized to yield N-[2-(2-hydroxyethoxy)ethyl]-2-(1H-indole-3-carbonyl)-N-methyl-thiazole-4-carboxamide (29.96 mg, 80.23 μmol, 23.0% yield, 100.0% purity) as white solid. ¹H NMR (400 MHz, DMSO-d₆) δ ppm 8.95 (s, 1H), 8.42-8.38 (m, 1H), 8.33 (d, J=8.5 Hz, 1H), 7.57 (d, J=5.8 Hz, 1H), 7.32-7.26 (m, 2H), 3.78-3.67 (m, 4H), 3.49 (d, J=16.3 Hz, 4H), 3.18 (s, 3H); ES-LCMS m/z 374.0 [M+H]⁺.

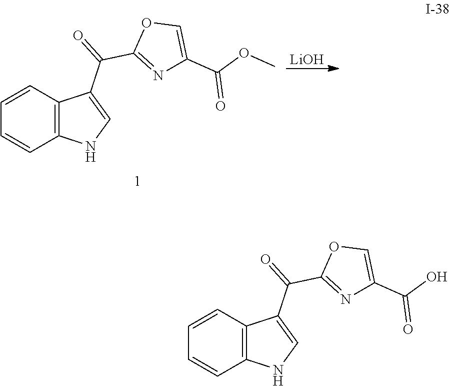

### Step 1: 2-(1H-Indole-3-carbonyl)oxazole-4-carboxylic acid

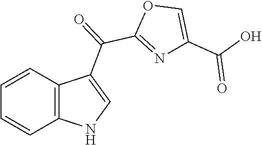 [SMILES: O=C(O)c1coc(C(=O)c2c[nH]c3ccccc23)n1]

To a solution of methyl 2-(1H-indole-3-carbonyl)oxazole-4-carboxylate (100 mg, 370.04 μmol, 1 eq) in MeOH (5 mL) and H₂O (5 mL) was added LiOH.H₂O (124.23 mg, 2.96 mmol, 8 eq). The mixture was stirred at 25° C. for 1 h. The reaction mixture was concentrated under reduced pressure to yield a residue which was purified by preparative HPLC (column: Welch Xtimate C18 150*30 mm*5 μm; mobile phase: [water (10 mM NH₄HCO₃)-ACN]; B %: 0%-28%, 10 min), followed by lyophilization to yield 2-(1H-indole-3-carbonyl)oxazole-4-carboxylic acid (55.76 mg, 217.63 μmol, 58.8% yield, 100% purity) as a yellow solid. ¹H NMR (400 MHz, DMSO-d₆) δ ppm 9.02 (s, 1H), 8.51 (s, 1H), 8.29 (dd, J=2.7, 5.9 Hz, 1H), 7.57 (d, J=6.7 Hz, 1H), 7.31-7.24 (m, 2H); ES-LCMS m/z 257.0 [M+H]⁺.

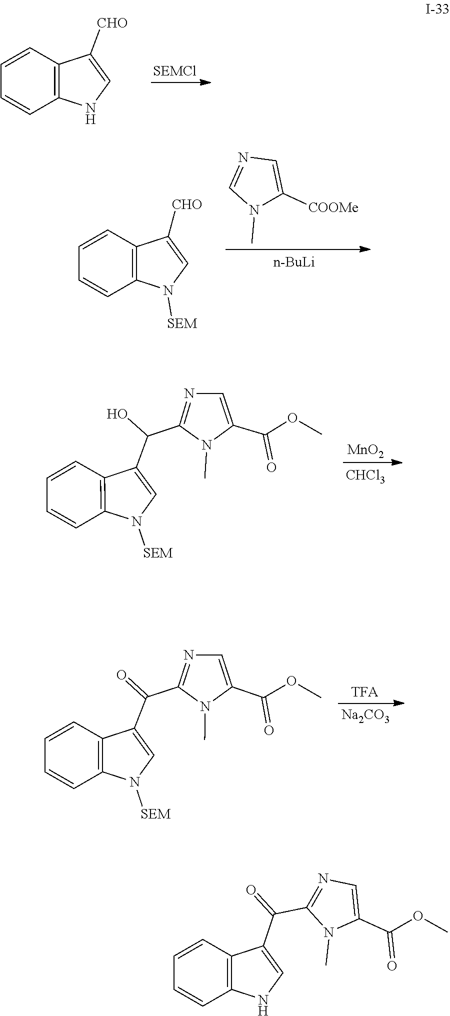

### Step 1: 1-(2-Trimethylsilylethoxymethyl)indole-3-carbaldehyde

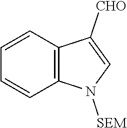 [SMILES: C[Si](C)(C)CCOCn1cc(C=O)c2ccccc21]

To a stirred solution of 1H-indole-3-carbaldehyde (7 g, 48.22 mmol, 1 eq) in THF (150 mL) was cooled to 0° C. then added NaH (2.89 g, 72.33 mmol, 60% in mineral oil, 1.5 eq) partwise under N₂ atmosphere. The reaction mixture was stirred at 0° C. for 30 min under N₂ atmosphere. SEM-Cl (9.65 g, 57.87 mmol, 10.24 mL, 1.2 eq) was added to the above mixture dropwise then stirred at 0° C. for 2 h under N₂ atmosphere. TLC (PE/EtOAc=3/1, R_{f}=0.40) showed starting material was consumed completely and one new spot was detected. The reaction mixture was diluted with water (150 mL) then extracted with EtOAc (200 mL×3). The combined organic layers were dried over Na₂SO₄, filtered and the filtrate was concentrated to yield a residue which was purified by flash silica gel chromatography (from PE/EtOAc=100/1 to 3/1, TLC:PE/EtOAc=3/1, R_{f}=0.40) to yield 1-(2-trimethylsilylethoxymethyl)indole-3-carbaldehyde (10.2 g, 37.03 mmol, 76.80% yield, 100% purity) as yellow oil. ¹H NMR (500 MHz, CDCl₃) δ ppm 10.07 (s, 1H), 8.34-8.31 (m, 1H), 7.81 (s, 1H), 7.56-7.53 (m, 1H), 7.38-7.35 (m, 2H), 5.55 (s, 2H), 3.53 (dd, J=7.7, 8.6 Hz, 2H), 0.94-0.90 (m, 2H), −0.03-−0.05 (m, 9H); ES-LCMS m/z 276.0 [M+H]⁺.

### Step 2: Methyl 2-[hydroxy-[1-(2-trimethylsilylethoxymethyl)indol-3-yl]methyl]-3-methyl-imidazole-4-carboxylate

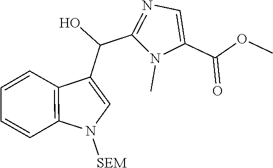 [SMILES: COC(=O)c1cnc(C(O)c2cn(COCC[Si](C)(C)C)c3ccccc23)n1C]

To a stirred solution of DIPA (734.81 mg, 7.26 mmol, 1.03 mL, 2 eq) in THF (30 mL) was cooled to −75° C. then added n-BuLi (2.5 M, 3.63 mL, 2.5 eq) dropwise under N₂ atmosphere. The reaction mixture was stirred at −75° C. for 30 min under N₂ atmosphere. Methyl 3-methylimidazole-4-carboxylate (500 mg, 3.39 mmol, 0.93 eq) was dissolved in THF (10 mL) then added to the above reaction mixture then stirred at −75° C. for 30 min under N₂ atmosphere. 1-(2-Trimethylsilylethoxymethyl)indole-3-carbaldehyde (1 g, 3.63 mmol, 1 eq) was dissolved in THF (10 mL) then added to the above reaction mixture. The reaction mixture was stirred at −75° C. for 2 h under N₂ atmosphere. The reaction mixture was concentrated to remove THF. The residue was dissolved in water (100 mL) then extracted with EtOAc (100 mL×3). The combined organic layers were dried over Na₂SO₄, filtered and the filtrate was concentrated to yield a residue which was purified by flash silica gel chromatography (from PE/EtOAc=100/1 to 1/100, TLC:PE/EtOAc=1/3, R_{f}=0.35) to yield methyl 2-[hydroxy-[1-(2-trimethylsilylethoxymethyl)indol-3-yl]methyl]-3-methyl-imidazole-4-carboxylate (170 mg, 378.00 μmol, 10.4% yield, 92.4% purity) as yellow oil. ¹H NMR (500 MHz, CDCl₃) δ ppm 7.80-7.77 (m, 1H), 7.52 (d, J=7.9 Hz, 1H), 7.48 (d, J=8.2 Hz, 1H), 7.27-7.24 (m, 1H), 7.16-7.12 (m, 1H), 7.07 (s, 1H), 6.17 (d, J=4.0 Hz, 1H), 5.44 (s, 2H), 3.85 (s, 3H), 3.72 (s, 3H), 3.50-3.43 (m, 2H), 0.90-0.86 (m, 2H), −0.05-−0.07 (m, 9H); ES-LCMS m/z 416.1 [M+H]⁺.

### Step 3: 3-Methyl-2-[1-(2-trimethylsilylethoxymethyl)indole-3-carbonyl]imidazole-4-carboxylate

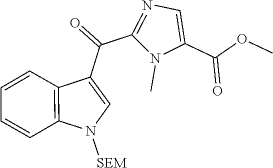 [SMILES: COC(=O)c1cnc(C(=O)c2cn(COCC[Si](C)(C)C)c3ccccc23)n1C]

To a stirred solution of methyl 2-[hydroxy-[1-(2-trimethylsilylethoxymethyl)indol-3-yl]methyl]-3-methyl-imidazole-4-carboxylate (160 mg, 355.76 μmol, 1 eq) in CHCl₃ (10 mL) was added MnO₂ (618.60 mg, 7.12 mmol, 20 eq). The reaction mixture was stirred at 50° C. for 12 h. The reaction mixture was filtered through a pad of celite and the filtered cake was washed with DCM (50 mL×3). The combined organic layers were concentrated to yield 3-methyl-2-[1-(2-trimethylsilylethoxymethyl)indole-3-carbonyl]imidazole-4-carboxylate (150 mg, 344.58 μmol, 96.9% yield, 95.0% purity) as yellow oil which was used in the next step without further purification. ¹H NMR (400 MHz, CDCl₃) δ ppm 8.81-8.76 (m, 1H), 8.57-8.52 (m, 1H), 7.82 (d, J=0.7 Hz, 1H), 7.60-7.55 (m, 1H), 7.40-7.35 (m, 2H), 5.57 (s, 2H), 4.36 (d, J=0.7 Hz, 3H), 3.93 (d, J=0.7 Hz, 3H), 3.58-3.51 (m, 2H), 0.91 (t, J=8.1 Hz, 2H), −0.05 (d, J=0.7 Hz, 8H); ES-LCMS m/z 414.1 [M+H]⁺.

### Step 4: Methyl 2-(1H-indole-3-carbonyl)-5-methyl-thiazole-4-carboxylate

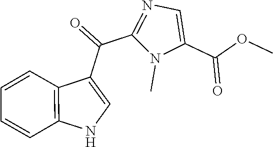 [SMILES: COC(=O)c1cnc(C(=O)c2c[nH]c3ccccc23)n1C]

To a stirred solution of methyl 3-methyl-2-[1-(2-trimethylsilylethoxymethyl)indole-3-carbonyl]imidazole-4-carboxylate (140 mg, 321.61 μmol, 1 eq) in DCM (1 mL) was added TFA (3.08 g, 27.01 mmol, 2.00 mL, 83.99 eq). The reaction mixture was stirred at 25° C. for 1 h. TLC (PE/EtOAc=1/1, R_{f}=0.20) showed starting material was consumed completely and one new spot was detected. The reaction mixture was concentrated at 25° C. to yield a residue which was dissolved in DCM (10 mL). The mixture was concentrated to yield a residue which was dissolved in MeOH (2 mL). The mixture was adjusted pH to 9 by saturated NaHCO₃ solution then stirred at 25° C. for 2 h. The reaction mixture was diluted with water (30 mL) then extracted with EtOAc (30 mL×3). The combined organic layers were concentrated to yield a residue which was purified by preparative HPLC (column: Agela DuraShell C18 150*25 mm*5 um; mobile phase: [water (0.04% NH₃.H₂O+10 mM NH₄HCO₃)-ACN]; B %: 28%-58%, 10 min). The desired fraction was lyophilized to yield methyl 2-(1H-indole-3-carbonyl)-3-methyl-imidazole-4-carboxylate (75.35 mg, 265.99 μmol, 82.7% yield, 100.0% purity) as white solid. ¹H NMR (400 MHz, CDCl₃) δ ppm 8.87-8.66 (m, 2H), 8.55 (d, J=7.1 Hz, 1H), 7.82 (d, J=2.0 Hz, 1H), 7.48-7.43 (m, 1H), 7.40-7.31 (m, 2H), 4.36 (d, J=2.0 Hz, 3H), 3.93 (d, J=1.7 Hz, 3H); ES-LCMS m/z 283.9 [M+H]⁺.

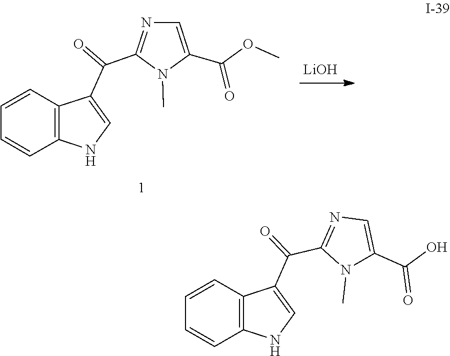

### Step 1: 2-(1H-Indole-3-carbonyl)-3-methyl-imidazole-4-carboxylic acid

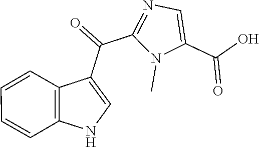 [SMILES: Cn1c(C(=O)O)cnc1C(=O)c1c[nH]c2ccccc12]

To a stirred solution of methyl 2-(1H-indole-3-carbonyl)-3-methyl-imidazole-4-carboxylate (60 mg, 211.80 μmol, 1 eq) in a solution of THF (5 mL) and water (1 mL) was added LiOH (40.58 mg, 1.69 mmol, 8 eq). The reaction mixture was stirred at 25° C. for 1 h. The reaction mixture was concentrated to yield a residue which was purified by preparative HPLC (column: Welch Xtimate C18 150*30 mm*5 um; mobile phase: [water (10 mM NH₄HCO₃)-ACN]; B %: 0%-25%, 10 min). The desired fraction was lyophilized to yield 2-(1H-indole-3-carbonyl)-3-methyl-imidazole-4-carboxylic acid (49.64 mg, 184.36 μmol, 87.0% yield, 100.0% purity) as white solid. ¹H NMR (400 MHz, DMSO-d₆) δ ppm 8.82-8.75 (m, 1H), 8.38-8.31 (m, 1H), 7.55-7.48 (m, 1H), 7.37 (s, 1H), 7.26-7.18 (m, 1H), 7.26-7.18 (m, 2H), 4.25 (s, 3H); ES-LCMS m/z 269.8 [M+H]⁺.

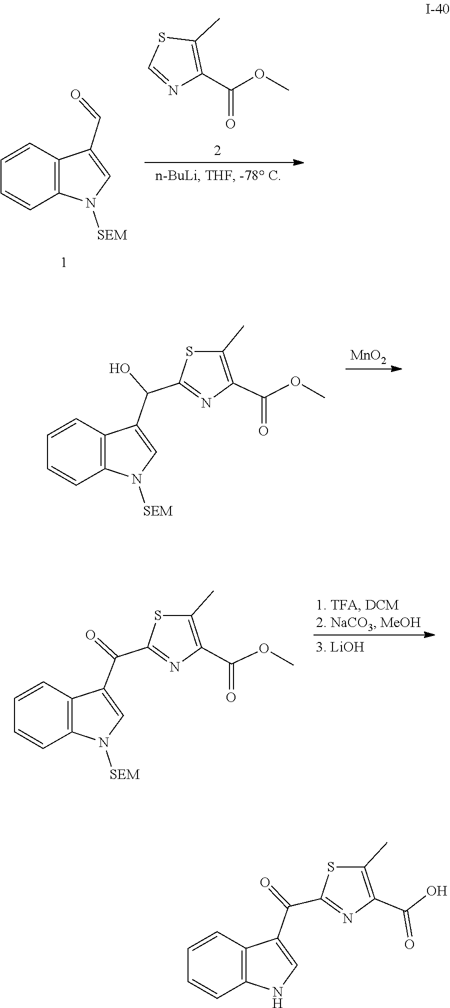

### Step 1: Methyl 2-[hydroxy-[1-(2-trimethylsilylethoxymethyl)indol-3-yl]methyl]-5-methyl-thiazole-4-carboxylate

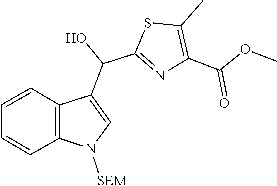 [SMILES: COC(=O)c1nc(C(O)c2cn(COCC[Si](C)(C)C)c3ccccc23)sc1C]

To a stirred solution of DIPA (551.11 mg, 5.45 mmol, 769.71 μL, 2 eq) in THF (30 mL) was cooled to −75° C. then added n-BuLi (2.5 M, 2.18 mL, 2 eq) dropwise under N₂ atmosphere. The reaction mixture was stirred at −75° C. for 30 min under N₂ atmosphere. Methyl 5-methylthiazole-4-carboxylate (500 mg, 3.18 mmol, 1.17 eq) and 1-(2-trimethylsilylethoxymethyl)indole-3-carbaldehyde (750 mg, 2.72 mmol, 1 eq) was dissolved in THF (20 mL). The LDA reaction mixture was added to the above mixture then stirred at −75° C. for 10 min under N₂ atmosphere. The reaction mixture was concentrated to remove THF. The residue was dissolved in water (100 mL) then extracted with EtOAc (100 mL×3). The combined organic layers were dried over Na₂SO₄, filtered and the filtrate was concentrated to yield a residue which was purified by flash silica gel chromatography (from PE/EtOAc=100/1 to 1/100, TLC:PE/EtOAc=1/3, R_{f}=0.35) to yield methyl 2-[hydroxy-[1-(2-trimethylsilylethoxymethyl)indol-3-yl]methyl]-5-methyl-thiazole-4-carboxylate (470 mg, 1.03 mmol, 37.9% yield, 95.0% purity) as yellow oil. ¹H NMR (500 MHz, CDCl₃) δ ppm 7.64 (d, J=7.9 Hz, 1H), 7.49 (d, J=8.2 Hz, 1H), 7.26 (s, 2H), 7.19-7.12 (m, 1H), 6.36 (d, J=3.2 Hz, 1H), 5.47 (d, J=0.9 Hz, 2H), 3.94 (s, 3H), 3.51-3.44 (m, 2H), 3.14 (d, J=3.7 Hz, 1H), 2.75-2.70 (m, 3H), 0.93-0.85 (m, 2H), −0.02-−0.09 (m, 9H); ES-LCMS m/z 433.1 [M+H]⁺.

### Step 2: 5-Methyl-2-[1-(2-trimethylsilylethoxymethyl)indole-3-carbonyl]thiazole-4-carboxylate

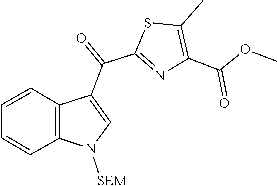 [SMILES: COC(=O)c1nc(C(=O)c2cn(COCC[Si](C)(C)C)c3ccccc23)sc1C]

To a stirred solution of methyl 2-[hydroxy-[1-(2-trimethylsilylethoxymethyl)indol-3-yl]methyl]-5-methyl-thiazole-4-carboxylate (470 mg, 1.03 mmol, 1 eq) in CHCl₃ (50 mL) was added MnO₂ (1.35 g, 15.48 mmol, 15 eq). The reaction mixture was stirred at 50° C. for 12 h. The reaction mixture was filtered through a pad of celite and the filtered cake was washed with DCM (50 mL×3). The combined organic layers were concentrated to yield 5-methyl-2-[1-(2-trimethylsilylethoxymethyl)indole-3-carbonyl]thiazole-4-carboxylate (450 mg, 992.82 μmol, 96.2% yield, 95.0% purity) as yellow solid which was used in the next step without further purification. ¹H NMR (400 MHz, CDCl₃) δ ppm 9.09 (s, 1H), 8.56-8.49 (m, 1H), 7.63-7.55 (m, 1H), 7.42-7.34 (m, 2H), 5.62 (s, 2H), 4.00 (s, 3H), 3.57 (t, J=8.1 Hz, 2H), 2.88 (s, 3H), 0.93 (t, J=8.1 Hz, 2H), −0.05 (s, 9H); ES-LCMS m/z 431.1 [M+H]⁺.

### Step 3: 2-(1H-Indole-3-carbonyl)-5-methyl-thiazole-4-carboxylic acid

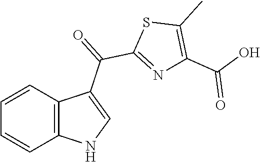 [SMILES: Cc1sc(C(=O)c2c[nH]c3ccccc23)nc1C(=O)O]

To a stirred solution of methyl 5-methyl-2-[1-(2-trimethylsilylethoxymethyl)indole-3-carbonyl]thiazole-4-carboxylate (450 mg, 992.82 μmol, 1 eq) in DCM (5 mL) was added TFA (7.70 g, 67.53 mmol, 5.00 mL, 68.02 eq). The reaction mixture was stirred at 25° C. for 1 h. TLC (PE/EtOAc=1/1, R_{f}=0.20) showed starting material was consumed completely and one new spot was detected. The reaction mixture was concentrated at 25° C. to yield a residue which was dissolved in DCM (10 mL). The mixture was concentrated to yield a residue which was dissolved in MeOH (10 mL). The mixture was adjusted pH to 9 by saturated Na₂CO₃ solution then stirred at 25° C. for 2 h. LiOH (237.78 mg, 9.93 mmol, 10 eq) was added the above mixture then stirred at 25° C. for 12 h. The reaction mixture was diluted with water (50 mL) then extracted with EtOAc (30 mL×3). The combined organic layers were concentrated to yield a residue which was purified by preparative HPLC (column: Welch Xtimate C18 150*30 mm*5 um; mobile phase: [water (10 mM NH₄HCO₃)-ACN]; B %: 5%-35%, 10 min). The desired fraction was lyophilized to yield 2-(1H-indole-3-carbonyl)-5-methyl-thiazole-4-carboxylic acid (200 mg, 698.55 μmol, 70.4% yield, 100.0% purity) as yellow solid. 50 mg of compound 2-(1H-indole-3-carbonyl)-5-methyl-thiazole-4-carboxylic acid was purified by preparative HPLC (column: Agela DuraShell C18 150*25 mm*5 um; mobile phase: [water (0.04% NH₃.H₂O+10 mM NH₄HCO₃)-ACN]; B %: 6%-36%, 10 min) to remove little DMSO then 15.67 mg was delivered by lyophilization. ¹H NMR (400 MHz, CD₃OD) δ ppm 9.39 (s, 1H), 8.40-8.29 (m, 1H), 7.54-7.45 (m, 1H), 7.31-7.20 (m, 2H), 2.83 (s, 3H); ES-LCMS m/z 286.9 [M+H]⁺. 1-41

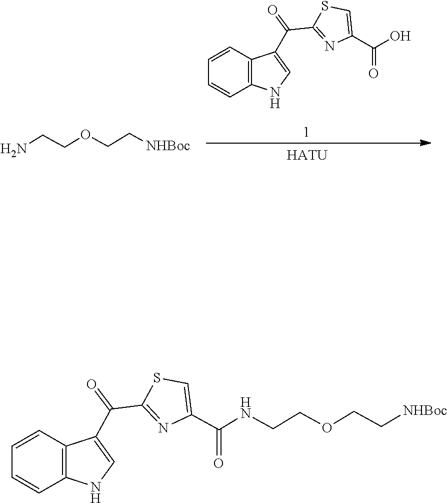 [SMILES: O=C(O)c1csc(C(=O)c2c[nH]c3ccccc23)n1 ; CC(C)(C)OC(=O)NCCOCCN ; "HATU" ; CC(C)(C)OC(=O)NCCOCCNC(=O)c1csc(C(=O)c2c[nH]c3ccccc23)n1]

### Step 1: tert-Butyl N-[2-[2-[[2-(1H-indole-3-carbonyl)thiazole-4-carbonyl]amino]ethoxy]ethyl]carbamate

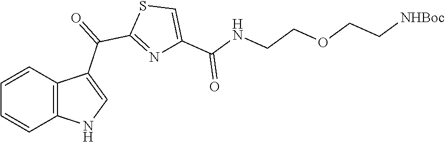 [SMILES: CC(C)(C)OC(=O)NCCOCCNC(=O)c1csc(C(=O)c2c[nH]c3ccccc23)n1]

To a solution of 2-(1H-indole-3-carbonyl)thiazole-4-carboxylic acid (420.94 mg, 1.47 mmol, 1 eq) in DMF (8 mL) was added DIEA (569.43 mg, 4.41 mmol, 767.43 μL, 3 eq), HATU (1.01 g, 2.64 mmol, 1.8 eq) and tert-butyl N-[2-(2-aminoethoxy)ethyl]carbamate (300 mg, 1.47 mmol, 1 eq). The mixture was stirred at 25° C. for 3 h. To the mixture was added water (100 mL) and extracted with ethyl acetate (100 mL×3). The combined organic phase was washed with brine (20 mL), dried with anhydrous Na₂SO₄, filtered and concentrated in vacuum to yield a residue which was purified by flash silica gel chromatography (from PE/EtOAc=200/1 to 0/1, TLC:PE/EtOAc=0/1, R_{f}=0.40) to yield a product. The product was purified by preparative HPLC (column: Agela DuraShell C18 150*25 mm*5 μm; mobile phase: [water (0.04% NH₃.H₂O+10 mM NH₄HCO₃)-ACN]; B %: 38%-68%, 10 min), followed by lyophilization to yield tert-butyl N-[2-[2-[[2-(1H-indole-3-carbonyl)thiazole-4-carbonyl]amino]ethoxy]ethyl]carbamate (230 mg, 496.59 μmol, 33.8% yield, 99% purity) as a yellow solid (17.5 mg of the product was used to delivery, 212.5 mg of the product was used in the next step). ¹H NMR (500 MHz, CD₃OD) δ ppm 9.31 (s, 1H), 8.50 (s, 1H), 8.40-8.36 (m, 1H), 7.56-7.50 (m, 1H), 7.33-7.27 (m, 2H), 3.71-3.65 (m, 4H), 3.57 (t, J=5.7 Hz, 2H), 3.29-3.26 (m, 2H), 1.43-1.32 (m, 9H); ES-LCMS m/z 459.1 [M+H]⁺.

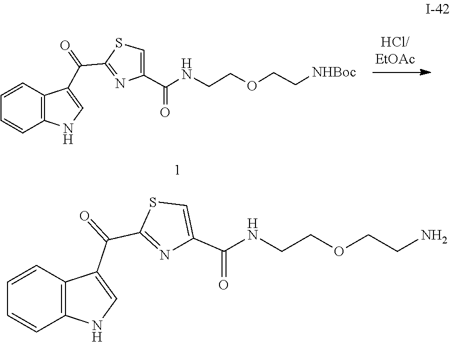 [SMILES: CC(C)(C)OC(=O)NCCOCCNC(=O)c1csc(C(=O)c2c[nH]c3ccccc23)n1 ; NCCOCCNC(=O)c1csc(C(=O)c2c[nH]c3ccccc23)n1]

### Step 1: N-[2-(2-Aminoethoxy)ethyl]-2-(1H-indole-3-carbonyl)thiazole-4-carboxamide

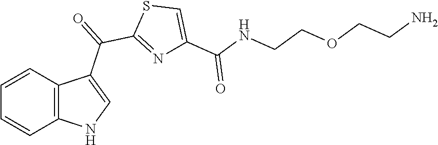 [SMILES: NCCOCCNC(=O)c1csc(C(=O)c2c[nH]c3ccccc23)n1]

A solution of tert-butyl N-[2-[2-[[2-(1H-indole-3-carbonyl)thiazole-4-carbonyl]amino]ethoxy]ethyl]carbamate (50 mg, 107.95 μmol, 1 eq) in HCl/EtOAc (2 mL) was stirred at 25° C. for 1 h. The reaction mixture was concentrated under reduced pressure to yield a residue which was purified by preparative HPLC (column: Phenomenex Synergi C18 150*30 mm*4 μm; mobile phase: [water (0.05% HCl)-ACN]; B %: 17%-37%, 10 min), followed by lyophilization to yield the product. The product was further purified by preparative HPLC (column: Agela DuraShell C18 150*25 mm*5 μm; mobile phase: [water (0.04% NH₃.H₂O+10 mM NH₄HCO₃)-ACN]; B %: 22%-52%, 10 min), followed by lyophilization to yield N-[2-(2-aminoethoxy)ethyl]-2-(1H-indole-3-carbonyl)thiazole-4-carboxamide (16.56 mg, 46.20 μmol, 42.8% yield, 100% purity) as a yellow solid. ¹H NMR (400 MHz, CD₃OD) δ ppm 9.29 (s, 1H), 8.50 (s, 1H), 8.42-8.34 (m, 1H), 7.56-7.50 (m, 1H), 7.32-7.27 (m, 2H), 3.71 (d, J=4.3, 8.6 Hz, 4H), 3.62-3.62 (m, 1H), 3.62 (t, J=5.3 Hz, 2H), 2.90 (t, J=5.1 Hz, 2H); ES-LCMS m/z 359.0 [M+H]⁺.

### I-34

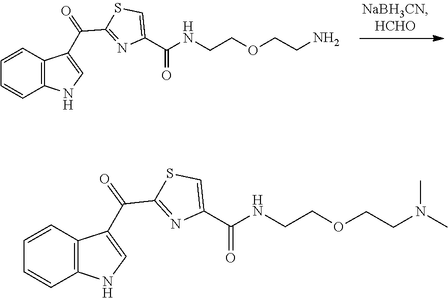 [SMILES: NCCOCCNC(=O)c1csc(C(=O)c2c[nH]c3ccccc23)n1 ; CN(C)CCOCCNC(=O)c1csc(C(=O)c2c[nH]c3ccccc23)n1]

### Step 1: N-[2-[2-(Dimethylamino)ethoxy]ethyl]-2-(1H-indole-3-carbonyl)thiazole-4-carboxamide

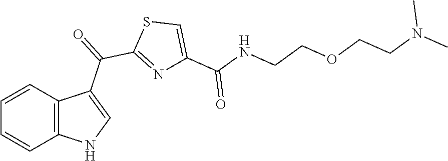 [SMILES: CN(C)CCOCCNC(=O)c1csc(C(=O)c2c[nH]c3ccccc23)n1]

To a solution of N-[2-(2-aminoethoxy)ethyl]-2-(1H-indole-3-carbonyl)thiazole-4-carboxamide (60 mg, 129.15 umol, 1 eq, HCl) in MeOH (5 mL) was added HCHO (38.79 mg, 1.29 mmol, 35.58 μL, 10 eq) and NaBH₃CN (40.58 mg, 645.77 μmol, 5 eq). The mixture was stirred at 25° C. for 12 h. The reaction mixture was filtered to yield the filter liquor which was purified by preparative HPLC (column: Agela DuraShell C18 150*25 mm*5 μm; mobile phase: [water (0.04% NH₃.H₂O+10 mM NH₄HCO₃)-ACN]; B %: 31%-51%, 10 min), followed by lyophilization to yield N-[2-[2-(dimethylamino)ethoxy]ethyl]-2-(1H-indole-3-carbonyl)thiazole-4-carboxamide (18.44 mg, 47.71 μmol, 36.9% yield, 100% purity) as a yellow solid. ¹H NMR (500 MHz, CD₃OD) δ ppm 9.29 (d, J=2.0 Hz, 1H), 8.48 (d, J=1.8 Hz, 1H), 8.37 (dd, J=2.1, 5.7 Hz, 1H), 7.55-7.49 (m, 1H), 7.32-7.24 (m, 2H), 3.70-3.62 (m, 6H), 2.59 (t, J=5.5 Hz, 2H), 2.27 (s, 6H); ES-LCMS m/z 387.0 [M+H]⁺.

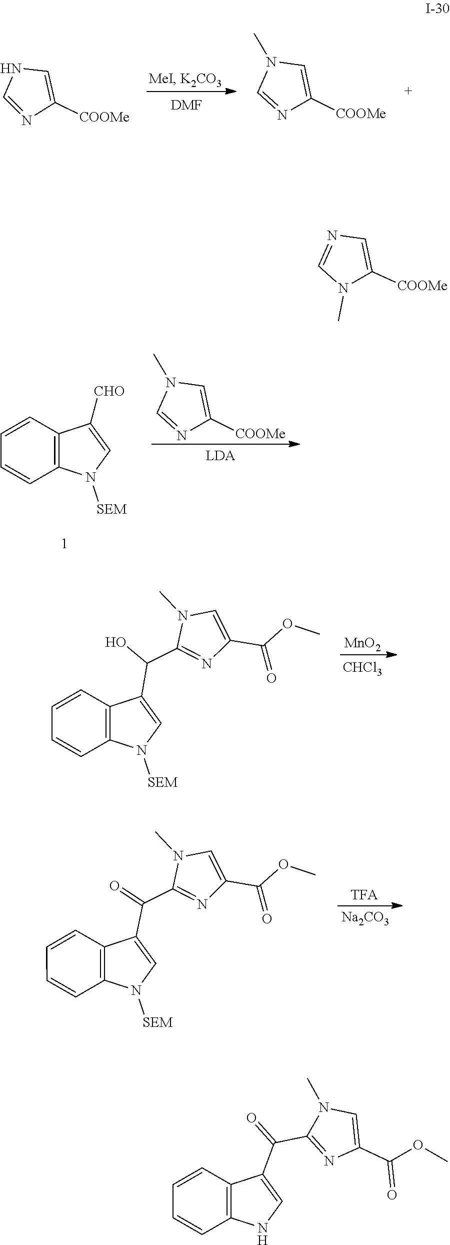

### Step 1: 3-Methylimidazole-4-carboxylate & Methyl 1-methylimidazole-4-carboxylate

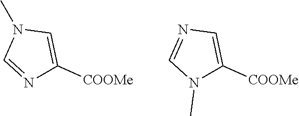

To a solution of methyl 1H-imidazole-4-carboxylate (10 g, 79.29 mmol, 1 eq) in DMF (75 mL) was added MeI (17.79 g, 125.33 mmol, 7.80 mL, 1.58 eq) and K₂CO₃ (27.40 g, 198.23 mmol, 2.5 eq). The mixture was stirred at 15° C. for 3 h. TLC (DCM/MeOH=10/1, R_{f}=0.36, 0.31) indicated most of the starting material was consumed and two new spots formed. The reaction mixture was filtered, concentrated to yield a residue which was purified by flash silica gel chromatography (from DCM/MeOH=1/0 to 10/1, TLC:DCM/MeOH=10/1, R_{f}=0.36, 0.31) to yield methyl 3-methylimidazole-4-carboxylate (3.5 g, 23.73 mmol, 29.9% yield, 95% purity) as a yellow solid, ¹H NMR (400 MHz, CDCl₃) δ ppm 7.71 (d, J=0.8 Hz, 1H), 7.54 (s, 1H), 3.90 (s, 3H), 3.85 (s, 3H), And methyl 1-methylimidazole-4-carboxylate (2.0 g, 13.56 mmol, 17.10% yield, 95% purity) as a yellow solid. ¹H NMR (400 MHz, CDCl₃) δ ppm 7.58 (d, J=1.2 Hz, 1H), 7.50 (s, 1H), 3.89 (s, 3H), 3.75 (s, 3H).

### Step 2: Methyl 2-[hydroxy-[1-(2-trimethylsilylethoxymethyl)indol-3-yl]methyl]-1-methyl-imidazole-4-carboxylate

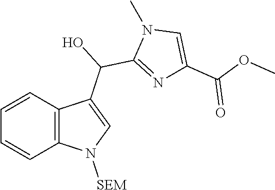 [SMILES: COC(=O)c1cn(C)c(C(O)c2cn(COCC[Si](C)(C)C)c3ccccc23)n1]

To a solution of i-Pr₂NH (1.37 g, 13.56 mmol, 1.92 mL, 2.0 eq) in THF (30 mL) was added n-BuLi (2.5 M, 5.42 mL, 2.0 eq) at −70° C. under N₂. After being stirred for 0.5 h, a solution of methyl 1-methylimidazole-4-carboxylate (1 g, 6.78 mmol, 1 eq) in THF (60 mL) was added slowly. After another 0.5 h, a solution of 1-(2-trimethylsilylethoxymethyl)indole-3-carbaldehyde (2.05 g, 7.46 mmol, 1.1 eq) in THF (5 mL) was added dropwise. The mixture was stirred at 15° C. for 12 h under N₂ atmosphere. The reaction mixture was quenched by addition of water (100 mL), extracted with EtOAc (100 mL×3). The combined organic layers were washed with brine (30 mL), dried over Na₂SO₄, filtered and concentrated under reduced pressure to yield a residue which was purified by flash silica gel chromatography (from DCM/MeOH=1/0 to 10/1, TLC:DCM /MeOH=10/1, R_{f}=0.22) to yield methyl 2-[hydroxy-[1-(2-trimethylsilylethoxymethyl)indol-3-yl]methyl]-1-methyl-imidazole-4-carboxylate (350 mg, 838.87 μmol, 12.3% yield, 99.6% purity) as yellow oil. ¹H NMR (500 MHz, CDCl₃) δ ppm 7.73-7.68 (m, 1H), 7.47 (d, J=8.2 Hz, 1H), 7.44 (s, 1H), 7.31-7.27 (m, 1H), 7.21-7.16 (m, 1H), 6.76 (s, 1H), 6.47 (d, J=7.2 Hz, 1H), 5.59-5.49 (m, 1H), 5.41-5.35 (m, 2H), 3.97-3.91 (m, 3H), 3.60-3.53 (m, 3H), 3.46-3.39 (m, 2H), 0.89-0.80 (m, 2H), −0.02-−0.12 (m, 9H); ES-LCMS m/z 416.1 [M+H]⁺.

### Step 3: Methyl 1-methyl-2-[1-(2-trimethylsilylethoxymethyl)indole-3-carbonyl]imidazole-4-carboxylate

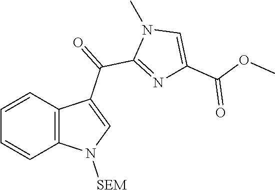 [SMILES: COC(=O)c1cn(C)c(C(=O)c2cn(COCC[Si](C)(C)C)c3ccccc23)n1]

To a solution of methyl 2-[hydroxy-[1-(2-trimethylsilylethoxymethyl)indol-3-yl]methyl]-1-methyl-imidazole-4-carboxylate (350 mg, 838.87 μmol, 1 eq) in CHCl₃ (35 mL) was added MnO₂ (1.09 g, 12.58 mmol, 15 eq). The mixture was stirred at 50° C. for 12 h. The residue was filtered, concentrated to yield methyl 1-methyl-2-[1-(2-trimethylsilylethoxymethyl)indole-3-carbonyl]imidazole-4-carboxylate (340 mg, 652.80 μmol, 77.8% yield, 79.4% purity) as brown oil which was used in the next step without further purification. ¹H NMR (400 MHz, CD₃OD) δ ppm 8.37 (dd, J=2.9, 6.1 Hz, 1H), 7.64-7.51 (m, 3H), 7.43-7.34 (m, 2H), 5.47 (s, 2H), 3.72 (s, 3H), 3.71-3.67 (m, 3H), 3.54-3.43 (m, 2H), 0.94-0.83 (m, 3H), −0.05 (s, 9H); ES-LCMS m/z 414.1 [M+H]⁺.

### Step 4: Methyl 2-(1H-indole-3-carbonyl)-1-methyl-imidazole-4-carboxylate

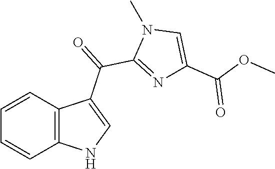 [SMILES: COC(=O)c1cn(C)c(C(=O)c2c[nH]c3ccccc23)n1]

To a solution of methyl 1-methyl-2-[1-(2-trimethylsilylethoxymethyl)indole-3-carbonyl]imidazole-4-carboxylate (140 mg, 268.80 μmol, 1 eq) in DCM (4 mL) was added TFA (6.16 g, 54.03 mmol, 4 mL, 200.99 eq). The mixture was stirred at 15° C. for 0.5 h. TLC (Plate 1: DCM/MeOH=10/1, R_{f}=0.33) indicated the starting material was consumed completely and one new spot formed. The solution was concentrated below 30° C. to yield a residue which was dissolved in MeOH (4 mL), adjusted pH to 9 by aq. Na₂CO₃. The resulting mixture was stirred at 15° C. for 1 h. The slurry was filtered, and the cake was rinsed with MeOH (2×10 mL). The filtrate was concentrated to yield a residue which was purified by preparative HPLC (column: Welch Xtimate C18 150*30 mm*5 μm; mobile phase: [water (10 mM NH₄HCO₃)-ACN]; B %: 20%-50%, 10 min), followed by lyophilization to yield methyl 2-(1H-indole-3-carbonyl)-1-methyl-imidazole-4-carboxylate (10.7 mg, 36.22 μmol, 13.4% yield, 95.9% purity) as a white solid. ¹H NMR (400 MHz, DMSO-d₆) δ ppm 8.13 (d, J=8.6 Hz, 1H), 7.84 (s, 1H), 7.77 (s, 1H), 7.52-7.45 (m, 1H), 7.31-7.19 (m, 2H), 3.58 (s, 3H), 3.50 (s, 3H); ES-LCMS m/z 284.0 [M+H]⁺.

### I-29

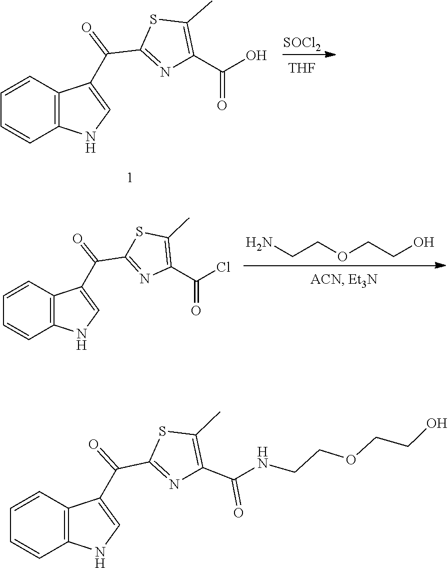

### Step 1: 2-(1H-Indole-3-carbonyl)-5-methyl-thiazole-4-carbonyl chloride

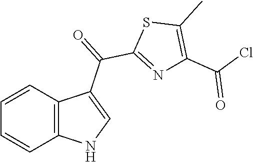 [SMILES: Cc1sc(C(=O)c2c[nH]c3ccccc23)nc1C(=O)Cl]

To a stirred solution of 2-(1H-indole-3-carbonyl)-5-methyl-thiazole-4-carboxylic acid (90 mg, 314.35 μmol, 1 eq) in THF (3 mL) was added SOCl₂ (863.16 mg, 7.26 mmol, 526.32 μL, 23.08 eq) and DMF (229.77 μg, 3.14 μmol, 0.01 eq). The reaction mixture was stirred at 25° C. for 1 h. The reaction mixture was concentrated to yield 2-(1H-indole-3-carbonyl)-5-methyl-thiazole-4-carbonyl chloride (90 mg, 295.32 μmol, 94.0% yield) as yellow solid which was used in the next step without further purification.

### Step 2: N-[2-(2-Hydroxyethoxy)ethyl]-2-(1H-indole-3-carbonyl)-5-methyl-thiazole-4-carboxamide

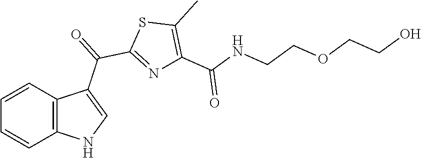 [SMILES: Cc1sc(C(=O)c2c[nH]c3ccccc23)nc1C(=O)NCCOCCO]

To a stirred solution of 2-(2-aminoethoxy)ethanol (294.96 mg, 2.81 mmol, 280.92 μL, 10 eq) in ACN (10 mL) was added Et₃N (85.17 mg, 841.67 μmol, 117.15 μL, 3 eq). 2-(1H-indole-3-carbonyl)-5-methyl-thiazole-4-carbonyl chloride (85.50 mg, 280.56 μmol, 1 eq) was added to the above reaction mixture then stirred at 25° C. for 1 h. The reaction mixture was concentrated to yield a residue which was purified by preparative HPLC (column: Welch Xtimate C18 150*30 mm*5 um; mobile phase: [water (10 mM NH₄HCO₃)-ACN]; B %: 28%-58%, 10 min). The desired fraction was lyophilized to yield N-[2-(2-hydroxyethoxy)ethyl]-2-(1H-indole-3-carbonyl)-5-methyl-thiazole-4-carboxamide (52.32 mg, 140.11 μmol, 49.9% yield, 100.0% purity) as yellow solid. ¹H NMR (400 MHz, DMSO-d₆) δ ppm 9.30 (s, 1H), 8.58 (t, J=5.9 Hz, 1H), 8.34-8.27 (m, 1H), 7.60-7.53 (m, 1H), 7.32-7.24 (m, 2H), 3.62-3.57 (m, 2H), 3.55-3.47 (m, 6H), 2.84 (s, 3H); ES-LCMS m/z 374.0 [M+H]⁺.

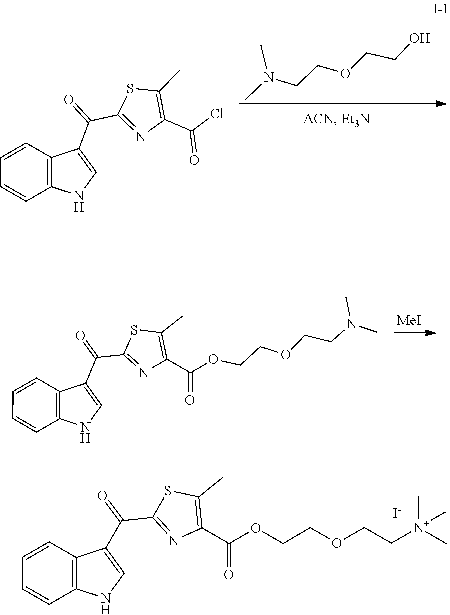

### Step 1: 2-[2-(Dimethylamino)ethoxy]ethyl 2-(1H-indole-3-carbonyl)-5-methyl-thiazole-4-carboxylate

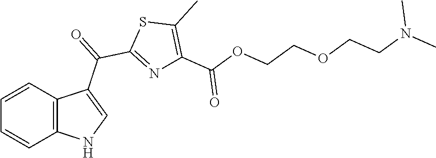 [SMILES: Cc1sc(C(=O)c2c[nH]c3ccccc23)nc1C(=O)OCCOCCN(C)C]

To a stirred solution of 2-[2-(dimethylamino)ethoxy]ethanol (961.49 mg, 7.22 mmol, 1.01 mL, 10 eq) in ACN (20 mL) was added Et₃N (219.15 mg, 2.17 mmol, 301.44 μL, 3 eq). 2-(1H-indole-3-carbonyl)-5-methyl-thiazole-4-carbonyl chloride (220 mg, 721.90 μmol, 1 eq) was added to the above reaction mixture then stirred at 25° C. for 1 h. The reaction mixture was concentrated to yield a residue which was purified by preparative HPLC (column: Agela DuraShell C18 150*25 mm*5 um; mobile phase: [water (0.04% NH₃.H₂O+10 mM NH₄HCO₃)-ACN]; B %: 38%-68%, 10 min). The desired fraction was lyophilized to yield 2-[2-(dimethylamino)ethoxy]ethyl 2-(1H-indole-3-carbonyl)-5-methyl-thiazole-4-carboxylate (40 mg, 99.63 μmol, 13.8% yield, 100.0% purity) as a yellow solid. ¹H NMR (400 MHz, DMSO-d₆) δ ppm 9.06 (s, 1H), 8.32-8.27 (m, 1H), 7.61-7.55 (m, 1H), 7.32-7.26 (m, 2H), 4.44 (dd, J=3.9, 5.4 Hz, 2H), 3.77 (dd, J=3.9, 5.4 Hz, 2H), 3.59 (t, J=5.9 Hz, 2H), 2.82 (s, 3H), 2.44 (t, J=5.9 Hz, 2H), 2.14 (s, 6H).

### Step 2: 2-(2-BLAHethoxy)ethyl 2-(1H-indole-3-carbonyl)-5-methyl-thiazole-4-carboxylate

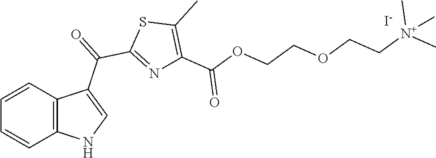 [SMILES: Cc1sc(C(=O)c2c[nH]c3ccccc23)nc1C(=O)OCCOCC[N+](C)(C)C ; [I-]]

To a stirred solution of 2-[2-(dimethylamino)ethoxy]ethyl 2-(1H-indole-3-carbonyl)-5-methyl-thiazole-4-carboxylate (40 mg, 99.63 μmol, 1 eq) in ACN (5 mL) was added MeI (70.71 mg, 498.16 μmol, 31.01 μL, 5 eq) dropwise at 0° C. under N₂ atmosphere. The reaction mixture was stirred at 25° C. for 1 h under N₂ atmosphere. The reaction mixture was concentrated and lyophilized to yield 2-(2-BLAHethoxy)ethyl 2-(1H-indole-3-carbonyl)-5-methyl-thiazole-4-carboxylate (38.95 mg, 71.68 μmol, 71.9% yield, 100.0% purity) as a yellow solid for delivery without further purification. ¹H NMR (400 MHz, DMSO-d₆) δ ppm 12.31 (s, 1H), 9.03 (d, J=2.9 Hz, 1H), 8.30 (dd, J=2.8, 6.0 Hz, 1H), 7.63-7.56 (m, 1H), 7.33-7.27 (m, 2H), 4.59-4.46 (m, 2H), 3.98-3.90 (m, 2H), 3.89-3.80 (m, 2H), 3.60-3.49 (m, 2H), 3.07 (s, 9H), 2.83 (s, 3H); ES-LCMS m/z 416.16 [M-I⁻]⁺.

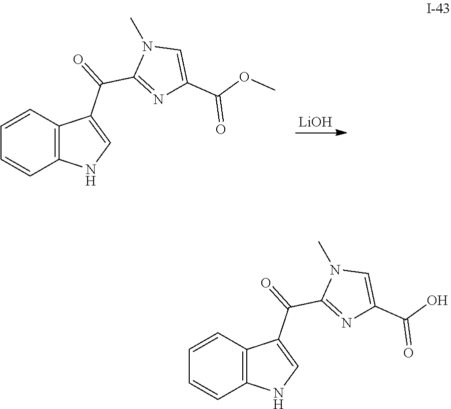

### Step 1: Methyl 2-(1H-indole-3-carbonyl)-1-methyl-imidazole-4-carboxylate

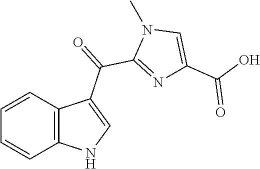 [SMILES: Cn1cc(C(=O)O)nc1C(=O)c1c[nH]c2ccccc12]

To a solution of methyl 2-(1H-indole-3-carbonyl)-1-methyl-imidazole-4-carboxylate (108 mg, 381.25 μmol, 1 eq) in THF (5 mL) and H₂O (1 mL) was added LiOH (45.65 mg, 1.91 mmol, 5 eq). The mixture was stirred at 20° C. for 1 h. The reaction mixture was concentrated to yield a residue which was purified by preparative HPLC (column: Welch Xtimate C18 150*30 mm*5 μm; mobile phase: [water (10 mM NH₄HCO₃)-ACN]; B %: 0%-30%, 10 min), followed by lyophilization to yield 2-(1H-indole-3-carbonyl)-1-methyl-imidazole-4-carboxylic acid (40.69 mg, 151.12 μmol, 39.6% yield, 100% purity) as a white solid, ¹H NMR (400 MHz, DMSO-d₆) δ ppm 8.14 (br s, 1H), 7.77 (br s, 2H), 7.48 (br s, 1H), 7.22 (br s, 2H), 3.55 (br s, 3H); ES-LCMS m/z 270.0 [M+H]⁺.

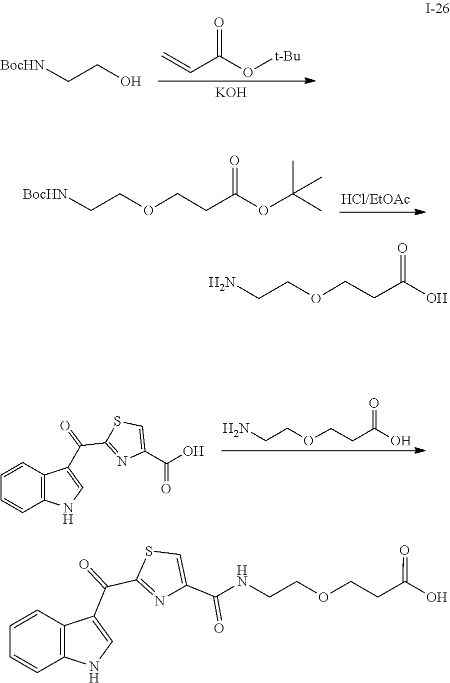

### Step 1: tert-Butyl 3-[2-(tert-butoxycarbonylamino)ethoxy]propanoate

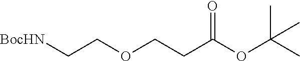 [SMILES: CC(C)(C)OC(=O)CCOCCNC(=O)OC(C)(C)C]

A mixture of tert-butyl N-(2-hydroxyethyl)carbamate (2 g, 12.41 mmol, 1.92 mL, 1 eq) and tert-butyl prop-2-enoate (3.18 g, 24.81 mmol, 3.60 mL, 2 eq) in 1,4-dioxane (5 mL) and 60% aqueous KOH solution (0.3 mL) was stirred at 25° C. for 12 h. TLC (PE/EtOAc=10/1, R_{f}=0.2) showed starting material was consumed and one major new spot was detected. The mixture was diluted with H₂O (30 mL) and extracted with EtOAc (60 mL×3). The combined organic layers were dried over anhydrous Na₂SO₄, filtered and concentrated yield a residue which was purified by flash silica gel chromatography (from pure PE to PE/EtOAc=4/1, TLC:PE/EtOAc=10/1, R_{f}=0.2) to yield tert-butyl 3-[2-(tert-butoxycarbonylamino)ethoxy]propanoate (2.8 g, 8.71 mmol, 70.2% yield, 90.0% purity) as colorless oil. ¹H NMR (500 MHz, DMSO-d₆) δ ppm 6.69 (br s, 1H), 3.56 (t, J=6.2 Hz, 2H), 3.34-3.26 (m, 2H), 3.04 (q, J=5.9 Hz, 2H), 2.40 (t, J=6.2 Hz, 2H), 1.41-1.35 (m, 18H).

### Step 2: 3-(2-Aminoethoxy)propanoic acid

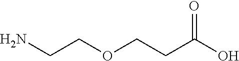

To a solution of tert-butyl 3-[2-(tert-butoxycarbonylamino)ethoxy]propanoate (1 g, 3.11 mmol, 1 eq) in MeOH (1.58 g, 49.42 mmol, 2 mL, 15.89 eq) was added HCl (12 M, 4 mL, 15.43 eq). The mixture was stirred at 25° C. for 12 h. TLC (PE/EtOAc=3/1, R_{f}=0) showed starting material was consumed completely and one major new spot was detected. The reaction mixture was concentrated to yield 3-(2-aminoethoxy)propanoic acid (350 mg, crude, HCl) as colorless oil. ¹H NMR (500 MHz, DMSO-d₆) δ ppm 12.31 (br s, 1H), 8.05 (br s, 2H), 3.68-3.62 (m, 2H), 3.61-3.58 (m, 2H), 2.97-2.88 (m, 2H), 2.58-2.52 (m, 2H).

### Step 3: 3-[2-[[2-(1H-Indole-3-carbonyl)thiazole-4-carbonyl]amino]ethoxy]propanoic acid

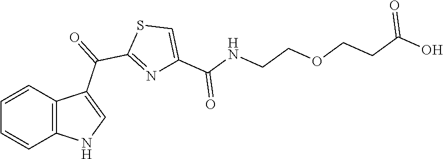 [SMILES: O=C(O)CCOCCNC(=O)c1csc(C(=O)c2c[nH]c3ccccc23)n1]

To a mixture of 2-(1H-indole-3-carbonyl)thiazole-4-carboxylic acid (200 mg, 712.50 μmol, 1 eq) and 3-(2-aminoethoxy)propanoic acid (104.35 mg, 783.75 μmol, 1.1 eq, HCl) in DMF (5 mL) was added HATU (325.10 mg, 855.00 μmol, 1.2 eq) and DIEA (184.17 mg, 1.43 mmol, 248.21 μL, 2 eq). The mixture was stirred at 60° C. for 12 h. TLC (EtOAc/MeOH=3/2, R_{f}=0.65) showed starting material was consumed and one major new spot was detected. The mixture was added H₂O (20 mL) and 1 N HCl (adjust pH<7) and extracted with EtOAc (20 mL×3). The combined organic layers were dried over anhydrous Na₂SO₄, filtered and concentrated to yield a residue which was purified by preparative TLC (EtOAc/MeOH=3/2, TLC:EtOAc/MeOH=3/2, R_{f}=0.65) and concentrated to yield a residue which was purified by preparative HPLC (HCl condition; column: Phenomenex Synergi C18 150*30 mm*4 μm; mobile phase: [water (0.05% HCl)-ACN]; B %: 35%-55%, 10 min) and lyophilized to yield 3-[2-[[2-(1H-indole-3-carbonyl)thiazole-4-carbonyl]amino]ethoxy]propanoic acid (15.08 mg, 37.84 μmol, 5.3% yield, 97.2% purity) as a yellow solid. ¹H NMR (500 MHz, DMSO-d₆) δ ppm 12.36 (br s, 1H), 9.41 (d, J=3.2 Hz, 1H), 8.76 (t, J=5.7 Hz, 1H), 8.62 (s, 1H), 8.38-8.29 (m, 1H), 7.60-7.56 (m, 1H), 7.33-7.26 (m, 2H), 3.67 (t, J=6.3 Hz, 2H), 3.60-3.56 (m, 2H), 3.50 (q, J=6.1 Hz, 2H), 2.50-2.42 (m, 2H); ES-LCMS m/z 388.1 [M+H]⁺.

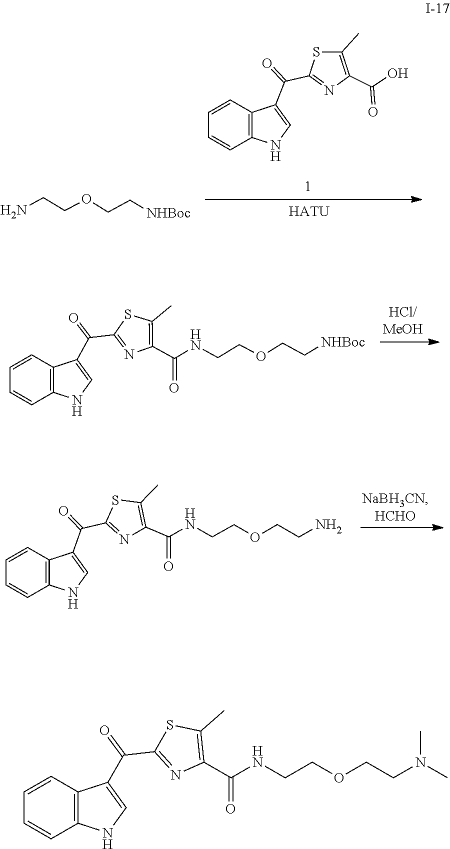 [SMILES: Cc1sc(C(=O)c2c[nH]c3ccccc23)nc1C(=O)O ; CC(C)(C)OC(=O)NCCOCCN ; "HATU" ; Cc1sc(C(=O)c2c[nH]c3ccccc23)nc1C(=O)NCCOCCNC(=O)OC(C)(C)C ; Cc1sc(C(=O)c2c[nH]c3ccccc23)nc1C(=O)NCCOCCN ; Cc1sc(C(=O)c2c[nH]c3ccccc23)nc1C(=O)NCCOCCN(C)C]

### Step 1: tert-Butyl N-[2-[2-[[2-(1H-indole-3-carbonyl)-5-methyl-thiazole-4-carbonyl]amino]ethoxy]ethyl]carbamate

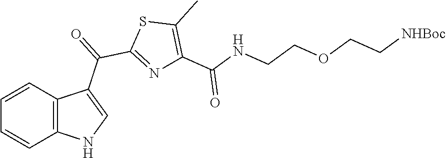 [SMILES: Cc1sc(C(=O)c2c[nH]c3ccccc23)nc1C(=O)NCCOCCNC(=O)OC(C)(C)C]

To a stirred solution of 2-(1H-indole-3-carbonyl)-5-methyl-thiazole-4-carboxylic acid (200 mg, 698.55 μmol, 1 eq) in DMF (5 mL) was added HATU (318.73 mg, 838.27 μmol, 1.2 eq) and tert-butyl N-[2-(2-aminoethoxy)ethyl]carbamate (171.23 mg, 838.27 μmol, 1.2 eq). DIEA (180.57 mg, 1.40 mmol, 243.35 μL, 2 eq) was added to the above reaction mixture then stirred at 25° C. for 1.5 h. The reaction mixture was diluted with water (30 mL) then extracted with EtOAc (50 mL×3). The combine organic layers were dried over Na₂SO₄, filtered and the filtrate was concentrated to yield a residue which was purified by flash silica gel chromatography (from PE/EtOAc=100/1 to 1/1, TLC:PE/EtOAc=1/1, R_{f}=0.35) to yield tert-butyl N-[2-[2-[[2-(1H-indole-3-carbonyl)-5-methyl-thiazole-4-carbonyl]amino]ethoxy]ethyl]carbamate (320 mg, 600.65 μmol, 86.0% yield, 88.7% purity) as yellow solid. ¹H NMR (400 MHz, DMSO-d₆) δ ppm 12.31 (d, J=2.7 Hz, 1H), 9.30 (d, J=3.2 Hz, 1H), 8.58 (t, J=5.9 Hz, 1H), 8.35-8.26 (m, 1H), 7.60-7.53 (m, 1H), 7.31-7.25 (m, 2H), 6.78 (t, J=5.5 Hz, 1H), 3.60-3.53 (m, 2H), 3.51-3.42 (m, 4H), 3.10 (q, J=6.0 Hz, 2H), 2.83 (s, 3H), 1.34 (s, 9H); ES-LCMS m/z 473.1 [M+H]⁺, 373.0 [M-Boc+H]⁺.

### Step 2: N-[2-(2-Aminoethoxy)ethyl]-2-(1H-indole-3-carbonyl)-5-methyl-thiazole-4-carboxamide

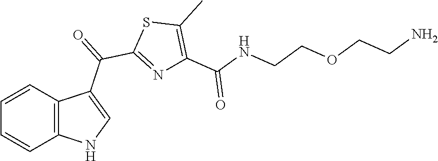 [SMILES: Cc1sc(C(=O)c2c[nH]c3ccccc23)nc1C(=O)NCCOCCN]

tert-Butyl N-[2-[2-[[2-(1H-indole-3-carbonyl)-5-methyl-thiazole-4-carbonyl]amino]ethoxy]ethyl]carbamate (310 mg, 581.88 μmol, 1 eq) was added to HCl/MeOH (4 M, 10 mL) then stirred at 25° C. for 2 h. TLC (PE/EtOAc=1/1, R_{f}=0.10) showed starting material was consumed completely and one new spot was detected. The reaction mixture was concentrated to yield N-[2-(2-aminoethoxy)ethyl]-2-(1H-indole-3-carbonyl)-5-methyl-thiazole-4-carboxamide (260 mg, 572.26 μmol, 98.4% yield, 90.0% purity, HCl) as yellow solid which was used in the next step without further purification. ¹H NMR (500 MHz, DMSO-d₆) δ ppm 12.48 (s, 1H), 9.34 (d, J=3.2 Hz, 1H), 8.67 (t, J=5.9 Hz, 1H), 8.34-8.27 (m, 1H), 7.99 (s, 2H), 7.62-7.55 (m, 1H), 7.31-7.25 (m, 2H), 3.69-3.67 (m, 2H), 3.65-3.63 (m, 1H), 3.54 (q, J=6.0 Hz, 2H), 3.04-2.98 (m, 2H), 2.84 (s, 3H).

### Step 3: N-[2-[2-(Dimethylamino)ethoxy]ethyl]-2-(1H-indole-3-carbonyl)-5-methyl-thiazole-4-carboxamide

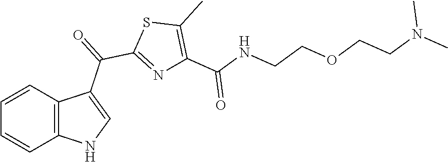 [SMILES: Cc1sc(C(=O)c2c[nH]c3ccccc23)nc1C(=O)NCCOCCN(C)C]

To a stirred solution of N-[2-(2-aminoethoxy)ethyl]-2-(1H-indole-3-carbonyl)-5-methyl-thiazole-4-carboxamide (150 mg, 330.15 μmol, 1 eq, HCl) and HCHO (99.13 mg, 3.30 mmol, 10 eq) in MeOH (5 mL) was added Et₃N (66.82 mg, 660.31 μmol, 91.91 μL, 2 eq). The reaction mixture was stirred at 25° C. for 30 min. NaBH₃CN (103.74 mg, 1.65 mmol, 5 eq) was added to the above mixture then stirred at 25° C. for 2.5 h. The reaction mixture was diluted with water (30 mL) then extracted with EtOAc (30 mL×5). The combined organic layers were dried over Na₂SO₄, filtered and the filtrate was concentrated to yield a residue which was purified by preparative HPLC (column: Welch Xtimate C18 150*25 mm*5 um; mobile phase: [water (10 mM NH₄HCO₃)-ACN]; B %: 40%-70%, 10 min). The desired fraction was lyophilized to yield N-[2-[2-(dimethylamino)ethoxy]ethyl]-2-(1H-indole-3-carbonyl)-5-methyl-thiazole-4-carb oxamide (76.23 mg, 182.92 μmol, 55.4% yield, 96.1% purity) as a yellow solid. ¹H NMR (400 MHz, DMSO-d₆) δ ppm 9.30 (s, 1H), 8.63-8.53 (m, 1H), 8.30 (dd, J=2.4, 6.1 Hz, 1H), 7.57 (dd, J=2.3, 6.2 Hz, 1H), 7.32-7.24 (m, 2H), 3.58-3.47 (m, 6H), 2.83 (s, 3H), 2.39 (t, J=6.0 Hz, 2H), 2.11 (s, 6H); ES-LCMS m/z 401.1 [M+H]⁺.

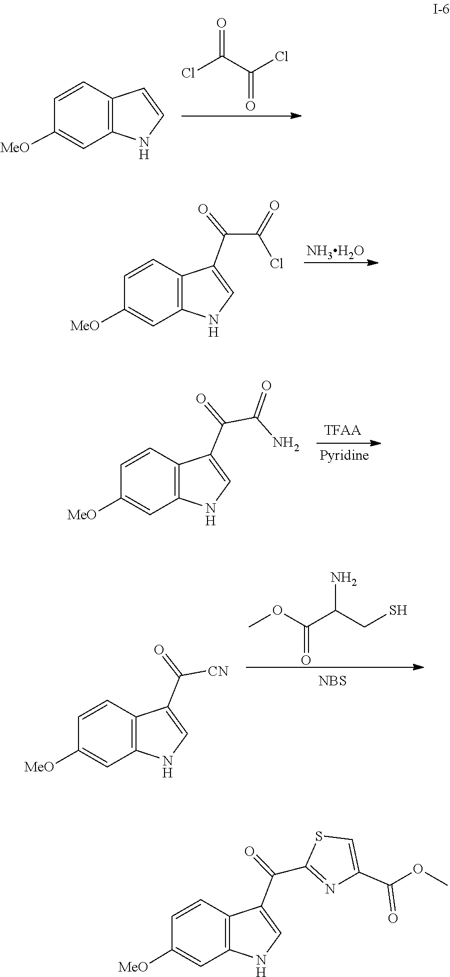

### Step 1: 2-(6-Methoxy-1H-indol-3-yl)-2-oxo-acetyl chloride

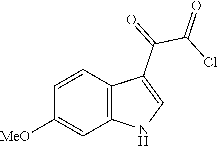 [SMILES: COc1ccc2c(C(=O)C(=O)Cl)c[nH]c2c1]

To a solution of 6-methoxy-1H-indole (1.26 g, 8.56 mmol, 1 eq) in THF (10 mL) was added oxalyl dichloride (1.11 g, 8.73 mmol, 764.40 μL, 1.02 eq) dropwise at 0° C. under N₂ atmosphere. The mixture was stirred at 5° C. for 3 h. The yellow slurry was filtered, and the cake was washed with PE (50 mL×2), dried under reduced pressure to yield 2-(6-methoxy-1H-indol-3-yl)-2-oxo-acetyl chloride (150 mg, 631.21 μmol, 7.4% yield, 100.0% purity) as yellow oil which was used in the next step without further purification. ¹H NMR (400 MHz, DMSO-d₆) δ ppm 12.20 (s, 1H), 8.28 (d, J=3.2 Hz, 1H), 8.01 (d, J=8.8 Hz, 1H), 7.03 (d, J=2.2 Hz, 1H), 6.89 (dd, J=2.3, 8.7 Hz, 1H), 3.79 (s, 3H).

### Step 2: 2-(6-Methoxy-1H-indol-3-yl)-2-oxo-acetamide

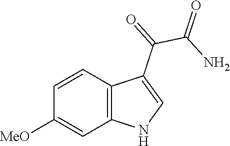 [SMILES: COc1ccc2c(C(=O)C(N)=O)c[nH]c2c1]

To a solution of NH₃.H₂O (790.14 mg, 6.31 mmol, 868.29 μL, 28% purity, 10 eq) in EtOH (3 mL) was added 2-(6-methoxy-1H-indol-3-yl)-2-oxo-acetyl chloride (150 mg, 631.21 μmol, 1 eq). The mixture was stirred at 0° C. for 2 h. The slurry was filtered, and the cake was washed with water (50 mL×2), dried under reduced pressure to yield 2-(6-methoxy-1H-indol-3-yl)-2-oxo-acetamide (30 mg, 130.61 μmol, 20.7% yield, 95.0% purity) as a yellow solid which was used in the next step without further purification. ¹H NMR (400 MHz, DMSO-d₆) δ ppm 11.98 (s, 1H), 8.55 (s, 1H), 8.12-7.97 (m, 2H), 7.66 (s, 1H), 7.01 (d, J=2.2 Hz, 1H), 6.88 (d, J=2.3, 8.7 Hz, 1H), 3.79 (s, 3H); ES-LCMS m/z 219.0 [M+H]⁺.

### Step 3: 6-Methoxy-1H-indole-3-carbonyl cyanide

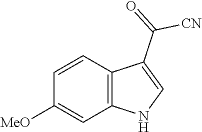 [SMILES: COc1ccc2c(C(=O)C#N)c[nH]c2c1]

To a solution of 2-(6-methoxy-1H-indol-3-yl)-2-oxo-acetamide (2.31 g, 10.32 mmol, 1 eq) in pyridine (5.5 mL) was added TFAA (6.50 g, 30.96 mmol, 4.31 mL, 3 eq). The mixture was stirred at 25° C. for 12 h. The reaction mixture was quenched by addition of sat. aq. NaHCO₃ (100 mL), extracted with EtOAc (80 mL×3). The combined organic layers were washed with aq. HCl (40 mL, 0.5 N), brine (40 mL), dried over Na₂SO₄, filtered and the filtrate was concentrated under reduced pressure to yield 6-methoxy-1H-indole-3-carbonyl cyanide (2.05 g, 10.13 mmol, 98.1% yield, 99.0% purity) as a yellow solid, which was used in the next step without further purification. ¹H NMR (500 MHz, DMSO-d₆) δ ppm 12.72 (s, 1H), 8.52 (d, J=3.4 Hz, 1H), 7.89 (d, J=8.5 Hz, 1H), 7.05 (d, J=2.1 Hz, 1H), 6.96 (dd, J=2.3, 8.7 Hz, 1H), 3.85-3.77 (m, 3H); ES-LCMS m/z 201.0 [M+H]⁺.

### Step 4: 2-(6-Methoxy-1H-indole-3-carbonyl)thiazole-4-carboxylate

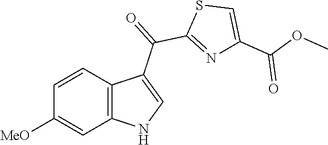 [SMILES: COC(=O)c1csc(C(=O)c2c[nH]c3cc(OC)ccc23)n1]

To a solution of 6-methoxy-1H-indole-3-carbonyl cyanide (200 mg, 989.05 μmol, 1 eq) in pyridine (1.5 mL) was added DBU (15.06 mg, 98.90 μmol, 14.91 μL, 0.1 eq) and methyl 2-amino-3-sulfanyl-propanoate (133.70 mg, 989.05 μmol, 1 eq). After being stirred at 40° C. for 2 h, the reaction mixture was diluted with DCM (30 mL), cooled to 0° C., added DBU (301.14 mg, 1.98 mmol, 298.16 μL, 2 eq), followed by NBS (193.64 mg, 1.09 mmol, 1.1 eq) portion-wise. The mixture was stirred at 0° C. for 1 h. The mixture was quenched with aq. HCl (10 mL, 1 N) and extracted with DCM (10 mL×2). The combined organic layers were washed with 1 N HCl solution (20 mL) and brine (20 mL) twice, dried over anhydrous Na₂SO₄. The residue was purified by flash silica gel chromatography (from PE/EtOAc=100/1 to 2/1, TLC:PE/EtOAc=1/1, R_{f}=0.65) which was re-purified by preparative HPLC (column: Agela DuraShell C18 150*25 mm*5 um; mobile phase: [water (0.04% NH₃.H₂O+10 mM NH₄HCO₃)-ACN]; B %: 45%-75%, 10 min). The desired fraction was lyophilized to yield methyl 2-(6-methoxy-1H-indole-3-carbonyl)thiazole-4-carboxylate (15.06 mg, 47.61 μmol, 4.8% yield, 100.0% purity) as yellow solid. ¹H NMR (500 MHz, DMSO-d₆) δ ppm 8.97 (s, 1H), 8.87 (s, 1H), 8.15 (d, J=8.7 Hz, 1H), 7.09 (d, J=2.1 Hz, 1H), 6.93 (d, J=2.3, 8.7 Hz, 1H), 3.92 (s, 3H), 3.81 (s, 3H); ES-LCMS m/z 316.9 [M+H]⁺.

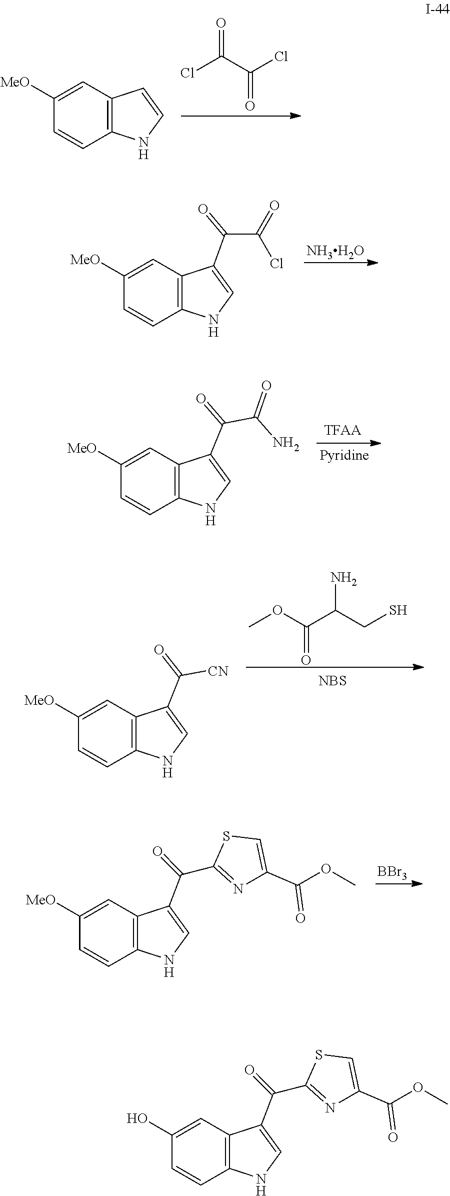

### Step 1: 2-(5-Methoxy-1H-indol-3-yl)-2-oxo-acetyl chloride

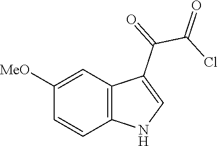 [SMILES: COc1ccc2[nH]cc(C(=O)C(=O)Cl)c2c1]

To a solution of 5-methoxy-1H-indole (2 g, 13.59 mmol, 1 eq) in THF (30 mL) was added oxalyl dichloride (1.81 g, 14.27 mmol, 1.25 mL, 1.05 eq). The mixture was stirred at 0° C. for 3 h. The reaction mixture was concentrated (below 30° C.) to yield 2-(5-methoxy-1H-indol-3-yl)-2-oxo-acetyl chloride (3.23 g, 13.59 mmol, 100.0% yield, crude) as a brown solid, which was used in the next step without further purification.

### Step 2: 2-(5-Methoxy-1H-indol-3-yl)-2-oxo-acetamide

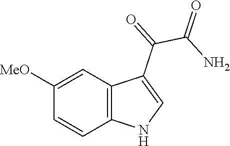 [SMILES: COc1ccc2[nH]cc(C(=O)C(N)=O)c2c1]

To a solution of EtOH (40 mL) and THF (20 mL) was added NH₃.H₂O (18.20 g, 145.41 mmol, 20 mL, 28%, 15.50 eq), 2-(5-methoxy-1H-indol-3-yl)-2-oxo-acetyl chloride (2.23 g, 9.38 mmol, 1 eq) at 0° C. slowly. After addition, the mixture was stirred at 25° C. for 3 h. TLC (PE/EtOAc=1/1, R_{f}=0.09) indicated the starting material was consumed completely and one new spot formed. The reaction mixture was concentrated to yield a residue which was quenched by addition of water (200 mL), extracted with EtOAc (150 mL×4). The combined organic layers were washed with brine (50 mL), dried over Na₂SO₄, filtered and concentrated under reduced pressure to yield 2-(5-methoxy-1H-indol-3-yl)-2-oxo-acetamide (1.5 g, 6.53 mmol, 69.5% yield, 95.0% purity) as a gray solid, which was used in the next step without further purification, ¹H NMR (400 MHz, DMSO-d₆) δ ppm 12.08 (br s, 1H), 8.62 (d, J=3.1 Hz, 1H), 8.02 (br s, 1H), 7.74 (d, J=2.3 Hz, 1H), 7.69 (s, 1H), 7.42 (d, J=9.0 Hz, 1H), 6.89 (dd, J=2.2, 8.8 Hz, 1H), 3.79 (s, 3H).

### Step 3: 5-Methoxy-1H-indole-3-carbonyl cyanide

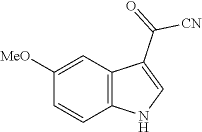 [SMILES: COc1ccc2[nH]cc(C(=O)C#N)c2c1]

To a solution of 2-(5-methoxy-1H-indol-3-yl)-2-oxo-acetamide (800 mg, 3.48 mmol, 1 eq) and pyridine (1.65 g, 20.90 mmol, 1.69 mL, 6 eq) in EtOAc (40 mL) was added TFAA (2.19 g, 10.45 mmol, 1.45 mL, 3 eq) under N₂ while the solution turned clarification. The mixture was stirred at 25° C. for 12 h. The reaction mixture was quenched by addition of NaHCO₃ (200 mL), extracted with EtOAc (200 mL×3). The combined organic layers were washed with 0.5N aq. HCl (50 mL), brine (50 mL), dried over Na₂SO₄, filtered and concentrated under reduced pressure to yield 5-methoxy-1H-indole-3-carbonyl cyanide (650 mg, 3.08 mmol, 88.5% yield, 95.0% purity) as a gray solid which was used in the next step without further purification, ¹H NMR (400 MHz, DMSO-d₆) δ ppm 12.86 (br s, 1H), 8.53 (s, 1H), 7.48 (s, 2H), 6.97 (s, 1H), 3.78 (s, 3H); ES-LCMS m/z 201.0 [M+H]⁺.

### Step 4: Methyl 2-(5-methoxy-1H-indole-3-carbonyl)thiazole-4-carboxylate

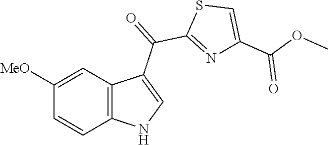 [SMILES: COC(=O)c1csc(C(=O)c2c[nH]c3ccc(OC)cc23)n1]

To a solution of 5-methoxy-1H-indole-3-carbonyl cyanide (650 mg, 3.08 mmol, 1 eq) in pyridine (5 mL) was added DBU (46.96 mg, 308.45 μmol, 46.49 μL, 0.1 eq) and methyl 2-amino-3-sulfanyl-propanoate (529.44 mg, 3.08 mmol, 1 eq, HCl). After stirring at 40° C. for 2 h, the reaction mixture was diluted with DCM (100 mL), cooled to 0° C., added DBU (939.18 mg, 6.17 mmol, 929.88 μL, 2.0 eq), followed by the addition of NBS (603.88 mg, 3.39 mmol, 1.1 eq) portion-wise. The mixture was stirred at 0° C. for 1 h. The reaction mixture was quenched by addition of water (100 mL), extracted with DCM (100 mL×4). The combined organic layers were washed with brine (50 mL), dried over Na₂SO₄, filtered and concentrated under reduced pressure to yield a residue which was purified by preparative HPLC (column: Agela DuraShell C18 150*25 mm*5 μm; mobile phase: [water (0.04% NH₃.H₂O+10 mM NH₄HCO₃)-ACN]; B %: 34%-64%, 10 min), followed by lyophilization to yield methyl 2-(5-methoxy-1H-indole-3-carbonyl)thiazole-4-carboxylate (400 mg, 1.26 mmol, 40.9% yield, 100.0% purity) as a yellow solid. ¹H NMR (500 MHz, DMSO-d₆) δ ppm 12.26 (br s, 1H), 9.02 (s, 1H), 8.87 (s, 1H), 7.82 (d, J=2.4 Hz, 1H), 7.49 (d, J=8.7 Hz, 1H), 6.93 (dd, J=2.5, 8.8 Hz, 1H), 3.97-3.87 (m, 3H), 3.85-3.77 (m, 3H); ES-LCMS m/z 316.9 [M+H]⁺.

### Step 5: Methyl 2-(5-hydroxy-1H-indole-3-carbonyl)thiazole-4-carboxylate

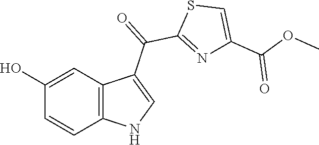 [SMILES: COC(=O)c1csc(C(=O)c2c[nH]c3ccc(O)cc23)n1]

To a solution of methyl 2-(5-methoxy-1H-indole-3-carbonyl)thiazole-4-carboxylate (150 mg, 474.19 μmol, 1 eq) in DCM (10 mL) was added BBr₃ (475.18 mg, 1.90 mmol, 182.76 μL, 4 eq) at −70° C. under N₂. The mixture was stirred at 20° C. for 2 h. The reaction mixture was quenched by addition of aq. NaHCO₃ (10 mL) while pH to 6, diluted with water (20 mL), extracted with EtOAc (30 mL×3). The combined organic layers were washed with brine (10 mL), dried over Na₂SO₄, filtered and concentrated under reduced pressure to yield a residue which was purified by preparative HPLC (column: Welch Xtimate C18 150*25 mm*5 μm; mobile phase: [water (10 mM NH₄HCO₃)-ACN]; B %: 22%-52%, 10 min), followed by lyophilization to yield methyl 2-(5-hydroxy-1H-indole-3-carbonyl)thiazole-4-carboxylate (85.32 mg, 276.59 μmol, 58.3% yield, 98.0% purity) as a yellow solid. ¹H NMR (400 MHz, DMSO-d₆) δ ppm 12.78-11.28 (m, 1H), 9.21 (br s, 1H), 8.98 (s, 1H), 8.87 (s, 1H), 7.71 (d, J=2.0 Hz, 1H), 7.38 (d, J=8.6 Hz, 1H), 6.78 (dd, J=2.4, 8.6 Hz, 1H), 3.92 (s, 3H); ES-LCMS m/z 302.9 [M+H]⁺.

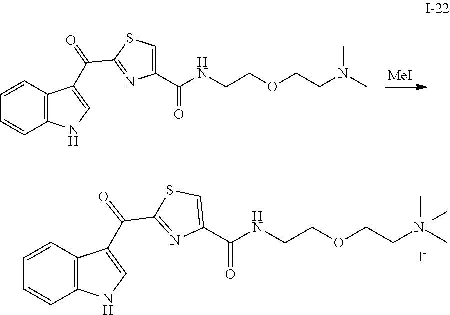 [SMILES: "MeI" ; CN(C)CCOCCNC(=O)c1csc(C(=O)c2c[nH]c3ccccc23)n1 ; C[N+](C)(C)CCOCCNC(=O)c1csc(C(=O)c2c[nH]c3ccccc23)n1 ; [I-]]

### Step 1: 2-(1H-indole-3-carbonyl)-N-[2-[2-[BLAH(trimethyl)-azanyl]ethoxy]ethyl]thiazole-4-carboxamide

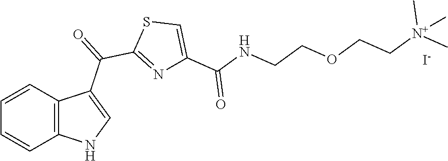 [SMILES: C[N+](C)(C)CCOCCNC(=O)c1csc(C(=O)c2c[nH]c3ccccc23)n1 ; [I-]]

To a solution of N-[2-[2-(dimethylamino)ethoxy]ethyl]-2-(1H-indole-3-carbonyl)thiazole-4-carboxamide (25 mg, 64.69 μmol, 1 eq) in MeCN (3 mL) was added MeI (210 mg, 1.48 mmol, 92.11 μL, 22.87 eq) in one portion at 25° C. under N₂. The mixture was stirred at 25° C. for 1 h. The solution was diluted with water (10 mL), then lyophilization to yield 2-(1H-indole-3-carbonyl)-N-[2-[2-[BLAH(trimethyl)-azanyl]ethoxy]ethyl]thiazole-4-carboxamide (17.59 mg, 33.29 μmol, 51.5% yield, 100% purity) as a yellow solid. ¹H NMR (500 MHz, DMSO-d₆) δ ppm 12.34 (s, 1H), 9.40 (d, J=3.1 Hz, 1H), 8.82 (t, J=5.8 Hz, 1H), 8.64-8.60 (m, 1H), 8.33 (d, J=6.9 Hz, 1H), 7.59 (d, J=7.0 Hz, 1H), 7.33-7.27 (m, 2H), 3.89 (s, 2H), 3.70-3.65 (m, 2H), 3.60-3.51 (m, 4H), 3.07 (s, 9H); ES-LCMS m/z 401.0 [M-I⁻]⁺.

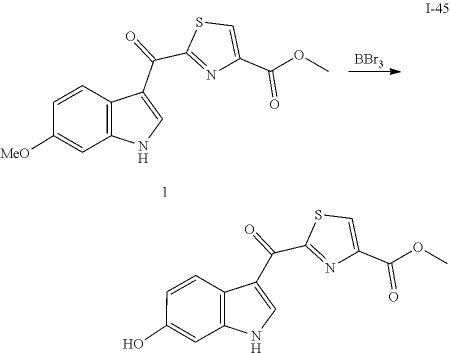

### Step 1: Methyl 2-(6-hydroxy-1H-indole-3-carbonyl)thiazole-4-carboxylate

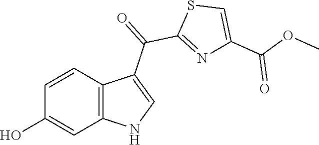 [SMILES: COC(=O)c1csc(C(=O)c2c[nH]c3cc(O)ccc23)n1]

To a solution of methyl 2-(6-methoxy-1H-indole-3-carbonyl)thiazole-4-carboxylate (150 mg, 237.09 μmol, 1 eq) in DCM (10 mL) was added BBr₃ (1 M, 711.28 μL, 3 eq) at 0° C. The mixture was stirred at 25° C. for 1 h. The reaction mixture was quenched with H₂O (20 mL) and filtered. The solid was purified by preparative HPLC (column: Welch Xtimate C18 150*30 mm*5 um; mobile phase: [water (10 mM NH₄HCO₃)-ACN]; B %: 27%-53%, 9 min) and lyophilized to yield methyl 2-(6-hydroxy-1H-indole-3-carbonyl)thiazole-4-carboxylate (21.51 mg, 68.48 μmol, 28.9% yield, 96.2% purity) as a yellow solid. ¹H NMR (500 MHz, DMSO-d₆) δ ppm 12.03 (br s, 1H), 9.42 (s, 1H), 8.93 (s, 1H), 8.87 (s, 1H), 8.07 (d, J=8.5 Hz, 1H), 6.93 (d, J=2.0 Hz, 1H), 6.79 (dd, J=2.1, 8.5 Hz, 1H), 3.92 (s, 3H); ES-LCMS m/z 302.9 [M+H]⁺.

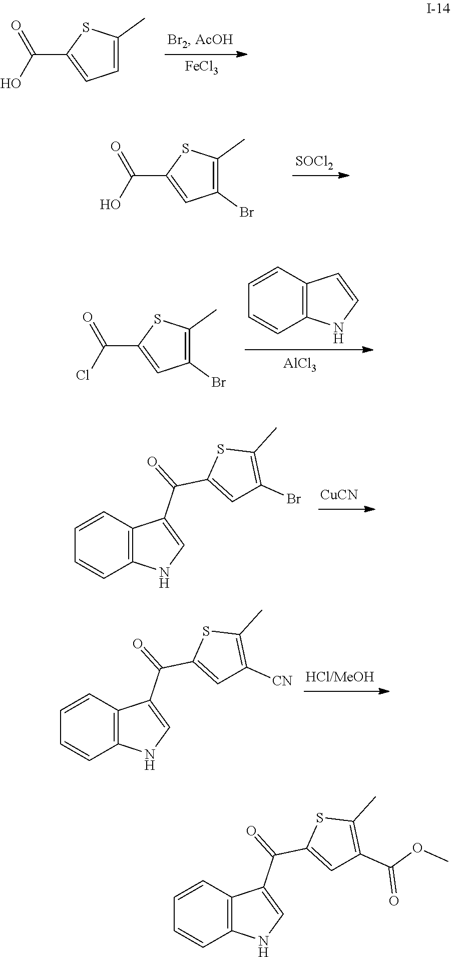

### Step 1: 4-Bromo-5-methyl-thiophene-2-carboxylic acid

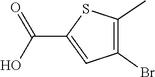

A solution of Br₂ (1.69 g, 10.55 mmol, 543.88 μL, eq) in AcOH (5 mL) was added dropwise to 5-methylthiophene-2-carboxylic acid (1.5 g, 10.55 mmol, 1 eq) and FeCl₃ (342.25 mg, 2.11 mmol, 122.23 μL, 0.2 eq) in AcOH (25 mL). The mixture was stirred at 25° C. for 5 h. The mixture was poured onto ice and the precipitate was filtered and washed with water affording the product which was dried to yield 4-bromo-5-methyl-thiophene-2-carboxylic acid (2 g, 9.05 mmol, 85.8% yield, 100% purity) as a yellow solid which was used in the next step without further purification. ¹H NMR (500 MHz, DMSO-d₆) δ ppm 13.32 (s, 1H), 7.60 (s, 1H), 2.41 (s, 3H).

### Step 2: 4-Bromo-5-methyl-thiophene-2-carbonyl chloride

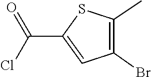

To a solution of 4-bromo-5-methyl-thiophene-2-carboxylic acid (500 mg, 2.26 mmol, 1 eq) in DCM (20 mL) was added DMF (16.53 mg, 226.17 mol, 17.40 μL, 0.1 eq) and (COCl)₂ (1.15 g, 9.05 mmol, 791.92 μL, 4 eq) at 0° C. The mixture was stirred at 25° C. for 1 h. The reaction mixture was concentrated under reduced pressure to yield 4-bromo-5-methyl-thiophene-2-carbonyl chloride (540 mg, 2.19 mmol, 96.7% yield, 97.0% purity) as a yellow solid which was used in the next step without further purification. ES-LCMS m/z 236.8 [M-Cl+OMe]⁺.

### Step 3: (4-Bromo-5-methyl-2-thienyl)-(1H-indol-3-yl)methanone

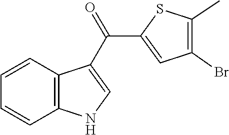 [SMILES: Cc1sc(C(=O)c2c[nH]c3ccccc23)cc1Br]

To a solution of 4-bromo-5-methyl-thiophene-2-carbonyl chloride (500 mg, 2.02 mmol, 1 eq) in DCM (8 mL) was added AlCl₃ (810.00 mg, 6.07 mmol, 331.97 μL, 3 eq) at 0° C. and stirred at 25° C. for 0.5 h. To the mixture was added dropwise a solution of indole (308.38 mg, 2.63 mmol, 1.3 eq) in DCM (2 mL). The mixture was stirred at 30° C. for 12.5 h. The mixture was quenched with 15 mL MeOH. The reaction mixture was concentrated under reduced pressure to yield a residue which was purified by flash silica gel chromatography (from PE/EtOAc=200/1 to 5/1, TLC:PE/EtOAc=5/1, R_{f}=0.25) to yield (4-bromo-5-methyl-2-thienyl)-(1H-indol-3-yl)methanone (220 mg, 611.49 μmol, 30.2% yield, 89% purity) as a yellow solid. ¹H NMR (400 MHz, DMSO-d₆) δ ppm 12.17 (s, 1H), 8.44 (d, J=3.5 Hz, 1H), 8.19 (d, J=7.0 Hz, 1H), 7.87 (s, 1H), 7.52 (d, J=7.0 Hz, 1H), 7.29-7.19 (m, 2H), 2.46 (s, 3H); ES-LCMS m/z 319.9 [M+H]⁺.

### Step 4: 5-(1H-Indole-3-carbonyl)-2-methyl-thiophene-3-carbonitrile

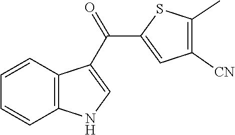 [SMILES: Cc1sc(C(=O)c2c[nH]c3ccccc23)cc1C#N]

To a solution of (4-bromo-5-methyl-2-thienyl)-(1H-indol-3-yl)methanone (200 mg, 555.90 μmol, 1 eq) in DMF (2 mL) was added CuCN (248.93 mg, 2.78 mmol, 607.15 μL, 5 eq). The mixture was stirred at 150° C. for 2 h under microwave (1 bar). To the mixture was added water (50 mL) and extracted with ethyl acetate (50 mL×5). The combined organic phase was washed with brine (20 mL), dried with anhydrous Na₂SO₄, filtered and concentrated in vacuum to yield 5-(1H-indole-3-carbonyl)-2-methyl-thiophene-3-carbonitrile (120 mg, 315.41 μmol, 56.7% yield, 70% purity) as a yellow solid which was used in the next step without further purification. ¹H NMR (500 MHz, DMSO-d₆) δ ppm 12.32 (s, 1H), 8.49 (s, 1H), 8.19 (s, 1H), 7.95 (s, 1H), 7.53 (d, J=8.1 Hz, 1H), 7.27-7.21 (m, 2H), 2.70 (s, 3H); ES-LCMS m/z 266.9 [M+H]⁺.

### Step 5: Methyl 5-(1H-indole-3-carbonyl)-2-methyl-thiophene-3-carboxylate

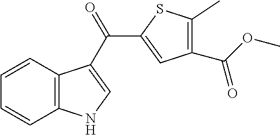 [SMILES: COC(=O)c1cc(C(=O)c2c[nH]c3ccccc23)sc1C]

A solution of 5-(1H-indole-3-carbonyl)-2-methyl-thiophene-3-carbonitrile (120 mg, 315.41 μmol, 1 eq) in HCl/MeOH (10 mL) was stirred at 80° C. for 12 h. The reaction mixture was concentrated under reduced pressure to yield a residue. To the residue was added sat. aq. NaHCO₃ (2 mL), water (30 mL) and extracted with ethyl acetate (30 mL×5). The combined organic phase was washed with brine (20 mL), dried with anhydrous Na₂SO₄, filtered and concentrated in vacuum to yield a residue which was purified by preparative HPLC (column: Agela DuraShell C18 150*25 mm*5 μm; mobile phase: [water (0.04% NH₃.H₂O+10 mM NH₄HCO₃)-ACN]; B %: 40%-70%, 10 min), followed by lyophilization to yield methyl 5-(1H-indole-3-carbonyl)-2-methyl-thiophene-3-carboxylate (55 mg, 183.74 μmol, 58.2% yield, 100% purity) as a yellow solid. ¹H NMR (500 MHz, DMSO-d₆) δ ppm 12.13 (s, 1H), 8.37 (s, 1H), 8.18 (d, J=8.1 Hz, 1H), 7.99 (s, 1H), 7.54 (d, J=7.9 Hz, 1H), 7.30-7.20 (m, 2H), 3.83 (s, 3H), 2.77 (s, 3H); ES-LCMS m/z 300.0 [M+H]⁺.

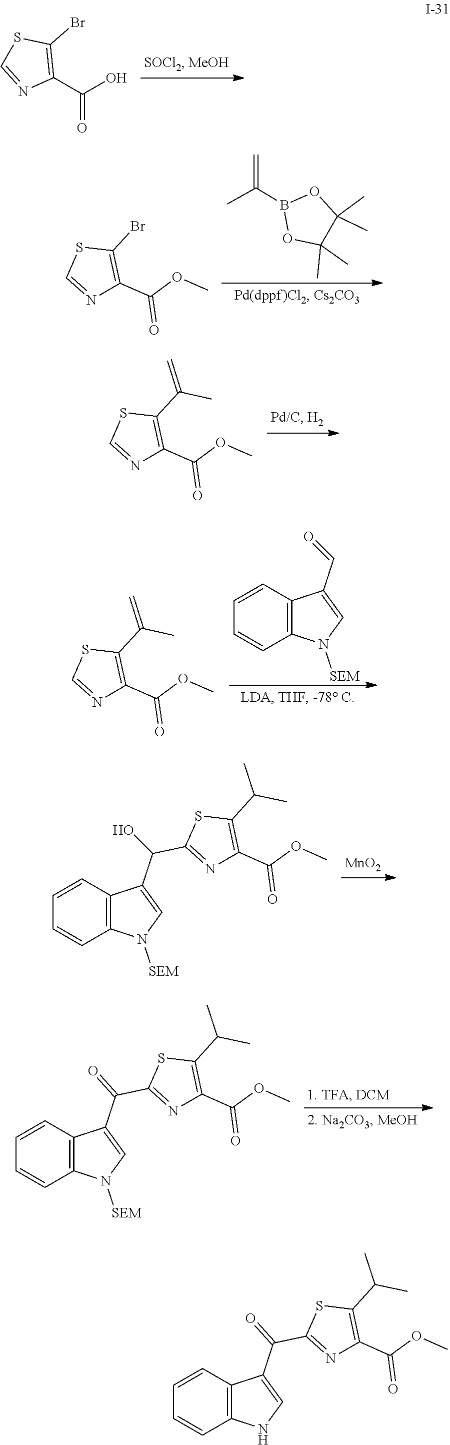

### Step 1: Methyl 5-bromothiazole-4-carboxylate

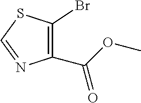

To a solution of 5-bromothiazole-4-carboxylic acid (1.8 g, 8.65 mmol, 1 eq) and DMF (95.00 mg, 1.30 mmol, 0.1 mL, 0.15 eq) in MeOH (20 mL) was added SOCl₂ (1.64 g, 13.78 mmol, 1 mL, 1.59 eq) dropwise at 20° C. The mixture was stirred at 20° C. for 2 h. TLC (PE/EtOAc=1/1, R_{f}=0.50) showed the starting material was consumed completely. The reaction mixture was concentrated under reduced pressure to yield methyl 5-bromothiazole-4-carboxylate (1.73 g, 7.79 mmol, 90.0% yield, 100.0% purity) as an off-white solid, which was used in the next step without further purification. ¹H NMR (400 MHz, CDCl₃) δ ppm 8.80 (s, 1H), 3.99 (s, 3H); ES-LCMS m/z 221.8, 223.8 [M+H]⁺.

### Step 2: Methyl 5-isopropenylthiazole-4-carboxylate

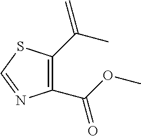

A mixture of methyl 5-bromothiazole-4-carboxylate (300 mg, 1.35 mmol, 1 eq), 2-isopropenyl-4,4,5,5-tetramethyl-1,3,2-dioxaborolane (300 mg, 1.79 mmol, 1.32 eq), Na₂CO₃ (450 mg, 4.25 mmol, 3.14 eq) and Pd(dppf)Cl₂ (50 mg, 68.33 umol, 5.06e-2 eq) in 1,4-dioxane (10 mL) and H₂O (2 mL) was stirred under N₂ atmosphere at 80° C. for 2 h. The reaction mixture was diluted with H₂O (50 mL) and extracted with EtOAc (50 mL×3). The organic layer was dried over Na₂SO₄, filtered and concentrated under reduced pressure to yield a residue which was purified by flash silica gel chromatography (from PE/EtOAc=100/1 to 8/1, TLC:PE/EtOAc=3/1, R_{f}=0.50) to yield methyl 5-isopropenylthiazole-4-carboxylate (200 mg, 1.03 mmol, 76.5% yield, 94.7% purity) as a colorless gum. ¹H NMR (400 MHz, CDCl₃) δ ppm 8.64 (s, 1H), 5.38 (s, 1H), 5.26 (s, 1H), 3.95 (s, 3H), 2.20 (s, 3H); ES-LCMS m/z 183.9 [M+H]⁺.

### Step 3: Methyl 5-isopropylthiazole-4-carboxylate

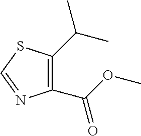

A mixture of methyl 5-isopropenylthiazole-4-carboxylate (200 mg, 1.03 mmol, 1 eq) and Pd/C (200 mg, 10% purity) in MeOH (10 mL) and EtOAc (10 mL) was stirred under H2 (15 psi) at 25° C. for 12 h. The reaction mixture was filtered. The filtrate was concentrated under reduced pressure to yield methyl 5-isopropylthiazole-4-carboxylate (140 mg, 744.43 μmol, 72.0% yield, 98.5% purity) as colorless oil, which was used in the next step without further purification. ¹H NMR (400 MHz, CDCl₃) δ ppm 8.62 (s, 1H), 4.21 (td, J=6.8, 13.6 Hz, 1H), 3.96 (s, 3H), 1.36 (d, J=6.8 Hz, 6H); ES-LCMS m/z 185.9 [M+H]⁺.

### Step 4: Methyl 2-[hydroxy-[1-(2-trimethylsilylethoxymethyl)indol-3-yl]methyl]-5-isopropyl-thiazole-4-carboxylate

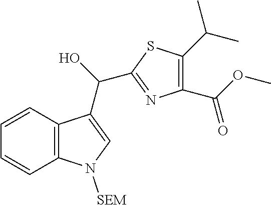 [SMILES: COC(=O)c1nc(C(O)c2cn(COCC[Si](C)(C)C)c3ccccc23)sc1C(C)C]

To a solution of i-Pr₂NH (143.20 mg, 1.42 mmol, 200 μL, 2.05 eq) in THF (10 mL) was added n-BuLi (2.5 M, 600 μL, 2.17 eq) under N₂ atmosphere at −78° C. The mixture was stirred under N₂ atmosphere at −78° C. for 0.5 h. A solution of methyl 5-isopropylthiazole-4-carboxylate (130 mg, 691.25 μmol, 1 eq) and 1-(2-trimethylsilylethoxymethyl)indole-3-carbaldehyde (220 mg, 718.91 μmol, 1.04 eq) in THF (3 mL) was added under N₂ atmosphere at −78° C. The mixture was stirred under N₂ atmosphere at −78° C. for 0.5 h. TLC (PE/EtOAc=3/1, R_{f}=0.08) showed the starting material was consumed completely. The reaction mixture was quenched with H₂O (50 mL) and extracted with EtOAc (50 mL×3). The organic layer was dried over Na₂SO₄, filtered and concentrated under reduced pressure to yield a residue which was purified by flash silica gel chromatography (from PE/EtOAc=100/1 to 1/1, TLC:PE/EtOAc=3/1, R_{f}=0.08) to yield methyl 2-[hydroxy-[1-(2-trimethylsilylethoxymethyl)indol-3-yl]methyl]-5-isopropyl-thiazole-4-carboxylate (100 mg, 176.27 μmol, 25.5% yield, 81.2% purity) as a white solid. ¹H NMR (400 MHz, CDCl₃) δ ppm 7.66 (d, J=7.8 Hz, 1H), 7.50 (d, J=8.2 Hz, 1H), 7.27-7.25 (m, 2H), 7.18-7.13 (m, 1H), 6.38 (d, J=2.7 Hz, 1H), 5.47 (s, 2H), 5.31 (s, 1H), 4.17-4.13 (m, 1H), 3.94 (s, 3H), 3.51-3.47 (m, 2H), 1.31-1.28 (m, 6H), 0.92-0.87 (m, 2H), −0.05 (s, 9H); ES-LCMS m/z 461.1 [M+H]⁺.

### Step 5: Methyl 5-isopropyl-2-[1-(2-trimethylsilylethoxymethyl)indole-3-carbonyl]thiazole-4-carboxylate

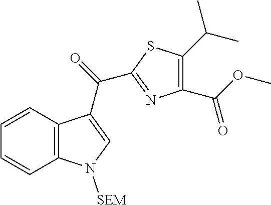 [SMILES: COC(=O)c1nc(C(=O)c2cn(COCC[Si](C)(C)C)c3ccccc23)sc1C(C)C]

A mixture of methyl 2-[hydroxy-[1-(2-trimethylsilylethoxymethyl)indol-3-yl]methyl]-5-isopropyl-thiazole-4-carboxylate (100 mg, 176.27 μmol, 1 eq) and MnO₂ (153.24 mg, 1.76 mmol, 10 eq) in DCM (10 mL) was stirred at 25° C. for 12 h. TLC (PE/EtOAc=2/1, R_{f}=0.39) showed the starting material was consumed completely. The reaction mixture was filtered. The filtrate was concentrated under reduced pressure to yield methyl 5-isopropyl-2-[1-(2-trimethylsilylethoxymethyl)indole-3-carbonyl]thiazole-4-carboxylate (60 mg, 109.23 μmol, 62.0% yield, 83.5% purity) as a white solid, which was used in the next step without further purification. ¹H NMR (400 MHz, CDCl₃) δ ppm 9.08 (s, 1H), 8.53 (d, J=8.6 Hz, 1H), 7.59 (d, J=9.4 Hz, 1H), 7.39-7.36 (m, 2H), 5.62 (s, 2H), 4.22-4.15 (m, 1H), 4.00 (s, 3H), 3.60-3.54 (m, 2H), 1.43 (d, J=7.0 Hz, 6H), 0.96-0.91 (m, 2H), −0.04 (s, 9H); ES-LCMS m/z 459.1 [M+H]⁺.

### Step 6: Methyl 2-(1H-indole-3-carbonyl)-5-isopropyl-thiazole-4-carboxylate

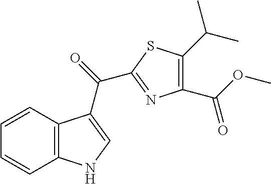 [SMILES: COC(=O)c1nc(C(=O)c2c[nH]c3ccccc23)sc1C(C)C]

To a solution of methyl 5-isopropyl-2-[1-(2-trimethylsilylethoxymethyl)indole-3-carbonyl]thiazole-4-carboxylate (55 mg, 100.13 μmol, 1 eq) in DCM (2 mL) was added TFA (1.29 g, 11.28 mmol, 835.00 μL, 112.63 eq) at 25° C. The mixture was stirred at 25° C. for 1 h. The mixture was concentrated under reduced pressure. The residue was dissolved in MeOH (2 mL) and basified with saturated aqueous Na₂CO₃ until pH=9. The mixture was stirred at 25° C. for 2 h. The reaction mixture was diluted with H₂O (20 mL) and extracted with EtOAc (20 mL×3). The organic layer was dried over Na₂SO₄, filtered and concentrated under reduced pressure to yield a residue which was purified by preparative HPLC (column: Welch Xtimate C18 150*25 mm*5 um; mobile phase: [water (10 mM NH₄HCO₃)-ACN]; B %: 46%-76%, 10 min) and lyophilized to yield methyl 2-(1H-indole-3-carbonyl)-5-isopropyl-thiazole-4-carboxylate (16.79 mg, 51.13 μmol, 51.1% yield, 100.0% purity) as a yellow solid. ¹H NMR (500 MHz, CDCl₃) δ ppm 9.18 (d, J=3.2 Hz, 1H), 8.86 (s, 1H), 8.53 (d, J=7.2 Hz, 1H), 7.46 (d, J=7.5 Hz, 1H), 7.38-7.31 (m, 2H), 4.23-4.14 (m, 1H), 4.00 (s, 3H), 1.43 (d, J=6.9 Hz, 6H); ES-LCMS m/z 329.0 [M+H]⁺.

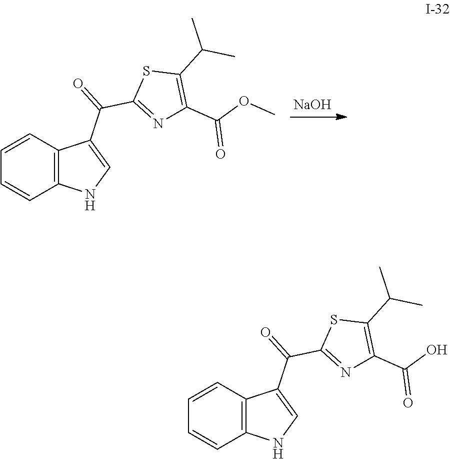

### Step 1: 2-(1H-indole-3-carbonyl)-5-isopropyl-thiazole-4-carboxylic acid

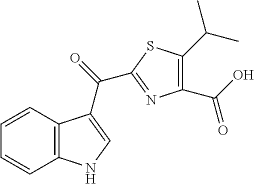 [SMILES: CC(C)c1sc(C(=O)c2c[nH]c3ccccc23)nc1C(=O)O]

A mixture of methyl 2-(1H-indole-3-carbonyl)-5-isopropyl-thiazole-4-carboxylate (100 mg, 304.52 μmol, 1 eq) and NaOH (100 mg, 2.50 mmol, 8.21 eq) in MeOH (3 mL), THF (3 mL) and H₂O (3 mL) was stirred at 25° C. for 3 h. TLC (PE/EtOAc=1/1, R_{f}=0.14) showed the starting material was consumed completely. The mixture was concentrated under reduced pressure. The residue was diluted with H₂O (10 mL) and extracted with DCM (10 mL×2). The organic layer was discarded. The aqueous layer was neutralized with aqueous HCl (1M) until pH=7 and extracted with EtOAc (10 mL×3). The organic layer was dried over Na₂SO₄, filtered and concentrated under reduced pressure to yield a residue which was dissolved in MeCN (50 mL) and water (50 mL) and lyophilized to yield 2-(1H-indole-3-carbonyl)-5-isopropyl-thiazole-4-carboxylic acid (40.38 mg, 128.45 μmol, 42.2% yield, 100.0% purity) as a white solid. ¹H NMR (400 MHz, DMSO-d₆) δ ppm 12.28 (br s, 1H), 9.13 (d, J=3.1 Hz, 1H), 8.30 (dd, J=2.9, 5.7 Hz, 1H), 7.67-7.51 (m, 1H), 7.36-7.22 (m, 2H), 4.12 (q, J=6.8 Hz, 1H), 1.35 (d, J=6.7 Hz, 6H); ES-LCMS m/z 315.0 [M+H]⁺.

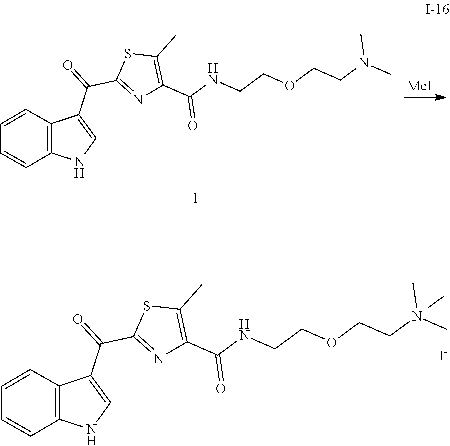 [SMILES: "MeI" ; Cc1sc(C(=O)c2c[nH]c3ccccc23)nc1C(=O)NCCOCCN(C)C ; Cc1sc(C(=O)c2c[nH]c3ccccc23)nc1C(=O)NCCOCC[N+](C)(C)C ; [I-]]

### Step 1: 2-(1H-Indole-3-carbonyl)-5-methyl-N-[2-[2-[BLAH(trimethyl)-azanyl]ethoxy]ethyl]thiazole-4-carboxamide

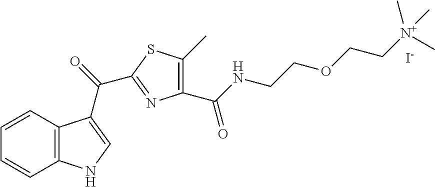 [SMILES: Cc1sc(C(=O)c2c[nH]c3ccccc23)nc1C(=O)NCCOCC[N+](C)(C)C ; [I-]]

To a stirred solution of N-[2-[2-(dimethylamino)ethoxy]ethyl]-2-(1H-indole-3-carbonyl)-5-methyl-thiazole-4-carboxamide (60 mg, 143.97 μmol, 1 eq) in ACN (5 mL) was added MeI (102.18 mg, 719.86 μmol, 44.81 μL, 5 eq). The reaction mixture was stirred at 25° C. for 1 h. The reaction mixture was concentrated to yield 2-(1H-indole-3-carbonyl)-5-methyl-N-[2-[2-[BLAH(trimethyl)-azanyl]ethoxy]ethyl]thiazole-4-carboxamide (69.85 mg, 123.88 μmol, 86.0% yield, 96.2% purity) as yellow solid which was lyophilized for delivery without further purification. ¹H NMR (500 MHz, DMSO-d₆) δ ppm 12.29 (s, 1H), 9.31 (s, 1H), 8.66 (t, J=5.8 Hz, 1H), 8.31 (d, J=7.0 Hz, 1H), 7.58 (d, J=7.2 Hz, 1H), 7.36-7.23 (m, 2H), 3.89 (s, 2H), 3.67 (t, J=5.6 Hz, 2H), 3.58-3.51 (m, 4H), 3.13-3.02 (m, 9H), 2.84 (s, 3H); ES-LCMS m/z 415.5 [M+H]⁺.

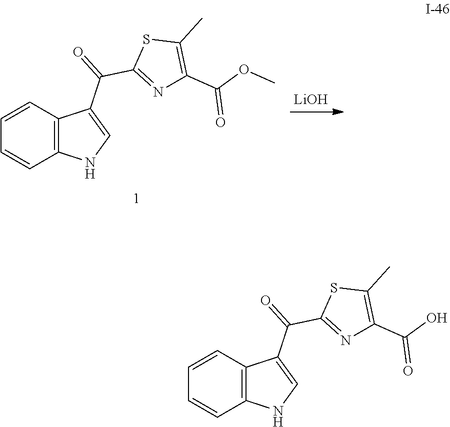

### Step 1: 5-(1H-Indole-3-carbonyl)-2-methyl-thiophene-3-carboxylic acid

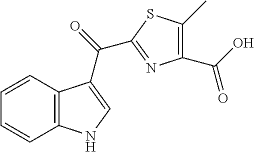 [SMILES: Cc1sc(C(=O)c2c[nH]c3ccccc23)nc1C(=O)O]

To a solution of methyl 5-(1H-indole-3-carbonyl)-2-methyl-thiophene-3-carboxylate (35 mg, 116.92 μmol, 1 eq) in EtOH (3 mL) and H₂O (3 mL) was added LiOH.H₂O (24.53 mg, 584.61 μmol, 5 eq). The mixture was stirred at 25° C. for 1 h. The reaction mixture was concentrated under reduced pressure to yield a residue. The residue was purified by preparative HPLC (column: Phenomenex Synergi C18 150*30 mm*4 μm; mobile phase: [water (0.05% HCl)-ACN]; B %: 34%-54%, 10 min), followed by lyophilization to yield 5-(1H-indole-3-carbonyl)-2-methyl-thiophene-3-carboxylic acid (18.93 mg, 66.35 μmol, 56.7% yield, 100% purity) as a yellow solid. ¹H NMR (500 MHz, DMSO-d₆) δ ppm 12.12 (s, 1H), 8.36 (d, J=3.1 Hz, 1H), 8.18 (d, J=7.6 Hz, 1H), 7.97 (s, 1H), 7.53 (d, J=7.8 Hz, 1H), 7.28-7.20 (m, 2H), 2.76 (s, 3H); ES-LCMS m/z 285.9[M+H]⁺.

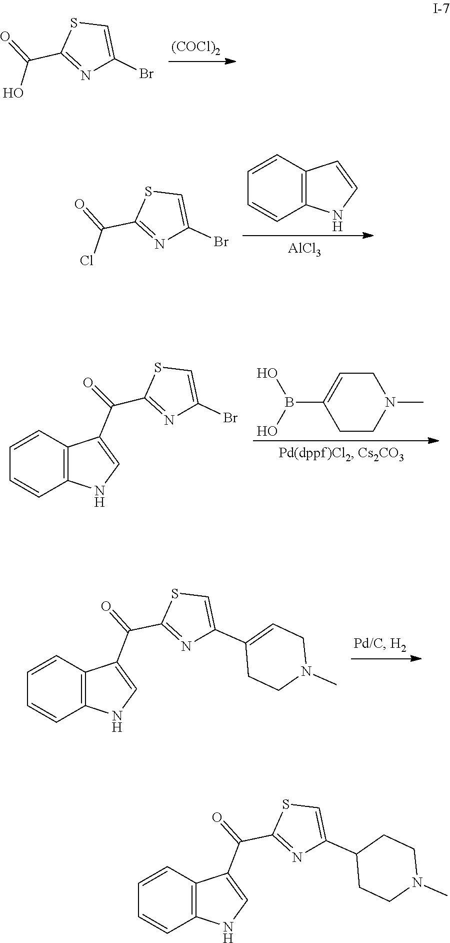

### Step 1: 4-Bromothiazole-2-carbonyl chloride

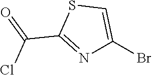

To a solution of 4-bromothiazole-2-carboxylic acid (1.4 g, 6.73 mmol, 1 eq) in DCM (20 mL) was added (COCl)₂ (1.71 g, 13.46 mmol, 1.18 mL, 2 eq) and DMF (245.95 mg, 3.36 mmol, 258.89 μL, 0.5 eq) at 0° C. under N₂. The mixture was stirred at 20° C. for 1 h. The reaction mixture was concentrated to yield 4-bromothiazole-2-carbonyl chloride (1.52 g, crude) as yellow oil which was used in the next step without further purification.

### Step 2: (4-Bromothiazol-2-yl)-(1H-indol-3-yl)methanone

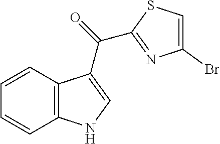 [SMILES: O=C(c1nc(Br)cs1)c1c[nH]c2ccccc12]

To a solution of indole (786.23 mg, 6.71 mmol, 1 eq) in DCM (30 mL) was added AlCl₃ (1.79 g, 13.42 mmol, 733.53 μL, 2 eq) at 0° C. under N₂. After being stirred at 0° C. for 0.5 h, a solution of 4-bromothiazole-2-carbonyl chloride (1.52 g, 6.71 mmol, 1 eq) in DCM (10 mL) was added. The resulting mixture was stirred at 25° C. for 12 h. The reaction mixture was quenched by addition of MeOH (50 mL) slowly at 0° C., concentrated to yield a residue which was purified by flash silica gel chromatography (from EtOAc/MeOH=1/0 to 5/1, TLC:EtOAc/MeOH=5/1, R_{f}=0.16) to yield (4-bromothiazol-2-yl)-(1H-indol-3-yl)methanone (1 g, 2.71 mmol, 40.4% yield, 83.3% purity) as a yellow solid. ¹H NMR (400 MHz, DMSO-d₆) δ ppm 12.31 (br s, 1H), 9.31 (s, 1H), 8.21-8.10 (m, 2H), 7.58-7.49 (m, 1H), 7.36-7.22 (m, 2H); ES-LCMS m/z 306.9, 308.8 [M+H]⁺.

### Step 3: 1H-Indol-3-yl-[4-(1-methyl-3,6-dihydro-2H-pyridin-4-yl)thiazol-2-yl]methanone

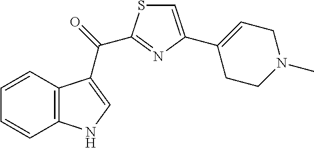 [SMILES: CN1CC=C(c2csc(C(=O)c3c[nH]c4ccccc34)n2)CC1]

A mixture of (4-bromothiazol-2-yl)-(1H-indol-3-yl)methanone (500 mg, 1.36 mmol, 1 eq), (1-methyl-3,6-dihydro-2H-pyridin-4-yl)boronic acid (191.16 mg, 1.36 mmol, 1 eq), Pd(dppf)Cl₂ (99.22 mg, 135.59 μmol, 0.1 eq) and Cs₂CO₃ (2 M, 2.03 mL, 3 eq) in 1,4-dioxane (20 mL) was de-gassed and then heated to 80° C. for 12 h under N₂. TLC (PE/EtOAc=3/1, R_{f}=0.49) indicated the starting material was consumed completely and one new major spot formed. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC:PE/EtOAc=3/1, R_{f}=0.49) to yield 1H-indol-3-yl-[4-(1-methyl-3,6-dihydro-2H-pyridin-4-yl)thiazol-2-yl]methanone (100 mg, 238.09 μmol, 17.5% yield, 77.0% purity) as a brown solid. ¹H NMR (500 MHz, CD₃OD) δ ppm 9.15 (s, 1H), 8.40 (dd, J=2.4, 6.3 Hz, 1H), 7.87 (s, 1H), 7.54-7.48 (m, 1H), 7.32-7.29 (m, 2H), 6.79 (br s, 1H), 3.59-3.48 (m, 2H), 3.01 (s, 5H), 2.89 (d, J=2.3 Hz, 2H); ES-LCMS m/z 324.0 [M+H]⁺.

### Step 4: 1H-Indol-3-yl-[4-(1-methyl-4-piperidyl)thiazol-2-yl]methanone

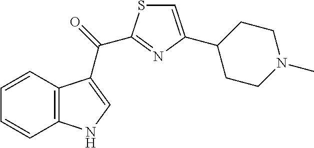 [SMILES: CN1CCC(c2csc(C(=O)c3c[nH]c4ccccc34)n2)CC1]

To a solution of 1H-indol-3-yl-[4-(1-methyl-3,6-dihydro-2H-pyridin-4-yl)thiazol-2-yl]methanone (100 mg, 238.09 μmol, 1 eq) in MeOH (10 mL) was added Pd/C (30 mg, 10% purity). The mixture was stirred at 50° C. for 12 h under H2 (50 psi). The residue was filtered, and the cake was rinsed with MeOH (2×30 mL). The solid was collected and dried in vacuo to yield a residue which was purified by preparative HPLC (column: Welch Xtimate C18 150*25 mm*5 μm; mobile phase: [water (10 mM NH₄HCO₃)-ACN]; B %: 45%-75%, 10 min), followed by lyophilization to yield 1H-indol-3-yl-[4-(1-methyl-4-piperidyl)thiazol-2-yl]methanone (11.78 mg, 36.20 μmol, 15.2% yield, 100% purity) as a white solid. ¹H NMR (400 MHz, DMSO-d₆) δ ppm 9.11 (s, 1H), 8.30-8.21 (m, 1H), 7.71-7.60 (m, 2H), 7.24-7.11 (m, 2H), 2.92-2.75 (m, 3H), 2.21 (s, 3H), 2.08-1.96 (m, 4H), 1.84-1.69 (m, 2H); ES-LCMS m/z 326.0 [M+H]⁺.

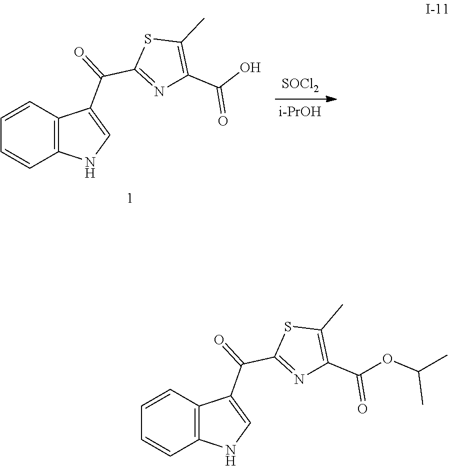

### Step 1: Isopropyl 2-(1H-indole-3-carbonyl)-5-methyl-thiazole-4-carboxylate

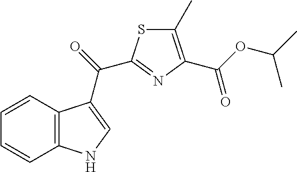 [SMILES: Cc1sc(C(=O)c2c[nH]c3ccccc23)nc1C(=O)OC(C)C]

To a stirred solution of 2-(1H-indole-3-carbonyl)-5-methyl-thiazole-4-carboxylic acid (50 mg, 157.17 μmol, 1 eq) in i-PrOH (2 mL) was added SOCl₂ (65.45 mg, 550.11 μmol, 39.91 μL, 3.5 eq) and DMF (1.15 mg, 15.72 μmol, 1.21 μL, 0.1 eq). The reaction mixture was stirred at 85° C. for 12 h. The reaction mixture was concentrated to yield a residue which was purified by preparative HPLC (column: Agela DuraShell C18 150*25 mm*5 um; mobile phase: [water (0.04% NH₃.H₂O+10 mM NH₄HCO₃)-ACN]; B %: 49%-79%, 10 min). The desired fraction was lyophilized to yield isopropyl 2-(1H-indole-3-carbonyl)-5-methyl-thiazole-4-carboxylate (27.88 mg, 84.90 μmol, 54.0% yield, 100.0% purity) as white solid. ¹H NMR (500 MHz, DMSO-d₆) δ ppm 12.34 (s, 1H), 9.07 (s, 1H), 8.34-8.25 (m, 1H), 7.61-7.52 (m, 1H), 7.33-7.24 (m, 2H), 5.22-5.15 (m, 1H), 2.81 (s, 3H), 1.38 (d, J=6.3 Hz, 6H); ES-LCMS m/z 328.9 [M+H]⁺.

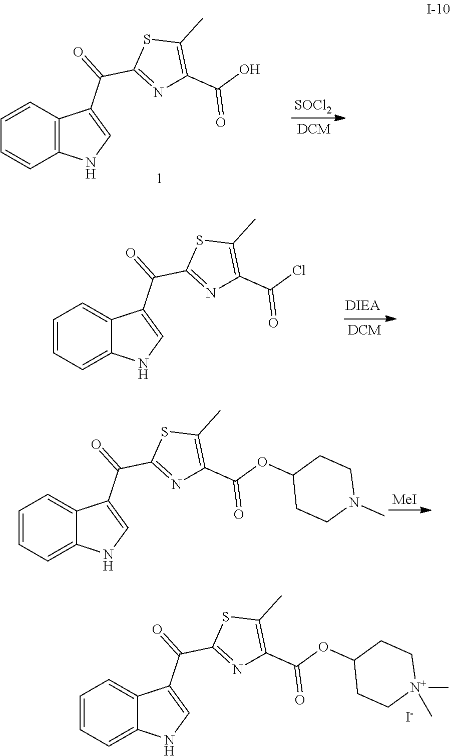

### Step 1: 2-(1H-Indole-3-carbonyl)-5-methyl-thiazole-4-carbonyl chloride

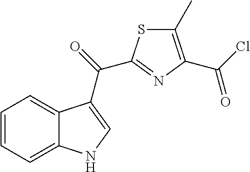 [SMILES: Cc1sc(C(=O)c2c[nH]c3ccccc23)nc1C(=O)Cl]

To a stirred solution of 2-(1H-indole-3-carbonyl)-5-methyl-thiazole-4-carboxylic acid (50 mg, 157.17 μmol, 1 eq) in DCM (5 mL) was added SOCl₂ (93.50 mg, 785.87 μmol, 57.01 μL, 5 eq) and DMF (1.15 mg, 15.72 μmol, 1.21 μL, 0.1 eq). The reaction mixture was stirred at 29° C. for 1 h. The reaction mixture was concentrated to yield 2-(1H-indole-3-carbonyl)-5-methyl-thiazole-4-carbonyl chloride (60 mg, crude, HCl) as yellow solid which was used in the next step without further purification.

### Step 2: (1-Methyl-4-piperidyl) 2-(1H-indole-3-carbonyl)-5-methyl-thiazole-4-carboxylate

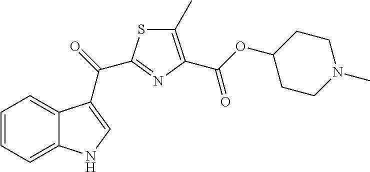 [SMILES: Cc1sc(C(=O)c2c[nH]c3ccccc23)nc1C(=O)OC1CCN(C)CC1]

To a stirred solution of 1-methylpiperidin-4-ol (60.76 mg, 527.53 μmol, 61.68 μL, 3 eq) in DCM (5 mL) was added DIEA (68.18 mg, 527.53 μmol, 91.88 μL, 3 eq) and 2-(1H-indole-3-carbonyl)-5-methyl-thiazole-4-carbonyl chloride (60 mg, 175.84 μmol, 1 eq, HCl). The reaction mixture was stirred at 29° C. for 50 min. The reaction mixture was concentrated to yield a residue which was purified by preparative HPLC (column: Agela DuraShell C18 150*25 mm*Sum; mobile phase: [water (0.04% NH₃.H₂O+10 mM NH₄HCO₃)-ACN]; B %: 41%-71%, 10 min). The desired fraction was lyophilized to yield (1-methyl-4-piperidyl) 2-(1H-indole-3-carbonyl)-5-methyl-thiazole-4-carboxylate (25 mg, 65.20 μmol, 37.1% yield, 100.0% purity) as yellow solid. ¹H NMR (500 MHz, DMSO-d₆) δ ppm 12.38 (s, 1H), 9.09 (s, 1H), 8.30 (dd, J=2.5, 6.0 Hz, 1H), 7.58 (dd, J=2.2, 6.2 Hz, 1H), 7.36-7.21 (m, 2H), 5.02 (s, 1H), 2.82 (s, 3H), 2.56 (d, J=10.4 Hz, 2H), 2.31 (s, 2H), 2.21 (s, 3H), 2.03-1.91 (m, 2H), 1.79 (dd, J=4.2, 8.2 Hz, 2H); ES-LCMS m/z 384.0 [M+H]⁺.

### Step 3: (1-BLAH-1,1-Dimethyl-1azinan-4-yl) 2-(1H-indole-3-carbonyl)-5-methyl-thiazole-4-carboxylate

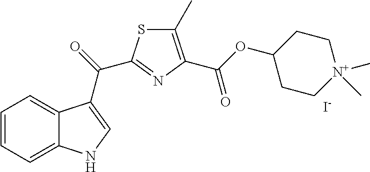 [SMILES: Cc1sc(C(=O)c2c[nH]c3ccccc23)nc1C(=O)OC1CC[N+](C)(C)CC1 ; [I-]]

To a stirred solution of (1-methyl-4-piperidyl) 2-(1H-indole-3-carbonyl)-5-methyl-thiazole-4-carboxylate (25 mg, 65.20 μmol, 1 eq) in ACN (3 mL) was added MeI (1.73 g, 12.19 mmol, 758.77 μL, 186.95 eq). The reaction mixture was stirred at 29° C. for 1 h. The reaction mixture was concentrated to yield (1-BLAH-1,1-dimethyl-1azinan-4-yl) 2-(1H-indole-3-carbonyl)-5-methyl-thiazole-4-carboxylate (28.72 mg, 53.57 μmol, 82.2% yield, 98.0% purity) as yellow solid which was lyophilized for delivery. ¹H NMR (400 MHz, DMSO-d₆) δ ppm 12.38 (s, 1H), 9.09 (s, 1H), 8.36-8.24 (m, 1H), 7.59 (d, J=6.4 Hz, 1H), 7.36-7.23 (m, 2H), 5.18 (s, 1H), 3.52 (s, 4H), 3.21 (s, 3H), 3.15 (s, 3H), 2.85 (s, 3H), 2.33 (s, 2H), 2.19 (s, 2H); ES-LCMS m/z 398.0 [M-I]⁺.

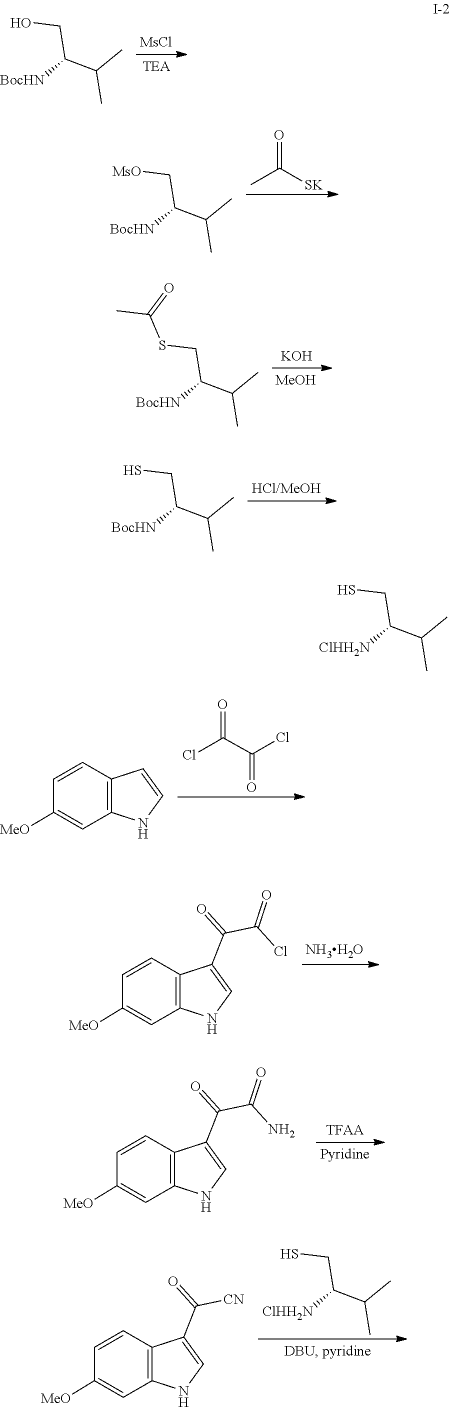

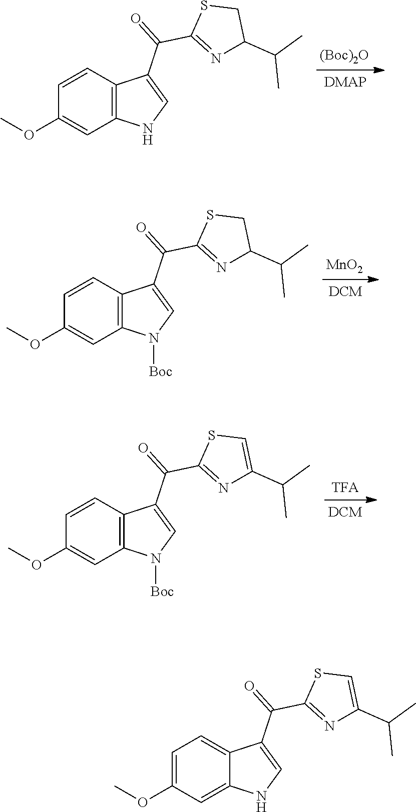

### Step 1: [(2S)-2-(tert-Butoxycarbonylamino)-3-methyl-butyl] methanesulfonate

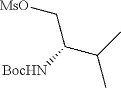 [SMILES: CC(C)[C@@H](COS(C)(=O)=O)NC(=O)OC(C)(C)C]

To a stirred solution of tert-butyl N-[(1S)-1-(hydroxymethyl)-2-methyl-propyl]carbamate (10 g, 49.19 mmol, 1 eq) and TEA (14.93 g, 147.58 mmol, 20.54 mL, 3 eq) in DCM (150 mL) was added MsCl (8.29 g, 72.37 mmol, 5.60 mL, 1.47 eq) at 0° C. The reaction mixture was stirred at 20° C. for 2 h. TLC (PE/EtOAc=1/1, R_{f}=0.29) showed starting material was consumed completely and one new spot was detected. The reaction mixture was diluted with water (150 mL) then extracted with EtOAc (200 mL×3). The combined organic layers were dried over Na₂SO₄, filtered and the filtrate was concentrated to yield[(2S)-2-(tert-butoxy carbonylamino)-3-methyl-butyl] methanesulfonate (14 g, 29.85 mmol, 60.7% yield, 60.0% purity) as a white solid. ¹H NMR (500 MHz, DMSO-d₆) δ ppm 6.93 (d, J=9.0 Hz, 1H), 4.19 (dd, J=4.2, 10.1 Hz, 1H), 4.08 (dd, J=7.6, 10.0 Hz, 1H), 3.54-3.51 (m, 1H), 3.17 (s, 3H), 1.76 (qd, J=6.8, 13.2 Hz, 1H), 1.40 (s, 9H), 0.88-0.84 (m, 6H).

### Step 2: S-[(2S)-2-(tert-Butoxycarbonylamino)-3-methyl-butyl] ethanethioate

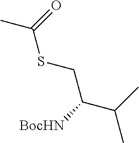 [SMILES: CC(=O)SC[C@@H](NC(=O)OC(C)(C)C)C(C)C]

To a solution of [(2S)-2-(tert-butoxycarbonylamino)-3-methyl-butyl] methanesulfonate (13 g, 27.72 mmol, 1 eq) in DMSO (100 mL) was added acetylsulfanylpotassium (4.75 g, 41.58 mmol, 1.5 eq). The mixture was stirred at 25° C. for 12 h. The reaction mixture was diluted with water (400 mL) then extracted with EtOAc (300 mL×3). The combined organic layers were dried over Na₂SO₄, filtered and the filterate was concentrated to yield a residue which was purified by flash silica gel chromatography (from PE/EtOAc=1/0 to 3/1, TLC:PE/EtOAc=3/1, R_{f}=0.55) to yield S-[(2S)-2-(tert-butoxycarbonylamino)-3-methyl-butyl] ethanethioate (5.4 g, 19.63 mmol, 70.8% yield, 95.0% purity) as a yellow solid. ¹H NMR (400 MHz, DMSO-d₆) δ ppm 6.71 (d, J=9.4 Hz, 1H), 3.31-3.25 (m, 1H), 3.10 (dd, J=3.7, 13.5 Hz, 1H), 2.69 (dd, J=9.8, 13.3 Hz, 1H), 2.29 (s, 3H), 1.71-1.62 (m, 1H), 1.36 (s, 9H), 0.82 (t, J=7.0 Hz, 6H); ES-LCMS m/z 262.0 [M+H]⁺.

### Step 3: tert-Butyl N-[(1S)-2-methyl-1-(sulfanylmethyl)propyl]carbamate

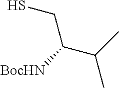

To a solution of S-[(2S)-2-(tert-butoxycarbonylamino)-3-methyl-butyl] ethanethioate (5.4 g, 19.63 mmol, 1 eq) in MeOH (40 mL) was added KOH (2.20 g, 39.25 mmol, 2 eq). The mixture was stirred at 25° C. for 30 min. TLC (PE/EtOAc=5/1, R_{f}=0.39) showed starting material was remained and one new spot was detected. The reaction mixture was quickly quenched with 50% citric acid (50 mL) then concentrated and extracted with DCM (100 mL×2). The combined organic layers were washed with 20% citric acid (20 mL×2), dried over Na₂SO₄ then concentrated to yield tert-butyl N-[(1S)-2-methyl-1-(sulfanylmethyl)propyl]carbamate (4.6 g, 17.83 mmol, 90.8% yield, 85.0% purity) as a yellow oil. ¹H NMR (500 MHz, DMSO-d₆) δ ppm 6.69-6.64 (m, 1H), 3.34-3.26 (m, 1H), 2.65-2.55 (m, 1H), 2.48-2.46 (m, 1H), 1.78 (t, J=6.5 Hz, 1H), 1.39 (s, 9H), 0.87-0.76 (m, 6H).

### Step 4: (2S)-2-Amino-3-methyl-butane-1-thiol; hydrochloride

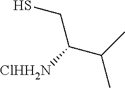

A mixture of tert-butyl N-[(1S)-2-methyl-1-(sulfanylmethyl)propyl]carbamate (4.6 g, 17.83 mmol, 1 eq) in HCl/MeOH (50 mL) was degassed and purged with N₂ for 3 times, and then the mixture was stirred at 80° C. for 2 h under N₂ atmosphere. TLC (PE/EtOAc=3/1, R_{f}=0) showed starting material was consumed completely and one new spot was detected. The reaction mixture was concentrated to yield (2S)-2-amino-3-methyl-butane-1-thiol; hydrochloride (3.1 g, 15.93 mmol, 89.36% yield, 80% purity) as yellow oil. ¹H NMR (400 MHz, DMSO-d₆) δ ppm 8.17 (s, 3H), 2.98 (d, J=5.7, 10.9 Hz, 1H), 2.86-2.76 (m, 1H), 2.74-2.64 (m, 1H), 2.05-1.94 (m, 1H), 1.00-0.88 (m, 6H).

### Step 5: 2-(6-Methoxy-1H-indol-3-yl)-2-oxo-acetyl chloride

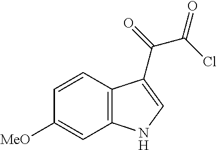 [SMILES: COc1ccc2c(C(=O)C(=O)Cl)c[nH]c2c1]

To a solution of 6-methoxy-1H-indole (2.3 g, 15.63 mmol, 1 eq) in THF (20 mL) was added oxalyl dichloride (2.18 g, 17.19 mmol, 1.50 mL, 1.1 eq) dropwise at 0-5° C. under N₂ atmosphere. The mixture was stirred at 0-5° C. for 3 h. The yellow slurry was filtered, and the cake was washed with PE (50 mL×2), dried under reduced pressure to yield 2-(6-methoxy-1H-indol-3-yl)-2-oxo-acetyl chloride (2.9 g, 10.69 mmol, 68.4% yield, 87.6% purity) as a yellow solid. ¹H NMR (500 MHz, DMSO-d₆) δ ppm 12.19 (s, 1H), 8.28 (d, J=3.2 Hz, 1H), 8.01 (d, J=8.7 Hz, 1H), 7.03 (d, J=2.1 Hz, 1H), 6.89 (dd, J=2.1, 8.7 Hz, 1H), 3.82 (s, 3H).

### Step 6: 2-(6-Methoxy-1H-indol-3-yl)-2-oxo-acetamide

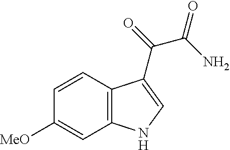 [SMILES: COc1ccc2c(C(=O)C(N)=O)c[nH]c2c1]

To a solution of NH₃.H₂O (15.28 g, 122.03 mmol, 16.79 mL, 28%, 10 eq) in EtOH (30 mL) was added 2-(6-methoxy-1H-indol-3-yl)-2-oxo-acetyl chloride (2.9 g, 12.20 mmol, 1 eq). The mixture was stirred at 0° C. for 2 h. The slurry was filtered, and the cake was washed with water (50 mL×2), dried under reduced pressure to yield 2-(6-methoxy-1H-indol-3-yl)-2-oxo-acetamide (2.1 g, 9.14 mmol, 74.9% yield, 95.0% purity) as a yellow solid. ¹H NMR (500 MHz, DMSO-d₆) δ ppm 11.94 (s, 1H), 8.55 (s, 1H), 8.12-7.97 (m, 2H), 7.66 (s, 1H), 7.01 (d, J=2.1 Hz, 1H), 6.88 (dd, J=2.3, 8.7 Hz, 1H), 3.79 (s, 3H).

### Step 7: 6-Methoxy-1H-indole-3-carbonyl cyanide

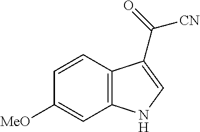 [SMILES: COc1ccc2c(C(=O)C#N)c[nH]c2c1]

To a solution of 2-(6-Methoxy-1H-indol-3-yl)-2-oxo-acetamide (2.1 g, 9.38 mmol, 1 eq) in pyridine (5.5 mL) was added TFAA (5.91 g, 28.15 mmol, 3.92 mL, 3 eq). The mixture was stirred at 25° C. for 12 h. The reaction mixture was quenched by addition of sat. aq. NaHCO₃ (100 mL), extracted with EtOAc (80 mL×3). The combined organic layers were washed with 0.5 N aq. HCl (40 mL), brine (40 mL), dried over Na₂SO₄, filtered and the filtrate was concentrated under reduced pressure to yield 6-methoxy-1H-indole-3-carbonyl cyanide (1.8 g, 8.90 mmol, 94.9% yield, 99.0% purity) as yellow solid. ¹H NMR (500 MHz, DMSO-d₆) δ ppm 12.72 (s, 1H), 8.52 (d, J=3.4 Hz, 1H), 7.89 (d, J=8.7 Hz, 1H), 7.05 (d, J=2.0 Hz, 1H), 6.96 (dd, J=2.1, 8.7 Hz, 1H), 3.81 (s, 3H); ES-LCMS m/z 201.0 [M+H]⁺.

### Step 8: (4-Isopropyl-4,5-dihydrothiazol-2-yl)-(6-methoxy-1H-indol-3-yl)methanone

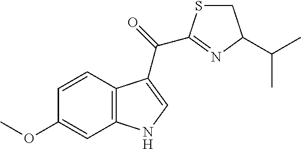 [SMILES: COc1ccc2c(C(=O)C3=NC(C(C)C)CS3)c[nH]c2c1]

To a solution of 6-Methoxy-1H-indole-3-carbonyl cyanide (1.7 g, 8.41 mmol, 1 eq) in pyridine (10 mL) was added DBU (127.99 mg, 840.69 μmol, 126.72 μL, 0.1 eq) and (2S)-2-amino-3-methyl-butane-1-thiol; hydrochloride (1.64 g, 8.41 mmol, 1 eq). The mixture was stirred at 25° C. for 2 h under N₂ atmosphere. The reaction mixture was filtered and the filtrate was concentrated to yield (4-isopropyl-4,5-dihydrothiazol-2-yl)-(6-methoxy-1H-indol-3-yl)methanone (1.1 g, 3.64 mmol, 43.3% yield, 100.0% purity) as a yellow solid. ¹H NMR (500 MHz, DMSO-d₆) δ ppm 11.99 (s, 1H), 8.53 (d, J=2.6 Hz, 1H), 8.06 (d, J=8.7 Hz, 1H), 7.04 (d, J=2.3 Hz, 1H), 6.89 (dd, J=2.3, 8.7 Hz, 1H), 4.58 (dt, J=6.4, 9.6 Hz, 1H), 3.80 (s, 3H), 3.39 (dd, J=9.2, 11.1 Hz, 1H), 3.04 (t, J=10.7 Hz, 1H), 2.07 (d, J=6.7, 13.3 Hz, 1H), 1.09 (d, J=6.7 Hz, 3H), 1.03 (d, J=6.7 Hz, 3H); ES-LCMS m/z 303.0 [M+H]⁺.

### Step 9: tert-Butyl 3-(4-isopropyl-4,5-dihydrothiazole-2-carbonyl)-6-methoxy-indole-1-carboxylate

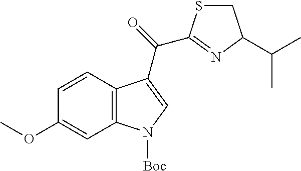 [SMILES: COc1ccc2c(C(=O)C3=NC(C(C)C)CS3)cn(C(=O)OC(C)(C)C)c2c1]

To a solution of (4-isopropyl-4,5-dihydrothiazol-2-yl)-(6-methoxy-1H-indol-3-yl)methanone (1 g, 3.31 mmol, 1 eq) in 1,4-dioxane (20 mL) was added (Boc)₂O (938.27 mg, 4.30 mmol, 987.66 μL, 1.3 eq) and DMAP (444.42 mg, 3.64 mmol, 1.1 eq). The mixture was stirred at 70° C. for 2 h under N₂ atmosphere. The reaction mixture was diluted with water (150 mL), extracted with EtOAc (50 mL×3). The combined organic layers were dried over Na₂SO₄, filtered and the filtrate was concentrated to yield a residue which was purified by flash silica gel chromatography (from PE/EtOAc=100/1 to 2/1, TLC:PE/EtOAc=3/1, R_{f}=0.65) to yield tert-butyl 3-(4-isopropyl-4,5-dihydrothiazole-2-carbonyl)-6-methoxy-indole-1-carboxylate (640 mg, 1.59 mmol, 48.1% yield, 100.0% purity) as a yellow solid. ¹H NMR (400 MHz, DMSO-d₆) δ ppm 8.96 (s, 1H), 8.22 (d, J=8.6 Hz, 1H), 7.84 (s, 1H), 7.73 (s, 1H), 4.59 (d, J=9.0 Hz, 1H), 3.83 (s, 3H), 3.51-3.42 (m, 2H), 3.08-3.07 (m, 1H), 1.67 (s, 9H), 1.11-1.05 (m, 6H); ES-LCMS m/z 403.1 [M+H]⁺.

### Step 10: tert-Butyl 3-(4-isopropylthiazole-2-carbonyl)-6-methoxy-indole-1-carboxylate

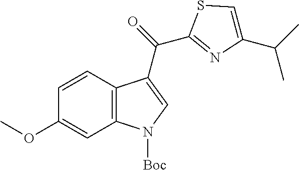 [SMILES: COc1ccc2c(C(=O)c3nc(C(C)C)cs3)cn(C(=O)OC(C)(C)C)c2c1]

To a solution of tert-butyl 3-(4-isopropyl-4,5-dihydrothiazole-2-carbonyl)-6-methoxy-indole-1-carboxylate (500 mg, 1.24 mmol, 1 eq) in 1,2-dichloroethane (10 mL) was added MnO₂ (1.62 g, 18.63 mmol, 15 eq). The mixture was stirred at 95° C. for 12 h under N₂ atmosphere. The reaction mixture was filtered through a pad of celite and the filtrate was concentrated to yield tert-butyl 3-(4-isopropylthiazole-2-carbonyl)-6-methoxy-indole-1-carboxylate (440 mg, 1.04 mmol, 84.0% yield, 95.0% purity) as a yellow solid. ¹H NMR (500 MHz, DMSO-d₆) δ ppm 9.37 (s, 1H), 8.22 (d, J=8.9 Hz, 1H), 7.84 (d, J=0.6 Hz, 1H), 7.72 (d, J=2.1 Hz, 1H), 7.05 (dd, J=2.4, 8.8 Hz, 1H), 3.84 (s, 3H), 3.18 (td, J=6.8, 13.5 Hz, 1H), 1.67 (s, 9H), 1.37 (d, J=6.9 Hz, 6H); ES-LCMS m/z 401.1 [M+H]⁺.

### Step 11: (4-Isopropylthiazol-2-yl)-(6-methoxy-1H-indol-3-yl)methanone

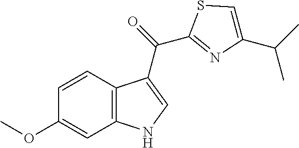 [SMILES: COc1ccc2c(C(=O)c3nc(C(C)C)cs3)c[nH]c2c1]

To a solution of tert-butyl 3-(4-isopropylthiazole-2-carbonyl)-6-methoxy-indole-1-carboxylate (440 mg, 1.04 mmol, 1 eq) in DCM (6 mL) was added TFA (3.08 g, 27.01 mmol, 2.00 mL, 25.88 eq). The mixture was stirred at 28° C. for 1 h under N₂ atmosphere. The reaction mixture was concentrated to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 55%-75%, 10 min) and lyophilized to yield (4-isopropylthiazol-2-yl)-(6-methoxy-1H-indol-3-yl)methanone (200 mg, 665.83 μmol, 63.8% yield, 100.0% purity) as a yellow solid. ¹H NMR (500 MHz, DMSO-d₆) δ ppm 11.98 (s, 1H), 8.97 (s, 1H), 8.16 (d, J=8.7 Hz, 1H), 7.70 (d, J=0.8 Hz, 1H), 7.06 (d, J=2.3 Hz, 1H), 6.90 (dd, J=2.3, 8.7 Hz, 1H), 3.81 (s, 3H), 3.21-3.16 (m, 1H), 1.35 (d, J=6.9 Hz, 6H); ES-LCMS m/z 301.1 [M+H]⁺.

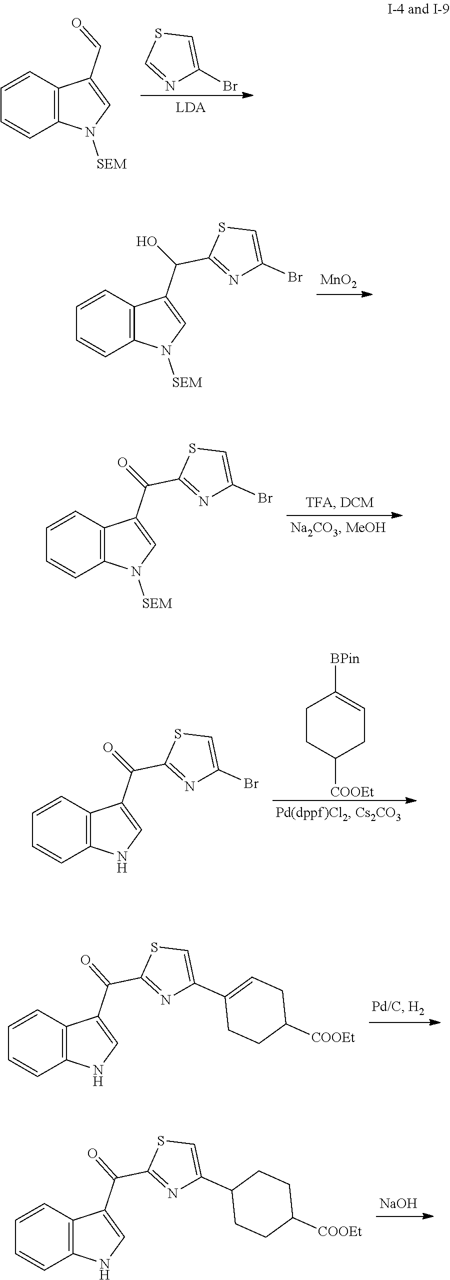

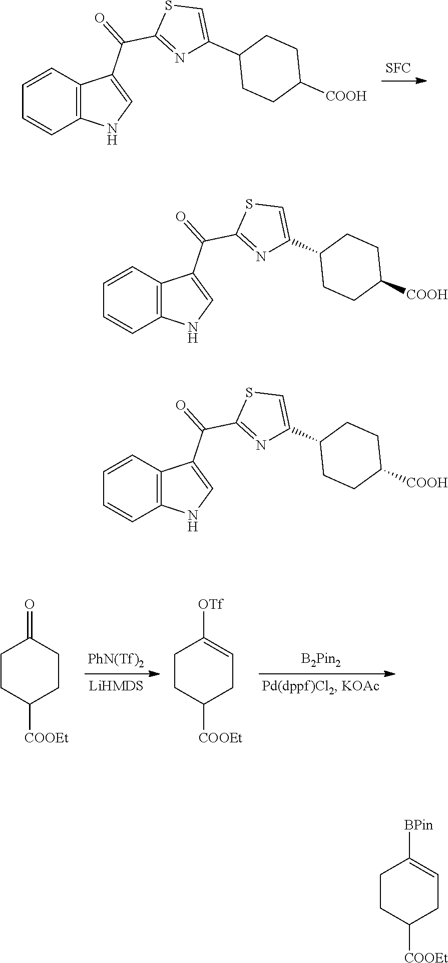

### Step 1: Ethyl 4-(trifluoromethylsulfonyloxy)cyclohex-3-ene-1-carboxylate

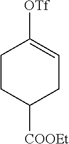 [SMILES: CCOC(=O)C1CC=C(OS(=O)(=O)C(F)(F)F)CC1]

To a solution of ethyl 4-oxocyclohexanecarboxylate (2 g, 11.75 mmol, 1.87 mL, 1 eq) in THF (50 mL) was added LiHMDS (1 M, 12.34 mL, 21%, 1.05 eq) drop-wise at −70° C. under N₂. During which the temperature was maintained below −70° C. for 1 h, A solution of 1,1,1-trifluoro-N-phenyl-N-(trifluoromethylsulfonyl)methanesulfonamide (4.41 g, 12.34 mmol, 1.05 eq) in THF (10 mL) was added dropwise at −70° C. The resulting mixture was stirred at 20° C. for another 12 h. TLC (PE/EtOAc=3/1, R_{f}=0.28) indicated the starting material was consumed completely and two new spots formed. The reaction mixture was quenched by addition of sat. aq. NaHCO₃ (100 mL), extracted with EtOAc (100 mL×3). The combined organic layers were washed with brine (30 mL), dried over Na₂SO₄, filtered and concentrated under reduced pressure to yield ethyl 4-(trifluoromethylsulfonyloxy)cyclohex-3-ene-1-carboxylate (3.55 g, crude) as a yellow solid, which was used in the next step without further purification. ¹H NMR (400 MHz, CDCl₃) δ ppm 5.87-5.78 (m, 1H), 4.21-4.14 (m, 2H), 2.67 (dt, J=3.1, 7.0 Hz, 1H), 2.54-2.45 (m, 4H), 2.26-2.14 (m, 1H), 2.08-1.88 (m, 2H), 1.35-1.32 (m, 3H); ES-LCMS m/z 302.9 [M+H]⁺.

### Step 2: Ethyl 4-(4,4,5,5-tetramethyl-1,3,2-dioxaborolan-2-yl)cyclohex-3-ene-1-carboxylate

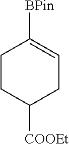

A mixture of ethyl 4-(trifluoromethylsulfonyloxy)cyclohex-3-ene-1-carboxylate (2.55 g, 8.44 mmol, 1 eq), 4,4,5,5-tetramethyl-2-(4,4,5,5-tetramethyl-1,3,2-dioxaborolan-2-yl)-1,3,2-dioxaborolane (3.21 g, 12.65 mmol, 1.5 eq), Pd(dppf)Cl₂ (617.29 mg, 843.63 μmol, 0.1 eq), KOAc (2.48 g, 25.31 mmol, 3 eq) in 1,4-dioxane (50 mL) was degassed and purged with N₂ for 3 times, then the mixture was stirred at 110° C. for 12 h under N₂ atmosphere. TLC (PE/EtOAc=3/1, R_{f}=0.60) indicated the starting material was consumed completely and one new major spot formed. The reaction mixture was quenched by addition of water (100 mL), extracted with EtOAc (100 mL×3). The combined organic layers were washed with brine (10 mL), dried over Na₂SO₄, filtered and concentrated under reduced pressure to give a residue. The residue was purified by flash silica gel chromatography (from PE/EtOAc=1/0 to 5/1, TLC:PE/EtOAc=5/1, R_{f}=0.6) to yield ethyl 4-(4,4,5,5-tetramethyl-1,3,2-dioxaborolan-2-yl)cyclohex-3-ene-1-carboxylate (1.1 g, 3.53 mmol, 41.8% yield, 90.0% purity) as yellow oil. ¹H NMR (500 MHz, CDCl₃) δ ppm 6.51 (br s, 1H), 4.20-4.04 (m, 2H), 2.55-2.42 (m, 1H), 2.36-2.21 (m, 3H), 2.15-1.93 (m, 2H), 1.64-1.50 (m, 1H), 1.24-1.22 (m, 15H); ES-LCMS m/z 281.1 [M+H]⁺.

### Step 3: (4-Bromothiazol-2-yl)-[1-(2-trimethylsilylethoxymethyl)indol-3-yl]methanol

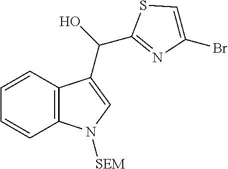 [SMILES: C[Si](C)(C)CCOCn1cc(C(O)c2nc(Br)cs2)c2ccccc21]

To a solution of n-BuLi (2.5 M, 2.32 mL, 2.0 eq) in THF (50 mL) was added i-Pr₂NH (586.08 mg, 5.79 mmol, 818.54 μL, 2.0 eq) at −70° C. under N₂. After being stirred for 0.5 h, a solution of 4-bromothiazole (500 mg, 2.90 mmol, 1 eq) and 1-(2-trimethylsilylethoxymethyl)indole-3-carbaldehyde (797.59 mg, 2.90 mmol, 1 eq) in THF (10) was added dropwise. After addition, the mixture was stirred at −70° C. for 2 h under N₂ atmosphere. The reaction mixture was quenched by addition of water (50 mL), extracted with EtOAc (50 mL×3). The combined organic layers were washed with brine (30 mL), dried over Na₂SO₄, filtered and concentrated under reduced pressure. The residue was purified by flash silica gel chromatography (from PE/EtOAc=1/0 to 3/1, TLC:PE/EtOAc=3/1, R_{f}=0.22) to yield a (4-bromothiazol-2-yl)-[1-(2-trimethylsilylethoxymethyl)indol-3-yl]methanol (680 mg, 1.19 mmol, 41.1% yield, 77.0% purity) as yellow oil. ¹H NMR (400 MHz, CDCl₃) δ ppm 7.69 (d, J=8.1 Hz, 1H), 7.56 (d, J=8.3 Hz, 1H), 7.46-7.39 (m, 1H), 7.32 (d, J=6.4 Hz, 2H), 7.25-7.18 (m, 1H), 6.41 (d, J=3.2 Hz, 1H), 5.55-5.49 (m, 2H), 3.56-3.54 (m, 2H), 0.96 (d, J=8.3 Hz, 2H), 0.01 (s, 9H); ES-LCMS m/z 420.9, 422.9 [M−H₂O+H]⁺.

### Step 4: (4-Bromothiazol-2-yl)-[1-(2-trimethylsilylethoxymethyl)indol-3-yl]methanone

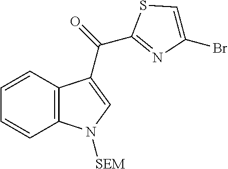 [SMILES: C[Si](C)(C)CCOCn1cc(C(=O)c2nc(Br)cs2)c2ccccc21]

To a solution or (4-bromothiazol-2-yl)-[1-(2-trimethylsilylethoxymethyl)indol-3-yl]methanol (680 mg, 1.19 mmol, 1 eq) in CHCl₃ (20 mL) was added MnO₂ (1.55 g, 17.87 mmol, 15 eq). The mixture was stirred at 50° C. for 12 h under N₂. The mixture was filtered through celite, and the cake was rinsed with DCM (2×30 mL). The filtrate was concentrated. The residue was purified by flash silica gel chromatography (from PE/EtOAc=1/0 to 10/1, TLC:PE/EtOAc=5/1, R_{f}=0.68) to yield (4-bromothiazol-2-yl)-[1-(2-trimethylsilylethoxymethyl)indol-3-yl]methanone (400 mg, 868.72 μmol, 72.9% yield, 95.0% purity) as a yellow solid. ¹H NMR (500 MHz, CDCl₃) δ ppm 9.06 (s, 1H), 8.57-8.49 (m, 1H), 7.63-7.54 (m, 2H), 7.44-7.35 (m, 2H), 5.63 (s, 2H), 3.62-3.55 (m, 2H), 0.99-0.91 (m, 2H), −0.03 (s, 9H); ES-LCMS m/z 436.9, 438.9 [M+H]⁺.

### Step 5: (4-Bromothiazol-2-yl)-(1H-indol-3-yl)methanone

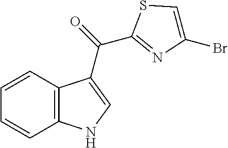 [SMILES: O=C(c1nc(Br)cs1)c1c[nH]c2ccccc12]

To a solution of (4-bromothiazol-2-yl)-[1-(2-trimethylsilylethoxymethyl)indol-3-yl]methanone (400 mg, 868.72 μmol, 1 eq) in DCM (5 mL) was added TFA (6.65 g, 58.32 mmol, 4.32 mL, 67.14 eq). The mixture was stirred at 25° C. for 1 h. TLC (PE/EtOAc=3/1, R_{f}=0.22) indicated the starting material was consumed completely and one new spot formed. The mixture was dissolved in MeOH (10 mL), adjusted pH to 9 by addition of Na₂CO₃ (92.07 mg, 868.72 μmol, 1 eq) in water (2 mL). The resulting mixture was stirred at 25° C. for 2 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield (4-bromothiazol-2-yl)-(1H-indol-3-yl)methanone (350 mg, 518.45 μmol, 59.6% yield, 45.5% purity) as a yellow solid, which was used in the next step without further purification. ¹H NMR (500 MHz, DMSO-d₆) δ ppm 9.04 (s, 1H), 8.36-8.33 (m, 2H), 7.67-7.61 (m, 1H), 7.36-7.32 (m, 2H); ES-LCMS m/z 306.8, 308.8 [M+H]⁺.

### Step 6: Ethyl 4-[2-(1H-indole-3-carbonyl)thiazol-4-yl]cyclohex-3-ene-1-carboxylate

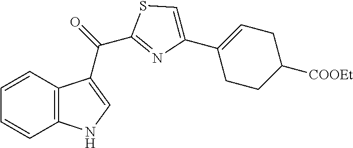 [SMILES: CCOC(=O)C1CC=C(c2csc(C(=O)c3c[nH]c4ccccc34)n2)CC1]

A mixture of (4-bromothiazol-2-yl)-(1H-indol-3-yl)methanone (300 mg, 444.39 μmol, 1 eq), ethyl 4-(4,4,5,5-tetramethyl-1,3,2-dioxaborolan-2-yl)cyclohex-3-ene-1-carboxylate (415.01 mg, 1.33 mmol, 3 eq), Pd(dppf)Cl₂ (32.52 mg, 44.44 umol, 0.1 eq) and Cs₂CO₃ (2 M, 444.39 μL, 2 eq) in 1,4-dioxane (10 mL) was de-gassed and then heated to 110° C. for 1 h under N₂. The reaction mixture was quenched by addition of water (50 mL), extracted with EtOAc (50 mL×3). The combined organic layers were washed with brine (20 mL), dried over Na₂SO₄, filtered and concentrated under reduced pressure. The residue was purified by flash silica gel chromatography (from PE/EtOAc=1/0 to 3/1, TLC:PE/EtOAc=3/1, R_{f}=0.28) to yield ethyl 4-[2-(1H-indole-3-carbonyl)thiazol-4-yl]cyclohex-3-ene-1-carboxylate (250 mg, 405.43 μmol, 91.2% yield, 61.7% purity) as a yellow solid. ¹H NMR (500 MHz, CDCl₃) δ ppm 9.19-9.05 (m, 2H), 8.53-8.45 (m, 1H), 7.45-7.38 (m, 1H), 7.35-7.28 (m, 3H), 6.74 (d, J=2.0 Hz, 1H), 4.20-4.09 (m, 2H), 2.66-2.41 (m, 5H), 2.23-2.12 (m, 1H), 1.90-1.83 (m, 1H), 1.26-1.22 (m, 3H); ES-LCMS m/z 380.9 [M+H]⁺.

### Step 7: Ethyl 4-[2-(1H-indole-3-carbonyl)thiazol-4-yl]cyclohexanecarboxylate

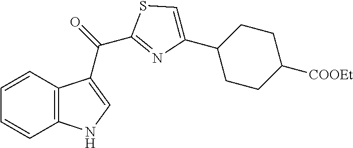 [SMILES: CCOC(=O)C1CCC(c2csc(C(=O)c3c[nH]c4ccccc34)n2)CC1]

To a solution of ethyl 4-[2-(1H-indole-3-carbonyl)thiazol-4-yl]cyclohex-3-ene-1-carboxylate (200 mg, 324.34 μmol, 1 eq) in THF (10 mL) was added Pd/C (50 mg, 10%) under H2. The mixture was stirred at 30° C. for 12 h under H2 (15 psi). The reaction mixture was filtered and the filtrate was concentrated. The residue was purified by flash silica gel chromatography (from PE/EtOAc=1/0 to 3/1, TLC:PE/EtOAc=3/1, R_{f}=0.29) to yield ethyl 4-[2-(1H-indole-3-carbonyl)thiazol-4-yl]cyclohexanecarboxylate (120 mg, 251.31 μmol, 77.4% yield, 80.1% purity) as a white solid. ¹H NMR (500 MHz, CD₃OD) δ ppm 9.18-9.09 (m, 1H), 8.43-8.33 (m, 1H), 7.57-7.45 (m, 2H), 7.35-7.22 (m, 2H), 4.23-4.08 (m, 2H), 3.11-2.89 (m, 1H), 2.79-2.64 (m, 1H), 2.30-2.11 (m, 2H), 2.10-1.87 (m, 3H), 1.86-1.52 (m, 3H), 1.30-1.25 (m, 3H); ES-LCMS m/z 383.0 [M+H]⁺.

### Step 8: (E) 4-[2-(1H-Indole-3-carbonyl)thiazol-4-yl]cyclohexanecarboxylic acid & (Z) 4-[2-(1H-Indole-3-carbonyl)thiazol-4-yl]cyclohexanecarboxylic acid

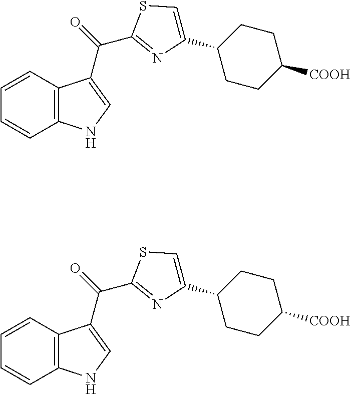 [SMILES: O=C(c1nc([C@H]2CC[C@H](C(=O)O)CC2)cs1)c1c[nH]c2ccccc12 ; O=C(c1nc([C@H]2CC[C@@H](C(=O)O)CC2)cs1)c1c[nH]c2ccccc12]

To a solution of ethyl 4-[2-(1H-indole-3-carbonyl)thiazol-4-yl]cyclohexanecarboxylate (120 mg, 251.31 μmol, 1 eq) in THF (3 mL) and MeOH (3 mL) was added NaOH (2 M, 628.28 μL, 5 eq). The mixture was stirred at 30° C. for 12 h. The reaction mixture was adjusted pH to 6 by 1N HCl solution, concentrated. The residue was purified by preparative HPLC (column: Phenomenex Synergi C18 150*30 mm*4 μm; mobile phase: [water (0.05% HCl)-ACN]; B %: 46%-66%, 10 min), followed by lyophilization to yield 4-[2-(1H-indole-3-carbonyl)thiazol-4-yl]cyclohexanecarboxylic acid (40 mg, 112.86 μmol, 44.9% yield, 100% purity) as a white solid, which was separated by SFC (column: DAICEL CHIRALPAK AD-H (250 mm*30 mm, 5 um); mobile phase: [0.1% NH₃.H₂O IPA]; B %: 45%-45%, min) to yield peak 1 (Rₜ=1.992) and peak 2 (Rₜ=2.147). Peak 1 was concentrated under reduced pressure to yield a residue which was dissolved in MeCN (20 mL) and H₂O (10 mL), lyophilized to yield (E) 4-[2-(1H-indole-3-carbonyl)thiazol-4-yl]cyclohexanecarboxylic acid (6.22 mg, 17.55 μmol, 15.5% yield, 100% purity) (ee=97.62%, Rₜ=1.991) as a white solid. ¹H NMR (500 MHz, DMSO-d₆) δ ppm 12.19 (br s, 1H), 9.08 (s, 1H), 8.42-8.21 (m, 1H), 7.72 (s, 1H), 7.57 (d, J=6.9 Hz, 1H), 7.34-7.19 (m, 2H), 2.85 (br s, 1H), 2.34-2.23 (m, 1H), 2.18 (d, J=10.7 Hz, 2H), 2.04 (d, J=10.8 Hz, 2H), 1.64-1.44 (m, 4H); ES-LCMS m/z 355.0 [M+H]⁺. Peak 2 was concentrated under reduced pressure to yield a residue which was dissolved in MeCN (20 mL) and H₂O (10 mL), lyophilized to yield (Z) 4-[2-(1H-indole-3-carbonyl)thiazol-4-yl]cyclohexanecarboxylic acid (13.99 mg, 39.47 μmol, 34.9% yield, 100% purity) (ee=98.92%, Rₜ=2.147) as a white solid. ¹H NMR (500 MHz, DMSO-d₆) δ ppm 12.20 (br s, 1H), 9.04 (s, 1H), 8.31 (dd, J=2.4, 6.1 Hz, 1H), 7.75 (s, 1H), 7.57 (dd, J=2.1, 6.3 Hz, 1H), 7.32-7.22 (m, 2H), 3.07-2.92 (m, 1H), 2.59 (d, J=4.0 Hz, 1H), 2.08-1.92 (m, 4H), 1.89-1.76 (m, 2H), 1.73-1.62 (m, 2H); ES-LCMS m/z 355.0 [M+H]⁺.

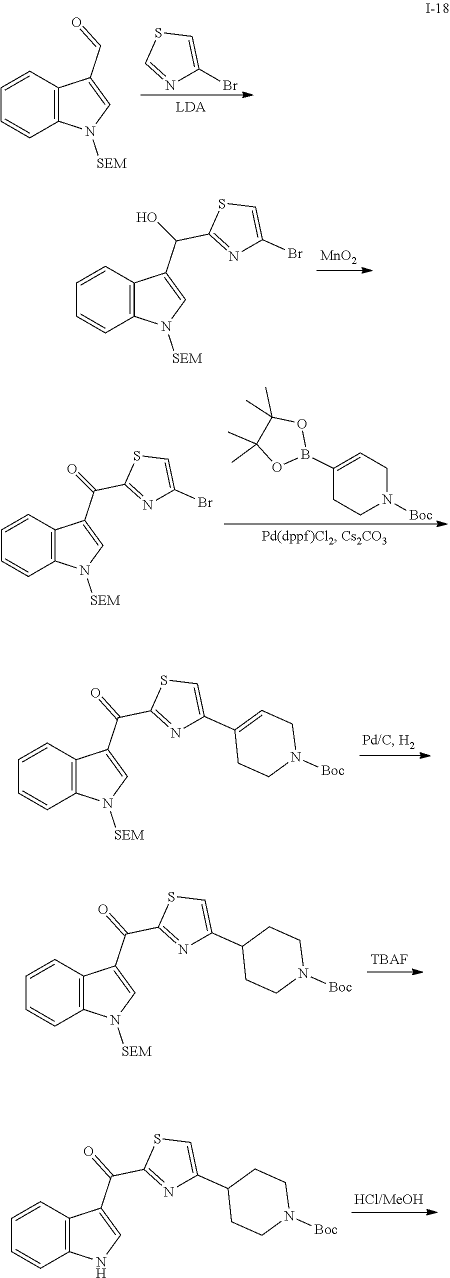

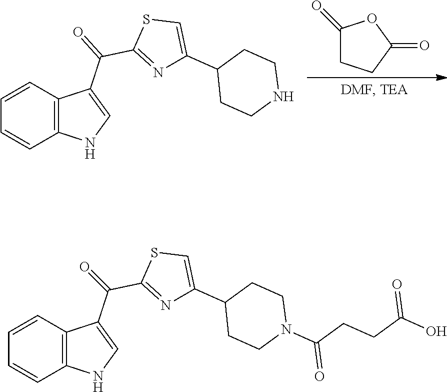 [SMILES: O=C(c1nc(C2CCNCC2)cs1)c1c[nH]c2ccccc12 ; O=C1CCC(=O)O1 ; O=C(O)CCC(=O)N1CCC(c2csc(C(=O)c3c[nH]c4ccccc34)n2)CC1]

### Step 1: (4-Bromothiazol-2-yl)-[1-(2-trimethylsilylethoxymethyl)indol-3-yl]methanol

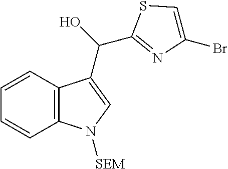 [SMILES: C[Si](C)(C)CCOCn1cc(C(O)c2nc(Br)cs2)c2ccccc21]

To a solution of n-BuLi (2.5 M, 13.41 mL, 2.5 eq) in THF (200 mL) was added i-Pr₂NH (3.39 g, 33.53 mmol, 4.74 mL, 2.5 eq) at −70° C. under N₂. After being stirred for 0.5 h, a solution of 4-bromothiazole (2.2 g, 13.41 mmol, 1 eq) and 1-(2-trimethylsilylethoxymethyl)indole-3-carbaldehyde (3.73 g, 13.41 mmol, 1 eq) in THF (40 mL) was added dropwise. After addition, the mixture was stirred at −70° C. for 2 h under N₂ atmosphere. The reaction mixture was quenched by addition of water (100 mL), extracted with EtOAc (100 mL×3). The combined organic layers were washed with brine (30 mL), dried over Na₂SO₄, filtered and concentrated under reduced pressure. The residue was purified by flash silica gel chromatography (from PE/EtOAc=1/0 to 3/1, TLC:PE/EtOAc=3/1, R_{f}=0.22) to yield (4-bromothiazol-2-yl)-[1-(2-trimethylsilylethoxymethyl)indol-3-yl]methanol (1.9 g, 3.48 mmol, 25.9% yield, 80.5% purity) as yellow oil. ¹H NMR (400 MHz, DMSO-d₆) δ ppm 7.75 (s, 1H), 7.53 (dd, J=8.1, 11.2 Hz, 2H), 7.44 (s, 1H), 7.16 (t, J=7.2 Hz, 1H), 7.06-6.99 (m, 1H), 6.73 (d, J=4.6 Hz, 1H), 6.17 (d, J=4.4 Hz, 1H), 5.52 (s, 2H), 3.44 (t, J=8.1 Hz, 2H), 0.82 (d, J=7.8 Hz, 2H), −0.09 (s, 9H); ES-LCMS m/z 302.9 [M+H]⁺.

### Step 2: (4-Bromothiazol-2-yl)-[1-(2-trimethylsilylethoxymethyl)indol-3-yl]methanone

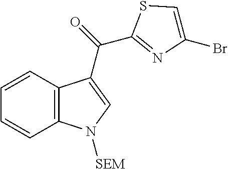 [SMILES: C[Si](C)(C)CCOCn1cc(C(=O)c2nc(Br)cs2)c2ccccc21]

To a solution of (4-bromothiazol-2-yl)-[1-(2-trimethylsilylethoxymethyl)indol-3-yl]methanol (1.9 g, 3.48 mmol, 1 eq) in CHCl₃ (100 mL) was added MnO₂ (3.03 g, 34.81 mmol, 10 eq). The mixture was stirred at 50° C. for 12 h under N₂. The mixture was filtered through celite, and the cake was rinsed with DCM (2×30 mL). The filtrate was concentrated. The residue was purified by flash silica gel chromatography (from PE/EtOAc=1/0 to 10/1, TLC:PE/EtOAc=5/1, R_{f}=0.68) to yield (4-bromothiazol-2-yl)-[1-(2-trimethylsilylethoxymethyl)indol-3-yl]methanone (1.3 g, 2.97 mmol, 85.3% yield, 100% purity) as a light yellow solid. ¹H NMR (500 MHz, CDCl₃) δ ppm 9.11-9.02 (m, 1H), 8.59-8.47 (m, 1H), 7.62-7.58 (m, 1H), 7.56 (s, 1H), 7.42-7.36 (m, 2H), 5.63 (s, 2H), 3.61-3.55 (m, 2H), 0.99-0.92 (m, 2H), −0.03 (s, 9H); ES-LCMS m/z 281.1 [M+H]⁺.

### Step 3: tert-Butyl 4-[2-[1-(2-trimethylsilylethoxymethyl)indole-3-carbonyl]thiazol-4-yl]-3,6-dihydro-2H-pyridine-1-carboxylate

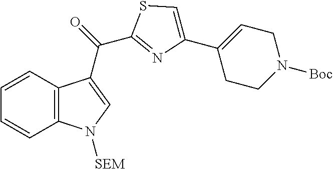 [SMILES: CC(C)(C)OC(=O)N1CC=C(c2csc(C(=O)c3cn(COCC[Si](C)(C)C)c4ccccc34)n2)CC1]

A mixture of (4-bromothiazol-2-yl)-[1-(2-trimethylsilylethoxymethyl)indol-3-yl]methanone (580 mg, 1.33 mmol, 1 eq), (1-tert-butoxycarbonyl-3,6-dihydro-2H-pyridin-4-yl)boronic acid (392.60 mg, 1.73 mmol, 1.3 eq), Pd(dppf)Cl₂ (97.32 mg, 133.00 μmol, 0.1 eq) and Cs₂CO₃ (1 M, 2.66 mL, 2 eq) in 1,4-dioxane (13 mL) was de-gassed and then heated to 100° C. for 1 h under N₂ under microwave (2 bar). TLC (PE/EtOAc=1/1, R_{f}=0.29) indicated the starting material was consumed completely and one new spot formed. The reaction mixture was quenched by addition of water (50 mL), extracted with EtOAc (50 mL×3). The combined organic layers were washed with brine (10 mL), dried over Na₂SO₄, filtered and concentrated under reduced pressure. The residue was purified by flash silica gel chromatography (from PE/EtOAc=1/0 to 3/1, TLC:PE/EtOAc=1/1, R_{f}=0.29) to yield tert-butyl 4-[2-[1-(2-trimethylsilylethoxymethyl)indole-3-carbonyl]thiazol-4-yl]-3,6-dihydro-2H-pyridine-1-carboxylate (600 mg, 1.09 mmol, 81.6% yield, 97.7% purity) as a yellow solid. ¹H NMR (400 MHz, CDCl₃) δ ppm 9.14-9.02 (m, 1H), 8.64-8.49 (m, 1H), 7.68-7.51 (m, 1H), 7.48-7.31 (m, 3H), 6.69 (br s, 1H), 5.62 (s, 2H), 4.19 (br s, 2H), 3.71 (t, J=5.7 Hz, 2H), 3.63-3.50 (m, 2H), 2.63 (br s, 2H), 1.52 (s, 9H), 0.98-0.92 (m, 2H), −0.04 (s, 9H); ES-LCMS m/z 420.9, 422.9 [M−H₂O+H]⁺.

### Step 4: tert-Butyl 4-[2-[1-(2-trimethylsilylethoxymethyl)indole-3-carbonyl]thiazol-4-yl]piperidine-1-carboxylate

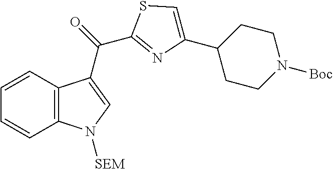 [SMILES: CC(C)(C)OC(=O)N1CCC(c2csc(C(=O)c3cn(COCC[Si](C)(C)C)c4ccccc34)n2)CC1]

To a solution of tert-butyl 4-[2-[1-(2-trimethylsilylethoxymethyl)indole-3-carbonyl]thiazol-4-yl]-3,6-dihydro-2H-pyridine-1-carboxylate (600 mg, 1.09 mmol, 1 eq) in THF (10 mL) and MeOH (10 mL) was added Pd/C (0.6 g, 10% purity) under H2. The mixture was stirred at 25° C. for 12 h. The reaction mixture was filtered through celite and the filtrate was concentrated to yield tert-butyl 4-[2-[1-(2-trimethylsilylethoxymethyl)indole-3-carbonyl]thiazol-4-yl]piperidine-1-carboxylate (450 mg, 701.86 μmol, 64.6% yield, 84.5% purity) as a yellow solid, which was used in the next step without further purification. ¹H NMR (500 MHz, DMSO-d₆) δ ppm 9.18-9.08 (m, 1H), 8.39-8.29 (m, 1H), 7.82 (s, 1H), 7.72 (d, J=7.5 Hz, 1H), 7.41-7.30 (m, 2H), 5.77 (s, 2H), 4.06 (br s, 2H), 3.60 (ddd, J=2.6, 4.2, 6.5 Hz, 2H), 3.54 (t, J=8.0 Hz, 2H), 3.11-3.03 (m, 1H), 2.09 (d, J=12.7 Hz, 2H), 1.61 (dq, J=4.1, 12.4 Hz, 2H), 1.42 (s, 9H), 0.86 (t, J=7.9 Hz, 2H), −0.07-−0.11 (m, 9H); ES-LCMS m/z 436.9, 438.9 [M+H]⁺.

### Step 5: tert-Butyl 4-[2-(1H-indole-3-carbonyl)thiazol-4-yl]piperidine-1-carboxylate

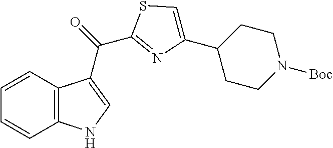 [SMILES: CC(C)(C)OC(=O)N1CCC(c2csc(C(=O)c3c[nH]c4ccccc34)n2)CC1]

To a solution of tert-butyl 4-[2-[1-(2-trimethylsilylethoxymethyl)indole-3-carbonyl]thiazol-4-yl]piperidine-1-carboxylate (440 mg, 686.26 μmol, 1 eq) in THF (10 mL) was added TBAF (1 M, 1.37 mL, 2 eq). The mixture was stirred at 80° C. for 2 h. TLC (PE/EtOAc=5/1, R_{f}=0.29) indicated the starting material was consumed completely and one new spot formed. The reaction mixture was quenched by addition of water (50 mL), extracted with EtOAc (30 mL×3). The combined organic layers were washed with brine (10 mL), dried over Na₂SO₄, filtered and concentrated under reduced pressure. The residue was purified by flash silica gel chromatography (from PE/EtOAc=1/0 to 5/1, TLC:PE/EtOAc=5/1, R_{f}=0.29) to yield tert-butyl 4-[2-(1H-indole-3-carbonyl)thiazol-4-yl]piperidine-1-carboxylate (210 mg, 508.78 μmol, 74.1% yield, 99.7% purity) as a yellow solid. ¹H NMR (500 MHz, CDCl₃) δ ppm 9.13 (d, J=3.1 Hz, 1H), 8.91 (br s, 1H), 8.59-8.52 (m, 1H), 7.52-7.46 (m, 1H), 7.39-7.31 (m, 2H), 7.24 (s, 1H), 4.27 (br s, 2H), 3.02 (tt, J=3.5, 11.8 Hz, 1H), 2.92 (br s, 2H), 2.10 (d, J=11.9 Hz, 2H), 1.77 (d, J=9.8 Hz, 2H), 1.54-1.44 (m, 9H); ES-LCMS m/z 306.8, 308.8 [M+H]⁺.

### Step 6: 1H-Indol-3-yl-[4-(4-piperidyl)thiazol-2-yl]methanone

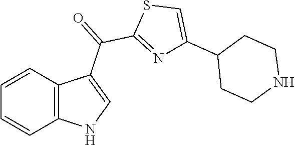 [SMILES: O=C(c1nc(C2CCNCC2)cs1)c1c[nH]c2ccccc12]

To a solution of tert-butyl 4-[2-(1H-indole-3-carbonyl)thiazol-4-yl]piperidine-1-carboxylate (200 mg, 484.55 mol, 1 eq) in DCM (5 mL) was added HCl/EtOAc (4 M, 5 mL). The mixture was stirred at 25° C. for 1 h. The reaction mixture was concentrated to yield 1H-indol-3-yl-[4-(4-piperidyl)thiazol-2-yl]methanone (168 mg, 434.66 mol, 89.7% yield, 90.0% purity, HCl) as a yellow solid, which was used in the next step without further purification. ¹H NMR (500 MHz, DMSO-d₆) δ ppm 12.34 (br s, 1H), 9.05 (d, J=3.2 Hz, 1H), 8.86 (br s, 1H), 8.39-8.19 (m, 1H), 7.83 (s, 1H), 7.58 (dd, J=1.9, 6.5 Hz, 1H), 7.40-7.17 (m, 2H), 3.47-3.40 (m, 2H), 3.25-3.17 (m, 1H), 3.11-3.02 (m, 2H), 2.25 (d, J=12.5 Hz, 2H), 2.01-1.90 (m, 2H); ES-LCMS m/z 380.9 [M+H]⁺.

### Step 7: 4-[4-[2-(1H-Indole-3-carbonyl)thiazol-4-yl]-1-piperidyl]-4-oxo-butanoic acid

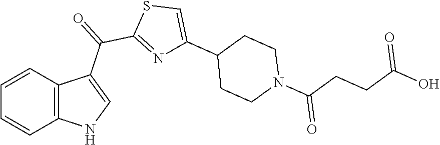 [SMILES: O=C(O)CCC(=O)N1CCC(c2csc(C(=O)c3c[nH]c4ccccc34)n2)CC1]

To a solution of 1H-indol-3-yl-[4-(4-piperidyl)thiazol-2-yl]methanone (40 mg, 103.49 μmol, 1 eq, HCl), tetrahydrofuran-2,5-dione (51.78 mg, 517.45 0181 μmol, 5 eq) in DMF (2 mL) was added TEA (52.36 mg, 517.45 μmol, 72.02 μL, 5 eq) and tetrahydrofuran-2,5-dione (51.78 mg, 517.45 μmol, 5 eq). The mixture was stirred at 25° C. for 12 h. The reaction mixture was concentrated. The residue was purified by preparative HPLC (column: Welch Xtimate C18 150*25 mm*5 μm; mobile phase: [water (10 mM NH₄HCO₃)-ACN]; B %: 10%-40%, 10 min), followed by lyophilization to yield 4-[4-[2-(1H-indole-3-carbonyl)thiazol-4-yl]-1-piperidyl]-4-oxo-butanoic acid (16.20 mg, 39.25 μmol, 37.9% yield, 99.7% purity) as a white solid. ¹H NMR (500 MHz, DMSO-d₆) δ ppm 9.09 (s, 1H), 8.37-8.25 (m, 1H), 7.81-7.74 (m, 1H), 7.63-7.53 (m, 1H), 7.35-7.20 (m, 2H), 4.50 (d, J=12.8 Hz, 1H), 4.01 (d, J=13.6 Hz, 1H), 3.16 (d, J=4.6 Hz, 1H), 3.15-3.11 (m, 1H), 2.77-2.69 (m, 1H), 2.60-2.55 (m, 2H), 2.47-2.41 (m, 2H), 2.09 (t, J=15.9 Hz, 2H), 1.77-1.65 (m, 1H), 1.63-1.51 (m, 1H); ES-LCMS m/z 383.0 [M+H]⁺.

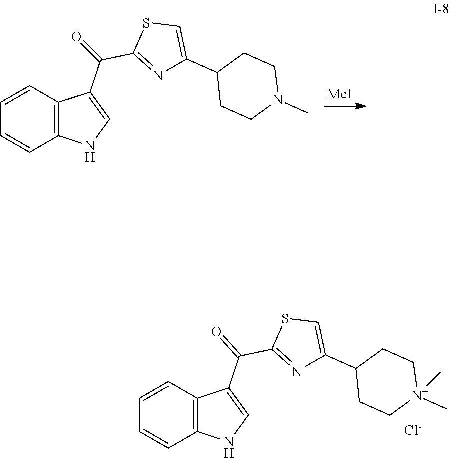 [SMILES: "MeI" ; CN1CCC(c2csc(C(=O)c3c[nH]c4ccccc34)n2)CC1 ; C[N+]1(C)CCC(c2csc(C(=O)c3c[nH]c4ccccc34)n2)CC1 ; [Cl-]]

### Step 1: [4-(1-BLAH-1,1-Dimethyl-1azinan-4-yl)thiazol-2-yl]-(1H-indol-3-yl)methanone

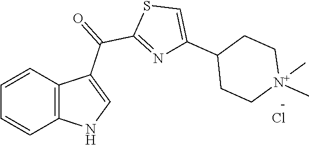 [SMILES: C[N+]1(C)CCC(c2csc(C(=O)c3c[nH]c4ccccc34)n2)CC1 ; Cl]

To a solution of 1H-indol-3-yl-[4-(1-methyl-4-piperidyl)thiazol-2-yl]methanone (30 mg, 92.19 μmol, 1 eq) in MeCN (5 mL) was added MeI (196.27 mg, 1.38 mmol, 86.09 μL, 15 eq). The mixture was stirred at 25° C. for 1 h. TLC (DCM/MeOH=10/1, R_{f}=0.00) indicated the starting material was consumed completely and one new spot formed. The reaction mixture was concentrated. The residue was purified by preparative HPLC (column: Phenomenex Synergi C18 150*30 mm*4 μm; mobile phase: [water (0.05% HCl)-ACN]; B %: 18%-38%, 10 min), followed by lyophilization to yield[4-(1-BLAH-1,1-dimethyl-1azinan-4-yl)thiazol-2-yl]-(1H-indol-3-yl)methanone (23.44 mg, 62.35 μmol, 67.6% yield, 100% purity) as a white solid. ¹H NMR (400 MHz, DMSO-d₆) δ ppm 12.30 (br s, 1H), 9.09 (d, J=3.1 Hz, 1H), 8.36-8.28 (m, 1H), 7.92 (s, 1H), 7.62-7.53 (m, 1H), 7.34-7.22 (m, 2H), 3.62-3.45 (m, 4H), 3.17 (d, J=15.7 Hz, 7H), 2.32-2.11 (m, 4H); ES-LCMS m/z 340.2 [M-Cl]⁺.

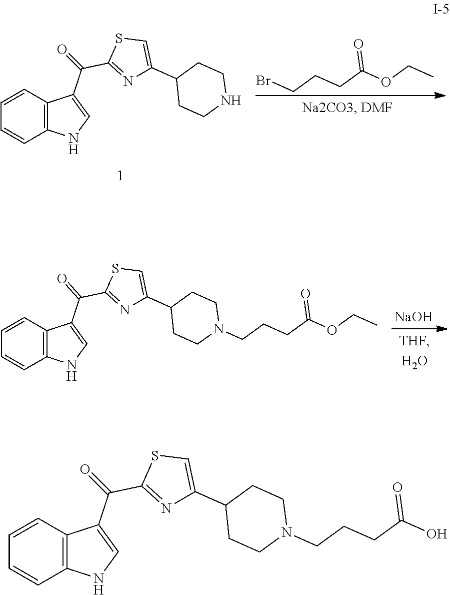

### Step 1: Ethyl 4-[4-[2-(1H-indole-3-carbonyl)thiazol-4-yl]-1-piperidyl]butanoate

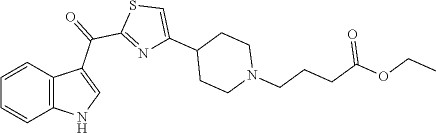 [SMILES: CCOC(=O)CCCN1CCC(c2csc(C(=O)c3c[nH]c4ccccc34)n2)CC1]

To a stirred solution of 1H-indol-3-yl-[4-(4-piperidyl)thiazol-2-yl]methanone (60 mg, 155.23 μmol, 1 eq, HCl) and ethyl 4-bromobutanoate (36.33 mg, 186.28 μmol, 26.72 μL, 1.2 eq) in DMF (1 mL) was added Na₂CO₃ (49.36 mg, 465.70 μmol, 3 eq). The reaction mixture was stirred at 25° C. for 48 h. The reaction mixture was diluted with H₂O (10 mL) and extracted with EtOAc (20 mL×3). The combined organic layers were washed with brine (15 mL), dried over Na₂SO₄, filtered and concentrated under reduced pressure to yield ethyl 4-[4-[2-(1H-indole-3-carbonyl)thiazol-4-yl]-1-piperidyl]butanoate (60 mg, 119.85 μmol, 77.2% yield, 85.0% purity) as a yellow solid, which was used in the next step without further purification. ¹H NMR (400 MHz, DMSO-d₆) δ ppm 12.16 (s, 1H), 9.07 (s, 1H), 8.31 (dd, J=2.8, 5.7 Hz, 1H), 7.74 (s, 1H), 7.57 (dd, J=2.6, 6.0 Hz, 1H), 7.31-7.23 (m, 2H), 4.06 (d, J=7.1 Hz, 2H), 2.96 (d, J=8.6 Hz, 2H), 2.87-2.80 (m, 1H), 2.43 (t, J=7.2 Hz, 2H), 2.34-2.30 (m, 2H), 2.07-2.00 (m, 4H), 1.80-1.68 (m, 4H), 1.18 (d, J=0.7 Hz, 3H); ES-LCMS m/z 426.2 [M+H]⁺.

### Step 2: 4-[4-[2-(1H-Indole-3-carbonyl)thiazol-4-yl]-1-piperidyl]butanoic acid

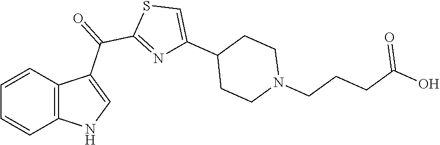 [SMILES: O=C(O)CCCN1CCC(c2csc(C(=O)c3c[nH]c4ccccc34)n2)CC1]

To a stirred solution of ethyl 4-[4-[2-(1H-indole-3-carbonyl)thiazol-4-yl]-1-piperidyl]butanoate (55 mg, 109.86 μmol, 1 eq) in THF (5 mL) and H₂O (1 mL) was added NaOH (35.15 mg, 878.88 μmol, 8 eq). The reaction mixture was stirred at 70° C. for 12 h. The reaction mixture was concentrated under reduced pressure to remove THF. The residue was adjust pH to 3 with 1 N HCl solution, filtered and the filter cake was purified by preparative HPLC (column: Welch Xtimate C18 150*25 mm*5 μm; mobile phase: [water (10 mM NH₄HCO₃)-ACN]; B %: 20%-50%, 10 min) to yield 4-[4-[2-(1H-indole-3-carbonyl)thiazol-4-yl]-1-piperidyl]butanoic acid (21.89 mg, 54.96 μmol, 50.0% yield, 99.8% purity) as a light yellow solid. ¹H NMR (500 MHz, DMSO-d₆) δ ppm 12.22 (s, 1H), 9.09 (s, 1H), 8.35-8.28 (m, 1H), 7.75 (s, 1H), 7.60-7.55 (m, 1H), 7.30-7.24 (m, 2H), 3.02 (d, J=11.0 Hz, 2H), 2.93-2.82 (m, 1H), 2.41 (t, J=6.7 Hz, 2H), 2.25 (t, J=6.9 Hz, 2H), 2.15 (t, J=11.2 Hz, 2H), 2.06 (d, J=11.3 Hz, 2H), 1.82-1.74 (m, 2H), 1.73-1.66 (m, 2H); ES-LCMS m/z 398.2 [M+H]⁺.

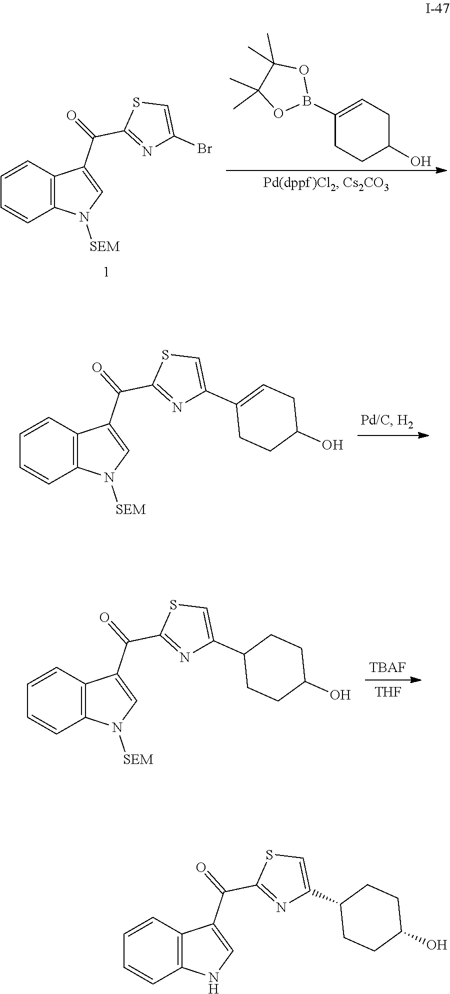

### Step 1: [4-(4-Hydroxycyclohexen-1-yl)thiazol-2-yl]-[1-(2-trimethylsilylethoxymethyl)indol-3-yl]methanone

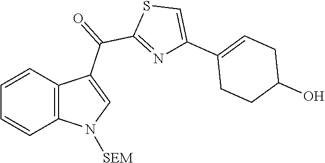 [SMILES: C[Si](C)(C)CCOCn1cc(C(=O)c2nc(C3=CCC(O)CC3)cs2)c2ccccc21]

To a solution of (4-bromothiazol-2-yl)-[1-(2-trimethylsilylethoxymethyl)indol-3-yl]methanone (370 mg, 837.40 μmol, 1 eq) and 4-(4,4,5,5-tetramethyl-1,3,2-dioxaborolan-2-yl)cyclohex-3-en-1-ol (187.66 mg, 837.40 μmol, 1 eq) in 1,4-dioxane (10 mL) and H₂O (1 mL) were added Pd(dppf)Cl₂ (61.27 mg, 83.74 μmol, 0.1 eq) and Cs₂CO₃ (545.68 mg, 1.67 mmol, 2 eq). The mixture was stirred at 90° C. for 16 h under N₂ atmosphere. TLC (PE/EtOAc=1/1, R_{f}=0.67) showed the starting material was consumed and a new spot was formed. The mixture was filtered and concentrated to give a residue which was purified by flash silica chromatography (PE/EtOAc=3/1 to 1/1) to yield[4-(4-hydroxycyclohexen-1-yl)thiazol-2-yl]-[1-(2-trimethylsilylethoxymethyl)indol-3-yl]methanone (356 mg, 767.35 μmol, 91.6% yield, 98.0% purity) as yellow gum. ¹H NMR (500 MHz, CDCl₃) δ ppm 9.09 (s, 1H), 8.58-8.53 (m, 1H), 7.59-7.55 (m, 1H), 7.39-7.36 (m, 2H), 7.35 (s, 1H), 6.70 (br s, 1H), 5.61 (s, 2H), 4.14-4.10 (m, 1H), 3.58-3.55 (m, 2H), 2.76-2.57 (m, 3H), 2.35-2.30 (m, 1H), 2.12-2.06 (m, 1H), 1.95-1.86 (m, 1H), 0.98-0.88 (m, 2H), −0.05 (s, 9H; ES-LCMS m/z 455.3 [M+H]⁺.

### Step 2: [4-(4-Hydroxycyclohexyl)thiazol-2-yl]-[1-(2-trimethylsilylethoxymethyl)indol-3-yl]methanone

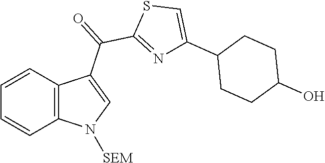 [SMILES: C[Si](C)(C)CCOCn1cc(C(=O)c2nc(C3CCC(O)CC3)cs2)c2ccccc21]

To a solution of [4-(4-hydroxycyclohexen-1-yl)thiazol-2-yl]-[1-(2-trimethylsilylethoxymethyl)indol-3-yl]methanone (356 mg, 767.35 μmol, 1 eq) in MeOH (10 mL) was added Pd/C (50 mg, 10% purity, 1.00 eq) under N₂. The suspension was degassed under vacuum and purged with H2 several times and stirred at 50° C. for 16 h under H2 (50 psi). TLC (PE/EtOAc=1/1, R_{f}=0.75) showed the starting material was remained and a new spot was formed. The mixture was filtered and the filter cake was washed with MeOH (15 mL×2). The filtrate was concentrated to give a residue which was purified by preparative TLC (PE/EtOAc=1/1) to yield [4-(4-hydroxycyclohexyl)thiazol-2-yl]-[1-(2-trimethylsilylethoxymethyl)indol-3-yl]methanone (201 mg, 440.14 μmol, 57.4% yield, 100% purity) as light yellow gum. ¹H NMR (500 MHz, CDCl₃) δ ppm 9.10 (s, 1H), 8.58-8.52 (m, 1H), 7.62-7.52 (m, 1H), 7.42-7.33 (m, 2H), 5.61 (s, 2H), 4.13-4.11 (m, 1H), 3.61-3.50 (m, 2H), 3.01-2.95 (m, 1H), 2.03-1.88 (m, 6H), 1.83-1.73 (m, 2H), 1.36-1.35 (m, 1H), 0.97-0.89 (m, 2H), −0.05 (s, 9H); ES-LCMS m/z 457.3 [M+H]⁺. and recycled [4-(4-hydroxycyclohexen-1-yl)thiazol-2-yl]-[1-(2-trimethylsilylethoxymethyl)indol-3-yl]methanone (90 mg) as light yellow gum.

### Step 3: [4-(4-Hydroxycyclohexyl)thiazol-2-yl]-(1H-indol-3-yl)methanone

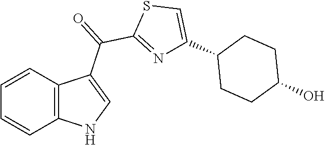 [SMILES: O=C(c1nc([C@H]2CC[C@@H](O)CC2)cs1)c1c[nH]c2ccccc12]

To a solution of [4-(4-hydroxycyclohexyl)thiazol-2-yl]-[1-(2-trimethylsilylethoxymethyl)indol-3-yl]methanone (181 mg, 396.35 umol, 1 eq) in THF (5 mL) was added TBAF (1 M, 1.19 mL, 3 eq). The mixture was stirred at 70° C. for 16 h. TLC (PE/EtOAC=1/2, R_{f}=0.36) showed the starting material was consumed and a new spot was formed. The solvent was removed and the residue was treated with water (20 mL), extracted with EtOAc (20 mL×2). The combined organic phases were washed with water (20 mL), brine (20 mL), dried over anhydrous Na₂SO₄, filtered and concentrated to give a residue which was purified by preparative TLC (PE/EtOAc=1/2) to yield compound [4-(4-hydroxycyclohexyl)thiazol-2-yl]-(1H-indol-3-yl)methanone (66.8 mg, 195.58 μmol, 49.4% yield, 95.6% purity) as a light yellow solid ¹H NMR (400 MHz, CD₃OD) δ ppm 9.16 (s, 1H), 8.44-8.34 (m, 1H), 7.52-7.50 (m, 2H), 7.36-7.19 (m, 2H), 4.02 (br s, 1H), 3.07-2.89 (m, 1H), 2.17-2.06 (m, 2H), 1.96-1.84 (m, 4H), 1.81-1.71 (m, 2H); ES-LCMS m/z 348.9 [M+Na]t

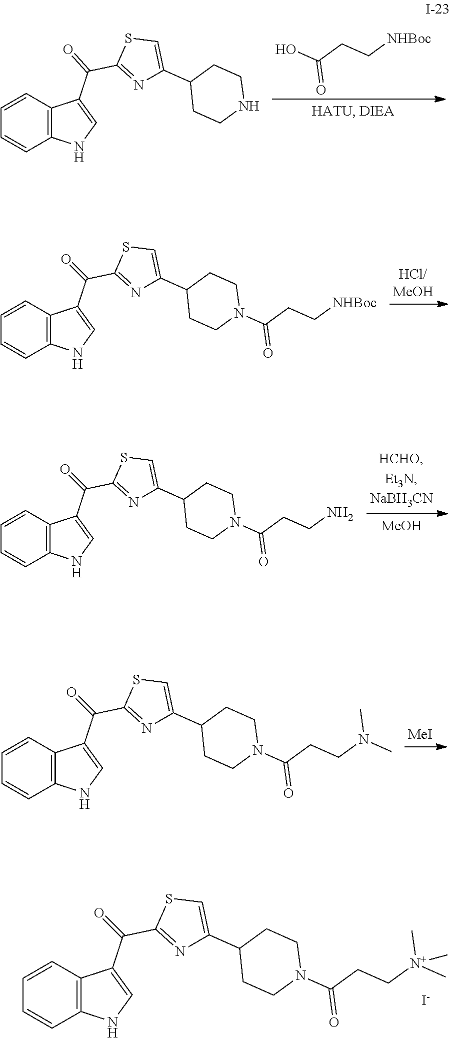

### Step 1: tert-Butyl N-[3-[4-[2-(1H-indole-3-carbonyl)thiazol-4-yl]-1-piperidyl]-3-oxo-propyl]carbamate

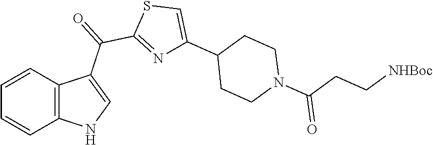 [SMILES: CC(C)(C)OC(=O)NCCC(=O)N1CCC(c2csc(C(=O)c3c[nH]c4ccccc34)n2)CC1]

To a solution of 1H-indol-3-yl-[4-(4-piperidyl)thiazol-2-yl]methanone (50 mg, 129.36 μmol, 1 eq, HCl) in THF (8 mL) was added HATU (73.78 mg, 194.04 μmol, 1.5 eq), 3-(tert-butoxycarbonylamino)propanoic acid (29.37 mg, 155.23 μmol, 1.2 eq) and DIEA (33.44 mg, 258.72 μmol, 45.06 μL, 2 eq). The mixture was stirred at 25° C. for 12 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 1/1, TLC:PE/EtOAc=1/1, R_{f}=0.20) to yield tert-butyl N-[3-[4-[2-(1H-indole-3-carbonyl)thiazol-4-yl]-1-piperidyl]-3-oxo-propyl]carbamate (62 mg, 126.55 μmol, 97.8% yield, 98.5% purity) as yellow solid. ¹H NMR (500 MHz, CDCl₃) δ ppm 9.13 (s, 2H), 8.55 (d, J=7.0 Hz, 1H), 7.52-7.46 (m, 1H), 7.35 (t, J=5.8 Hz, 2H), 7.24 (s, 1H), 4.76 (d, J=12.5 Hz, 1H), 4.13 (q, J=7.2 Hz, 1H), 3.97 (d, J=13.3 Hz, 1H), 3.51-3.46 (m, 2H), 3.21 (t, J=12.7 Hz, 1H), 3.11 (t, J=11.8 Hz, 1H), 2.84-2.74 (m, 2H), 2.60 (s, 2H), 2.15 (s, 2H), 2.05 (s, 2H), 1.81 (d, J=10.7 Hz, 2H), 1.60 (s, 9H); ES-LCMS m/z 483.2 [M+H]⁺.

### Step 2: 3-Amino-1-[4-[2-(1H-indole-3-carbonyl)thiazol-4-yl]-1-piperidyl]propan-1-one

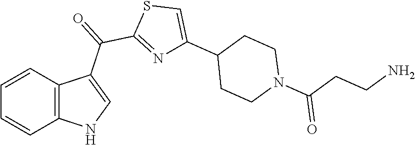 [SMILES: NCCC(=O)N1CCC(c2csc(C(=O)c3c[nH]c4ccccc34)n2)CC1]

To a solution of tert-butyl N-[3-[4-[2-(1H-indole-3-carbonyl)thiazol-4-yl]-1-piperidyl]-3-oxo-propyl]carbamate (62 mg, 125.90 μmol, 1 eq) was added HCl/MeOH (4 M, 4.13 mL, 131.32 eq). The mixture was stirred at 25° C. for 1 h. The mixture was evaporated to yield a residue which was used in the next step without further purification to yield 3-amino-1-[4-[2-(1H-indole-3-carbonyl)thiazol-4-yl]-1-piperidyl]propan-1-one (52 mg, 124.12 μmol, 98.6% yield, 100% purity, HCl) as a white solid. ¹H NMR (400 MHz, DMSO-d₆) δ ppm 12.27 (s, 1H), 9.06 (d, J=3.2 Hz, 1H), 8.31 (dd, J=2.8, 6.2 Hz, 1H), 7.79 (s, 1H), 7.75 (s, 2H), 7.28 (td, J=3.1, 6.1 Hz, 2H), 4.53 (d, J=13.2 Hz, 1H), 3.92 (d, J=13.4 Hz, 1H), 3.27-3.16 (m, 2H), 3.06-3.00 (m, 2H), 2.81 (t, J=11.5 Hz, 1H), 2.77-2.71 (m, 2H), 2.69 (s, 1H), 2.19-2.12 (m, 2H), 1.73-1.58 (m, 2H); ES-LCMS m/z 383.2 [M+H]⁺.

### Step 3: 3-(Dimethylamino)-1-[4-[2-(1H-indole-3-carbonyl)thiazol-4-yl]-1-piperidyl]propan-1-one

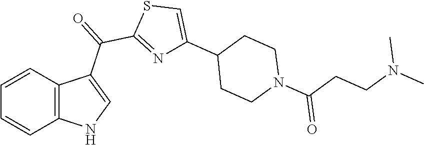 [SMILES: CN(C)CCC(=O)N1CCC(c2csc(C(=O)c3c[nH]c4ccccc34)n2)CC1]

To a stirred solution of 3-amino-1-[4-[2-(1H-indole-3-carbonyl)thiazol-4-yl]-1-piperidyl]propan-1-one (60 mg, 143.22 μmol, 1 eq, HCl) in MeOH (5 mL) was added HCHO (43.00 mg, 1.43 mmol, 39.45 μL, 10 eq) and Et₃N (72.46 mg, 716.09 μmol, 99.67 μL, 5 eq). The reaction mixture was stirred at 30° C. for 30 min. NaBH₃CN (54.00 mg, 859.31 μmol, 6 eq) was added to the above reaction mixture then stirred at 30° C. for 1 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 32%-62%, 10 min) followed by lyophilized to yield 3-(dimethylamino)-1-[4-[2-(1H-indole-3-carbonyl)thiazol-4-yl]-1-piperidyl]propan-1-one (26 mg, 63.33 μmol, 44.2% yield, 100% purity) as a white solid. ¹H NMR (400 MHz, DMSO-d₆) δ ppm 12.19 (s, 1H), 9.07 (s, 1H), 8.31 (dd, J=2.7, 5.9 Hz, 1H), 7.78 (s, 1H), 7.61-7.55 (m, 1H), 7.32-7.23 (m, 2H), 4.52 (d, J=13.2 Hz, 1H), 4.02 (d, J=13.7 Hz, 1H), 3.32-3.28 (m, 4H), 3.26-3.15 (m, 2H), 2.78-2.68 (m, 1H), 2.16 (s, 6H), 2.14-2.04 (m, 2H), 1.75-1.64 (m, 1H), 1.63-1.50 (m, 1H); ES-LCMS m/z 411.2 [M+H]⁺.

### Step 4: 1-[4-[2-(1H-indole-3-carbonyl)thiazol-4-yl]-1-piperidyl]-3-[BLAH(trimethyl)-azanyl]propan-1-one

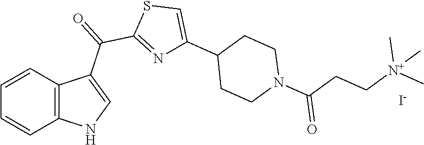 [SMILES: C[N+](C)(C)CCC(=O)N1CCC(c2csc(C(=O)c3c[nH]c4ccccc34)n2)CC1 ; [I-]]

To a stirred solution of 3-(dimethylamino)-1-[4-[2-(1H-indole-3-carbonyl)thiazol-4-yl]-1-piperidyl]propan-1-one (21 mg, 51.15 μmol, 1 eq) in ACN (5 mL) was added MeI (36.30 mg, 255.77 μmol, 15.92 μL, 5 eq) slowly. The reaction mixture was stirred at 30° C. for 1 h. The solvent was removed to yield a residue which was used in the next step without further purification to yield 1-[4-[2-(1H-indole-3-carbonyl)thiazol-4-yl]-1-piperidyl]-3-[BLAH(trimethyl)-azanyl]propan-1-one (20 mg, 34.39 μmol, 67.2% yield, 95% purity) as green solid. ¹H NMR (400 MHz, DMSO-d₆) δ ppm 12.22 (s, 1H), 9.05 (s, 1H), 8.31 (dd, J=2.6, 6.0 Hz, 1H), 7.79 (s, 1H), 7.58 (dd, J=2.4, 6.1 Hz, 1H), 7.31-7.25 (m, 2H), 4.51 (d, J=12.5 Hz, 1H), 4.08 (d, J=13.2 Hz, 1H), 3.57 (t, J=7.9 Hz, 2H), 3.27-3.16 (m, 2H), 3.09 (s, 9H), 2.96 (dd, J=4.9, 8.3 Hz, 2H), 2.79 (t, J=11.5 Hz, 1H), 2.15 (s, 2H), 1.80-1.67 (m, 1H), 1.65-1.52 (m, 1H); ES-LCMS m/z 425.1 [M-I]⁺.

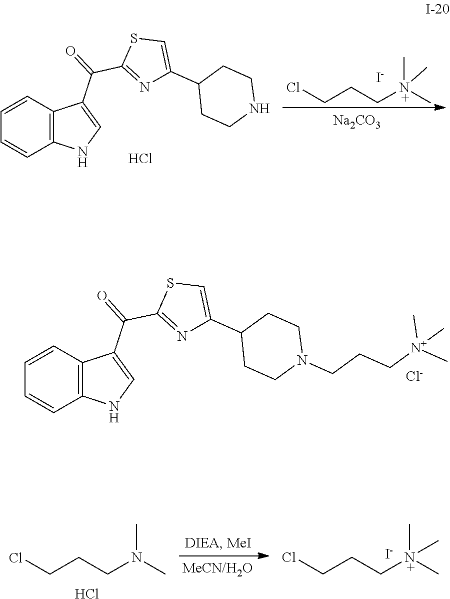

### Step 1: 1-Chloro-3-[BLAH(trimethyl)-azanyl]propane

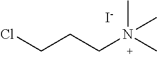

To a stirred solution of 3-chloro-N, N-dimethyl-propan-1-amine (100 mg, 632.64 μmol, 1 eq, HCl) in MeCN (5 mL) was added DIEA (245.29 mg, 1.90 mmol, 330.57 μL, 3 eq). The reaction mixture was stirred at 25° C. for 2 h. Then added MeI (897.96 mg, 6.33 mmol, 393.84 μL, 10 eq) at 25° C. and stirred for 2 h under N₂ atmosphere. The reaction mixture was by lyophilization to yield a residue which was used in the next step without further purification to yield 1-chloro-3-[BLAH(trimethyl)-azanyl]propane (250 mg, 474.30 μmol, 74.9% yield, 50.0% purity) as a white solid. ¹H NMR (500 MHz, DMSO-d₆) δ ppm 3.73 (t, J=6.3 Hz, 2H), 3.13-3.10 (m, 2H), 3.08 (s, 9H), 2.23-2.16 (m, 2H); ES-LCMS no desired mass was detected.

### Step 2: 1H-Indol-3-yl-[4-[1-[3-[BLAH(trimethyl)-azanyl]propyl]-4-piperidyl]thiazol-2-yl]methanone

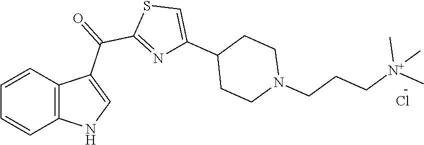 [SMILES: C[N+](C)(C)CCCN1CCC(c2csc(C(=O)c3c[nH]c4ccccc34)n2)CC1 ; Cl]

To a stirred solution of 1H-indol-3-yl-[4-(4-piperidyl)thiazol-2-yl]methanone (50 mg, 122.17 μmol, 1 eq, HCl) and 1-chloro-3-[BLAH(trimethyl)-azanyl]propane (83.71 mg, 158.82 μmol, 1.3 eq) in DMF (4 mL) was added Na₂CO₃ (38.85 mg, 366.51 μmol, 3 eq). The reaction mixture was stirred at 60° C. for 48 h under N₂ atmosphere. The reaction mixture was concentrated under reduced pressure to yield a residue which was purified by preparative HPLC (column: Phenomenex Synergi C18 150*30 mm*4 μm; mobile phase: [water (0.05% HCl)-ACN]; B %: 12%-32%, 9 min) to yield 1H-indol-3-yl-[4-[1-[3-[BLAH(trimethyl)-azanyl]propyl]-4-piperidyl]thiazol-2-yl]methanone (21.22 mg, 43.71 μmol, 35.8% yield, 99.6% purity, HCl) as a yellow solid. ¹H NMR (500 MHz, DMSO-d₆) δ ppm 12.38 (s, 1H), 11.09 (s, 1H), 9.13 (d, J=3.2 Hz, 1H), 8.33-8.30 (m, 1H), 7.84 (s, 1H), 7.62-7.54 (m, 1H), 7.31-7.25 (m, 2H), 3.65 (d, J=11.4 Hz, 2H), 3.52-3.48 (m, 2H), 3.20-3.14 (m, 2H), 3.11 (s, 9H), 3.08 (s, 1H), 2.48-2.45 (m, 2H), 2.35-2.19 (m, 6H); ES-LCMS m/z 411.3 [M-Cl]⁺.

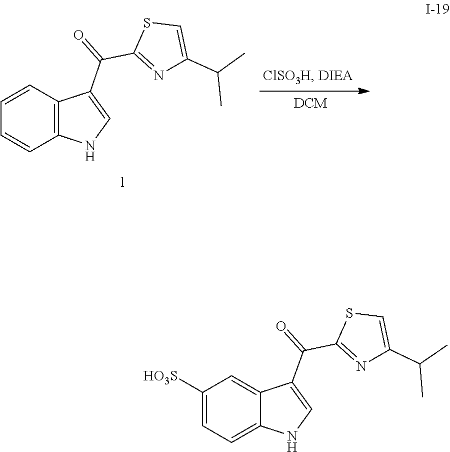 [SMILES: CC(C)c1csc(C(=O)c2c[nH]c3ccccc23)n1 ; CC(C)c1csc(C(=O)c2c[nH]c3ccc(S(=O)(=O)O)cc23)n1]

### Step 1: 3-(4-Isopropylthiazole-2-carbonyl)-1H-indole-5-sulfonic acid

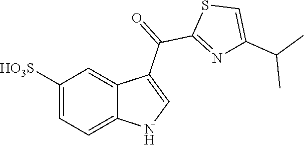 [SMILES: CC(C)c1csc(C(=O)c2c[nH]c3ccc(S(=O)(=O)O)cc23)n1]

To a stirred solution of 1H-indol-3-yl-(4-isopropylthiazol-2-yl)methanone (200 mg, 688.00 μmol, 1 eq) in DCM (10 mL) was added sulfurochloridic acid (400.84 mg, 3.44 mmol, 229.05 μL, 5 eq) and DIEA (177.84 mg, 1.38 mmol, 239.67 μL, 2 eq). The reaction mixture was stirred at 30° C. for 2 h. The reaction mixture was quenched by the addition of saturated aqueous NaHCO₃ solution (3 ml) and concentrated to yield a residue which was purified by preparative HPLC (column: Phenomenex Synergi C18 150*30 mm*4 um; mobile phase: [water (0.05% HCl)-ACN]; B %: 34%-54%, 10 min). The desired fraction was lyophilized to yield 3-(4-isopropylthiazole-2-carbonyl)-1H-indole-5-sulfonic acid (104.37 mg, 290.40 μmol, 42.2% yield, 97.5% purity) as a yellow solid. ¹H NMR (400 MHz, DMSO-d₆) δ ppm 12.22 (s, 1H), 9.10 (d, J=2.9 Hz, 1H), 8.62 (s, 1H), 7.72 (s, 1H), 7.57 (d, J=8.1 Hz, 1H), 7.51-7.45 (m, 1H), 3.25-3.15 (m, 1H), 1.36 (d, J=7.1 Hz, 6H); ES-LCMS m/z 350.9 [M+H]⁺.

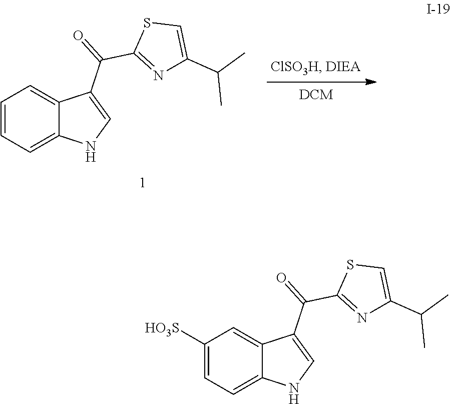 [SMILES: CC(C)c1csc(C(=O)c2c[nH]c3ccccc23)n1 ; CC(C)c1csc(C(=O)c2c[nH]c3ccc(S(=O)(=O)O)cc23)n1]

### Step 1: 4-[2-[1-(2-Trimethylsilylethoxymethyl)indole-3-carbonyl]thiazol-4-yl]cyclohex-3-en-1-one

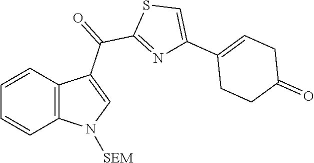 [SMILES: C[Si](C)(C)CCOCn1cc(C(=O)c2nc(C3=CCC(=O)CC3)cs2)c2ccccc21]

To a solution of [4-(4-hydroxycyclohexen-1-yl)thiazol-2-yl]-[1-(2-trimethylsilylethoxymethyl)indol-3-yl]methanone (531 mg, 1.05 mmol, 1 eq) in DCM (10 mL) was added Dess-Martin periodinane (891.65 mg, 2.10 mmol, 2 eq) at 0° C. The mixture was stirred at 20° C. for 1 h. TLC (PE/EtOAc=1/1, R_{f}=0.69) indicated the starting material was consumed completely and one new spot formed. The reaction was quenched by sat.aq. Na₂S₂O₃ (5 mL) and sat.aq. NaHCO₃ (5 mL), treated with water (20 mL), extracted with DCM (20 mL×2). The combined organic phases were washed with brine (15 mL), dried over anhydrous Na₂SO₄, filtered and concentrated to give a residue which was purified by silica gel chromatography (from PE/EtOAc=1/0 to 3/1) to yield 4-[2-[1-(2-trimethylsilylethoxymethyl)indole-3-carbonyl]thiazol-4-yl]cyclohex-3-en-1-one (350 mg, 734.58 μmol, 69.9% yield, 95.0% purity) as a light yellow solid. ¹H NMR (400 MHz, CDCl₃) δ ppm 9.11 (s, 1H), 8.64-8.57 (m, 1H), 7.66-7.59 (m, 1H), 7.49 (s, 1H), 7.47-7.41 (m, 2H), 6.84 (t, J=3.9 Hz, 1H), 5.66 (s, 2H), 3.66-3.59 (m, 2H), 3.25-3.21 (m, 2H), 3.10-3.03 (m, 2H), 2.77 (t, J=7.0 Hz, 2H), 1.01-0.95 (m, 2H), 0.00 (s, 9H).

### Step 2: 4-[2-[1-(2-Trimethylsilylethoxymethyl)indole-3-carbonyl]thiazol-4-yl]cyclohexanone

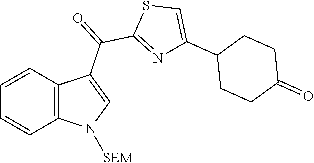 [SMILES: C[Si](C)(C)CCOCn1cc(C(=O)c2nc(C3CCC(=O)CC3)cs2)c2ccccc21]

To a solution of 4-[2-[1-(2-trimethylsilylethoxymethyl)indole-3-carbonyl]thiazol-4-yl]cyclohex-3-en-1-one (350 mg, 734.58 μmol, 1 eq) in MeOH (3 mL) and THF (3 mL) was added Pd/C (50 mg, 734.58 μmol, 10% purity, 1.00 eq) under N₂ atmosphere. The suspension was degassed under vacuum and purged with H2 several times and stirred at 30° C. for 3 h. The reaction mixture was filtered and the filter cake was washed with MeOH (10 mL×2). The filtrate was concentrated to give a residue which was purified by silica gel chromatography (from PE/EtOAc=1/0 to 3/1, TLC:PE/EtOAc=1/1, R_{f}=0.62) to yield 4-[2-[1-(2-trimethylsilylethoxymethyl)indole-3-carbonyl]thiazol-4-yl]cyclohexanone (270 mg, 564.16 μmol, 76.8% yield, 95.0% purity) as a light yellow solid. ¹H NMR (500 MHz, CDCl₃) δ ppm 9.02 (s, 1H), 8.57-8.52 (m, 1H), 7.58-7.54 (m, 1H), 7.42-7.34 (m, 2H), 7.30 (s, 1H), 5.61 (s, 2H), 3.60-3.53 (m, 2H), 3.41-3.36 (m, 1H), 2.61-2.53 (m, 4H), 2.52-2.44 (m, 2H), 2.17-2.06 (m, 2H), 0.97-0.88 (m, 2H), −0.05 (s, 9H); ES-LCMS m/z 477.1 [M+Na]⁺.

### Step 3: [4-(4-Hydroxycyclohexyl)thiazol-2-yl]-[1-(2-trimethylsilylethoxymethyl)indol-3-yl]methanone

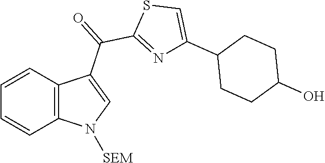 [SMILES: C[Si](C)(C)CCOCn1cc(C(=O)c2nc(C3CCC(O)CC3)cs2)c2ccccc21]

To a solution of 4-[2-[1-(2-trimethylsilylethoxymethyl)indole-3-carbonyl]thiazol-4-yl]cyclohexanone (250 mg, 522.37 μmol, 1 eq) in MeOH (3 mL) and THF (3 mL) was added NaBH₄ (19.76 mg, 522.37 μmol, 1.0 eq) at 0° C. and stirred at 0° C. for 10 min. TLC (PE/EA=1/1, R_{f}=0.55) indicated the starting material was consumed completely and one new spot formed. The reaction was quenched by sat.aq. NH₄Cl (10 mL) and the MeOH was removed. The mixture was diluted with H₂O (10 mL), extracted with EtOAc (15 mL×2). The combined organic phases were washed with brine (10 mL), dried over anhydrous Na₂SO₄, filtered and concentrated to give a residue which was purified by flash silica gel chromatography (from PE/EtOAc=3/1 to 1/1) to yield [4-(4-hydroxycyclohexyl)thiazol-2-yl]-[1-(2-trimethylsilylethoxymethyl)indol-3-yl]methanone (180 mg, 394.16 μmol, 75.5% yield, 100.0% purity) as light yellow gum. ¹H NMR (500 MHz, CDCl₃) δ ppm 9.06 (s, 1H), 8.57-8.51 (m, 1H), 7.59-7.53 (m, 1H), 7.40-7.33 (m, 2H), 7.21 (s, 1H), 5.61 (s, 2H), 3.79-3.68 (m, 1H), 3.62-3.49 (m, 2H), 2.89-2.84 (m, 1H), 2.30-2.21 (m, 2H), 2.16 (d, J=9.9 Hz, 2H), 1.68-1.51 (m, 4H), 0.96-0.90 (m, 2H), −0.05 (s, 9H); ES-LCMS m/z 457.2 [M+H]⁺.

### Step 4: [4-(4-Hydroxycyclohexyl)thiazol-2-yl]-(1H-indol-3-yl)methanone

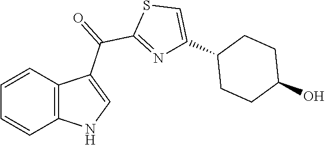 [SMILES: O=C(c1nc([C@H]2CC[C@H](O)CC2)cs1)c1c[nH]c2ccccc12]

To a solution of [4-(4-hydroxycyclohexyl)thiazol-2-yl]-[1-(2-trimethylsilylethoxymethyl)indol-3-yl]methanone (180 mg, 394.16 μmol, 1 eq) in THF (3 mL) was added TBAF (1.18 mL, 3 eq, 1 M) and the mixture was stirred at 70° C. for 16 h. TLC (PE/EtOAc=1/1, R_{f}=0.24) showed the starting material was consumed and a new spot was formed. The solvent was removed and the residue was treated with water (50 mL), extracted with EtOAc (30 mL×3). The combined organic layers were washed with brine (30 mL), dried over Na₂SO₄, filtered and concentrated under reduced pressure to give a residue which was triturated with MeOH (5 mL) to yield [4-(4-hydroxycyclohexyl)thiazol-2-yl]-(1H-indol-3-yl)methanone (52.89 mg, 162.03 μmol, 41.1% yield, 100.0% purity) as a white solid. ¹H NMR (500 MHz, CD₃OD) δ ppm 9.12 (s, 1H), 8.42-8.32 (m, 1H), 7.56-7.46 (m, 2H), 7.32-7.22 (m, 2H), 3.70-3.60 (m, 1H), 2.90-2.84 (m, 1H), 2.20 (d, J=12.8 Hz, 2H), 2.11 (d, J=9.9 Hz, 2H), 1.76-1.61 (m, 2H), 1.53-1.40 (m, 2H); ES-LCMS m/z 326.9 [M+H]⁺.

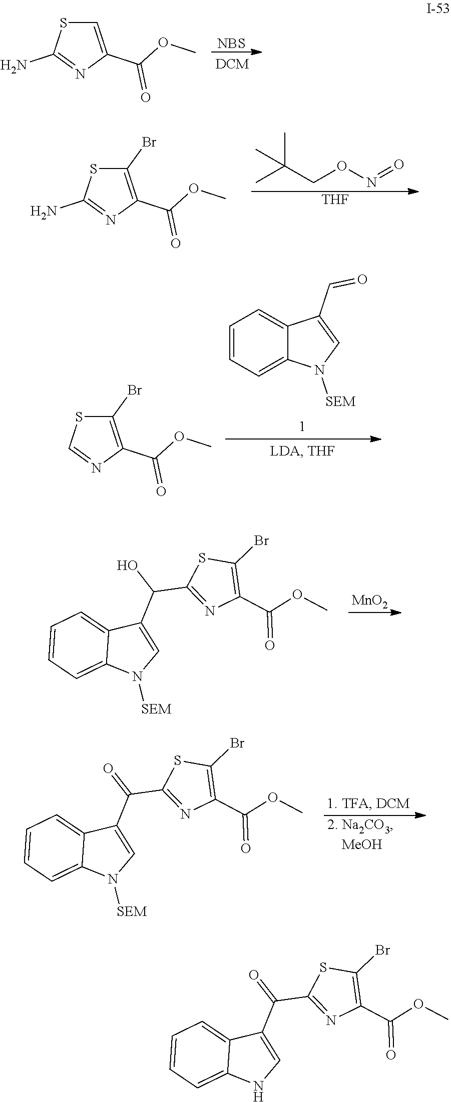

### Step 1: Methyl 2-amino-5-bromo-thiazole-4-carboxylate

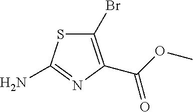

To a solution of methyl 2-aminothiazole-4-carboxylate (1.5 g, 9.48 mmol, 1 eq) in DCM (25 mL) was added NBS (2.03 g, 11.38 mmol, 1.2 eq). The mixture was stirred at 20° C. for 12 h under N₂. TLC (PE/EtOAc=1/1, R_{f}=0.22) indicated starting material was consumed completely and one new spot formed. The mixture was concentrated and then water (80 mL) was added. The mixture was extracted with EtOAc (50 mL×3). The combined organic layers were washed with brine (50 mL), dried over Na₂SO₄, filtered and concentrated to yield a residue which was purified by flash silica gel chromatography (From PE/EtOAc=1/0 to 1/1, R_{f}=0.22) to yield methyl 2-amino-5-bromo-thiazole-4-carboxylate (2 g, 7.82 mmol, 82.5% yield, 92.7% purity) as a red solid. ¹H NMR (400 MHz, CDCl₃) δ ppm 3.91 (s, 3H); ES-LCMS m/z 237.1, 238.1 [M+H]⁺.

### Step 2: Methyl 5-bromothiazole-4-carboxylate

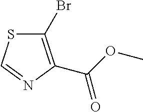

To a solution of methyl 2-amino-5-bromo-thiazole-4-carboxylate (2 g, 7.82 mmol, 92.7% purity, 1 eq) in DMF (15 mL) was added tert-butyl nitrite (1.29 g, 12.51 mmol, 1.49 mL, 1.6 eq). The mixture was stirred at 80° C. for 12 h under N₂. TLC (PE/EtOAc=3/1, R_{f}=0.55) indicated starting material was consumed completely and two new spots formed. The residue was partitioned between EtOAc (100 mL) and water (50 mL). The organic layer was washed with brine (50 mL), dried over Na₂SO₄, filtered and concentrated to yield a residue which was purified by flash silica gel chromatography (From PE/EtOAc=1/0 to 3/1, R_{f}=0.55) to yield methyl 5-bromothiazole-4-carboxylate (1.1 g, 4.85 mmol, 62.1% yield, 98.0% purity) as a red solid. ¹H NMR (400 MHz, CDCl₃) δ ppm 8.79 (s, 1H), 3.98 (s, 3H); ES-LCMS m/z 222.1, 224.1 [M+H]⁺.

### Step 3: Methyl 5-bromo-2-[hydroxy-[1-(2-trimethylsilylethoxymethyl)indol-3-yl]methyl]thiazole-4-carboxylate

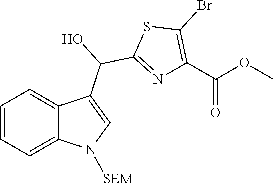 [SMILES: COC(=O)c1nc(C(O)c2cn(COCC[Si](C)(C)C)c3ccccc23)sc1Br]

To a stirred solution of DIPA (893.15 mg, 8.83 mmol, 1.25 mL, 2 eq) in THF (10 mL) was cooled to −75° C. then added n-BuLi (2.5 M, 3.53 mL, 2 eq) dropwise under N₂ atmosphere. The reaction mixture was stirred under N₂ atmosphere at −75° C. for 30 min. Methyl 5-bromothiazole-4-carboxylate (1 g, 4.41 mmol, 98% purity, 1 eq) and 1-(2-trimethylsilylethoxymethyl)indole-3-carbaldehyde (1.59 g, 5.30 mmol, 92% purity, 1.2 eq) was dissolved in THF (10 mL). The LDA reaction mixture was added to the above mixture, then stirred under N₂ atmosphere at −75° C. for 30 min. TLC (PE/EtOAc=3/1, R_{f}=0.30) indicated starting material was consumed completely and many new spots formed. The mixture was concentrated and then water (80 mL) was added. The mixture was extracted with EtOAc (50 mL×3). The combined organic layers were washed with brine (50 mL), dried over Na₂SO₄, filtered and concentrated to yield a residue which was purified by flash silica gel chromatography (From PE/EtOAc=1/0 to 3/1, R_{f}=0.30) to yield methyl 5-bromo-2-[hydroxy-[1-(2-trimethylsilylethoxymethyl)indol-3-yl]methyl]thiazole-4-carboxylate (350 mg, 287.75 μmol, 6.5% yield, 40.9% purity) as red oil. ¹H NMR (400 MHz, CDCl₃) δ ppm 7.63 (d, J=7.8 Hz, 1H), 7.51 (d, J=8.2 Hz, 2H), 7.19-7.15 (m, 1H), 6.36 (d, J=3.5 Hz, 1H), 5.47 (s, 3H), 3.96 (s, 3H), 3.50-3.47 (m, 2H), 0.90 (s, 2H), −0.05 (s, 9H); ES-LCMS m/z 481.1 [M-OH]⁺.

### Step 4: Methyl 5-bromo-2-[1-(2-trimethylsilylethoxymethyl)indole-3-carbonyl]thiazole-4-carboxylate

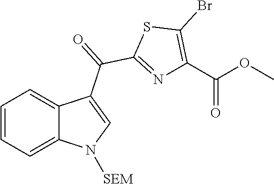 [SMILES: COC(=O)c1nc(C(=O)c2cn(COCC[Si](C)(C)C)c3ccccc23)sc1Br]

To a solution of methyl 5-bromo-2-[hydroxy-[1-(2-trimethylsilylethoxymethyl)indol-3-yl]methyl]thiazole-4-carboxylate (350 mg, 287.75 μmol, 40.9% purity, 1 eq) in CHCl₃ (25 mL) was added MnO₂ (500.34 mg, 5.76 mmol, 20 eq). The mixture was stirred at 60° C. for 12 h. The mixture was filtered, washed with EtOAc (50 mL×2). The filtrate was concentrated to yield a residue which was purified by flash silica gel chromatography (From PE/EtOAc=1/0 to 3/1, R_{f}=0.68) to yield methyl 5-bromo-2-[1-(2-trimethylsilylethoxymethyl)indole-3-carbonyl]thiazole-4-carboxylate (150 mg, 280.34 μmol, 97.4% yield, 92.6% purity) as a red solid. ¹H NMR (400 MHz, CDCl₃) δ ppm 9.07 (s, 1H), 8.52-8.46 (m, 1H), 7.63-7.57 (m, 1H), 7.42-7.36 (m, 2H), 5.62 (s, 2H), 4.03 (s, 3H), 3.61-3.52 (m, 2H), 0.98-0.91 (m, 2H), −0.04 (s, 9H); ES-LCMS m/z 495.1, 497.1 [M+H]⁺.

### Step 5: Methyl 5-bromo-2-(1H-indole-3-carbonyl)thiazole-4-carboxylate

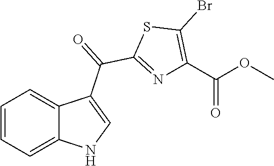 [SMILES: COC(=O)c1nc(C(=O)c2c[nH]c3ccccc23)sc1Br]

To a stirred solution of methyl 5-bromo-2-[1-(2-trimethylsilylethoxymethyl)indole-3-carbonyl]thiazole-4-carboxylate (120 mg, 224.28 μmol, 92.6% purity, 1 eq) in DCM (3 mL) was added TFA (4.28 g, 37.52 mmol, 2.78 mL, 167.30 eq) The reaction mixture was stirred at 25° C. for 1 h. TLC (PE/EtOAc=3/1, R_{f}=0.31) showed starting material was consumed completely and one new spot was detected. The reaction mixture was concentrated at 25° C. to yield a residue which was dissolved in DCM (10 mL). The mixture was concentrated to yield a residue which was dissolved in MeOH (5 mL). The mixture was adjusted pH to 9 by saturated Na₂CO₃ solution then stirred at 25° C. for 2 h. The mixture was concentrated and water (80 mL) was added. The mixture was extracted with DCM (50 mL×3). The combined organic layers were washed with brine (50 mL), dried over Na₂SO₄, filtered and concentrated to yield a residue which was purified by preparative HPLC (column: Agela DuraShell C18 150*25 mm*5 um; mobile phase: [water (0.05% NH₃.H₂O+10 mM NH₄HCO₃)-ACN]; B %: 52%-82%, 10 min) to yield methyl 5-bromo-2-(1H-indole-3-carbonyl)thiazole-4-carboxylate (36.84 mg, 97.35 μmol, 43.4% yield, 96.5% purity) as a yellow solid. ¹H NMR (400 MHz, DMSO-d₆) δ ppm 12.41 (s, 1H), 9.02 (s, 1H), 8.31-8.23 (m, 1H), 7.63-7.55 (m, 1H), 7.35-7.25 (m, 2H), 3.94 (s, 3H); ES-LCMS m/z 365.0, 367.0 [M+H]⁺.

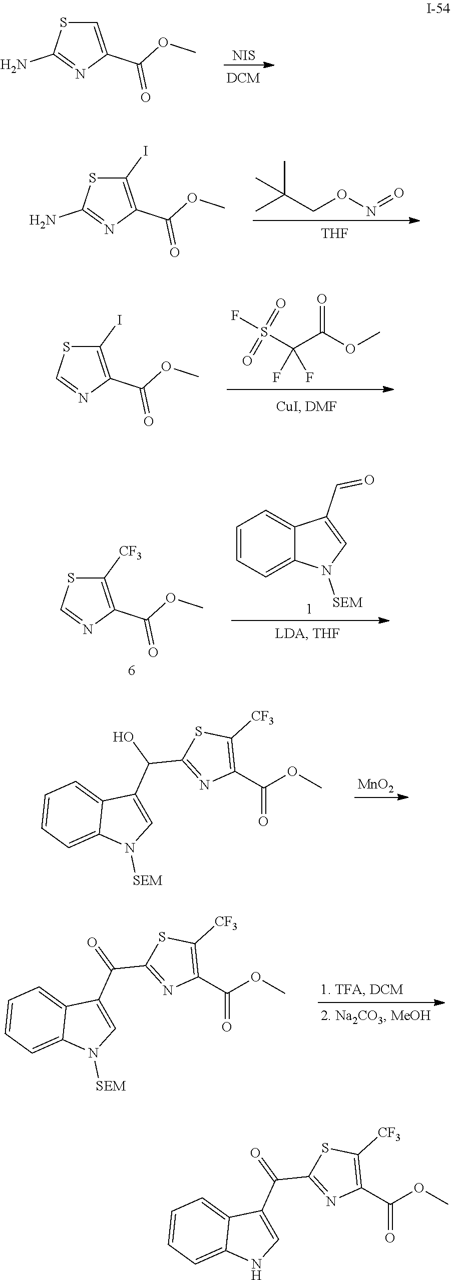

### Step 1: Methyl 2-amino-5-iodo-thiazole-4-carboxylate

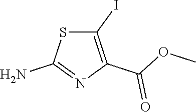

To a solution of methyl 2-aminothiazole-4-carboxylate (5 g, 31.61 mmol, 1 eq) in DCM (50 mL) was added NIS (8.53 g, 37.93 mmol, 1.2 eq). The mixture was stirred at 20° C. for 12 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield methyl 2-amino-5-iodo-thiazole-4-carboxylate (18 g, crude) as red oil, which was used in the next step without further purification. ¹H NMR (400 MHz, CDCl₃) δ ppm 5.68 (s, 3H); ES-LCMS m/z 285.0, 286.9 [M+H]⁺.

### Step 2: Methyl 5-iodothiazole-4-carboxylate

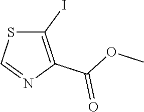

To a solution of methyl 2-amino-5-iodo-thiazole-4-carboxylate (18 g, 58.80 mmol, 92.8%, 1 eq) in THF (20 mL) was added tert-butyl nitrite (9.10 g, 88.20 mmol, 10.49 mL, 1.5 eq). The mixture was stirred at 60° C. for 1 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC:PE/EtOAc=3/1, R_{f}=0.50) to yield methyl 5-iodothiazole-4-carboxylate (5.7 g, 18.37 mmol, 31.2% yield, 86.7% purity) as a yellow solid. ¹H NMR (400 MHz, CDCl₃) δ ppm 8.96 (s, 1H), 3.99 (s, 3H); ES-LCMS m/z 270.0 271.9 [M+H]⁺.

### Step 3: Methyl 5-(trifluoromethyl)thiazole-4-carboxylate

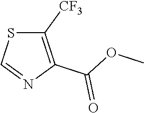

To a solution of methyl 5-iodothiazole-4-carboxylate (2 g, 6.44 mmol, 86.7%, 1 eq) in DMF (18 mL) was added CuI (2.45 g, 12.89 mmol, 2 eq) and methyl 2,2-difluoro-2-fluorosulfonyl-acetate (1.86 g, 9.67 mmol, 1.23 mL, 1.5 eq). The mixture was stirred at 80° C. for 12 h. The reaction mixture was quenched by addition of water (100 mL), extracted with EtOAc (50 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC:PE/EtOAc=3/1, R_{f}=0.40) to yield methyl 5-(trifluoromethyl)thiazole-4-carboxylate (865 mg, 3.89 mmol, 60.4% yield, 95.0% purity) as a white solid. ¹H NMR (400 MHz, CDCl₃) δ ppm 8.92 (s, 1H), 4.03 (s, 3H).

### Step 4: Methyl2-[hydroxy-[1-(2-trimethylsilylethoxymethyl)indol-3-yl]methyl]-5-(trifluoromethyl)thiazole-4-carboxylate

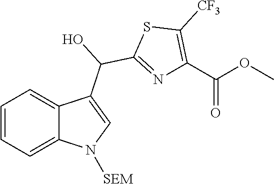 [SMILES: COC(=O)c1nc(C(O)c2cn(COCC[Si](C)(C)C)c3ccccc23)sc1C(F)(F)F]

To a stirred solution of DIPA (1.11 g, 10.97 mmol, 1.55 mL, 2.5 eq) in THF (50 mL) was cooled to −75° C. then added n-BuLi (2.5 M, 4.39 mL, 2.5 eq) dropwise under N₂ atmosphere. The reaction mixture was stirred at −75° C. for 30 min under N₂ atmosphere. 1-(2-trimethylsilylethoxymethyl)indole-3-carbaldehyde (1.31 g, 4.39 mmol, 92%, 1 eq) and methyl 5-(trifluoromethyl)thiazole-4-carboxylate (975 mg, 4.39 mmol, 95%, 1 eq) was dissolved in THF (50 mL). The LDA reaction mixture was added to the above mixture then stirred at −75° C. for 10 min under N₂ atmosphere. TLC (PE/EtOAc=3:1, R_{f}=0.35) showed the starting material was remained and one new spot was detected. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC:PE/EtOAc=3/1, R_{f}=0.30) to yield methyl2-[hydroxy-[1-(2-trimethylsilylethoxymethyl)indol-3-yl]methyl]-5-(trifluoromethyl)thiazole-4-carboxylate (600 mg, 1.17 mmol, 26.7% yield, 95.1% purity) as a yellow solid. ¹H NMR (400 MHz, CDCl₃) δ ppm 7.65 (d, J=8.1 Hz, 1H), 7.52 (d, J=8.3 Hz, 1H), 7.33-7.27 (m, 2H), 7.22-7.16 (m, 1H), 6.41 (s, 1H), 5.47 (s, 2H), 3.97 (s, 3H), 3.54-3.44 (m, 2H), 3.07 (s, 1H), 0.92-0.87 (m, 2H), −0.05 (s, 9H); ES-LCMS m/z 469.2 [M-OH]⁺.

### Step 5: Methyl 5-(trifluoromethyl)-2-[1-(2-trimethylsilylethoxymethyl)indole-3-carbonyl]thiazole-4-carboxylate

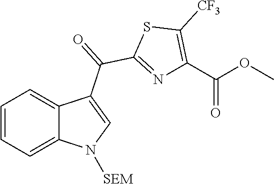 [SMILES: COC(=O)c1nc(C(=O)c2cn(COCC[Si](C)(C)C)c3ccccc23)sc1C(F)(F)F]

To a solution of methyl 2-[hydroxy-[1-(2-trimethylsilylethoxymethyl)indol-3-yl]methyl]-5-(trifluoromethyl)thiazole-4-carboxylate (600 mg, 1.17 mmol, 95.1%, 1 eq) in CHCl₃ (40 mL) was added MnO₂ (2.04 g, 23.45 mmol, 20 eq). The mixture was stirred at 60° C. for 12 h. TLC (PE/EtOAc=3:1, R_{f}=0.35) showed half of the starting material was remained and one new spot was detected. The reaction mixture was quenched by addition of water (50 mL), extracted with EtOAc (30 mL×3). The combined organic layers were washed with brine (10 mL), dried over Na₂SO₄, filtered and concentrated under reduced pressure to yield methyl 5-(trifluoromethyl)-2-[1-(2-trimethylsilylethoxymethyl)indole-3-carbonyl]thiazole-4-carboxylate (467 mg, 948.33 μmol, 80.8% yield, 98.4% purity) as a green solid, which was used in the next step without further purification. ¹H NMR (400 MHz, CDCl₃) δ ppm 9.10 (s, 1H), 8.54-8.47 (m, 1H), 7.63-7.57 (m, 1H), 7.44-7.39 (m, 2H), 5.63 (s, 2H), 4.06 (s, 3H), 3.58 (t, J=8.1 Hz, 2H), 0.94 (t, J=8.2 Hz, 2H), −0.04 (s, 9H); ES-LCMS m/z 485.1 [M+H]⁺.

### Step 6: Methyl 2-(1H-indole-3-carbonyl)-5-(trifluoromethyl)thiazole-4-carboxylate

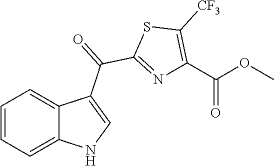 [SMILES: COC(=O)c1nc(C(=O)c2c[nH]c3ccccc23)sc1C(F)(F)F]

To a stirred solution of methyl 5-(trifluoromethyl)-2-[1-(2-trimethylsilylethoxymethyl)indole-3-carbonyl]thiazole-4-carboxylate (467 mg, 948.33 μmol, 98.4%, 1 eq) in DCM (5 mL) was added TFA (9.57 g, 83.97 mmol, 6.22 mL, 88.54 eq). The reaction mixture was stirred at 20° C. for 1.5 h. TLC (PE/EtOAc=3/1, R_{f}=0.60) showed starting material was consumed completely and one new spot was detected. The reaction mixture was concentrated at 20° C. to yield a residue which was dissolved in DCM (25 mL). The mixture was concentrated to yield a residue which was dissolved in MEOH (8 mL) and THF (6 mL). The mixture was adjusted pH to 9 by saturated Na₂HCO₃ solution then stirred at 20° C. for 2 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 55%-85%, 10 min) and lyophilizated to yield methyl 2-(1H-indole-3-carbonyl)-5-(trifluoromethyl)thiazole-4-carboxylate (114.52 mg, 291.00 μmol, 30.7% yield, 90.0% purity) as a green solid. ¹H NMR (400 MHz, DMSO-d₆) δ ppm 12.89-11.88 (m, 1H), 9.04 (s, 1H), 8.32-8.26 (m, 1H), 7.64-7.59 (m, 1H), 7.36-7.30 (m, 2H), 3.99 (s, 3H); ES-LCMS m/z 355.1 [M+H]⁺.

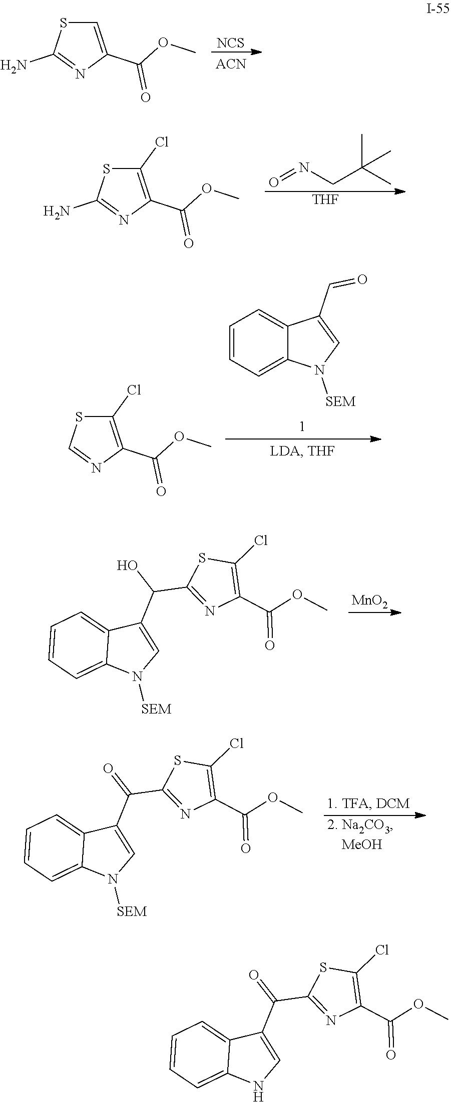

### Step 1: Methyl 2-amino-5-chloro-thiazole-4-carboxylate

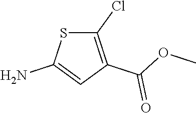

To a solution of methyl 2-aminothiazole-4-carboxylate (2 g, 12.64 mmol, 1 eq) in ACN (40 mL) was added NCS (3.38 g, 25.29 mmol, 2 eq). The mixture was stirred at 80° C. for 12 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 1/1, TLC:PE/EtOAc=1/1, R_{f}=0.30) to yield methyl 2-amino-5-chloro-thiazole-4-carboxylate (2.1 g, 7.95 mmol, 62.9% yield, 72.9% purity) as a yellow solid. ¹H NMR (400 MHz, CDCl₃) δ ppm 8.73-8.42 (m, 2H), 0.07 (s, 3H); ES-LCMS m/z 193.2, 195.2 [M+H]⁺.

### Step 2: Methyl 5-chlorothiazole-4-carboxylate

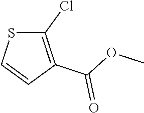

To a solution of methyl 2-amino-5-chloro-thiazole-4-carboxylate (1.89 g, 7.14 mmol, 72.9%, 1 eq) in THF (30 mL) was added tert-butyl nitrite (1.10 g, 10.71 mmol, 1.27 mL, 1.5 eq). The mixture was stirred at 60° C. for 1 h. TLC (PE/EtOAc=3:1, R_{f}=0.1) showed starting material was remained and one new spot was detected. The reaction mixture was quenched by addition of water (100 mL), extracted with EtOAc (50 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC:PE/EtOAc=3/1, R_{f}=0.50) to yield methyl 5-chlorothiazole-4-carboxylate (580 mg, 3.10 mmol, 43.4% yield, 95.0% purity) as a yellow solid. ¹H NMR (400 MHz, CDCl₃) δ ppm 8.67 (s, 1H), 3.99 (s, 3H).

### Step 3: Methyl5-chloro-2-[hydroxy-[1-(2-trimethylsilylethoxymethyl)indol-3-yl]methyl]thiazole-4-carboxylate

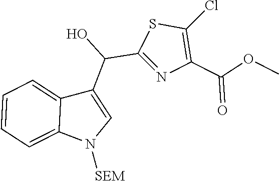 [SMILES: COC(=O)c1nc(C(O)c2cn(COCC[Si](C)(C)C)c3ccccc23)sc1Cl]

To a stirred solution of DIPA (595.37 mg, 5.88 mmol, 831.53 μL, 2 eq) in THF (15 mL) was cooled to −75° C. then added n-BuLi (2.5 M in hexane, 2.35 mL, 2 eq) dropwise under N₂ atmosphere. The reaction mixture was stirred at −75° C. for 30 min under N₂ atmosphere. 1-(2-trimethylsilylethoxymethyl)indole-3-carbaldehyde (880.70 mg, 2.94 mmol, 92%, 1 eq) and methyl 5-chlorothiazole-4-carboxylate (550 mg, 2.94 mmol, 95%, 1 eq) was dissolved in THF (15 mL). The LDA reaction mixture was added to the above mixture, and then stirred at −75° C. for 10 min under N₂ atmosphere. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by flash silica gel chromatography (from PE/EtOAc=1/0 to 3/1, TLC:PE/EtOAc=3/1, R_{f}=0.30) to yield methyl5-chloro-2-[hydroxy-[1-(2-trimethylsilylethoxymethyl)indol-3-yl]methyl]thiazole-4-carboxylate (350 mg, 647.42 μmol, 22.0% yield, 83.8% purity) as yellow oil. ¹H NMR (400 MHz, CDCl₃) δ ppm 7.63 (d, J=7.8 Hz, 1H), 7.51 (d, J=8.3 Hz, 1H), 7.31-7.27 (m, 2H), 7.20-7.14 (m, 1H), 6.33 (s, 1H), 5.47 (s, 2H), 3.95 (s, 3H), 3.51-3.46 (m, 2H), 3.04 (s, 1H), 0.92-0.87 (m, 2H), −0.05 (s, 9H); ES-LCMS m/z 435.2, 437.2 [M-OH]⁺.

### Step 4: Methyl 5-chloro-2-[1-(2-trimethylsilylethoxymethyl)indole-3-carbonyl]thiazole-4-carboxylate

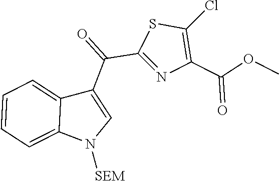 [SMILES: COC(=O)c1nc(C(=O)c2cn(COCC[Si](C)(C)C)c3ccccc23)sc1Cl]

To a solution of methyl 5-chloro-2-[hydroxy-[1-(2-trimethylsilylethoxymethyl)indol-3-yl]methyl]thiazole-4-carboxylate (307 mg, 567.88 μmol, 83.8%, 1 eq) in CHCl₃ (20 mL) was added MnO₂ (987.44 mg, 11.36 mmol, 20 eq). The mixture was stirred at 60° C. for 12 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was used in the next step without further purification to yield methyl 5-chloro-2-[1-(2-trimethylsilylethoxymethyl)indole-3-carbonyl]thiazole-4-carboxylate (374 mg, crude) as a yellow solid. ¹H NMR (400 MHz, CDCl₃) δ ppm 9.07 (s, 1H), 8.52-8.46 (m, 1H), 7.62-7.57 (m, 1H), 7.42-7.37 (m, 2H), 5.62 (s, 2H), 4.03 (s, 3H), 3.60-3.55 (m, 2H), 0.96-0.92 (m, 2H), −0.04 (s, 9H); ES-LCMS m/z 451.2, 453.2 [M+H]⁺.

### Step 5: Methyl 5-chloro-2-(1H-indole-3-carbonyl)thiazole-4-carboxylate

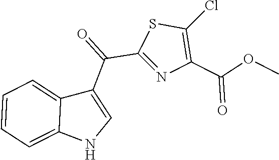 [SMILES: COC(=O)c1nc(C(=O)c2c[nH]c3ccccc23)sc1Cl]

To a stirred solution of methyl 5-chloro-2-[1-(2-trimethylsilylethoxymethyl)indole-3-carbonyl]thiazole-4-carboxylate (344 mg, 762.73 μmol, 100%, 1 eq) in DCM (5 mL) was added TFA (7.70 g, 67.53 mmol, 5 mL, 88.54 eq). The reaction mixture was stirred at 20° C. for 1.5 h. TLC (PE/EtOAc=3/1, R_{f}=0.60) showed the starting material was consumed completely and one new spot was detected. The reaction mixture was concentrated at 20° C. to yield a residue which was dissolved in DCM (25 mL). The mixture was concentrated to yield a residue which was dissolved in CH₃OH (8 mL) and THF (6 mL). The mixture was adjusted pH to 9 by saturated NaHCO₃ solution then stirred at 20° C. for 2 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 50%-80%, 10 min) and lyophilized to yield methyl 5-chloro-2-(1H-indole-3-carbonyl)thiazole-4-carboxylate (80.22 mg, 250.10 μmol, 32.8% yield, 100.0% purity) as white solid. ¹H NMR (400 MHz, DMSO-d₆) δ ppm 9.01 (s, 1H), 8.26 (dd, J=2.9, 5.6 Hz, 1H), 7.63-7.56 (m, 1H), 7.33-7.27 (m, 2H), 3.94 (s, 3H); ES-LCMS m/z 321.1, 323.1 [M+H]⁺.

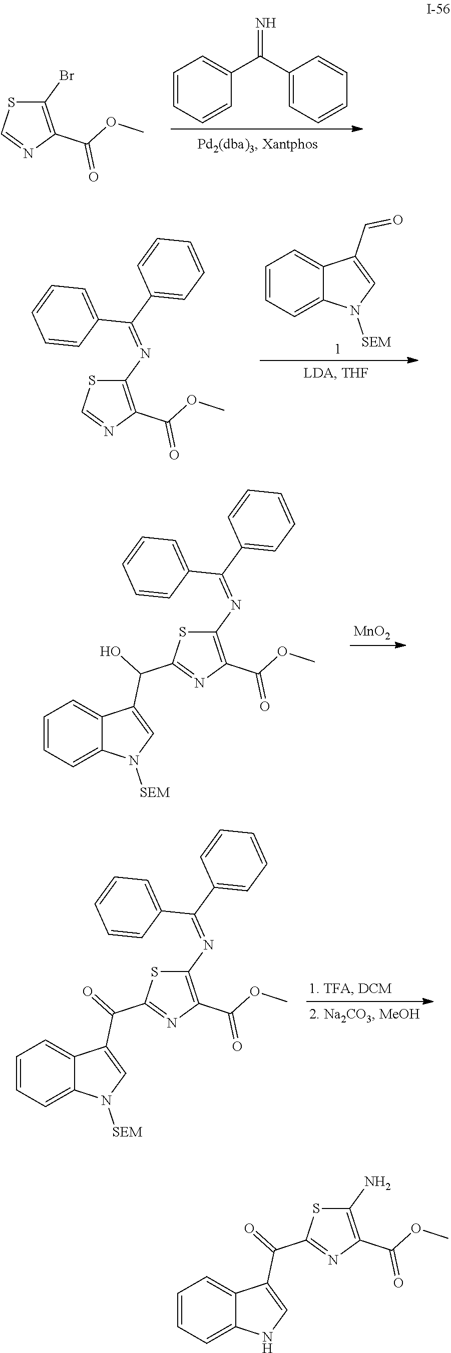

### Step 1: Methyl 5-(benzhydrylideneamino)thiazole-4-carboxylate

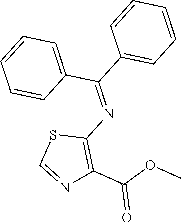 [SMILES: COC(=O)c1ncsc1N=C(c1ccccc1)c1ccccc1]

To a solution of methyl 5-bromothiazole-4-carboxylate (2 g, 6.30 mmol, 70% purity, 1 eq) in toluene (30 mL) were added diphenylmethanimine (1.49 g, 8.20 mmol, 1.38 mL, 1.3 eq), Cs₂CO₃ (4.52 g, 13.87 mmol, 2.2 eq), (5-diphenylphosphanyl-9,9-dimethyl-xanthen-4-yl)-diphenyl-phosphane (656.63 mg, 1.13 mmol, 0.18 eq) and Pd₂(dba)₃ (346.40 mg, 378.28 μmol, 0.06 eq). The mixture was stirred at 80° C. for 12 h under N₂. TLC (PE/EtOAc=3/1, R_{f}=0.20) indicated starting material was consumed completely and many new spots formed. The mixture was concentrated and then water (80 mL) was added. The mixture was extracted with EtOAc (50 mL×3). The combined organic layers were washed with brine (50 mL), dried over Na₂SO₄, filtered and concentrated to yield a residue which was purified by flash silica gel chromatography (From PE/EtOAc=1/0 to 3/1, R_{f}=0.20) to yield methyl 5-(benzhydrylideneamino)thiazole-4-carboxylate (1.84 g, 4.79 mmol, 76.0% yield, 84.0% purity) as a yellow oil. ¹H NMR (400 MHz, CDCl₃) δ ppm 8.31 (s, 1H), 7.84 (s, 2H), 7.42 (s, 6H), 7.27-7.15 (m, 2H), 3.90 (s, 3H); ES-LCMS m/z 323.2 [M+H]⁺.

### Step 2: Methyl 5-(benzhydrylideneamino)-2-[hydroxy-[1-(2-trimethylsilylethoxymethyl)indol-3-yl]methyl]thiazole-4-carboxylate

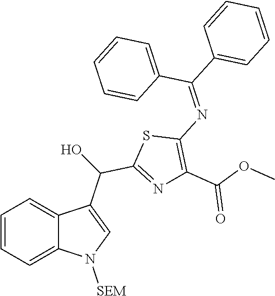 [SMILES: COC(=O)c1nc(C(O)c2cn(COCC[Si](C)(C)C)c3ccccc23)sc1N=C(c1ccccc1)c1ccccc1]

To a solution of methyl 5-(benzhydrylideneamino)thiazole-4-carboxylate (1.84 g, 4.79 mmol, 84% purity, 1 eq) in THF (20 mL) was added LDA (2 M, 11.99 mL, 5 eq) and 1-(2-trimethylsilylethoxymethyl)indole-3-carbaldehyde (1.44 g, 4.79 mmol, 92% purity, 1 eq). The mixture was stirred at −75° C. for 1 h under N₂. TLC (PE/EtOAc=3/1, R_{f}=0.10) indicated starting material was consumed completely and many new spots formed. The residue was partitioned between EtOAc (100 mL) and water (50 mL). The organic layer was washed with brine (50 mL), dried over Na₂SO₄, filtered and concentrated to yield a residue which was purified by flash silica gel chromatography (From PE/EtOAc=1/0 to 3/1, R_{f}=0.10) to yield methyl 5-(benzhydrylideneamino)-2-[hydroxy-[1-(2-trimethylsilylethoxymethyl)indol-3-yl]methyl]thiazole-4-carboxylate (630 mg, 763.00 μmol, 15.9% yield, 72.4% purity) as yellow oil. ¹H NMR (400 MHz, CDCl₃) δ ppm 7.47-7.33 (m, 14H), 7.14-7.07 (m, 2H), 6.18 (d, J=3.1 Hz, 1H), 5.42 (s, 2H), 3.87-3.81 (m, 3H), 3.49-3.43 (m, 2H), 0.88 (s, 2H), −0.06 (s, 9H); ES-LCMS m/z 598.2 [M-OH]⁺.

### Step 3: Methyl 5-(benzhydrylideneamino)-2-[1-(2-trimethylsilylethoxymethyl)indole-3-carbonyl]thiazole-4-carboxylate

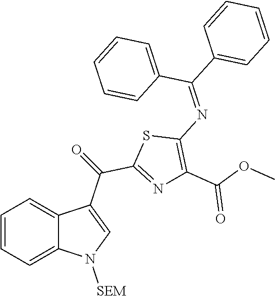 [SMILES: COC(=O)c1nc(C(=O)c2cn(COCC[Si](C)(C)C)c3ccccc23)sc1N=C(c1ccccc1)c1ccccc1]

To a solution of methyl 5-(benzhydrylideneamino)-2-[hydroxy-[1-(2-trimethylsilylethoxymethyl)indol-3-yl]methyl]thiazole-4-carboxylate (630 mg, 763.00 μmol, 72.4% purity, 1 eq) in CHCl₃ (40 mL) was added MnO₂ (1.66 g, 19.08 mmol, 25 eq). The mixture was stirred at 70° C. for 12 h under N₂. TLC (PE/EtOAc=3/1, R_{f}=0.60) indicated starting material was consumed completely and two new spots formed. The mixture was filtered, washed with EtOAc (50 mL×2). The filtrate was concentrated to yield a residue which was purified by flash silica gel chromatography (From PE/EtOAc=1/0 to 3/1, Rf=0.52) to yield methyl 5-(benzhydrylideneamino)-2-[1-(2-trimethylsilylethoxymethyl)indole-3-carbonyl]thiazole-4-carboxylate (280 mg, 418.27 μmol, 54.8% yield, 89.0% purity) as yellow oil. ¹H NMR (400 MHz, CDCl₃) δ ppm 9.03 (s, 1H), 8.50-8.42 (m, 1H), 7.59-7.30 (m, 13H), 5.59 (s, 2H), 3.91 (s, 3H), 3.58-3.51 (m, 2H), 0.93 (d, J=8.2 Hz, 2H), −0.05 (s, 9H); ES-LCMS m/z 596.2 [M+H]⁺.

### Step 4: Methyl 5-amino-2-(1H-indole-3-carbonyl)thiazole-4-carboxylate

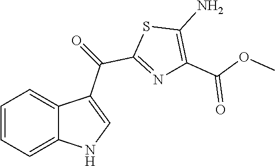 [SMILES: COC(=O)c1nc(C(=O)c2c[nH]c3ccccc23)sc1N]

To a stirred solution of methyl 5-(benzhydrylideneamino)-2-[1-(2-trimethylsilylethoxymethyl)indole-3-carbonyl]thiazole-4-carboxylate (280 mg, 418.27 μmol, 89% purity, 1 eq) in DCM (4 mL) was added TFA (9.46 g, 82.93 mmol, 6.14 mL, 198.26 eq). The reaction mixture was stirred at 25° C. for 1 h. TLC (PE/EtOAc=3/1, R_{f}=0.34) showed starting material was consumed completely and many new spots were detected. The reaction mixture was concentrated at 25° C. to yield a residue which was dissolved in DCM (5 mL). The mixture was concentrated to yield a residue which was dissolved in MeOH (5 mL). The mixture was adjusted to pH 9 by saturated Na₂CO₃ solution then stirred at 25° C. for 1 h. The mixture was concentrated and water (80 mL) was added. The mixture was extracted with EtOAc (30 mL×3). The combined organic layers were washed with brine (50 mL), dried over Na₂SO₄, filtered and concentrated to yield a residue which was purified by preparative HPLC (column: Agela DuraShell C18 150*25 mm*5 um; mobile phase: [water (0.05% NH₃.H₂O+10 mM NH₄HCO₃)-ACN]; B %: 29%-59%, 10 min) to yield methyl 5-amino-2-(1H-indole-3-carbonyl)thiazole-4-carboxylate (16.39 mg, 54.39 mol, 13.0% yield, 100.0% purity) as a yellow solid. ¹H NMR (400 MHz, DMSO-d₆) δ ppm 12.12 (s, 1H), 8.93 (s, 1H), 8.30-8.23 (m, 1H), 8.01 (s, 2H), 7.58-7.52 (m, 1H), 7.30-7.20 (m, 2H), 3.84 (s, 3H); ES-LCMS m/z 302.2 [M+H]⁺.

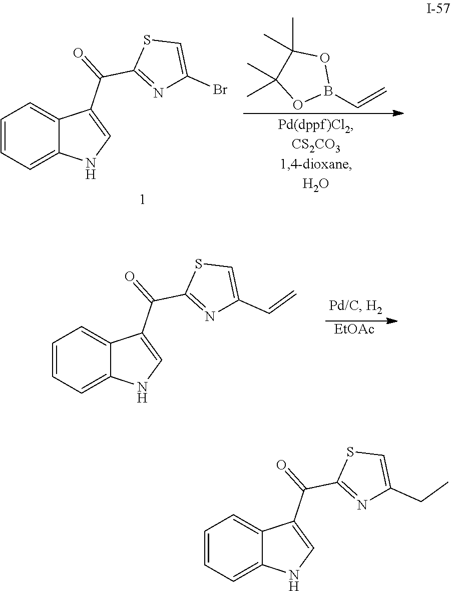

### Step 1: 1H-Indol-3-yl-(4-vinylthiazol-2-yl)methanone

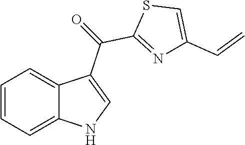 [SMILES: C=Cc1csc(C(=O)c2c[nH]c3ccccc23)n1]

To a stirred solution of (4-bromothiazol-2-yl)-(1H-indol-3-yl)methanone (100 mg, 319.05 μmol, 98.0% purity, 1 eq) and 4,4,5,5-tetramethyl-2-vinyl-1,3,2-dioxaborolane (73.71 mg, 478.57 μmol, 81.17 μL, 1.5 eq) in 1,4-dioxane (6 mL) and H₂O (2 mL) was added CS₂CO₃ (207.90 mg, 638.09 μmol, 2 eq) and Pd(dppf)Cl₂ (23.34 mg, 31.90 μmol, 0.1 eq). The reaction mixture was stirred at 100° C. for 3 h under N₂ atmosphere. The reaction mixture was diluted with H₂O (15 mL) and extracted with EtOAc (30 mL×3). The combined organic layers were washed with brine (30 mL), dried over Na₂SO₄, filtered and concentrated under reduced pressure to yield a residue which was purified by flash silica gel chromatography (from PE/EtOAc=100/1 to 3/1, TLC:PE/EtOAc=3/1, R_{f}=0.47) to yield 1H-indol-3-yl-(4-vinylthiazol-2-yl)methanone (80 mg, 292.56 μmol, 91.7% yield, 93.0% purity) as a yellow solid. ¹H NMR (400 MHz, DMSO-d₆) δ ppm 12.24 (s, 1H), 9.17 (s, 1H), 8.37-8.28 (m, 1H), 8.03 (s, 1H), 7.63-7.52 (m, 1H), 7.32-7.24 (m, 2H), 6.95-6.88 (m, 1H), 6.20 (dd, J=1.7, 17.4 Hz, 1H), 5.50 (dd, J=1.6, 10.9 Hz, 1H); ES-LCMS m/z 255.2 [M+H]⁺.

### Step 2: (4-Ethylthiazol-2-yl)-(1H-indol-3-yl)methanone

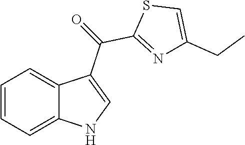 [SMILES: CCc1csc(C(=O)c2c[nH]c3ccccc23)n1]

To a stirred solution of 1H-indol-3-yl-(4-vinylthiazol-2-yl)methanone (80 mg, 292.56 μmol, 93.0% purity, 1 eq) in EtOAc (10 mL) was added Pd/C (100 mg, 10%). The reaction mixture was stirred at 25° C. for 1 h. The reaction mixture was concentrated under reduced pressure to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 48%-78%, 10 min) to yield (4-ethylthiazol-2-yl)-(1H-indol-3-yl)methanone (32.81 mg, 128.00 μmol, 43.8% yield, 100.0% purity) as a light yellow solid. ¹H NMR (400 MHz, DMSO-d₆) δ ppm 11.58 (s, 1H), 9.10 (s, 1H), 8.31 (dd, J=2.9, 5.9 Hz, 1H), 7.72 (s, 1H), 7.61-7.54 (m, 1H), 7.32-7.22 (m, 2H), 2.89 (q, J=7.4 Hz, 2H), 1.34 (t, J=7.6 Hz, 3H); ES-LCMS m/z 257.2 [M+H]⁺.

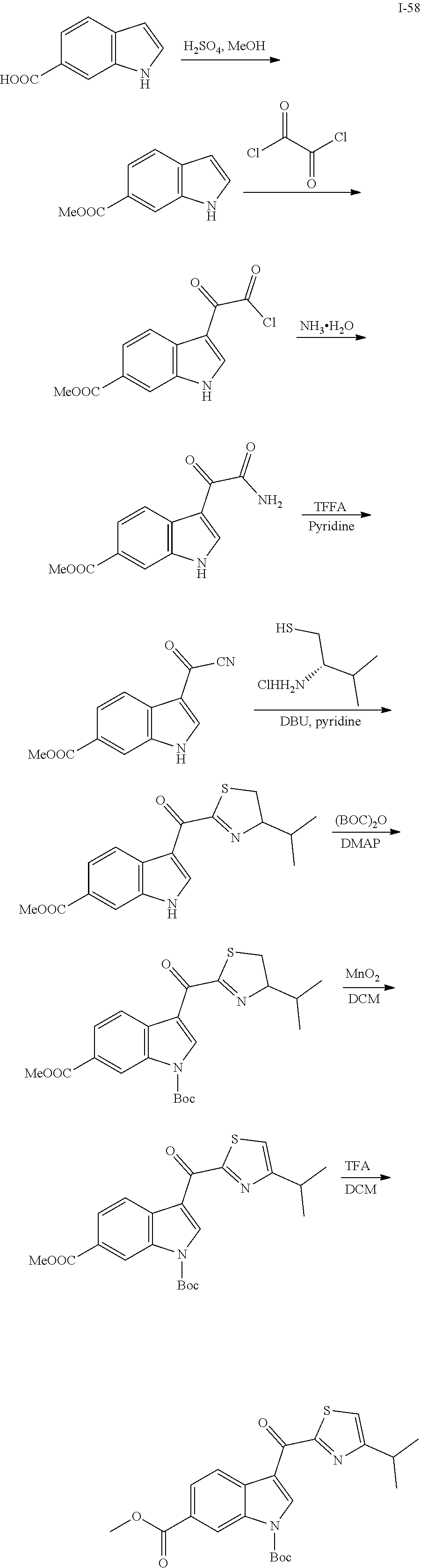

### Step 1: Methyl 1H-indole-6-carboxylate

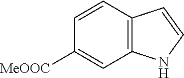 [SMILES: COC(=O)c1ccc2cc[nH]c2c1]

To a solution of 1H-indole-6-carboxylic acid (2 g, 12.41 mmol, 1 eq) in MeOH (100 mL) was added H2504 (1.84 g, 18.76 mmol, 1 mL, 98%, 1.51 eq). The mixture was stirred at 70° C. for 15 h. TLC (PE/EtOAc=3/1, R_{f}=0.47) showed the starting material was consumed completely. The reaction mixture was quenched with sat. aq NaHCO₃ (50 mL) and extracted with EtOAc (100 mL×3). The combined organic layers dried over Na₂SO₄, filtered and concentrated under reduced pressure to yield a residue which was purified by flash silica gel chromatography (from PE/EtOAc=100/1 to 3/1, TLC:PE/EtOAc=3/1, R_{f}=0.43) to yield methyl 1H-indole-6-carboxylate (2 g, 11.42 mmol, 91.9% yield, 100.0% purity) as a white solid. ¹H NMR (500 MHz, DMSO-d₆) δ ppm 11.50 (s, 1H), 8.08 (s, 1H), 7.64-7.52 (m, 3H), 6.53 (t, J=1.9 Hz, 1H), 3.87-3.82 (m, 3H); ES-LCMS m/z 176.3 [M+H]⁺.

### Step 2: Methyl 3-(2-chloro-2-oxo-acetyl)-1H-indole-6-carboxylate

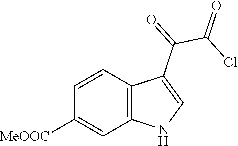 [SMILES: COC(=O)c1ccc2c(C(=O)C(=O)Cl)c[nH]c2c1]

To a solution of methyl 1H-indole-6-carboxylate (1.9 g, 10.85 mmol, 100%, 1 eq) in THF (12 mL) was added oxalyl dichloride (1.58 g, 12.47 mmol, 1.09 mL, 1.15 eq). The mixture was stirred at 0° C. for 3 h under N₂. The reaction mixture was filtered and concentrated under reduced pressure to yield methyl 3-(2-chloro-2-oxo-acetyl)-1H-indole-6-carboxylate (1.7 g, 6.40 mmol, 59.0% yield, 100.0% purity) as a light yellow solid. ¹H NMR (500 MHz, DMSO-d₆) δ ppm 12.68 (s, 1H), 8.63 (s, 1H), 8.26 (d, J=8.4 Hz, 1H), 8.17 (s, 1H), 7.87 (d, J=8.2 Hz, 1H), 3.88 (s, 3H).

### Step 3: Methyl 3-oxamoyl-1H-indole-6-carboxylate

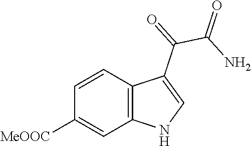 [SMILES: COC(=O)c1ccc2c(C(=O)C(N)=O)c[nH]c2c1]

To a solution of methyl 3-(2-chloro-2-oxo-acetyl)-1H-indole-6-carboxylate (1.7 g, 6.40 mmol, 100%, 1 eq) in THF (12 mL) was added NH₃.H₂O (8.01 g, 63.99 mmol, 8.80 mL, 28%, 10 eq). The mixture was stirred at 0° C. for 1.5 h. The reaction mixture was filtered and the cake was washed with H₂O (30 mL×3) and concentrated under reduced pressure to yield methyl 3-oxamoyl-1H-indole-6-carboxylate (1.5 g, 6.09 mmol, 95.2% yield, 100.0% purity) as a white solid. ¹H NMR (400 MHz, DMSO-d₆) δ ppm 8.30 (s, 1H), 8.23 (d, J=8.6 Hz, 1H), 8.11 (s, 1H), 7.77 (d, J=8.6 Hz, 1H), 7.37 (s, 3H), 3.86 (s, 3H); ES-LCMS m/z 247.2 [M+H]⁺.

### Step 4: Methyl 3-carbonocyanidoyl-1H-indole-6-carboxylate

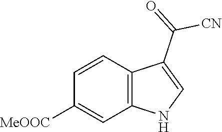 [SMILES: COC(=O)c1ccc2c(C(=O)C#N)c[nH]c2c1]

To a solution of methyl 3-oxamoyl-1H-indole-6-carboxylate (1.5 g, 6.09 mmol, 100%, 1 eq) in Pyridine (6 mL) was added TFAA (3.84 g, 18.28 mmol, 2.54 mL, 3 eq). The mixture was stirred at 25° C. for 12 h. The reaction mixture was quenched by addition of sat. aq. NaHCO₃ (100 mL), extracted with EtOAc (80 mL×3). The combined organic layers were washed with 0.5 N aq. HCl (40 mL), brine (40 mL), dried over Na₂SO₄, filtered and the filtrate was concentrated under reduced pressure to yield methyl 3-carbonocyanidoyl-1H-indole-6-carboxylate (1.3 g, 4.56 mmol, 74.8% yield, 80.0% purity) as a brown solid. ¹H NMR (400 MHz, DMSO-d₆) δ ppm 13.17 (s, 1H), 8.82 (s, 1H), 8.18-8.05 (m, 2H), 7.92 (d, J=8.3 Hz, 1H), 3.88 (s, 3H); ES-LCMS m/z 229.2 [M+H]⁺.

### Step 5: Methyl 3-(4-isopropyl-4,5-dihydrothiazole-2-carbonyl)-1H-indole-6-carboxylate

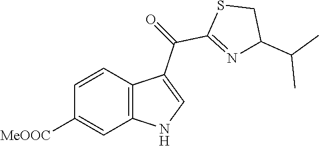 [SMILES: COC(=O)c1ccc2c(C(=O)C3=NC(C(C)C)CS3)c[nH]c2c1]

To a solution of methyl 3-carbonocyanidoyl-1H-indole-6-carboxylate (500 mg, 2.19 mmol, 100%, 1 eq) in pyridine (6 mL) was added DBU (33.36 mg, 219.10 μmol, 33.03 μL, 0.1 eq) and (2S)-2-amino-3-methyl-butane-1-thiol; hydrochloride (341.12 mg, 2.19 mmol, 100%, 1 eq). The mixture was stirred at 55° C. for 12 h under N₂. The reaction mixture was filtered and concentrated under reduced pressure to yield methyl 3-(4-isopropyl-4,5-dihydrothiazole-2-carbonyl)-1H-indole-6-carboxylate (500 mg, 1.21 mmol, 55.2% yield, 80.0% purity) as a brown solid. ¹H NMR (400 MHz, DMSO-d₆) δ ppm 11.91 (s, 1H), 8.84 (s, 2H), 8.09 (s, 1H), 7.80-7.75 (m, 1H), 7.41-7.36 (m, 1H), 3.86 (s, 2H), 3.65 (s, 1H), 2.45-2.41 (m, 1H), 2.13-2.06 (m, 2H), 0.87 (d, J=6.6 Hz, 6H); ES-LCMS m/z 331.2 [M+H]⁺.

### Step 6: O1-tert-Butyl 06-methyl 3-(4-isopropyl-4,5-dihydrothiazole-2-carbonyl)indole-1,6-dicarboxylate

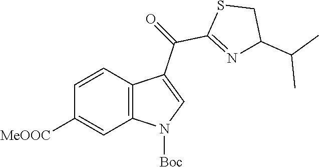 [SMILES: COC(=O)c1ccc2c(C(=O)C3=NC(C(C)C)CS3)cn(C(=O)OC(C)(C)C)c2c1]

To a solution of methyl 3-(4-isopropyl-4,5-dihydrothiazole-2-carbonyl)-1H-indole-6-carboxylate (0.3 g, 907.99 μmol, 100%, 1 eq) in 1,4-dioxane (6 mL) was added (Boc)₂O (260 mg, 1.19 mmol, 1.31 eq) and DMAP (130 mg, 1.06 mmol, 1.17 eq). The mixture was stirred at 70° C. for 2 h under N₂. The reaction mixture was diluted with water (20 mL), extracted with EtOAc (50 mL×3). The combined organic layers were dried over Na₂SO₄, filtered and concentrated. The residue was purified by flash silica gel chromatography (from PE/EtOAc=100/1 to 3/1, TLC:PE/EtOAc=3/1, R_{f}=0.65) to yield O1-tert-butyl 06-methyl 3-(4-isopropyl-4,5-dihydrothiazole-2-carbonyl)indole-1,6-dicarboxylate (240 mg, 501.72 μmol, 55.2% yield, 90.0% purity) as a yellow solid. ¹H NMR (400 MHz, DMSO-d₆) δ ppm 9.20 (s, 1H), 8.81 (s, 1H), 8.36 (d, J=8.3 Hz, 1H), 8.01 (dd, J=1.3, 8.4 Hz, 1H), 3.91 (s, 3H), 3.65-3.59 (m, 1H), 3.11 (d, J=11.0 Hz, 2H), 2.09-2.02 (m, 1H), 1.66 (s, 9H), 1.16-1.08 (m, 6H); ES-LCMS m/z 431.2 [M+H]⁺.

### Step 7: O1-tert-Butyl 06-methyl 3-(4-isopropylthiazole-2-carbonyl)indole-1,6-dicarboxylate

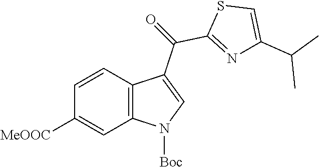 [SMILES: COC(=O)c1ccc2c(C(=O)c3nc(C(C)C)cs3)cn(C(=O)OC(C)(C)C)c2c1]

To a solution of O1-tert-butyl 06-methyl 3-(4-isopropyl-4,5-dihydrothiazole-2-carbonyl)indole-1,6-dicarboxylate (240 mg, 501.72 μmol, 90%, 1 eq) in 1,2-dichloroethane (8 mL) was added MnO₂ (872.40 mg, 10.03 mmol, 20 eq). The mixture was stirred at 95° C. for 12 h under N₂. The reaction mixture was filtered and concentrated to yield O1-tert-butyl 06-methyl 3-(4-isopropylthiazole-2-carbonyl)indole-1,6-dicarboxylate (200 mg, 350.06 μmol, 69.7% yield, 75.0% purity) as a yellow solid. ¹H NMR (400 MHz, DMSO-d₆) δ ppm 9.60 (s, 1H), 8.84 (s, 1H), 8.45 (d, J=8.3 Hz, 1H), 8.04-8.01 (m, 1H), 7.89-7.88 (m, 1H), 3.91 (s, 3H), 3.24-3.18 (m, 1H), 1.69 (s, 9H), 1.37-1.36 (m, 6H); ES-LCMS m/z 429.2 [M+H]⁺.

### Step 8: Methyl 3-(4-isopropylthiazole-2-carbonyl)-1H-indole-6-carboxylate

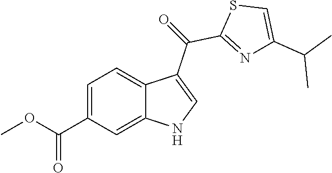 [SMILES: COC(=O)c1ccc2c(C(=O)c3nc(C(C)C)cs3)c[nH]c2c1]

To a solution of O1-tert-butyl 06-methyl 3-(4-isopropylthiazole-2-carbonyl) indole-1,6-dicarboxylate (60 mg, 126.02 μmol, 90%, 1 eq) in DCM (2 mL) was added TFA (14.37 mg, 126.02 μmol, 9.33 L, eq). The mixture was stirred at 20° C. for 2 h. The reaction mixture was concentrated to yield a residue. The residue was purified by preparative HPLC (column: Agela DuraShell C18 150*25 mm*5 μm; mobile phase: [water (0.05% NH₃.H₂O+10 mM NH₄HCO₃)-ACN]; B %: 54%-84%, 10 min), followed by lyophilization to yield methyl 3-(4-isopropylthiazole-2-carbonyl)-1H-indole-6-carboxylate (17.65 mg, 53.75 μmol, 42.6% yield, 100.0% purity) as a white solid. ¹H NMR (500 MHz, CDCl₃) δ ppm 9.33 (d, J=3.2 Hz, 1H), 8.92 (s, 1H), 8.60 (d, J=8.4 Hz, 1H), 8.23 (s, 1H), 8.04 (dd, J=1.2, 8.4 Hz, 1H), 7.25 (s, 1H), 3.97 (s, 3H), 3.22 (td, J=6.8, 13.7 Hz, 1H), 1.42 (s, 3H), 1.41 (s, 3H); ES-LCMS m/z 329.1 [M+H]⁺.

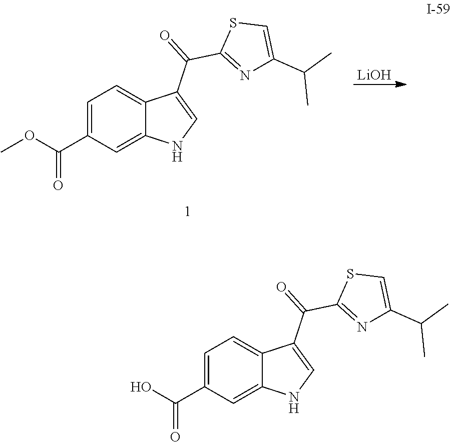

### Step 1: 3-(4-Isopropylthiazole-2-carbonyl)-1H-indole-6-carboxylic acid

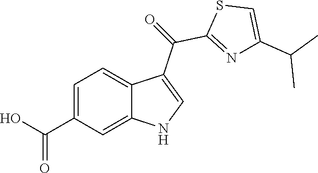 [SMILES: CC(C)c1csc(C(=O)c2c[nH]c3cc(C(=O)O)ccc23)n1]

To a solution of methyl 3-(4-isopropylthiazole-2-carbonyl)-1H-indole-6-carboxylate (60 mg, 146.17 μmol, 80%, 1 eq) in THF (2 mL) and MeOH (2 mL) was added aq. LiOH (1 M, 438.51 μL, 3 eq). The mixture was stirred at 60° C. for 2 h. The reaction mixture was concentrated to yield a residue. The residue was purified by preparative HPLC (column: Agela DuraShell C18 150*25 mm*5 μm; mobile phase: [water (0.05% NH₃.H₂O+10 mM NH₄HCO₃)-ACN]; B %: 15%-45%, 10 min), followed by lyophilization to yield 3-(4-isopropylthiazole-2-carbonyl)-1H-indole-6-carboxylic acid (21.6 mg, 67.89 μmol, 46.4% yield, 98.8% purity) as a white solid. ¹H NMR (500 MHz, DMSO-d₆) δ ppm 9.17 (s, 1H), 8.28 (d, J=8.4 Hz, 1H), 8.15 (s, 1H), 7.85 (dd, J=1.3, 8.3 Hz, 1H), 7.73 (d, J=0.6 Hz, 1H), 3.20 (td, J=6.9, 13.8 Hz, 1H), 1.37 (s, 3H), 1.36 (s, 3H); ES-LCMS m/z 315.1 [M+H]⁺.

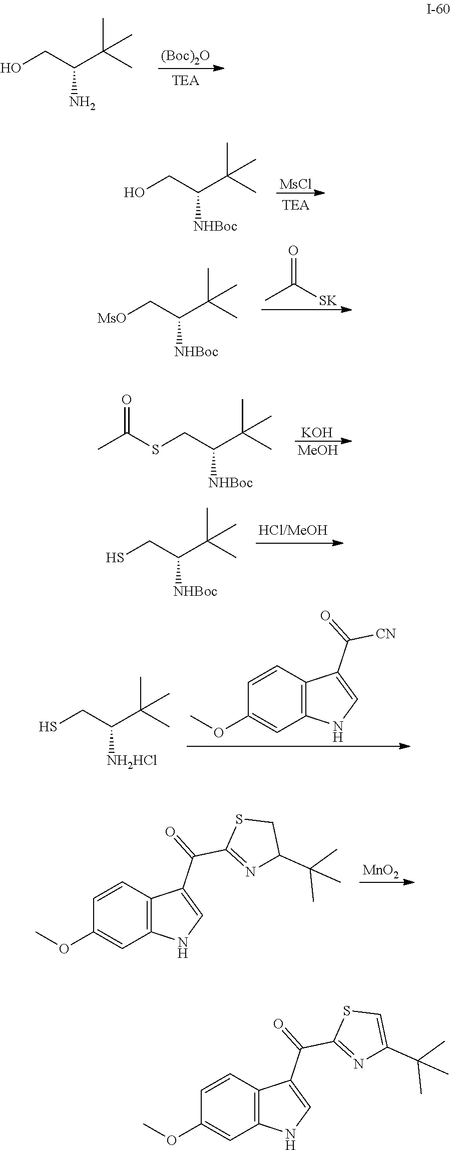

### Step 1: tert-Butyl N-[(1S)-1-(hydroxymethyl)-2,2-dimethyl-propyl]carbamate

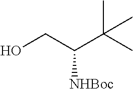

To a solution of (2S)-2-amino-3,3-dimethyl-butan-1-ol (1.50 g, 12.80 mmol, 1.56 mL, 1 eq) in THF (50 mL) was added TEA (1.30 g, 12.80 mmol, 1.78 mL, 1 eq) and tert-butoxycarbonyl tert-butyl carbonate (2.79 g, 12.80 mmol, 2.94 mL, 1 eq) at 0° C. The mixture was stirred at 15° C. for 12 h. TLC (PE/EtOAc=1/1, R_{f}=0.19) showed that one new point was formed and the start material was consumed completely. The reaction mixture was concentrated under reduced pressure. The residue was diluted with water (50 mL) and extracted with EtOAc (50 mL×3). The combined organic layers were washed with brine (50 mL) dried over Na₂SO₄, filtered and concentrated under reduced pressure to yield tert-butyl N-[(1S)-1-(hydroxymethyl)-2,2-dimethyl-propyl]carbamate (2.9 g, 12.01 mmol, 93.8% yield, 90.0% purity) as a white solid. ¹H NMR (400 MHz, DMSO-d₆) δ ppm 6.40 (d, J=8.2 Hz, 1H), 4.33 (t, J=5.2 Hz, 1H), 3.56-3.47 (m, 1H), 3.29-3.19 (m, 2H), 1.38 (s, 9H), 0.82 (s, 9H).

### Step 2: [(2S)-2-(tert-Butoxycarbonylamino)-3,3-dimethyl-butyl] methanesulfonate

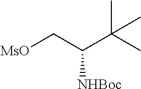 [SMILES: CC(C)(C)OC(=O)N[C@H](COS(C)(=O)=O)C(C)(C)C]

To a solution of tert-butyl N-[(1S)-1-(hydroxymethyl)-2,2-dimethyl-propyl]carbamate (2.8 g, 11.60 mmol, 90.0% 1 eq) and MsCl (2.09 g, 18.25 mmol, 1.41 mL, 1.57 eq) in DCM (50 mL) was added TEA (3.52 g, 34.79 mmol, 4.84 mL, 3 eq) at 0° C. The mixture was stirred at 15° C. for 2 h. TLC (PE/EtOAc=1/1, R_{f}=0.30) showed that one new point was formed and the starting material was consumed completely. The reaction mixture was quenched by aqueous NaHCO₃ solution (50 mL) and extracted with DCM (40 mL×3). The combined organic layers was dried over Na₂SO₄, filtered and concentrated under reduced pressure to yield [(2S)-2-(tert-butoxy carbonylamino)-3,3-dimethyl-butyl] methanesulfonate (3.5 g, 10.66 mmol, 92.0% yield, 90.0% purity) as yellow oil. ¹H NMR (400 MHz, DMSO-d₆) δ ppm 6.93 (d, J=9.8 Hz, 1H), 4.35 (dd, J=3.2, 10.4 Hz, 1H), 4.00 (t, J=10.0 Hz, 1H), 3.48-3.58 (m, 1H), 3.16 (s, 3H), 1.39 (s, 9H), 0.87 (s, 9H).

### Step 3: S-[(2S)-2-(tert-Butoxycarbonylamino)-3,3-dimethyl-butyl] ethanethioate

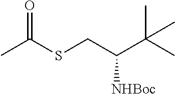 [SMILES: CC(=O)SC[C@@H](NC(=O)OC(C)(C)C)C(C)(C)C]

To a solution of [(2S)-2-(tert-butoxycarbonylamino)-3,3-dimethyl-butyl] methanesulfonate (3 g, 9.14 mmol, 90.0%, 1 eq) in DMF (80 mL) was added Cs₂CO₃ (4.47 g, 13.71 mmol, 1.5 eq) and acetylsulfanylpotassium (1.57 g, 13.71 mmol, 1.5 eq). The mixture was stirred at 40° C. for 12 h. TLC (PE/EtOAc=3/1, R_{f}=0.30) showed that one new point was formed and the starting material was consumed completely. The reaction mixture was quenched by addition of H₂O (100 mL) and extracted with EtOAc (100 mL×3). The combined organic layers were washed with brine (100 mL), dried over Na₂SO₄, filtered and concentrated under reduced pressure to yield S-[(2S)-2-(tert-butoxycarbonylamino)-3,3-dimethyl-butyl] ethanethioate (2 g, 6.90 mmol, 75.5% yield, 95.0% purity) as light yellow oil. ¹H NMR (400 MHz, CDCl₃) δ ppm 4.49-4.35 (m, 1H), 3.56-3.44 (m, 1H), 3.09 (dd, J=3.2, 13.8 Hz, 1H), 2.87 (dd, J=12.0, 13.4 Hz, 1H), 2.34 (s, 3H), 1.43 (s, 9H), 0.96 (s, 9H).

### Step 4: tert-Butyl N-[(1S)-2,2-dimethyl-1-(sulfanylmethyl)propyl]carbamate

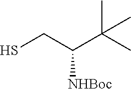

To a solution of S-[(2S)-2-(tert-butoxycarbonylamino)-3,3-dimethyl-butyl] ethanethioate (2 g, 6.90 mmol, 95.0%, 1 eq) in MeOH (30 mL) was added KOH (1.94 g, 34.49 mmol, 5 eq). The mixture was stirred at 15° C. for 2 h. TLC (PE/EtOAc=5/1, R_{f}=0.52) showed that one new point was formed and the starting material was consumed completely. The mixture was concentrated and then water (50 mL) was added. The mixture was extracted with EtOAc (50 mL×3). The combined organic layers were washed with brine (50 mL), dried over Na₂SO₄, filtered and concentrated under reduced pressure to yield tert-butyl N-[(1S)-2,2-dimethyl-1-(sulfanylmethyl)propyl]carbamate (1.7 g, 6.56 mmol, 95.0% yield, 90.0% purity) as a white solid. ¹H NMR (400 MHz, DMSO-d₆) δ ppm 6.68-6.58 (m, 1H), 3.29-3.18 (m, 1H), 2.77-2.65 (m, 1H), 2.32-2.39 (m, 1H), 1.97-1.87 (m, 1H), 1.39 (s, 9H), 0.83-0.78 (m, 9H).

### Step 5: (S)-2-Amino-3,3-dimethylbutane-1-thiolhydrochloride

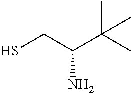

To a solution of tert-butyl N-[(1S)-2,2-dimethyl-1-(sulfanylmethyl)propyl]carbamate (1.7 g, 6.56 mmol, 90% purity, 1 eq) in HCl/MeOH (4 M, 1.64 mL, 1 eq) under N₂ atmosphere. The mixture was stirred at 15° C. for 2 h. TLC (PE/EtOAc=3/1, R_{f}=0.62) showed that one new point was formed and the starting material was consumed completely. The mixture was concentrated to yield (2S)-2-amino-3,3-dimethyl-butane-1-thiolhydrochloride (1.2 g, 5.53 mmol, 84.3% yield, 95.0% purity, HCl) as light yellow oil. ¹H NMR (400 MHz, DMSO-d₆) δ ppm 8.13 (s, 2H), 3.39-3.29 (m, 1H), 2.91-2.84 (m, 2H), 2.63-2.53 (m, 1H), 0.95 (s, 9H).

### Step 6: (4-(tert-Butyl)-4,5-dihydrothiazol-2-yl)(6-methoxy-1H-indol-3-yl)methanone

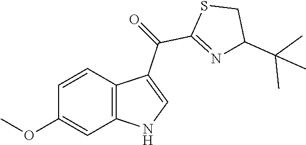 [SMILES: COc1ccc2c(C(=O)C3=NC(C(C)(C)C)CS3)c[nH]c2c1]

To a solution of (2S)-2-amino-3,3-dimethyl-butane-1-thiolhydrochloride (500 mg, 2.30 mmol, 95.0%, 1 eq, HCl) in pyridine (15 mL) was added DBU (35.07 mg, 230.39 μmol, 34.73 μL, 0.1 eq) and 6-methoxy-1H-indole-3-carbonyl cyanide (512.46 mg, 2.30 mmol, 90.0%, 1 eq). The mixture was stirred at 20° C. for 2 h. TLC (PE/EtOAc=1/1, R_{f}=0.12) showed that one new point was formed and the starting material was consumed completely. The mixture was concentrated to yield the yellow solid which was added MeOH (20 mL) and stirred at 20° C. for 0.5 h. The suspension was filtered and solid was collected, treated under vacuum to yield (4-tert-butyl-4,5-dihydrothiazol-2-yl)-(6-methoxy-1H-indol-3-yl)methanone (500 mg, 1.42 mmol, 61.7% yield, 90.0% purity) as a yellow solid. ¹H NMR (400 MHz, DMSO-d₆) δ ppm 11.94 (s, 1H), 8.52 (s, 1H), 8.06 (d, J=8.6 Hz, 1H), 7.06-7.02 (m, 1H), 6.89 (dd, J=2.2, 8.6 Hz, 1H), 4.52 (dd, J=9.4, 11.1 Hz, 1H), 3.80 (s, 3H), 3.29 (s, 1H), 3.21-3.02 (m, 1H), 1.05 (s, 9H).

### Step 7: (4-(tert-Butyl)thiazol-2-yl)(6-methoxy-1H-indol-3-yl)methanone

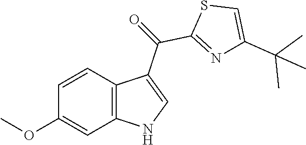 [SMILES: COc1ccc2c(C(=O)c3nc(C(C)(C)C)cs3)c[nH]c2c1]

To a solution of (4-tert-butyl-4,5-dihydrothiazol-2-yl)-(6-methoxy-1H-indol-3-yl)methanone (200 mg, 568.87 μmol, 90.0%, 1 eq) in 1,2-dichloroethane (10 mL) was added MnO₂ (494.56 mg, 5.69 mmol, 10 eq) under N₂ atmosphere. The mixture was stirred at 60° C. for 12 h. The reaction mixture was filtered and the filtrate was concentrate to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 59%-74%, 14 min), followed by lyophilization to yield (4-tert-butylthiazol-2-yl)-(6-methoxy-1H-indol-3-yl)methanone (15.91 mg, 49.53 μmol, 8.7% yield, 97.9% purity) as a yellow solid. ¹H NMR (400 MHz, DMSO-d₆) δ ppm 11.98 (s, 1H), 8.97 (s, 1H), 8.16 (d, J=9.0 Hz, 1H), 7.72 (s, 1H), 7.06 (d, J=2.0 Hz, 1H), 6.90 (dd, J=2.4, 8.6 Hz, 1H), 3.91-3.76 (m, 3H), 1.40 (s, 9H); ES-LCMS m/z 314.9 [M+H]⁺.

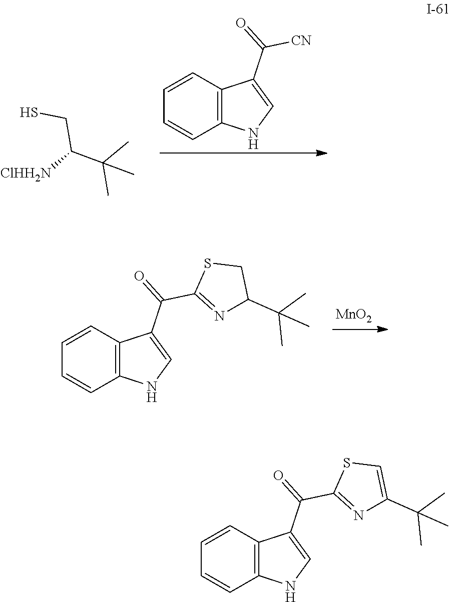

### Step 1: (4-(tert-Butyl)-4,5-dihydrothiazol-2-yl)(1H-indol-3-yl)methanone

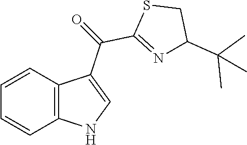 [SMILES: CC(C)(C)C1CSC(C(=O)c2c[nH]c3ccccc23)=N1]

To a solution of (2S)-2-amino-3,3-dimethyl-butane-1-thiol; hydrochloride (500 mg, 2.30 mmol, 95.0%, 1 eq, HCl) in pyridine (10 mL) was added DBU (35.07 mg, 230.39 μmol, 34.73 μL, 0.1 eq) and 1H-indole-3-carbonyl cyanide (412.67 mg, 2.30 mmol, 95.0%, 1 eq). The mixture was stirred at 60° C. for 12 h. The mixture was concentrated. The light yellow solid was added MeOH (15 mL) and stirred at 20° C. for 0.5 h. The suspension was filtered and solid was collected, treated under vacuum to yield (4-tert-butyl-4,5-dihydrothiazol-2-yl)-(1H-indol-3-yl)methanone (250 mg, 785.64 μmol, 34.1% yield, 90.0% purity) as a light yellow solid. ¹H NMR (400 MHz, DMSO-d₆) δ ppm 12.16 (s, 1H), 8.65 (s, 1H), 8.28-8.18 (m, 1H), 7.55 (dd, J=2.4, 6.1 Hz, 1H), 7.35-7.21 (m, 2H), 4.61-4.45 (m, 1H), 3.35 (s, 1H), 3.21-3.05 (m, 1H), 1.06 (s, 9H).

### Step 2: (4-(tert-Butyl)thiazol-2-yl)(1H-indol-3-yl)methanone

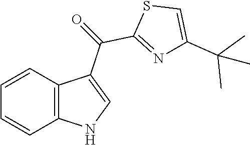 [SMILES: CC(C)(C)c1csc(C(=O)c2c[nH]c3ccccc23)n1]

To a solution of (4-tert-butyl-4,5-dihydrothiazol-2-yl)-(1H-indol-3-yl)methanone (200 mg, 628.51 μmol, 90.0%, 1 eq) in 1,2-dichloroethane (10 mL) was added MnO₂ (546.41 mg, 6.29 mmol, 10 eq) under N₂ atmosphere. The mixture was stirred at 60° C. for 12 h. The reaction mixture was filtered and the filtrate was concentrated to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 58%-88%, 10 min), followed by lyophilization to yield (4-tert-butylthiazol-2-yl)-(1H-indol-3-yl)methanone (49.15 mg, 169.72 μmol, 27.0% yield, 98.2% purity) as a light yellow solid. ¹H NMR (400 MHz, DMSO-d₆) δ ppm 12.19 (s, 1H), 9.09 (s, 1H), 8.40-8.18 (m, 1H), 7.76-7.68 (m, 1H), 7.64-7.48 (m, 1H), 7.32-7.23 (m, 2H), 1.41 (s, 9H); ES-LCMS m/z 284.9 [M+H]⁺.

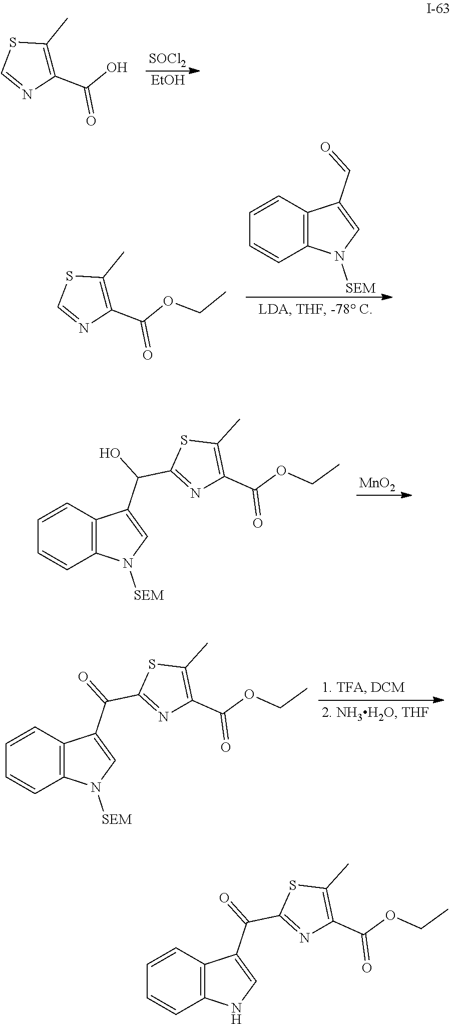

### Step 1: Ethyl 5-methylthiazole-4-carboxylate

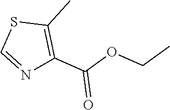

To a stirred solution of 5-methylthiazole-4-carboxylic acid (1 g, 6.99 mmol, 1 eq) in EtOH (20 mL) was added DMF (51.06 mg, 698.50 μmol, 53.74 μL, 0.1 eq) dropwise. Then SOCl₂ (4.16 g, 34.93 mmol, 2.54 mL, 5 eq) was added to the above mixture. The reaction mixture was stirred at 85° C. for 12 h. The reaction mixture was concentrated to yield a residue which was dissolved in NaHCO₃ solution (50 mL) then extracted with EtOAc (50 mL×3). The combined organic layers were dried over Na₂SO₄, filtered and the filtrate was concentrated to yield ethyl 5-methylthiazole-4-carboxylate (1 g, 5.46 mmol, 78.1% yield, 93.4% purity) as a yellow solid which was used in the next step without further purification. ¹H NMR (400 MHz, CDCl₃) δ ppm 8.58 (s, 1H), 4.43 (q, J=7.2 Hz, 2H), 2.81 (s, 3H), 1.43 (t, J=7.1 Hz, 3H); ES-LCMS m/z 172.2 [M+H]⁺.

### Step 2: Ethyl 2-[hydroxy-[1-(2-trimethylsilylethoxymethyl)indol-3-yl]methyl]-5-methyl-thiazole-4-carboxylate

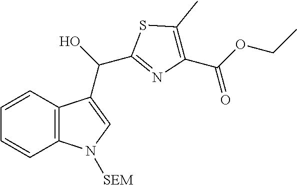 [SMILES: CCOC(=O)c1nc(C(O)c2cn(COCC[Si](C)(C)C)c3ccccc23)sc1C]

To a stirred solution of ethyl 5-methylthiazole-4-carboxylate (1 g, 5.46 mmol, 93.4%, 1 eq) and 1-(2-trimethylsilylethoxymethyl)indole-3-carbaldehyde (1.97 g, 6.57 mmol, 92%, 1.2 eq) in THF (50 mL) was cooled to −65° C. then added LDA (2 M, 8.18 mL, 3 eq) dropwise. The reaction mixture was stirred at −65° C. for 30 min. The reaction mixture was diluted with water (50 mL) then extracted with EtOAc (50 mL×3). The combined organic layers were dried over Na₂SO₄, filtered and the filtrate was concentrated to yield a residue which was purified by flash silica gel chromatography (from PE/EtOAc=100/1 to 1/100, TLC:PE/EtOAc=3/1, R_{f}=0.35) to yield ethyl 2-[hydroxy-[1-(2-trimethylsilylethoxymethyl)indol-3-yl]methyl]-5-methyl-thiazole-4-carboxylate (400 mg, 806.03 μmol, 14.8% yield, 90.0% purity) as yellow solid. ¹H NMR (400 MHz, CDCl₃) δ ppm 7.64 (d, J=7.8 Hz, 1H), 7.49 (d, J=8.3 Hz, 1H), 7.27-7.24 (m, 2H), 7.18-7.12 (m, 1H), 6.36 (s, 1H), 5.47 (s, 2H), 4.42 (q, J=7.1 Hz, 2H), 3.50-3.45 (m, 2H), 2.77-2.65 (m, 3H), 1.42 (t, J=7.2 Hz, 3H), 0.91-0.87 (m, 2H), −0.05 (s, 9H); ES-LCMS m/z 429.2 [M-OH]⁺.

### Step 3: Ethyl 5-methyl-2-[1-(2-trimethylsilylethoxymethyl)indole-3-carbonyl]thiazole-4-carboxylate

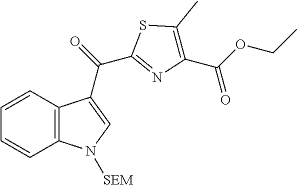 [SMILES: CCOC(=O)c1nc(C(=O)c2cn(COCC[Si](C)(C)C)c3ccccc23)sc1C]

To a stirred solution of ethyl 2-[hydroxy-[1-(2-trimethylsilylethoxymethyl)indol-3-yl]methyl]-5-methyl-thiazole-4-carboxylate (400 mg, 806.03 μmol, 90%, 1 eq) in CHCl₃ (20 mL) was added MnO₂ (2.80 g, 32.24 mmol, 40 eq). The reaction mixture was stirred at 80° C. for 12 h. The reaction mixture was filtered through a pad of celite. The filtered cake was washed with DCM (100 mL×3). The combined organic layers were dried over Na₂SO₄, filtered and the filtrate was concentrated to yield ethyl 5-methyl-2-[1-(2-trimethylsilylethoxymethyl)indole-3-carbonyl]thiazole-4-carboxylate (350 mg, 747.83 μmol, 92.8% yield, 95.0% purity) as a yellow solid which was used in the next step without further purification. ¹H NMR (400 MHz, CDCl₃) δ ppm 9.15 (s, 1H), 8.52 (d, J=5.6 Hz, 1H), 7.59 (d, J=5.6 Hz, 1H), 7.42-7.34 (m, 2H), 5.61 (s, 2H), 4.47 (q, J=6.9 Hz, 2H), 3.56 (t, J=7.8 Hz, 2H), 2.87 (s, 3H), 1.48 (t, J=7.0 Hz, 3H), 0.93 (t, J=7.8 Hz, 2H), −0.05 (s, 9H); ES-LCMS m/z 445.3 [M+H]⁺.

### Step 4: Ethyl 2-(1H-indole-3-carbonyl)-5-methyl-thiazole-4-carboxylate

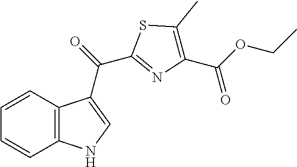 [SMILES: CCOC(=O)c1nc(C(=O)c2c[nH]c3ccccc23)sc1C]

To a stirred solution of ethyl 5-methyl-2-[1-(2-trimethylsilylethoxymethyl)indole-3-carbonyl]thiazole-4-carboxylate (50 mg, 106.83 μmol, 95%, 1 eq) in DCM (1 mL) was added TFA (1.54 g, 13.51 mmol, 1 mL, 126.43 eq). The reaction mixture was stirred at 25° C. for 1 h. TLC (PE/EtOAc=1/1, R_{f}=0.20) showed starting material was consumed completely and one new spot was detected. The reaction mixture was concentrated to yield a residue which was dissolved in THF (2 mL) then added NH₃.H₂O (910.00 mg, 7.01 mmol, 1 mL, 27% 65.62 eq). The mixture was stirred at 25° C. for 1 h. The reaction mixture was concentrated to yield a residue which was purified by preparative HPLC (column: Agela DuraShell C18 150*25 mm*5 um; mobile phase: [water (0.05% NH₃.H₂O+10 mM NH₄HCO₃)-ACN]; B %: 48%-78%, 10 min). The desired fraction was lyophilized to yield ethyl 2-(1H-indole-3-carbonyl)-5-methyl-thiazole-4-carboxylate (13.75 mg, 43.74 μmol, 40.9% yield, 100.0% purity) as white solid. ¹H NMR (400 MHz, DMSO-d₆) δ ppm 12.34 (s, 1H), 9.06 (s, 1H), 8.29 (dd, J=2.9, 5.6 Hz, 1H), 7.58 (dd, J=2.6, 6.0 Hz, 1H), 7.34-7.24 (m, 2H), 4.38 (q, J=7.1 Hz, 2H), 2.81 (s, 3H), 1.37 (t, J=7.1 Hz, 3H); ES-LCMS m/z 315.1 [M+H]⁺.

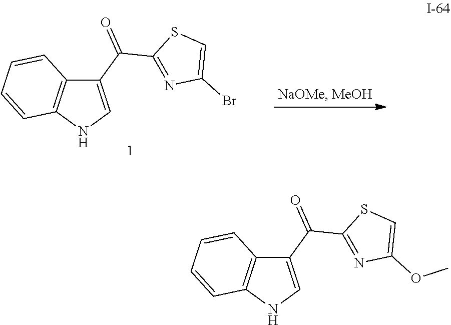 [SMILES: O=C(c1nc(Br)cs1)c1c[nH]c2ccccc12 ; COc1csc(C(=O)c2c[nH]c3ccccc23)n1]

### Step 1: 1H-Indol-3-yl-(4-methoxythiazol-2-yl)methanone

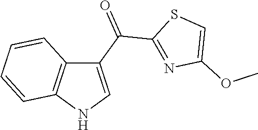 [SMILES: COc1csc(C(=O)c2c[nH]c3ccccc23)n1]

To a stirred solution of (4-bromothiazol-2-yl)-(1H-indol-3-yl)methanone (200 mg, 605.54 μmol, 93.0% purity, 1 eq) in MeOH (8 mL) was added NaOMe (327.13 mg, 6.06 mmol, 10 eq) and CuI (115.32 mg, 605.54 μmol, 1 eq). The reaction mixture was stirred under microwave (6 Bar) at 105° C. for 1 h. The reaction mixture was concentrated under reduced pressure to remove MeOH. The residue was diluted with H₂O (20 mL) and extracted with EtOAc (20 mL×3). The combined organic layers were washed with brine (20 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 37%-67%, 10 min) to yield 1H-indol-3-yl-(4-methoxythiazol-2-yl)methanone (45.37 mg, 173.80 μmol, 28.7% yield, 98.9% purity) as a yellow solid. ¹H NMR (500 MHz, DMSO-d₆) δ ppm 12.23 (s, 1H), 9.02 (s, 1H), 8.32-8.27 (m, 1H), 7.58-7.54 (m, 1H), 7.30-7.25 (m, 2H), 7.08 (s, 1H), 3.96 (s, 3H); ES-LCMS m/z 259.2 [M+H]⁺.

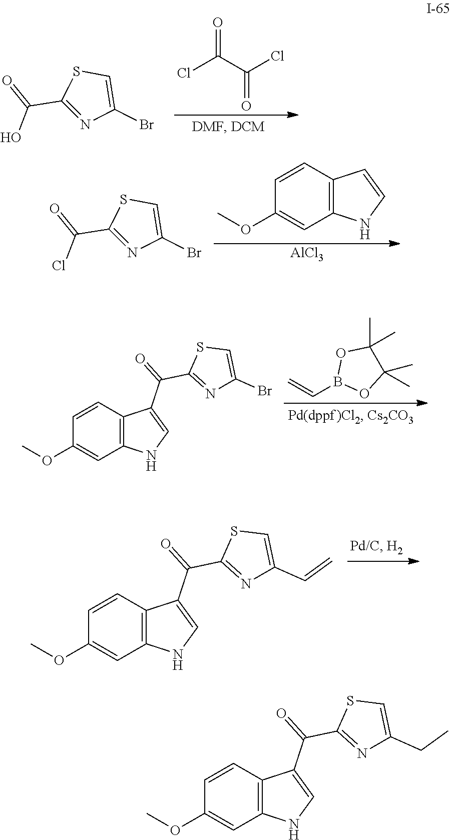

### Step 1: 4-Bromothiazole-2-carbonyl chloride

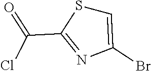

To a solution of 4-bromothiazole-2-carboxylic acid (200 mg, 961.39 μmol, 1 eq) in DCM (10 mL) was added DMF (35.14 mg, 480.69 μmol, 36.98 μL, 0.5 eq), oxalyl dichloride (244.05 mg, 1.92 mmol, 168.31 μL, 2.0 eq) at 0° C. The mixture was stirred under N₂ atmosphere at 20° C. for 2 h. TLC (PE/EtOAc=3/1, R_{f}=0.48) showed the reaction was completed. The reaction mixture was concentrated under reduced pressure to yield 4-bromothiazole-2-carbonyl chloride (200 mg, 883.08 μmol, 91.9% yield, crude) as a white solid, which was used in the next step without further purification.

### Step 2: (4-Bromothiazol-2-yl)(6-methoxy-1H-indol-3-yl)methanone

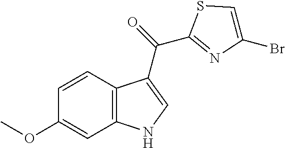 [SMILES: COc1ccc2c(C(=O)c3nc(Br)cs3)c[nH]c2c1]

To a solution of 6-methoxy-1H-indole (129.97 mg, 883.08 μmol, 1 eq) in DCM (5 mL) was added AlCl₃ (235.50 mg, 1.77 mmol, 2.0 eq) at 0° C. The mixture was stirred at 0° C. for 0.5 h. 4-Bromothiazole-2-carbonyl chloride (200 mg, 883.08 μmol, 1 eq) in DCM (3 mL) was added. The mixture was stirred at 25° C. for 15.5 h. The mixture was filtered and concentrated to yield a residue which was purified on silica gel column chromatography (from PE/EtOAc=10/1 to 3/1, TLC:PE/EtOAc=3/1, R_{f}=0.27) to yield (4-bromothiazol-2-yl)-(6-methoxy-1H-indol-3-yl)methanone (70 mg, 186.84 μmol, 21.2% yield, 90.0% purity) as a red solid. ¹H NMR (400 MHz, CDCl₃) δ ppm 9.04 (d, J=3.2 Hz, 1H), 8.65 (s, 1H), 8.38 (d, J=8.8 Hz, 1H), 7.55 (s, 1H), 7.01 (dd, J=2.2, 8.8 Hz, 1H), 6.96 (d, J=2.2 Hz, 1H), 3.89 (s, 3H); ES-LCMS m/z 337.1, 339.3 [M+H]⁺.

### Step 3: (6-Methoxy-1H-indol-3-yl)(4-vinylthiazol-2-yl)methanone

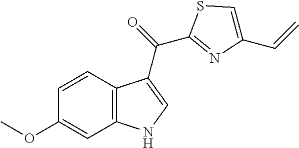 [SMILES: C=Cc1csc(C(=O)c2c[nH]c3cc(OC)ccc23)n1]

To a solution of (4-bromothiazol-2-yl)-(6-methoxy-1H-indol-3-yl)methanone (40 mg, 106.76 μmol, 90%, 1 eq) in 1,4-dioxane (10 mL), Water (2 mL) was added Pd(dppf)Cl₂ (3.91 mg, 5.34 μmol, 0.05 eq), 4,4,5,5-tetramethyl-2-vinyl-1,3,2-dioxaborolane (19.73 mg, 128.12 μmol, 21.73 μL, 1.2 eq), Cs₂CO₃ (69.57 mg, 213.53 μmol, 2.0 eq). The mixture was stirred under N₂ atmosphere at 80° C. for 2 h. TLC (PE/EtOAc=3/1, R_{f}=0.45) showed the reaction was completed. The mixture was added water (10 mL) and extracted with EtOAc (20 mL×3). The combined organic layers were washed with brine (20 mL), dried over Na₂SO₄, filtered and concentrated to yield a residue which was purified by silica gel column chromatography (from PE/EtOAc=10/1 to 5/1, TLC:PE/EtOAc=3/1, R_{f}=0.45) to yield (6-methoxy-1H-indol-3-yl)-(4-vinylthiazol-2-yl)methanone (30 mg, 94.96 mol, 88.9% yield, 90.0% purity) as a white solid. ¹H NMR (400 MHz, CDCl₃) δ ppm 9.11 (d, J=3.1 Hz, 1H), 8.63 (s, 1H), 8.42 (d, J=8.6 Hz, 1H), 7.42 (s, 1H), 7.01 (dd, J=2.2, 8.8 Hz, 1H), 6.96 (d, J=2.3 Hz, 1H), 6.83 (dd, J=11.0, 17.2 Hz, 1H), 6.16 (dd, J=1.4, 17.4 Hz, 1H), 5.47 (dd, J=1.6, 11.0 Hz, 1H), 3.89 (s, 3H); ES-LCMS m/z 285.2 [M+H]⁺.

### Step 4: (4-Ethylthiazol-2-yl)(6-methoxy-1H-indol-3-yl)methanone

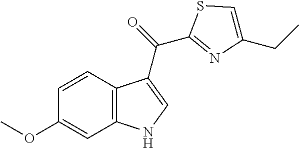 [SMILES: CCc1csc(C(=O)c2c[nH]c3cc(OC)ccc23)n1]

To a solution of (6-methoxy-1H-indol-3-yl)-(4-vinylthiazol-2-yl)methanone (30 mg, 94.96 mol, 90%, 1 eq) in EtOAc (10 mL) was added Pd/C (0.1 g, 10%, 1.00 eq). The mixture was stirred under H2 (15 Psi) atmosphere at 20° C. for 1 h. The reaction mixture was filtered and concentrated under reduced pressure to yield (4-ethylthiazol-2-yl)-(6-methoxy-1H-indol-3-yl)methanone (20.55 mg, 70.47 μmol, 74.2% yield, 98.2% purity) as a white solid. ¹H NMR (400 MHz, CDCl₃) δ ppm 9.07 (d, J=3.2 Hz, 1H), 8.59 (s, 1H), 8.42 (d, J=8.8 Hz, 1H), 7.21 (s, 1H), 7.00 (dd, J=2.2, 8.8 Hz, 1H), 6.95 (d, J=2.0 Hz, 1H), 3.89 (s, 3H), 2.93 (q, J=7.5 Hz, 2H), 1.40 (t, J=7.5 Hz 3141. ES-LCMS m/z 287.2 [M+H].

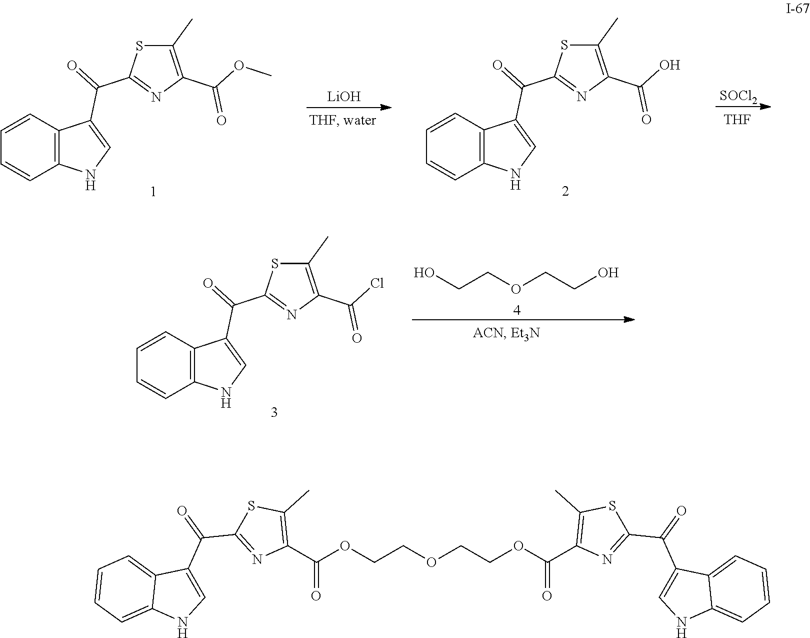

### Step 1: 2-(1H-Indole-3-carbonyl)-5-methyl-thiazole-4-carboxylic acid

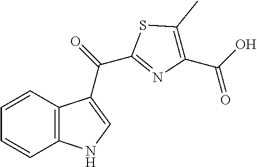 [SMILES: Cc1sc(C(=O)c2c[nH]c3ccccc23)nc1C(=O)O]

To a stirred solution of ethyl 2-(1H-indole-3-carbonyl)-5-methyl-thiazole-4-carboxylate (160 mg, 508.97 μmol, 1 eq) in THF (2 mL) and water (2 mL) was added LiOH (73.14 mg, 3.05 mmol, 6 eq). The reaction mixture was stirred at 60° C. for 12 h. The reaction mixture was concentrated to yield 2-(1H-indole-3-carbonyl)-5-methyl-thiazole-4-carboxylic acid (100 mg, crude) as a yellow solid which was used in the next step without further purification. ¹H NMR (400 MHz, DMSO-d₆) δ ppm 9.28 (s, 1H), 8.25 (d, J=5.1 Hz, 1H), 7.50 (d, J=7.1 Hz, 1H), 7.20 (d, J=3.4 Hz, 2H), 2.71 (s, 3H); ES-LCMS m/z 287.3 [M+H]⁺.

### Step 2: 2-(1H-Indole-3-carbonyl)-5-methyl-thiazole-4-carbonyl chloride

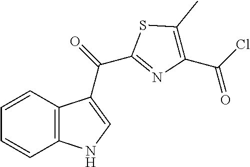 [SMILES: Cc1sc(C(=O)c2c[nH]c3ccccc23)nc1C(=O)Cl]

To a stirred solution of 2-(1H-indole-3-carbonyl)-5-methyl-thiazole-4-carboxylic acid (100 mg, 349.28 μmol, 1 eq) in THF (5 mL) was added SOCl₂ (415.54 mg, 3.49 mmol, 253.38 μL, 10 eq). The reaction mixture was stirred at 60° C. for 1 h. The reaction mixture was concentrated to yield 2-(1H-indole-3-carbonyl)-5-methyl-thiazole-4-carbonyl chloride (110 mg, crude, HCl) as a yellow solid, which was used in the next step without further purification. ES-LCMS m/z 305.1, 307.1 [M+H]⁺.

### Step 3: 2-[2-[2-(1H-Indole-3-carbonyl)-5-methyl-thiazole-4-carbonyl]oxyethoxy]ethyl 2-(1H-indole-3-carbonyl)-5-methyl-thiazole-4-carboxylate

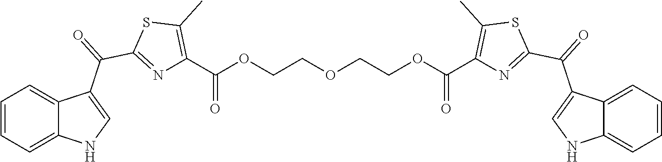 [SMILES: Cc1sc(C(=O)c2c[nH]c3ccccc23)nc1C(=O)OCCOCCOC(=O)c1nc(C(=O)c2c[nH]c3ccccc23)sc1C]

To a stirred solution of 2-(2-hydroxyethoxy)ethanol (8 mg, 75.39 μmol, 7.14 μL, 3.67e⁻μL, 1 eq) in THF (3 mL) was added Et₃N (62.28 mg, 615.45 μmol, 85.66 μL, 3 eq) then added 2-(1H-indole-3-carbonyl)-5-methyl-thiazole-4-carbonyl chloride (70 mg, 205.15 N/A purity, 1 eq, HCl) and Et₃N (62.28 mg, 615.45 μmol, 85.66 μL, 3 eq). The reaction mixture was stirred at 60° C. for 1 h under N₂ atmosphere. The reaction mixture was diluted with H₂O (20 mL) and extracted with EtOAc (50 mL×3). The combined organic layers dried over Na₂SO₄, filtered and concentrated under reduced pressure to yield a residue which was purified by preparative HPLC (column: Welch Xtimate C18 150×25 mm×5 μm; mobile phase: [water (10 mM NH₄HCO₃)-ACN]; B %: 51%-81%, 10 min). The desired fraction was lyophilized to yield 2-[2-[2-(1H-indole-3-carbonyl)-5-methyl-thiazole-4-carbonyl]oxyethoxy]ethyl-2-(1H-indole-3-carbonyl)-5-methyl-thiazole-4-carboxylate (10.67 mg, 54.29 μmol, 26.5% yield, 100.0%) as a yellow solid. ¹H NMR (400 MHz, CDCl₃) δ ppm 9.23 (d, J=3.4 Hz, 2H), 8.86 (s, 2H), 8.52 (d, J=6.8 Hz, 2H), 7.48 (d, J=8.6 Hz, 2H), 7.35 (s, 4H), 4.44 (s, 4H), 3.76-3.68 (m, 4H), 2.88 (s, 6H); ES-LCMS m/z 643.1 [M+H]⁺.

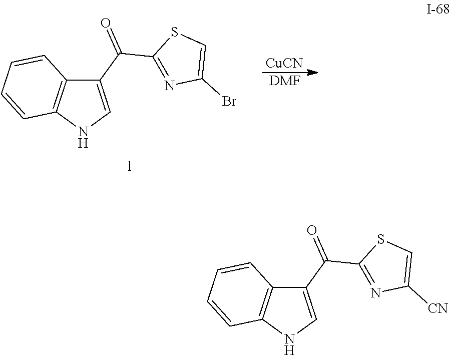

### Step 1: 2-(1H-Indole-3-carbonyl)thiazole-4-carbonitrile

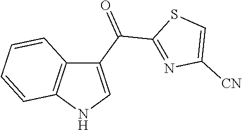 [SMILES: N#Cc1csc(C(=O)c2c[nH]c3ccccc23)n1]

To a solution of (4-bromothiazol-2-yl)-(1H-indol-3-yl)methanone (200 mg, 618.56 μmol, 95.0% purity, 1 eq) in DMF (2 mL) was added CuCN (276.99 mg, 3.09 mmol, 5 eq). The mixture was stirred at 150° C. for 2 h under microwave (0 Bar). The reaction mixture was diluted with H₂O (20 mL) and extracted with EtOAc (30 mL×3). The combined organic layers were washed with brine (20 mL), dried over Na₂SO₄, filtered and concentrated under reduced pressure to yield a residue which was purified by preparative HPLC (column: Phenomenex Synergi C18 150*30 mm*4 μm; mobile phase: [water (0.05% HCl)-ACN]; B %: 42%-62%, 10 min) to yield 2-(1H-indole-3-carbonyl)thiazole-4-carbonitrile (53.46 mg, 211.07 μmol, 34.1% yield, 100.0% purity) as a yellow solid. ¹H NMR (400 MHz, DMSO-d₆) δ ppm 12.42 (s, 1H), 9.18 (s, 1H), 9.03 (s, 1H), 8.39-8.22 (m, 1H), 7.63-7.56 (m, 1H), 7.34-7.26 (m, 2H); ES-LCMS m/z 254.2 [M+H]⁺.

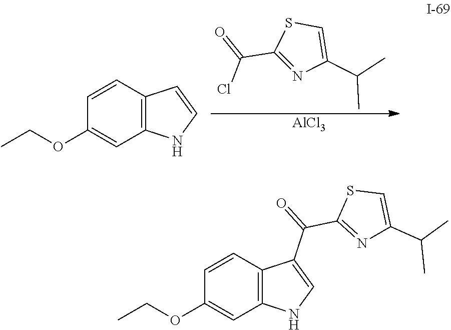

### Step 1: (6-Ethoxy-1H-indol-3-yl)-(4-isopropylthiazol-2-yl)methanone

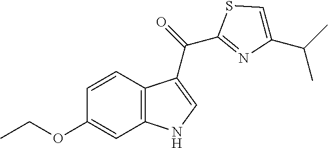 [SMILES: CCOc1ccc2c(C(=O)c3nc(C(C)C)cs3)c[nH]c2c1]

To a solution of 6-ethoxy-1H-indole (100 mg, 620.35 μmol, 1 eq) in DCM (2 mL) was added chloro(diethyl)alumane (89.74 mg, 744.42 μmol, 1.2 eq) under N₂ atmosphere at 0° C. The mixture was stirred at 0° C. for 30 min. 4-Isopropylthiazole-2-carbonyl chloride (141.19 mg, 744.42 μmol, 1.2 eq) in DCM (2 mL) was added and the mixture was stirred at 25° C. for 16 h. TLC (PE/EtOAc=3/1, R_{f}=0.4) showed a new spot was detected. The mixture was diluted with water (50 mL) and extracted with EtOAc (50 mL×3). The organic layer was washed with brine (50 mL), dried over Na₂SO₄, filtered and concentrated to yield a residue which was purified by preparative HPLC (column: Welch Xtimate C18 150*25 mm*5 um; mobile phase: [water (10 mM NH₄HCO₃)-ACN]; B %: 45%-75%, 10 min) to yield (6-ethoxy-1H-indol-3-yl)-(4-isopropylthiazol-2-yl)methanone (13.33 mg, 42.40 mol, 6.8% yield, 100.0% purity) as a yellow solid. ¹H NMR (400 MHz, CDCl₃) δ ppm 9.06 (d, J=3.1 Hz, 1H), 8.41 (d, J=8.6 Hz, 1H), 7.21 (d, J=0.8 Hz, 1H), 6.99 (dd, J=2.2, 8.8 Hz, 1H), 6.94 (d, J=2.0 Hz, 1H), 4.11 (q, J=7.0 Hz, 2H), 3.21 (t, J=6.8 Hz, 1H), 1.46 (t, J=6.8 Hz, 3H), 1.41 (d, J=6.7 Hz, 6H); ES-LCMS m/z 315.2 [M+H]⁺.

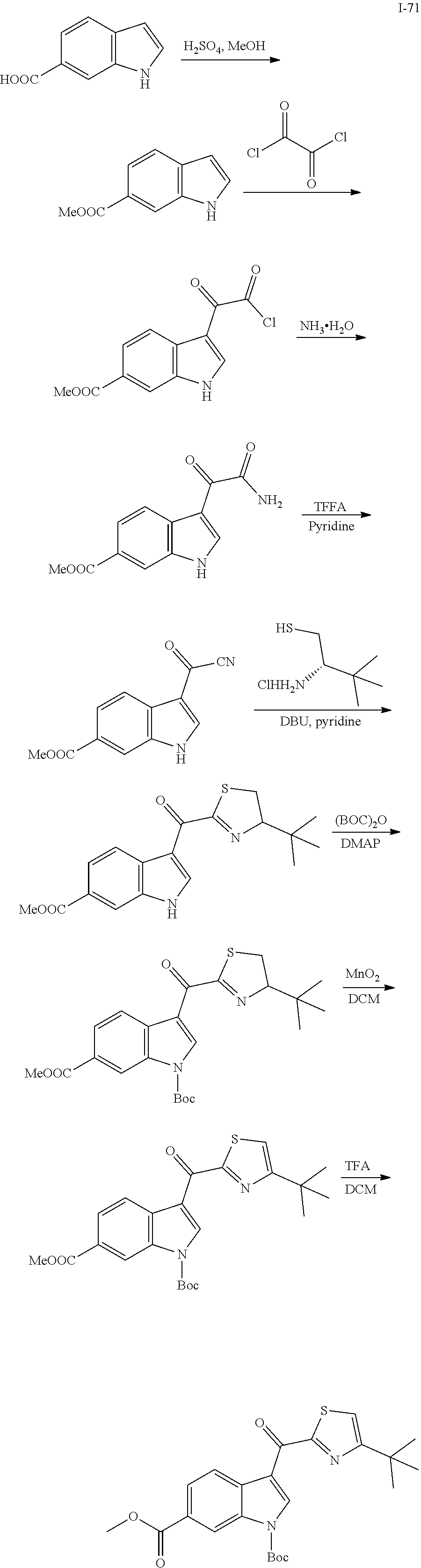

### Step 1: Methyl 1H-indole-6-carboxylate

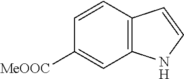 [SMILES: COC(=O)c1ccc2cc[nH]c2c1]

To a solution of 1H-indole-6-carboxylic acid (8.5 g, 52.74 mmol, 1 eq) in MeOH (150 mL) was added H2504 (7.76 g, 79.12 mmol, 4.22 mL, 1.5 eq). The mixture was stirred at 70° C. for 15 h. The reaction mixture was quenched with sat.aq NaHCO₃ (50 mL) and extracted with EtOAc (100 mL×3). The combined organic layers dried over Na₂SO₄, filtered and concentrated under reduced pressure to yield a residue which was purified by flash silica gel chromatography (from PE/EtOAc=100/1 to 3/1, TLC:PE/EtOAc=3/1, R_{f}=0.65) to yield methyl 1H-indole-6-carboxylate (7.4 g, 42.24 mmol, 80.0% yield, 100.0%) as a white solid which was purified by flash silica gel chromatography (from PE/EtOAc=100/1 to 3/1, TLC:PE/EtOAc=10/1, R_{f}=0.40). ¹H NMR (400 MHz, DMSO-d₆) δ ppm 11.52 (s, 1H), 8.12 (s, 1H), 7.69-7.56 (m, 3H), 6.54 (d, J=0.7 Hz, 1H), 3.85 (s, 3H); ES-LCMS m/z 176.3 [M+H]⁺.

### Step 2: Methyl 3-(2-chloro-2-oxo-acetyl)-1H-indole-6-carboxylate

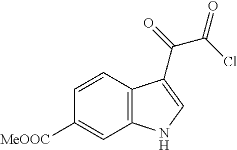 [SMILES: COC(=O)c1ccc2c(C(=O)C(=O)Cl)c[nH]c2c1]

To a solution of methyl 1H-indole-6-carboxylate (2.1 g, 11.99 mmol, 100%, 1 eq) in THF (20 mL) was added oxalyl dichloride (1.58 g, 12.47 mmol, 1.09 mL, 1.04 eq) at 0° C. The mixture was stirred at 0-5° C. for 3 h under N₂. TLC (PE/EtOAc=3/1, R_{f}=0.07) showed the starting material was consumed completely. The yellow slurry was filtered, and the cake was washed with PE (50 mL×2), dried under reduced pressure to yield methyl 3-(2-chloro-2-oxo-acetyl)-1H-indole-6-carboxylate (2.2 g, 8.28 mmol, 69.0% yield) as a yellow solid, which was used in the next step without further purification. ¹H NMR (400 MHz, DMSO-d₆) δ ppm 12.73 (s, 1H), 8.63 (d, J=3.5 Hz, 1H), 8.25 (d, J=8.6 Hz, 1H), 8.17 (d, J=0.8 Hz, 1H), 7.87 (dd, J=1.4, 8.4 Hz, 1H), 3.88 (s, 3H).

### Step 3: Methyl 3-oxamoyl-1H-indole-6-carboxylate

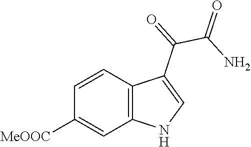 [SMILES: COC(=O)c1ccc2c(C(=O)C(N)=O)c[nH]c2c1]

To a solution of methyl 3-(2-chloro-2-oxo-acetyl)-1H-indole-6-carboxylate (2.2 g, 8.28 mmol, N/A, 1 eq) in THF (20 mL) was added NH₃.H₂O (11.61 g, 82.82 mmol, 12.76 mL, 25% in H₂O, 10 eq). The mixture was stirred at 0° C. for 1.5 h. The slurry was filtered, and the cake was washed with water (10 mL×2), dried under reduced pressure to yield methyl 3-oxamoyl-1H-indole-6-carboxylate (1.5 g, 5.48 mmol, 66.2% yield, 90.0% purity) as a white solid, which was used in the next step without further purification. ¹H NMR (400 MHz, DMSO-d₆) δ ppm 8.34 (s, 1H), 8.24 (d, J=8.1 Hz, 1H), 8.12 (s, 1H), 7.77 (d, J=8.1 Hz, 1H), 7.61-7.10 (m, 2H), 3.86 (s, 3H); ES-LCMS m/z 248.2 [M+H]⁺.

### Step 4: Methyl 3-carbonocyanidoyl-1H-indole-6-carboxylate

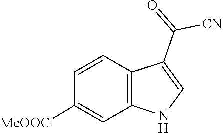 [SMILES: COC(=O)c1ccc2c(C(=O)C#N)c[nH]c2c1]

To a solution of methyl 3-oxamoyl-1H-indole-6-carboxylate (0.9 g, 3.58 mmol, 98%, 1 eq) in EtOAc (3 mL) was added TFAA (2.72 g, 12.94 mmol, 1.80 mL, 3.61 eq) and pyridine (1.76 g, 22.30 mmol, 1.80 mL, 6.23 eq) at 0° C. The mixture was stirred at 0-20° C. for 12 h under N₂. The mixture was stirred at 25° C. for 12 h. The reaction mixture was quenched by addition of sat. aq. NaHCO₃ (100 mL), extracted with EtOAc (80 mL×3). The combined organic layers were washed with 0.5 N aq. HCl (40 mL), brine (40 mL), dried over Na₂SO₄, filtered and the filtrate was concentrated under reduced pressure to yield methyl 3-carbonocyanidoyl-1H-indole-6-carboxylate (400 mg, 1.14 mmol, 31.8% yield, 65.0% purity) as a brown solid, which was used in the next step without further purification. ¹H NMR (400 MHz, DMSO-d₆) δ ppm 13.91-12.25 (m, 1H), 8.82 (s, 1H), 8.17 (s, 1H), 8.14-8.07 (m, 1H), 7.98-7.89 (m, 1H), 3.88 (s, 3H); ES-LCMS m/z 229.2 [M+H]⁺.

### Step 5: Methyl 3-(4-tert-butyl-4,5-dihydrothiazole-2-carbonyl)-1H-indole-6-carboxylate

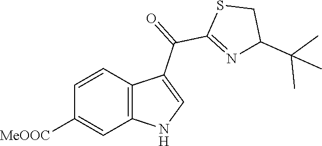 [SMILES: COC(=O)c1ccc2c(C(=O)C3=NC(C(C)(C)C)CS3)c[nH]c2c1]

To a solution of methyl 3-carbonocyanidoyl-1H-indole-6-carboxylate (350 mg, 996.92 μmol, 65%, 1 eq) in pyridine (6 mL) was added DBU (15.18 mg, 99.69 μmol, 15.03 μL, 0.1 eq) and (2S)-2-amino-3,3-dimethyl-butane-1-thiol; hydrochloride (170 mg, 1.00 mmol, 1.00 eq). The mixture was stirred at 20° C. for 2 h under N₂. The reaction mixture was filtered and concentrated under reduced pressure to yield methyl 3-(4-tert-butyl-4,5-dihydrothiazole-2-carbonyl)-1H-indole-6-carboxylate (340 mg, 463.96 μmol, 46.5% yield, 47.0% purity) as a yellow solid, which was used in the next step without further purification. ¹H NMR (400 MHz, CD₃OD) δ ppm 8.85 (s, 1H), 8.37 (d, J=8.6 Hz, 1H), 8.19 (s, 1H), 7.93 (dd, J=1.5, 8.3 Hz, 1H), 4.63-4.60 (m, 1H), 4.60 (s, 2H), 3.94 (s, 3H), 1.13 (s, 9H); ES-LCMS m/z 345.2 [M+H]⁺.

### Step 6: O1-tert-Butyl 06-methyl 3-(4-tert-butyl-4,5-dihydrothiazole-2-carbonyl)indole-1,6-dicarboxylate

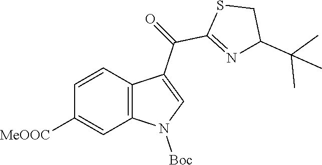

To a solution of methyl 3-(4-tert-butyl-4,5-dihydrothiazole-2-carbonyl)-1H-indole-6-carboxylate (340 mg, 463.96 μmol, 47%, 1 eq) in 1,4-dioxane (15 mL) was added (Boc)₂O (150 mg, 687.29 μmol, 157.89 μL, 1.48 eq) and DMAP (113.36 mg, 927.92 μmol, 2 eq). The mixture was stirred at 20° C. for 2 h. TLC (PE/EtOAc=3/1, R_{f}=0.67) showed the starting material was consumed completely. The reaction mixture was quenched with H₂O (30 mL) and extracted with EtOAc (50 mL×3). The combined organic layers dried over Na₂SO₄, filtered and concentrated under reduced pressure to yield a residue which was purified by flash silica gel chromatography (from PE/EtOAc=100/1 to 3/1, TLC:PE/EtOAc=3/1, R_{f}=0.76) to yield O1-tert-butyl 06-methyl 3-(4-tert-butyl-4,5-dihydrothiazole-2-carbonyl)indole-1,6-dicarboxylate (200 mg, 359.92 μmol, 77.5% yield, 80.0% purity) as a yellow solid. ¹H NMR (400 MHz, DMSO-d₆) δ ppm 8.80-8.73 (m, 2H), 8.33 (d, J=8.6 Hz, 1H), 8.00-7.96 (m, 1H), 3.81 (s, 3H), 3.62 (d, J=9.5 Hz, 1H), 3.00-2.81 (m, 2H), 1.62 (s, 9H), 0.85 (s, 9H); ES-LCMS m/z 445.3 [M+H]⁺.

### Step 7: O1-tert-Butyl 06-methyl 3-(4-tert-butylthiazole-2-carbonyl)indole-1,6-dicarboxylate

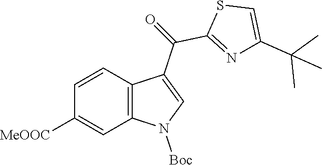 [SMILES: COC(=O)c1ccc2c(C(=O)c3nc(C(C)(C)C)cs3)cn(C(=O)OC(C)(C)C)c2c1]

To a solution of O1-tert-butyl 06-methyl 3-(4-tert-butyl-4,5-dihydrothiazole-2-carbonyl)indole-1,6-dicarboxylate (200 mg, 359.92 μmol, 80%, 1 eq) in 1,2-dichloroethane (3 mL) was added MnO₂ (600.00 mg, 6.90 mmol, 19.17 eq). The mixture was stirred at 80° C. for 4 h. The reaction mixture was filtered through a pad of celite and the filtrate was concentrated under reduce pressure to yield O1-tert-butyl 06-methyl 3-(4-tert-butylthiazole-2-carbonyl)indole-1,6-dicarboxylate (150 mg, 305.07 μmol, 84.7% yield, 90% purity) as a yellow solid. ¹H NMR (400 MHz, DMSO-d₆) δ ppm 9.23 (s, 1H), 8.83 (s, 1H), 8.37 (d, J=8.3 Hz, 1H), 8.24-8.17 (m, 1H), 8.03 (s, 1H), 3.89 (s, 3H), 1.09-1.06 (m, 9H), 0.83-0.79 (m, 9H); ES-LCMS m/z 443.2 [M+H]⁺.

### Step 8: Methyl 3-(4-tert-butylthiazole-2-carbonyl)-1H-indole-6-carboxylate

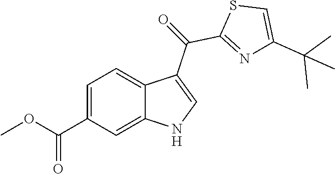 [SMILES: COC(=O)c1ccc2c(C(=O)c3nc(C(C)(C)C)cs3)c[nH]c2c1]

To a solution of O1-tert-butyl 06-methyl 3-(4-tert-butylthiazole-2-carbonyl)indole-1,6-dicarboxylate (40 mg, 81.35 μmol, 90%, 1 eq) in DCM (6 mL) was added TFA (2.46 g, 21.61 mmol, 1.60 mL, 265.64 eq). The mixture was stirred at 20° C. for 2 h. The reaction mixture was filtered and concentrated under reduced pressure to yield a residue which was purified by preparative HPLC (column: Welch Xtimate C18 150×25 mm×5 μm; mobile phase: [water (10 mM NH₄HCO₃)-ACN]; B %: 57%-87%, 10 min), followed by lyophilization to yield methyl 3-(4-tert-butylthiazole-2-carbonyl)-1H-indole-6-carboxylate (8.02 mg, 23.42 μmol, 28.7% yield, 100.0% purity) as a yellow solid. ¹H NMR (400 MHz, DMSO-d₆) δ ppm 9.25 (s, 1H), 8.39 (d, J=8.6 Hz, 1H), 8.22 (d, J=0.7 Hz, 1H), 7.88 (dd, J=1.5, 8.3 Hz, 1H), 7.77 (s, 1H), 3.88 (s, 3H), 1.41 (s, 9H); ES-LCMS m/z 343.2 [M+H]⁺.

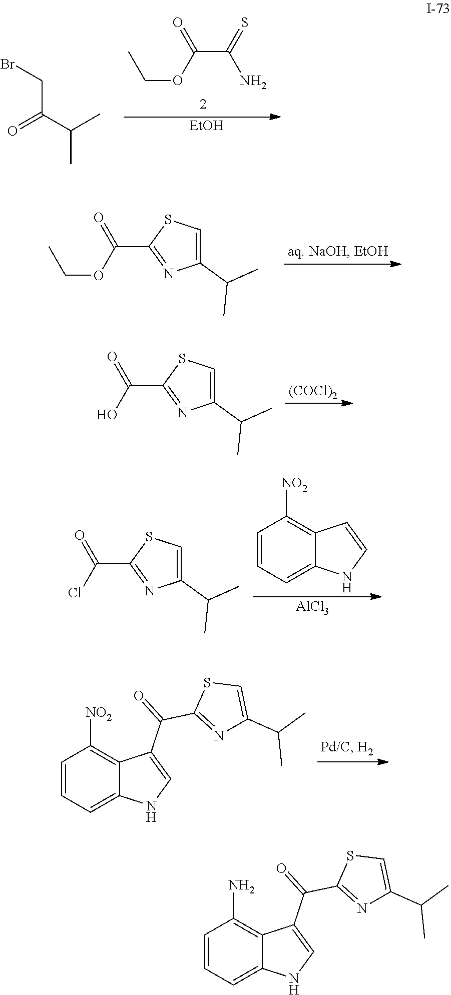

### Step 1: Ethyl 4-isopropylthiazole-2-carboxylate

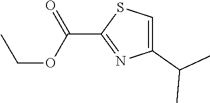

A mixture of ethyl 2-amino-2-thioxo-acetate (13 g, 97.62 mmol, 1 eq) and 1-bromo-3-methyl-butan-2-one (20.00 g, 121.19 mmol, 1.24 eq) in EtOH (200 mL) was stirred at 100° C. for 12 h. TLC (PE/EtOAc=5/1, R_{f}=0.54) showed a main spot formed. The reaction mixture was concentrated under reduced pressure to yield a residue which was purified by flash silica gel chromatography (from PE/EtOAc=100/1 to 20/1, TLC:PE/EtOAc=5/1, R_{f}=0.54) to yield ethyl 4-isopropylthiazole-2-carboxylate (15 g, 75.27 mmol, 77.1% yield, N/A purity) as yellow oil. ¹H NMR (400 MHz, CDCl₃) δ ppm 7.20 (s, 1H), 4.49 (q, J=7.0 Hz, 2H), 3.30-3.20 (m, 1H), 1.44 (t, J=7.2 Hz, 3H), 1.35 (d, J=7.0 Hz, 6H).

### Step 2: 4-Isopropylthiazole-2-carboxylic acid

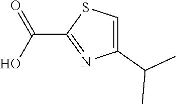

A mixture of ethyl 4-isopropylthiazole-2-carboxylate (3 g, 15.05 mmol, N/A purity, 1 eq) and LiOH.H₂O (695 mg, 16.56 mmol, 1.1 eq) in MeOH (10 mL) and THF (30 mL) was stirred at 25° C. for 12 h. TLC (PE/EtOAc=5/1, R_{f}=0.05) showed the starting material was consumed completely. The reaction mixture was acidified with aqueous HCl (1 M) until pH=2, diluted with EtOAc (500 mL), dried over Na₂SO₄, filtered and concentrated under reduced pressure to yield 4-isopropylthiazole-2-carboxylic acid (1.8 g, 10.51 mmol, 69.8% yield, N/A purity) as an yellow gum, which was used in the next step without further purification. ¹H NMR (400 MHz, DMSO-d₆) δ ppm 7.51 (s, 1H), 3.12-2.99 (m, 1H), 1.23 (d, J=7.0 Hz, 6H).

### Step 3: 4-Isopropylthiazole-2-carbonyl chloride

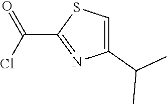

To a mixture of 4-isopropylthiazole-2-carboxylic acid (200 mg, 1.17 mmol, N/A purity, 1 eq) in DMF (0.01 mL) and DCM (3 mL) was added (COCl)₂ (725.00 mg, 5.71 mmol, 500 μL, 4.89 eq) dropwise at 25° C. The mixture was stirred at 25° C. for 1.5 h. TLC (PE/EtOAc=5/1, R_{f}=0.50) showed the starting material was consumed completely. The reaction mixture was concentrated under reduced pressure to yield 4-isopropylthiazole-2-carbonyl chloride (220 mg, 1.16 mmol, 99.30% yield, N/A purity) as a yellow solid, which was used in the next step without further purification.

### Step 4: (4-Isopropylthiazol-2-yl)-(4-nitro-1H-indol-3-yl)methanone

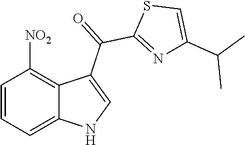 [SMILES: CC(C)c1csc(C(=O)c2c[nH]c3cccc([N+](=O)[O-])c23)n1]

To a solution of 4-isopropylthiazole-2-carbonyl chloride (220 mg, 1.16 mmol, N/A purity, 1 eq) in DCM (4 mL) was added AlCl₃ (850 mg, 6.37 mmol, 5.50 eq). The mixture was stirred under N₂ atmosphere at 25° C. for 0.25 h. 4-Nitro-1H-indole (200 mg, 1.23 mmol, 1.06 eq) was added. The mixture was stirred under N₂ atmosphere at 60° C. for 12 h. The reaction mixture was quenched with MeOH (20 mL) and concentrated under reduced pressure to yield a residue which was purified by flash silica gel chromatography (from PE/EtOAc=100/1 to 1/4, TLC:PE/EtOAc=0/1, R_{f}=0.10) to yield (4-isopropylthiazol-2-yl)-(4-nitro-1H-indol-3-yl)methanone (100 mg, 306.65 μmol, 26.4% yield, 96.7% purity) as a yellow solid. ¹H NMR (400 MHz, CDCl₃) δ ppm 9.10 (br s, 1H), 8.83 (d, J=2.7 Hz, 1H), 7.78 (d, J=7.8 Hz, 1H), 7.72 (d, J=8.1 Hz, 1H), 7.44-7.37 (m, 1H), 7.26 (s, 1H), 3.16 (td, J=7.0, 13.6 Hz, 1H), 1.35 (d, J=6.8 Hz, 6H); ES-LCMS m/z 316.2 [M+H]⁺.

### Step 5: (4-Amino-1H-indol-3-yl)-(4-isopropylthiazol-2-yl)methanone

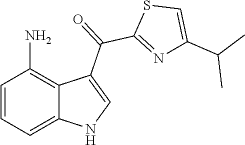 [SMILES: CC(C)c1csc(C(=O)c2c[nH]c3cccc(N)c23)n1]

A mixture of (4-isopropylthiazol-2-yl)-(4-nitro-1H-indol-3-yl)methanone (50 mg, 153.32 μmol, 96.7% purity, 1 eq) and Pd/C (50 mg, 10% purity) in MeOH (20 mL) was stirred under H2 (15 Psi) at 25° C. for 1 h. The reaction mixture was filtered. The filtrate was concentrated under reduced pressure to yield a residue which was purified by preparative TLC ((PE/EtOAc=3/1, R_{f}=0.34) to yield (4-amino-1H-indol-3-yl)-(4-isopropylthiazol-2-yl)methanone (13.01 mg, 43.68 μmol, 28.5% yield, 95.8% purity) as a yellow solid. ¹H NMR (400 MHz, CDCl₃) δ ppm 9.23 (d, J=3.5 Hz, 1H), 8.69 (s, 1H), 7.22 (s, 1H), 7.09 (t, J=7.8 Hz, 1H), 6.74 (dd, J=0.8, 7.8 Hz, 1H), 6.48 (d, J=7.4 Hz, 1H), 3.21 (td, J=7.3, 14.0 Hz, 1H), 1.40 (d, J=7.0 Hz, 6H); ES-LCMS m/z 286.3 [M+H]⁺.

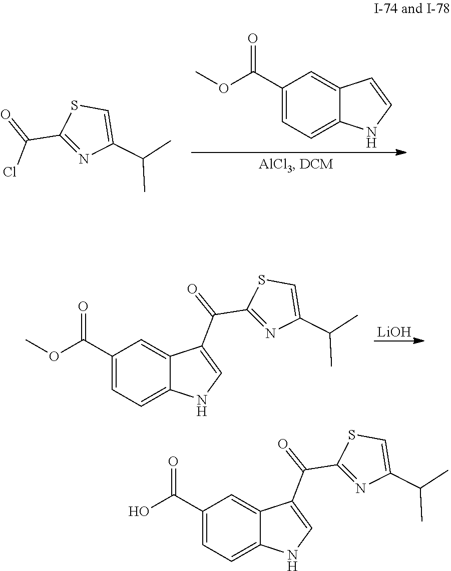

### Step 1: Methyl 3-(4-isopropylthiazole-2-carbonyl)-1H-indole-5-carboxylate

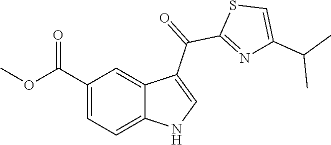 [SMILES: COC(=O)c1ccc2[nH]cc(C(=O)c3nc(C(C)C)cs3)c2c1]

To a solution of 4-isopropylthiazole-2-carbonyl chloride (300 mg, 1.58 mmol, 1 eq) in DCM (5 mL) was added AlCl₃ (1.16 g, 8.70 mmol, 5.5 eq) at 25° C. After being stirring for 0.25 h, methyl 1H-indole-5-carboxylate (415.65 mg, 2.37 mmol, 1.5 eq) was added. The mixture was stirred at 60° C. for 12 h. The mixture was quenched with MeOH (2 mL) and concentrated under reduced pressure to yield a residue which was purified by flash silica gel chromatography (from PE/EtOAc=100/1 to 1/1, TLC:PE/EtOAc=1/1, R_{f}=0.20) and preparative TLC (PE/EtOAc=1/1, R_{f}=0.20) to yield methyl 3-(4-isopropylthiazole-2-carbonyl)-1H-indole-5-carboxylate (80 mg, 230.46 μmol, 14.6% yield, 94.6% purity) as a yellow solid. ¹H NMR (400 MHz, DMSO-d₆) δ ppm 12.48 (s, 1H), 9.18 (d, J=2.7 Hz, 1H), 9.01 (s, 1H), 7.90 (dd, J=1.8, 8.4 Hz, 1H), 7.77 (d, J=0.8 Hz, 1H), 7.67 (d, J=8.6 Hz, 1H), 3.89 (s, 3H), 1.37 (d, J=7.0 Hz, 6H); ES-LCMS m/z 329.2 [M+H]⁺.

### Step 2: 3-(4-Isopropylthiazole-2-carbonyl)-1H-indole-5-carboxylic acid

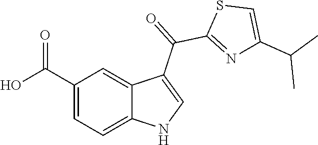 [SMILES: CC(C)c1csc(C(=O)c2c[nH]c3ccc(C(=O)O)cc23)n1]

To a solution of methyl 3-(4-isopropylthiazole-2-carbonyl)-1H-indole-5-carboxylate (50 mg, 144.04 μmol, 94.6%, 1 eq) in MeOH (1 mL) and H₂O (1 mL) was added LiOH.H₂O (30.22 mg, 720.19 μmol, 5 eq). The mixture was stirred at 25° C. for 12 h. The mixture was acidified by aqueous HCl (1 M) to adjust pH=3-4 and extracted with ethyl acetate (30 mL×3). The combined organic phase was washed with brine (20 mL), dried over anhydrous Na₂SO₄, filtered and concentrated in vacuum to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 8%-38%, 10 min), followed by lyophilization to yield 3-(4-isopropylthiazole-2-carbonyl)-1H-indole-5-carboxylic acid (17.63 mg, 56.08 μmol, 38.9% yield, 100% purity) as a yellow solid. ¹H NMR (500 MHz, DMSO-d₆) δ ppm 9.17 (s, 1H), 8.98 (d, J=1.1 Hz, 1H), 7.89 (dd, J=1.7, 8.5 Hz, 1H), 7.75 (s, 1H), 7.64 (d, J=8.5 Hz, 1H), 3.21 (dt, J=7.1, 13.8 Hz, 1H), 1.36 (d, J=6.9 Hz, 6H); ES-LCMS m/z 315.2 [M+H]⁺.

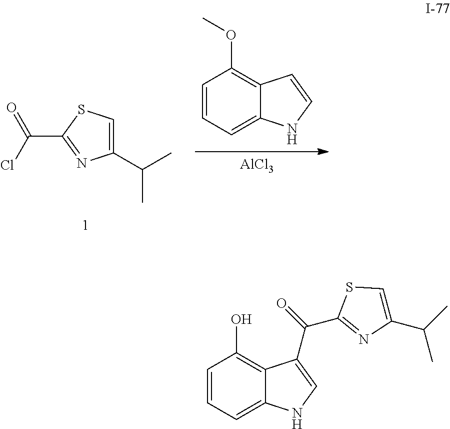

### Step 1: (4-Hydroxy-1H-indol-3-yl)-(4-isopropylthiazol-2-yl)methanone

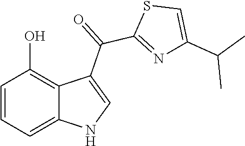 [SMILES: CC(C)c1csc(C(=O)c2c[nH]c3cccc(O)c23)n1]

To a solution of 4-isopropylthiazole-2-carbonyl chloride (220 mg, 1.16 mmol, N/A purity, 1 eq) in DCM (4 mL) was added AlCl₃ (850.68 mg, 6.38 mmol, 5.5 eq). The mixture was stirred under N₂ atmosphere at 25° C. for 0.5 h. 4-Methoxy-1H-indole (256.08 mg, 1.74 mmol, 1.5 eq) was added. The mixture was stirred under N₂ atmosphere at 60° C. for 11.5 h. The reaction mixture was concentrated under reduced pressure to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 52%-82%, 10 min) to yield (4-hydroxy-1H-indol-3-yl)-(4-isopropylthiazol-2-yl)methanone (7.05 mg, 23.35 μmol, 2.0% yield, 94.9% purity) as a red solid. ¹H NMR (400 MHz, DMSO-d₆) δ ppm 11.36 (s, 1H), 9.23 (s, 1H), 7.84 (s, 1H), 7.18-7.12 (m, 1H), 7.01 (d, J=7.3 Hz, 1H), 6.57 (d, J=7.3 Hz, 1H), 3.25-3.19 (m, 1H), 1.36 (d, J=6.8 Hz, 6H); ES-LCMS m/z 287.1 [M+H]⁺.

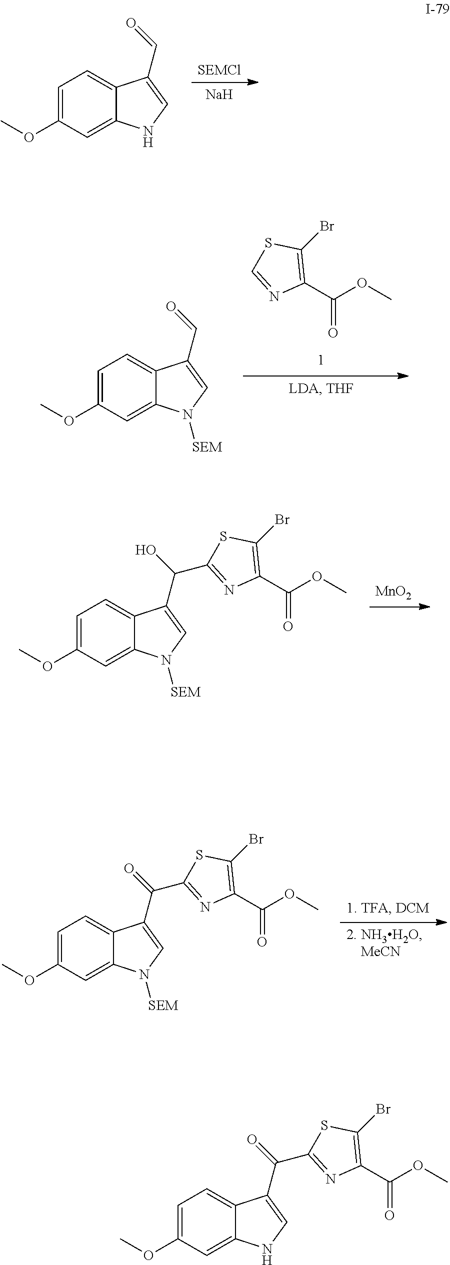

### Step 1: 6-Methoxy-1-(2-trimethylsilylethoxymethyl)indole-3-carbaldehyde

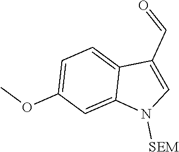 [SMILES: COc1ccc2c(C=O)cn(COCC[Si](C)(C)C)c2c1]

To a solution of 6-methoxy-1H-indole-3-carbaldehyde (500 mg, 2.85 mmol, 1 eq) in THF (8 mL) was added NaH (285.39 mg, 7.14 mmol, 60%, 2.5 eq) at 0° C. The reaction mixture was stirred at 0° C. for 30 min under N₂, and then SEM-Cl (951.69 mg, 5.71 mmol, 1.01 mL, 2 eq) was added to the above mixture dropwise. The resulting mixture was stirred at 0° C. for 2 h under N₂. The reaction mixture was quenched by addition of water (50 mL), extracted with EtOAc (30 mL×3). The combined organic layers were washed with brine (10 mL), dried over Na₂SO₄, filtered and concentrated under reduced pressure to give a residue which was purified by flash silica gel chromatography (from PE/EtOAc=1/0 to 1/1, TLC:PE/EtOAc=1/1, R_{f}=0.38) to yield 6-methoxy-1-(2-trimethylsilylethoxymethyl)indole-3-carbaldehyde (600 mg, 1.87 mmol, 65.3% yield, 95.0% purity) as yellow oil. ¹H NMR (400 MHz, CDCl₃) δ ppm 9.99 (s, 1H), 8.20-8.16 (m, 1H), 7.69 (s, 1H), 7.01-6.97 (m, 2H), 5.49 (s, 2H), 3.89 (s, 3H), 3.55-3.50 (m, 2H), 0.94-0.89 (m, 2H), −0.04 (s, 9H); ES-LCMS m/z 306.2 [M+H]⁺.

### Step 2: Methyl 5-bromo-2-[hydroxy-[6-methoxy-1-(2-trimethylsilylethoxymethyl)indol-3-yl]methyl]thiazole-4-carboxylate

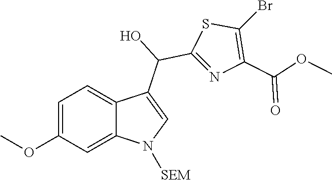

To a solution of methyl 5-bromothiazole-4-carboxylate (422.85 mg, 1.87 mmol, 98%, 1 eq), 6-methoxy-1-(2-trimethylsilylethoxymethyl)indole-3-carbaldehyde (600 mg, 1.87 mmol, 95%, 1 eq) in THF (10 mL) was added LDA (1 M, 3.73 mL, 2 eq) at −78° C. The mixture was stirred at −78° C. for 1 h. The reaction mixture was diluted with water (20 mL), extracted with EtOAc (50 mL×3). The combined organic layers were dried over Na₂SO₄, filtered and concentrated which was purified by flash silica gel chromatography (from PE/EtOAc=1/0 to 5/1, TLC:PE/EtOAc=5/1, R_{f}=0.25) to yield methyl 5-bromo-2-[hydroxy-[6-methoxy-1-(2-trimethylsilylethoxymethyl)indol-3-yl]methyl]thiazole-4-carboxylate (180 mg, 204.74 μmol, 10.9% yield, 60.0% purity) as a yellow oil. ¹H NMR (400 MHz, CDCl₃) δ ppm 7.49 (d, J=8.8 Hz, 1H), 7.14 (s, 1H), 6.96 (d, J=2.0 Hz, 1H), 6.83 (dd, J=2.1, 8.7 Hz, 1H), 6.30 (d, J=3.2 Hz, 1H), 5.41 (s, 2H), 3.95 (s, 3H), 3.87 (s, 3H), 3.51-3.47 (m, 2H), 0.93-0.88 (m, 2H), −0.04 (s, 9H); ES-LCMS m/z 527.1, 529.1 [M+H]⁺.

### Step 3: Methyl 5-bromo-2-[6-methoxy-1-(2-trimethylsilylethoxymethyl)indole-3-carbonyl]thiazole-4-carboxylate

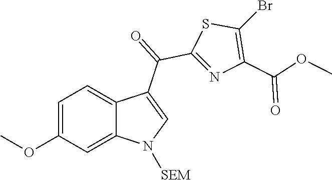

To a solution of methyl 5-bromo-2-[hydroxy-[6-methoxy-1-(2-trimethylsilylethoxymethyl)indol-3-yl]methyl]thiazole-4-carboxylate (170 mg, 193.36 μmol, 60%, 1 eq) in 1,2-dichloroethane (5 mL) was added MnO₂ (336.21 mg, 3.87 mmol, 20 eq). The mixture was stirred at 80° C. for 1 h. The mixture was filtered, and the filter cake was rinsed with PE (10 mL×2), dried to yield methyl 5-bromo-2-[6-methoxy-1-(2-trimethylsilylethoxymethyl)indole-3-carbonyl]thiazole-4-carboxylate (60 mg, 97.05 μmol, 50.1% yield, 85.0% purity) as a white solid, which was used in the next step without further purification. ¹H NMR (400 MHz, CDCl₃) δ ppm 8.94 (s, 1H), 8.35 (d, J=8.8 Hz, 1H), 7.06-7.00 (m, 2H), 5.57 (s, 2H), 4.02 (s, 3H), 3.90 (s, 3H), 3.57 (t, J=8.2 Hz, 2H), 3.50 (d, J=5.1 Hz, 2H), 0.98-0.90 (m, 2H), −0.04 (s, 9H); ES-LCMS m/z 525.1, 527.1 [M+H]⁺.

### Step 4: Methyl 5-bromo-2-(6-methoxy-1H-indole-3-carbonyl)thiazole-4-carboxylate

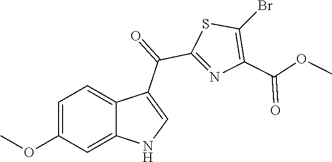

To a solution or methyl 5-bromo-2-[6-methoxy-1-(2-trimethylsilylethoxymethyl)indole-3-carbonyl]thiazole-4-carboxylate (60 mg, 97.05 μmol, 85%, 1 eq) in DCM (2 mL) was added TFA (616.00 mg, 5.40 mmol, 0.4 mL, 55.66 eq). The mixture was stirred at 20° C. for 2 h. The reaction mixture was concentrated at 25° C. to yield a residue. The mixture was concentrated to yield a residue which was dissolved in CH₃CN (2 mL). The mixture was adjusted pH to 9 by saturated NH₃.H₂O (273.00 mg, 2.18 mmol, 0.3 mL, 28%, 22.47 eq) solution then stirred at 25° C. for 1 h. The reaction mixture was concentrated to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 45%-75%, 10 min), followed by lyophilization to yield methyl 5-bromo-2-(6-methoxy-1H-indole-3-carbonyl)thiazole-4-carboxylate (8.16 mg, 20.65 μmol, 21.2% yield, 100.0% purity) as a white solid. ¹H NMR (400 MHz, CDCl₃) δ ppm 9.06 (d, J=3.2 Hz, 1H), 8.69 (s, 1H), 8.35 (d, J=8.6 Hz, 1H), 7.01 (dd, J=2.1, 8.7 Hz, 1H), 6.96 (d, J=2.0 Hz, 1H), 4.02 (s, 3H), 3.89 (s, 3H); ES-LCMS m/z 394.7, 396.7 [M+H]⁺.

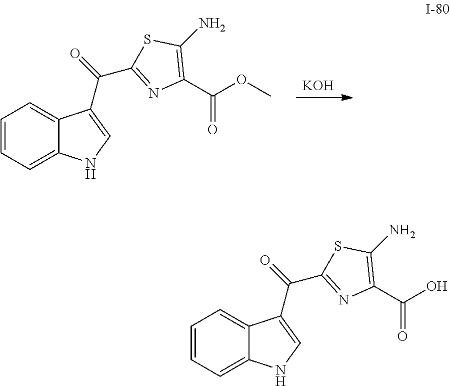

### Step 1: 5-Amino-2-(1H-indole-3-carbonyl)thiazole-4-carboxylic acid

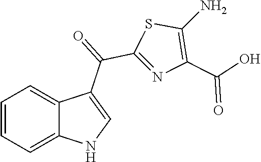 [SMILES: Nc1sc(C(=O)c2c[nH]c3ccccc23)nc1C(=O)O]

To a stirred solution of methyl 5-amino-2-(1H-indole-3-carbonyl)thiazole-4-carboxylate (30 mg, 96.58 μmol, 97% purity, 1 eq) in THF (0.5 mL), MeOH (0.5 mL), H₂O (0.5 mL) was added KOH (16.26 mg, 289.73 μmol, 3 eq). The mixture was stirred at 25° C. for 16 h. The mixture was concentrated to yield a residue which was purified by preparative HPLC: ([water (10 mM NH₄HCO₃)-ACN]; B %: 0%-40%, 10 min), followed by lyophilization to yield 5-amino-2-(1H-indole-3-carbonyl)thiazole-4-carboxylic acid (23.0 mg, 77.49 μmol, 80.2% yield, 96.8% purity) as a yellow solid. ¹H NMR (400 MHz, DMSO-d₆) δ ppm 9.05 (s, 1H), 8.35-8.21 (m, 1H), 7.96 (s, 2H), 7.56-7.47 (m, 1H), 7.27-7.15 (m, 2H); ES-LCMS m/z 288.2 [M+H]⁺.

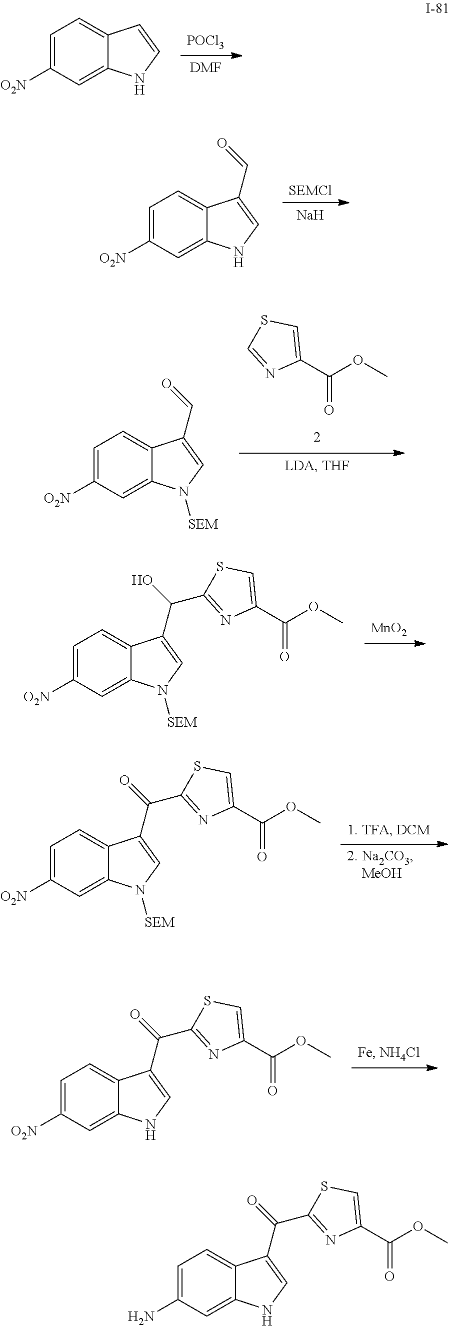

### Step 1: 6-Nitro-1H-indole-3-carbaldehyde

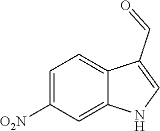 [SMILES: O=Cc1c[nH]c2cc([N+](=O)[O-])ccc12]

POCl₃ (16.42 g, 107.12 mmol, 9.95 mL, 2.32 eq) was slowly added dropwise to DMF (50 mL). The mixture was stirred at 0° C. for 30 min. Then, DMF (12.43 g, 170.06 mmol, 13.08 mL, 3.68 eq) solution of 6-nitro-1H-indole (7.5 g, 46.25 mmol, 1 eq) was added dropwise to the reaction system. The mixture was stirred at 25° C. for 2 h. The reaction mixture was quenched by addition of ice water (50 mL) and 10% aq. NaOH, adjusted the reaction system pH to 7-8 and continue to stir to a lot of white solid precipitation, filtered to give a residue which was added PE/EA (5/1, 500 mL), and stirred at 15° C. for 2 h. The slurry was filtered, and the cake was rinsed with PE (2×30 mL). The solid was collected and dried in vacuo to yield 6-nitro-1H-indole-3-carbaldehyde (7 g, 35.14 mmol, 76.0% yield, 95.5% purity) as a yellow solid. ES-LCMS m/z 191.2 [M+H]⁺.

### Step 2: 6-Nitro-1-(2-trimethylsilylethoxymethyl)indole-3-carbaldehyde

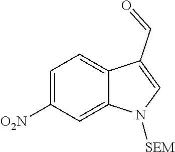 [SMILES: C[Si](C)(C)CCOCn1cc(C=O)c2ccc([N+](=O)[O-])cc21]

To a stirred solution of 6-nitro-1H-indole-3-carbaldehyde (2.7 g, 13.32 mmol, 93.8%, 1 eq) in THF (50 mL) was cooled to 0° C. then NaH (1.07 g, 26.63 mmol, 60%, 2 eq) partwise under N₂ atmosphere. The reaction mixture was stirred at 0° C. for 30 min under N₂ atmosphere. SEM-Cl (3.33 g, 19.98 mmol, 3.54 mL, 1.5 eq) was added to the above mixture dropwise then stirred at 0° C. for 2 h under N₂ atmosphere. The reaction mixture was quenched by addition of water (50 mL), extracted with EtOAc (30 mL×3). The combined organic layers were washed with brine (10 mL), dried over Na₂SO₄, filtered and concentrated under reduced pressure to give a residue which was added PE/EA (5/1, 500 mL), and stirred at 15° C. for 2 h. The slurry was filtered, and the cake was rinsed with PE (2×30 mL). The solid was collected and dried in vacuo to yield 6-nitro-1-(2-trimethylsilylethoxymethyl)indole-3-carbaldehyde (4.2 g, 11.80 mmol, 88.5% yield, 90.0% purity) as a yellow solid. ¹H NMR (400 MHz, CDCl₃) δ ppm 10.11 (s, 1H), 8.51 (d, J=2.0 Hz, 1H), 8.43 (d, J=8.6 Hz, 1H), 8.25 (dd, J=2.0, 8.6 Hz, 1H), 8.06 (s, 1H), 5.63 (s, 2H), 3.60-3.55 (m, 2H), 0.97-0.93 (m, 2H), 0.00 (s, 9H); ES-LCMS m/z 321.2 [M+H]⁺.

### Step 3: Methyl 2-[hydroxy-[6-nitro-1-(2-trimethylsilylethoxymethyl)indol-3-yl]methyl]thiazole-4-carboxylate

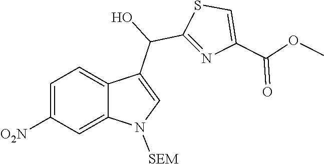 [SMILES: COC(=O)c1csc(C(O)c2cn(COCC[Si](C)(C)C)c3cc([N+](=O)[O-])ccc23)n1]

To a solution of 6-nitro-1-(2-trimethylsilylethoxymethyl)indole-3-carbaldehyde (4.2 g, 11.80 mmol, 90%, 1 eq) and methyl thiazole-4-carboxylate (1.69 g, 11.80 mmol, 1 eq) in THF (10 mL) was added LDA (1 M, 23.59 mL, 2 eq) at −75° C. The mixture was stirred at 25° C. for 2 h. The reaction mixture was quenched by addition of water (50 mL), extracted with EtOAc (50 mL×3). The combined organic layers were washed with brine (50 mL), dried over Na₂SO₄, filtered and concentrated under reduced pressure to give a residue which was purified by flash silica gel chromatography (from PE/EtOAc=1/0 to 1/1, TLC:PE/EtOAc=1/1, R_{f}=0.49) to yield methyl 2-[hydroxy-[6-nitro-1-(2-trimethylsilylethoxymethyl)indol-3-yl]methyl]thiazole-4-carboxylate (2.34 g, 1.51 mmol, 12.8% yield, 30.0% purity) as a yellow oil. ES-LCMS m/z 464.2 [M+H]⁺.

### Step 4: Methyl 2-[6-nitro-1-(2-trimethylsilylethoxymethyl)indole-3-carbonyl]thiazole-4-carboxylate

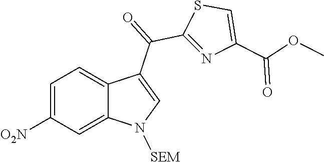 [SMILES: COC(=O)c1csc(C(=O)c2cn(COCC[Si](C)(C)C)c3cc([N+](=O)[O-])ccc23)n1]

To a solution of methyl 2-[hydroxy-[6-nitro-1-(2-trimethylsilylethoxymethyl)indol-3-yl]methyl]thiazole-4-carboxylate (2.33 g, 1.51 mmol, 30%, 1 eq) in BrCH₂CH₂Br (15 mL) was added MnO₂ (1.31 g, 15.08 mmol, 10 eq). The mixture was stirred at 80° C. for 4 h. The reaction mixture was filtered and concentrated under reduced pressure to give a residue which was purified by flash silica gel chromatography (from PE/EtOAc=1/0 to 2/1, TLC:PE/EtOAc=1/1, R_{f}=0.43) to yield methyl 2-[6-nitro-1-(2-trimethylsilylethoxymethyl)indole-3-carbonyl]thiazole-4-carboxylate (800 mg, 1.20 mmol, 79.3% yield, 69.0% purity) as a yellow oil. ES-LCMS m/z 462.2 [M+H]⁺.

### Step 5: Methyl 2-(6-nitro-1H-indole-3-carbonyl)thiazole-4-carboxylate

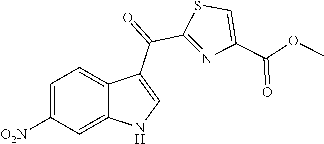 [SMILES: COC(=O)c1csc(C(=O)c2c[nH]c3cc([N+](=O)[O-])ccc23)n1]

To a solution of methyl 2-[6-nitro-1-(2-trimethylsilylethoxymethyl)indole-3-carbonyl]thiazole-4-carboxylate (500 mg, 747.03 μmol, 68.9%, 1 eq) in DCM (5 mL) was added TFA (26.55 g, 232.84 mmol, 17.24 mL, 311.69 eq). The mixture was stirred at 25° C. for 12 h. The reaction mixture was quenched by addition of water (50 mL), extracted with EtOAc (30 mL×3). The combined organic layers were washed with brine (10 mL), dried over Na₂SO₄, filtered and concentrated under reduced pressure to give methyl 2-(6-nitro-1H-indole-3-carbonyl)thiazole-4-carboxylate (450 mg, crude) as a yellow solid. ¹H NMR (400 MHz, DMSO-d₆) δ ppm 12.82 (br s, 1H), 9.35 (s, 1H), 8.94 (s, 1H), 8.53 (d, J=1.7 Hz, 1H), 8.47 (d, J=8.8 Hz, 1H), 8.18 (dd, J=2.0, 8.8 Hz, 1H), 3.93 (s, 3H); ES-LCMS m/z 332.0 [M+H]⁺.

### Step 6: Methyl 2-(6-amino-1H-indole-3-carbonyl)thiazole-4-carboxylate

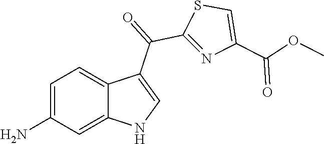 [SMILES: COC(=O)c1csc(C(=O)c2c[nH]c3cc(N)ccc23)n1]

To a solution of methyl 2-(6-nitro-1H-indole-3-carbonyl)thiazole-4-carboxylate (250 mg, 754.60 μmol, 1 eq) in EtOH (5 mL) and H₂O (5 mL) was added Fe (210.70 mg, 3.77 mmol, 5 eq) and NH₄Cl (403.64 mg, 7.55 mmol, 10 eq). The mixture was stirred at 80° C. for 2 h. The reaction mixture was quenched by addition of water (50 mL), extracted with EtOAc (30 mL×3). The combined organic layers were washed with brine (30 mL), dried over Na₂SO₄, filtered and concentrated under reduced pressure to give a residue which was purified by preparative HPLC (column: Agela DuraShell C₁₈ 150*25 mm*5 μm; mobile phase: [water (0.05% NH₃.H₂O+10 mM NH₄HCO₃)-ACN]; B %: 20%-50%, 10 min), followed by lyophilization to yield methyl 2-(6-amino-1H-indole-3-carbonyl)thiazole-4-carboxylate (23.13 mg, 76.76 μmol, 10.2% yield, 100.0% purity) as a yellow solid. ¹H NMR (400 MHz, DMSO-d₆) δ ppm 11.83 (br s, 1H), 8.84 (s, 1H), 8.80 (d, J=2.9 Hz, 1H), 7.91 (d, J=8.6 Hz, 1H), 6.69 (d, J=1.5 Hz, 1H), 6.61 (dd, J=1.8, 8.4 Hz, 1H), 5.09 (s, 2H), 3.91 (s, 3H); ES-LCMS m/z 302.1 [M+H]⁺.

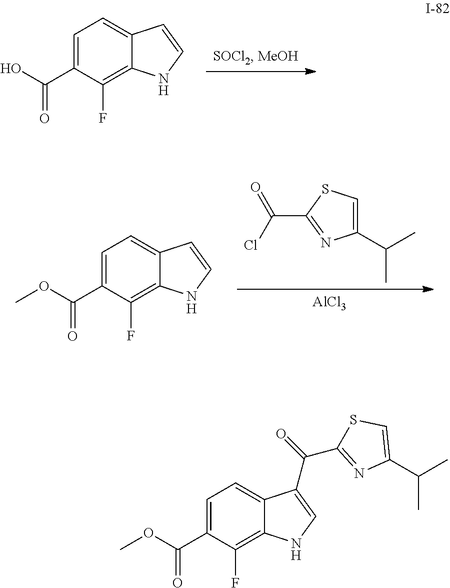

### Step 1: Methyl 7-fluoro-1H-indole-6-carboxylate

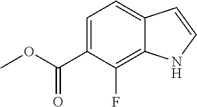

To a solution of 7-fluoro-1H-indole-6-carboxylic acid (250 mg, 1.40 mmol, 1 eq) in DMF (0.01 mL) and MeOH (5 mL) was added SOCl₂ (410.00 mg, 3.45 mmol, 0.25 mL, 2.47 eq) dropwise at 25° C. The mixture was stirred at 25° C. for 12 h. TLC (PE/EtOAc=2/1, R_{f}=0.50) showed the starting material was consumed completely. The reaction mixture was concentrated under reduced pressure to yield a residue which was purified by flash silica gel chromatography (from PE/EtOAc=100/1 to 5/1, TLC:PE/EtOAc=2/1, R_{f}=0.50) to yield methyl 7-fluoro-1H-indole-6-carboxylate (250 mg, 1.25 mmol, 89.5% yield, 96.5% purity) as a white solid. ¹H NMR (400 MHz, CDCl₃) δ ppm 8.63 (br s, 1H), 7.66 (dd, J=6.4, 8.3 Hz, 1H), 7.41 (d, J=8.6 Hz, 1H), 7.39 (t, J=2.7 Hz, 1H), 6.64-6.60 (m, 1H), 3.96 (s, 3H); ES-LCMS m/z 194.3 [M+H]⁺.

### Step 2: Methyl 7-fluoro-3-(4-isopropylthiazole-2-carbonyl)-1H-indole-6-carboxylate

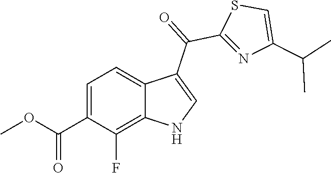 [SMILES: COC(=O)c1ccc2c(C(=O)c3nc(C(C)C)cs3)c[nH]c2c1F]

To a mixture of 4-isopropylthiazole-2-carbonyl chloride (240 mg, 1.27 mmol, N/A purity, 1 eq) in DCM (5 mL) was added AlCl₃ (1 g, 7.50 mmol, 5.93 eq) at 25° C. The mixture was stirred at 25° C. for 0.25 h. Methyl 7-fluoro-1H-indole-6-carboxylate (200 mg, 897.64 μmol, 86.7% purity, 7.09e-1 eq) was added. The mixture was stirred at 60° C. for 12 h. The reaction mixture was quenched with MeOH (30 mL) and concentrated under reduced pressure to yield a residue which was purified by flash silica gel chromatography (from PE/EtOAc=100/1 to 2/1, TLC:PE/EtOAc=2/1, R_{f}=0.40) to yield methyl 7-fluoro-3-(4-isopropylthiazole-2-carbonyl)-1H-indole-6-carboxylate (23.01 mg, 66.43 μmol, 5.3% yield, 100.0% purity) as an off-white solid. ¹H NMR (500 MHz, CDCl₃) δ ppm 9.31 (d, J=3.2 Hz, 1H), 9.12 (s, 1H), 8.32 (d, J=8.5 Hz, 1H), 7.88 (dd, J=6.6, 8.3 Hz, 1H), 3.98 (s, 3H), 3.22 (td, J=7.0, 13.7 Hz, 1H), 1.41 (d, J=7.0 Hz, 6H); ES-LCMS m/z 347.1 [M+H]⁺.

### I-84

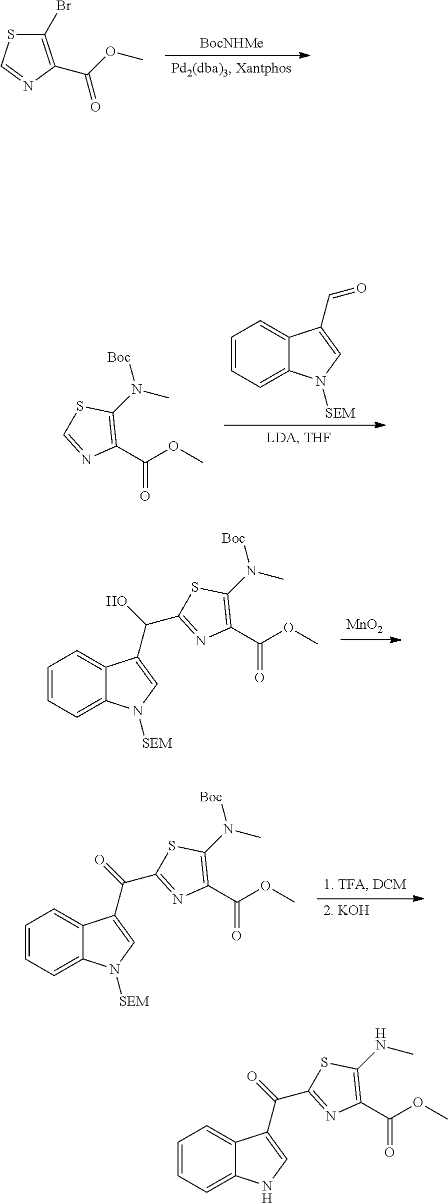

### Step 1: Methyl 5-((tert-butoxycarbonyl)(methyl)amino)thiazole-4-carboxylate

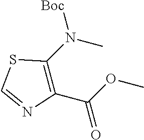 [SMILES: COC(=O)c1ncsc1N(C)C(=O)OC(C)(C)C]

To a solution of methyl 5-bromothiazole-4-carboxylate (1 g, 4.41 mmol, 98.0%, 1 eq) in toluene (15 mL) was added tert-butyl N-methylcarbamate (752.01 mg, 5.73 mmol, 1.3 eq), (5-diphenylphosphanyl-9,9-dimethyl-xanthen-4-yl)-diphenyl-phosphane (459.64 mg, 793.80 μmol, 0.18 eq), Cs₂CO₃ (3.16 g, 9.70 mmol, 2.2 eq) and Pd₂(dba)₃ (242.48 mg, 264.60 μmol, 0.06 eq). The mixture was stirred under N₂ atmosphere at 80° C. for 12 h. The mixture were filtered. The filtrate was diluted with water (100 mL) and extracted with EtOAc (200 mL×3). The combined organic layers were washed with brine (100 mL×3), dried over Na₂SO₄, filtered and concentrated to yield a residue which was purified by flash silica gel chromatography (from pure PE to PE/EtOAc=200/1 to 5/1, R_{f}=0.10) to yield 5-[tert-butoxy carbonyl(methyl)amino]thiazole-4-carboxylate (1 g, 3.16 mmol, 71.6% yield, 86.0% purity) as yellow oil. ¹H NMR (400 MHz, CDCl₃) δ ppm 8.62 (s, 1H), 3.92 (s, 3H), 3.23 (s, 3H), 1.38 (s, 9H); ES-LCMS m/z 273.3 [M+H]⁺.

### Step 2: Methyl 5-((tert-butoxycarbonyl)(methyl)amino)-2-(hydroxy(1-((2-(trimethylsilyl)ethoxy)methyl)-1H-indol-3-yl)methyl)thiazole-4-carboxylate

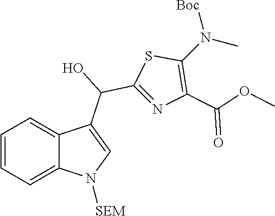 [SMILES: COC(=O)c1nc(C(O)c2cn(COCC[Si](C)(C)C)c3ccccc23)sc1N(C)C(=O)OC(C)(C)C]

To a solution of methyl 5-[tert-butoxycarbonyl(methyl)amino]thiazole-4-carboxylate (1 g, 3.16 mmol, 86.0%, 1 eq) and 1-(2-trimethylsilylethoxymethyl)indole-3-carbaldehyde (1.07 g, 3.79 mmol, 98.0%, 1.2 eq) in THF (15 mL) was added LDA (2 M, 3.16 mL, 2 eq) slowly at −78° C. The mixture was stirred under N₂ atmosphere at −78° C. for 20 min. TLC (PE/EtOAc=3/1, R_{f}=0.10) showed the starting material was consumed completely and one new spot was detected. The reaction mixture was quenched by addition saturated NH₄Cl (50 mL) at 0° C., diluted with water (50 mL) and extracted with EtOAc (200 mL×3). The combined organic layers were washed with brine (100 mL×3), dried over Na₂SO₄, filtered and concentrated under reduced pressure to yield a residue which was purified by silica gel column chromatography (from pure PE to PE/EtOAc=3/1, TLC:PE/EtOAc=3/1, R_{f}=0.10) to yield methyl 5-[tert-butoxycarbonyl(methyl)amino]-2-[hydroxy-[1-(2-trimethylsilylethoxymethyl)indol-3-yl]methyl]thiazole-4-carboxylate (1 g, 1.24 mmol, 39.3% yield, 68.0% purity) as yellow oil. ¹H NMR (400 MHz, DMSO-d₆) δ ppm 7.53 (d, J=8.2 Hz, 2H), 7.47 (s, 1H), 7.16 (t, J=7.6 Hz, 1H), 7.02 (d, J=6.3 Hz, 1H), 6.75 (d, J=4.3 Hz, 1H), 6.09 (d, J=4.3 Hz, 1H), 5.53 (d, J=2.7 Hz, 2H), 3.73 (s, 3H), 3.48-3.43 (m, 2H), 3.11 (s, 3H), 1.28 (s, 9H), 0.85-0.79 (m, 2H), 0.09 (s, 9H); ES-LCMS m/z 548.3 [M+H]⁺.

### Step 3: Methyl 5-((tert-butoxycarbonyl)(methyl)amino)-2-(1-((2-(trimethylsilyl)ethoxy)methyl)-1H-indole-3-carbonyl)thiazole-4-carboxylate

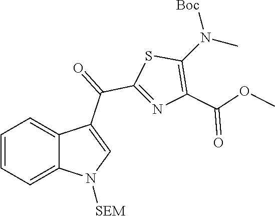 [SMILES: COC(=O)c1nc(C(=O)c2cn(COCC[Si](C)(C)C)c3ccccc23)sc1N(C)C(=O)OC(C)(C)C]

To a solution of methyl 5-[tert-butoxycarbonyl(methyl)amino]-2-[hydroxy-[1-(2-trimethylsilylethoxymethyl)indol-3-yl]methyl]thiazole-4-carboxylate (1 g, 1.83 mmol, 1 eq) in CHCl₃ (20 mL) was added MnO₂ (4.76 g, 54.77 mmol, 30 eq). The mixture was stirred at 60° C. for 2 h. The reaction mixture was filtered and concentrated under reduced pressure to yield a residue which was purified by silica gel column chromatography (from pure PE to PE/EtOAc=200/1 to 5/1, TLC:PE/EtOAc=5/1, R_{f}=0.28) to yield methyl 5-[tert-butoxycarbonyl(methyl)amino]-2-[1-(2-trimethylsilylethoxymethyl)indole-3-carbonyl]thiazole-4-carboxylate (0.5 g, 907.06 μmol, 49.7% yield, 99.0% purity) as yellow oil. ¹H NMR (500 MHz, CDCl₃) δ ppm 9.07 (s, 1H), 8.50-8.48 (m, 1H), 7.58-7.56 (m, 1H), 7.37-7.35 (m, 2H), 5.60 (s, 2H), 3.96 (s, 3H), 3.55 (t, J=8.0 Hz, 2H), 3.30 (s, 3H), 1.42 (s, 9H), 0.92 (t, J=8.0 Hz, 2H), 0.06 (s, 9H); ES-LCMS m/z 546.2 [M+H]⁺.

### Step 4: Methyl 2-(1H-indole-3-carbonyl)-5-(methylamino)thiazole-4-carboxylate

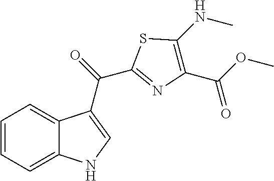 [SMILES: CNc1sc(C(=O)c2c[nH]c3ccccc23)nc1C(=O)OC]

To a solution of methyl 5-[tert-butoxycarbonyl(methyl)amino]-2-[1-(2-trimethylsilylethoxymethyl)indole-3-carbonyl]thiazole-4-carboxylate (200 mg, 366.49 μmol, 1 eq) in DCM (1 mL) was added TFA (1.54 g, 13.51 mmol, 1 mL, 36.85 eq). The mixture was stirred at 25° C. for 1 h. The reaction mixture was adjusted pH=7-8 by addition saturated NaHCO₃ solution at 0° C., diluted with water (30 mL) and extracted with EtOAc (50 mL×3). The combined organic layers were washed with brine (60 mL), dried over Na₂SO₄, filtered and concentrated under reduced pressure to give a residue. Then a stirred solution of the residue in THF (5 mL) was added KOH (19.50 mg, 347.45 μmol, 0.1 mL, 1 eq). The reaction mixture was stirred at 25° C. for 5 min. The reaction mixture was partitioned between water (30 mL) and EtOAc (50×3 mL). The organic phase was separated, washed with brine (30 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 36%-66%, 10 min) to yield methyl 2-(1H-indole-3-carbonyl)-5-(methylamino)thiazole-4-carboxylate (28 mg, 88.79 μmol, 25.6% yield, 100.0% purity) as a yellow solid. ¹H NMR (400 MHz, DMSO-d₆) δ ppm 12.14 (s, 1H), 8.94 (s, 1H), 8.29-8.27 (m, 1H), 8.22-8.21 (m, 1H), 7.56-7.54 (m, 1H), 7.28-7.22 (m, 2H), 3.85 (s, 3H), 3.04 (d, J=4.8 Hz, 3H); ES-LCMS m/z 316.2 [M+H]⁺.

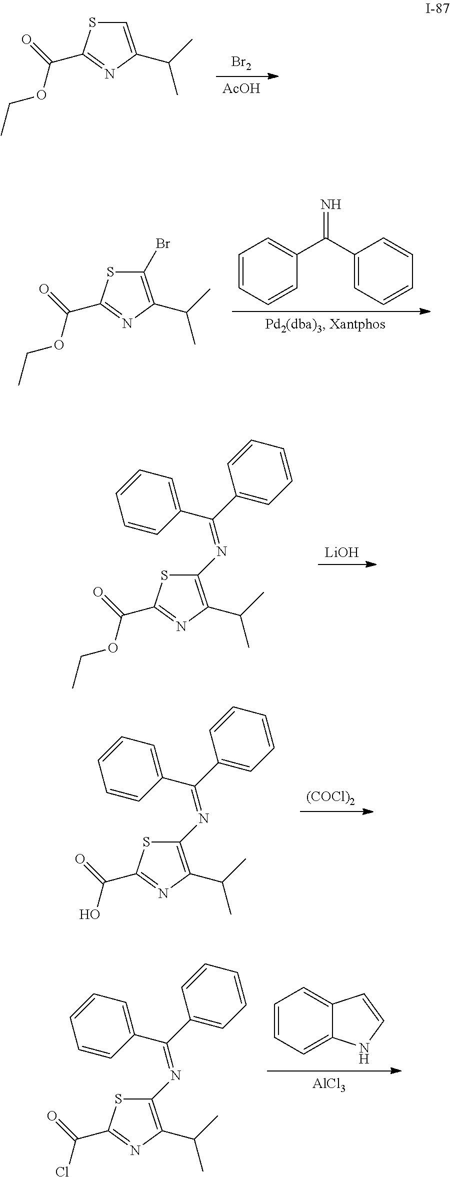

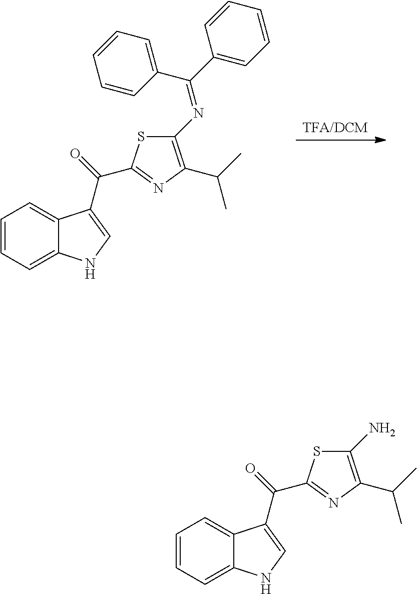 [SMILES: CC(C)c1nc(C(=O)c2c[nH]c3ccccc23)sc1N=C(c1ccccc1)c1ccccc1 ; "TFA/DCM" ; CC(C)c1nc(C(=O)c2c[nH]c3ccccc23)sc1N]

### Step 1: Ethyl 5-bromo-4-isopropyl-thiazole-2-carboxylate

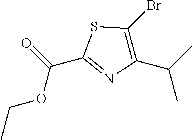

To a solution of ethyl 4-isopropylthiazole-2-carboxylate (1 g, 5.02 mmol, 1 eq) in AcOH (10 mL) was added Br₂ (4.01 g, 25.09 mmol, 1.29 mL, 5 eq) at 0° C. The mixture was stirred at 0° C. for 1 h. TLC (PE/EtOAc=10/1, R_{f}=0.48) indicated 30% of the starting materials was remained and one new spot formed. The reaction mixture was concentrated under reduced pressure to yield a residue. The mixture was diluted with water (100 mL), basified with aqueous Na₂CO₃ until pH=7-8 and extracted with ethyl acetate (80 mL×3). The combined organic phase was washed with brine (50 mL), dried over anhydrous Na₂SO₄, filtered and concentrated in vacuum to yield ethyl 5-bromo-4-isopropyl-thiazole-2-carboxylate (1 g, crude) as yellow oil, which was used in the next step without further purification. ¹H NMR (500 MHz, CDCl₃) δ ppm 4.54-4.42 (m, 2H), 3.35-3.22 (m, 1H), 1.43 (q, J=7.3 Hz, 3H), 1.37-1.31 (m, 6H).

### Step 2: Ethyl 5-(benzhydrylideneamino)-4-isopropyl-thiazole-2-carboxylate

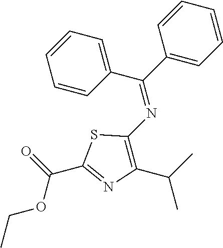 [SMILES: CCOC(=O)c1nc(C(C)C)c(N=C(c2ccccc2)c2ccccc2)s1]

To a solution of ethyl 5-bromo-4-isopropyl-thiazole-2-carboxylate (800 mg, 2.88 mmol, 1 eq, crude) and diphenylmethanimine (625.47 mg, 3.45 mmol, 579.14 μL, 1.2 eq) in toluene (5 mL) was added (5-diphenylphosphanyl-9,9-dimethylxanthen-4-yl)-diphenylphosphane (299.54 mg, 517.68 μmol, 0.18 eq), Cs₂CO₃ (2.06 g, 6.33 mmol, 2.2 eq) and Pd₂(dba)₃ (158.02 mg, 172.56 μmol, 0.06 eq). The mixture was stirred under N₂ atmosphere at 80° C. for 12 h. TLC (PE/EtOAc=3/1, R_{f}=0.52) indicated the starting material was consumed completely and three new spots formed. The reaction mixture was filtered and concentrated under reduced pressure to yield a residue which was purified by flash silica gel chromatography (from PE/EtOAc=100/1 to 3/1, TLC:PE/EtOAc=3/1, R_{f}=0.17) to yield ethyl 5-(benzhydrylideneamino)-4-isopropyl-thiazole-2-carboxylate (400 mg, 813.77 μmol, 28.3% yield, 77.0% purity) as a yellow solid. ¹H NMR (500 MHz, DMSO-d₆) δ ppm 7.75-7.72 (m, 2H), 7.68-7.62 (m, 3H), 7.56-7.47 (m, 3H), 7.34-7.30 (m, 2H), 4.26 (q, J=7.2 Hz, 2H), 3.81-3.74 (m, 1H), 1.31 (d, J=7.0 Hz, 6H), 1.24 (t, J=7.1 Hz, 3H); ES-LCMS m/z 379.7 [M+H]⁺.

### Step 3: 5-(Benzhydrylideneamino)-4-isopropyl-thiazole-2-carboxylic acid

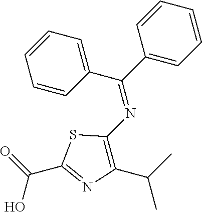 [SMILES: CC(C)c1nc(C(=O)O)sc1N=C(c1ccccc1)c1ccccc1]

To a solution of ethyl 5-(benzhydrylideneamino)-4-isopropyl-thiazole-2-carboxylate (300 mg, 610.33 μmol, 77.0%, 1 eq) in THF (6 mL) and MeOH (2 mL) was added LiOH.H₂O (35.86 mg, 854.46 μmol, 1.1 eq). The mixture was stirred at 25° C. for 12 h. The reaction mixture was concentrated under reduced pressure to yield 5-(benzhydrylideneamino)-4-isopropyl-thiazole-2-carboxylic acid (270 mg, crude) as a yellow solid which was used in the next step without further purification. ¹H NMR (400 MHz, DMSO-d₆) δ ppm 7.66 (dd, J=1.6, 7.8 Hz, 2H), 7.62-7.57 (m, 3H), 7.49-7.44 (m, 3H), 7.29-7.25 (m, 2H), 3.80-3.71 (m, 1H), 1.27 (d, J=7.0 Hz, 6H); ES-LCMS m/z 351.4 [M+H]⁺.

### Step 4: 5-(Benzhydrylideneamino)-4-isopropyl-thiazole-2-carbonyl chloride

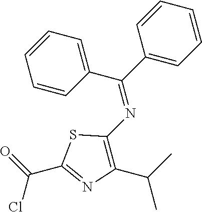 [SMILES: CC(C)c1nc(C(=O)Cl)sc1N=C(c1ccccc1)c1ccccc1]

To a solution of 5-(benzhydrylideneamino)-4-isopropyl-thiazole-2-carboxylic acid (200 mg, 485.11 μmol, 1 eq) in DCM (5 mL) was added (COCl)₂ (92.36 mg, 727.67 μmol, 63.70 μL, 1.5 eq) and DMF (3.55 mg, 48.51 μmol, 3.73 μL, 0.1 eq) at 0° C. The mixture was stirred at 25° C. for 1 h. The reaction mixture was concentrated under reduced pressure to yield 5-(benzhydrylideneamino)-4-isopropyl-thiazole-2-carbonyl chloride (180 mg, crude) as a yellow solid, which was used in the next step without further purification. ES-LCMS m/z 364.9 [M-Cl+OMe+H]⁺.

### Step 5: [5-(Benzhydrylideneamino)-4-isopropyl-thiazol-2-yl]-(1H-indol-3-yl)methanone

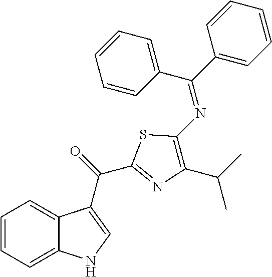 [SMILES: CC(C)c1nc(C(=O)c2c[nH]c3ccccc23)sc1N=C(c1ccccc1)c1ccccc1]

To a solution of 5-(benzhydrylideneamino)-4-isopropyl-thiazole-2-carbonyl chloride (180 mg, 487.96 μmol, 1 eq) in DCM (5 mL) was added AlCl₃ (357.86 mg, 2.68 mmol, 5.5 eq) at 0° C. After being stirring for 0.25 h, indole (114.33 mg, 975.93 μmol, 2 eq) was added. The mixture was stirred at 60° C. for 3 h. The reaction mixture was quenched by addition MeOH (5 mL) at 25° C. which was purified by flash silica gel chromatography (from PE/EtOAc=100/1 to 3/1, TLC:PE/EtOAc=3/1, R_{f}=0.32) to yield [5-(benzhydrylideneamino)-4-isopropyl-thiazol-2-yl]-(1H-indol-3-yl)methanone (100 mg, 166.83 μmol, 34.2% yield, 75.0% purity) as a yellow solid. ¹H NMR (500 MHz, DMSO-d₆) δ ppm 12.14 (s, 1H), 9.01 (d, J=3.1 Hz, 1H), 8.21 (d, J=7.9 Hz, 1H), 7.76 (d, J=7.2 Hz, 2H), 7.69-7.64 (m, 3H), 7.56-7.49 (m, 4H), 7.36-7.33 (m, 2H), 7.27-7.20 (m, 2H), 3.83 (td, J=6.8, 13.8 Hz, 1H), 1.42 (d, J=6.9 Hz, 6H); ES-LCMS m/z 450.2 [M+H]⁺.

### Step 6: (5-Amino-4-isopropyl-thiazol-2-yl)-(1H-indol-3-yl)methanone

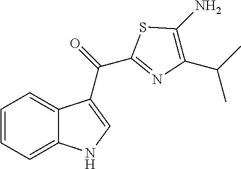 [SMILES: CC(C)c1nc(C(=O)c2c[nH]c3ccccc23)sc1N]

To a solution of [5-(benzhydrylideneamino)-4-isopropyl-thiazol-2-yl]-(1H-indol-3-yl)methanone (70 mg, 116.78 μmol, 75.0%, 1 eq) in THF (3 mL) was added HCl (3 M, 77.85 μL, 2 eq). The mixture was stirred at 25° C. for 1 h. TLC (PE/EtOAc=3/1, R_{f}=0.40) indicated the starting material was consumed completely and two new spots formed. The mixture was quenched with sat. aq. NaHCO₃ (20 mL) and extracted with ethyl acetate (30 mL×3). The combined organic phase was washed with brine (20 mL), dried over anhydrous Na₂SO₄, filtered and concentrated in vacuum to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 35%-65%, 10 min), followed by lyophilization to yield (5-amino-4-isopropyl-thiazol-2-yl)-(1H-indol-3-yl)methanone (13.78 mg, 47.32 μmol, 40.5% yield, 98.0% purity) as a yellow solid. ¹H NMR (400 MHz, DMSO-d₆) δ ppm 11.91 (s, 1H), 8.91 (s, 1H), 8.29 (d, J=6.3 Hz, 1H), 7.50 (d, J=7.0 Hz, 1H), 7.24-7.15 (m, 2H), 6.47 (s, 2H), 3.08 (td, J=6.9, 13.5 Hz, 1H), 1.25 (d, J=6.7 Hz, 6H); ES-LCMS m/z 286.1 [M+H]⁺.

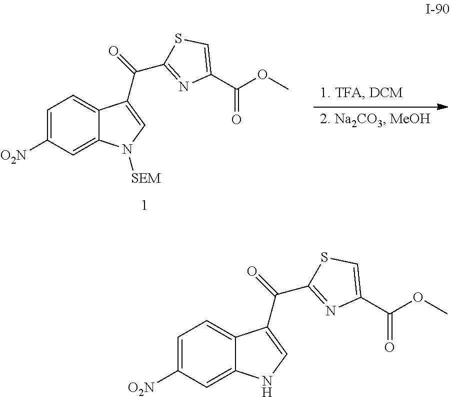

### Step 1: Methyl 2-(6-nitro-1H-indole-3-carbonyl)thiazole-4-carboxylate

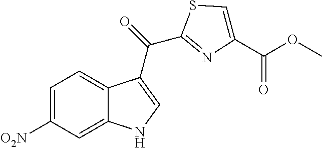 [SMILES: COC(=O)c1csc(C(=O)c2c[nH]c3cc([N+](=O)[O-])ccc23)n1]

To a solution of methyl 2-[6-nitro-1-(2-trimethylsilylethoxymethyl)indole-3-carbonyl]thiazole-4-carboxylate (290 mg, 433.31 μmol, 69%, 1 eq) in DCM (5 mL) was added TFA (1.54 g, 13.50 mmol, 1.0 mL, 31.17 eq). The mixture was stirred at 25° C. for 12 h. The reaction mixture was concentrated to yield a residue, which was dissolved in MeCN (10 mL), adjusted to pH=9 by aq. Na₂CO₃. The mixture was stirred at 25° C. for 1 h. The mixture was filtered and concentrated under reduced pressure to give a residue which was purified by preparative HPLC (column: Agela DuraShell C₁₈ 150*25 mm*5 μm; mobile phase: [water (0.05% NH₃.H₂O+10 mM NH₄HCO₃)-ACN]; B %: 43%-73%, 10 min), followed by lyophilization to yield methyl 2-(6-nitro-1H-indole-3-carbonyl)thiazole-4-carboxylate (24.25 mg, 70.02 μmol, 16.1% yield, 95.6% purity) as a gray solid. ¹H NMR (400 MHz, DMSO-d₆) δ ppm 12.82 (br s, 1H), 9.35 (s, 1H), 8.94 (s, 1H), 8.53 (d, J=1.7 Hz, 1H), 8.47 (d, J=8.8 Hz, 1H), 8.18 (dd, J=2.0, 8.8 Hz, 1H), 3.93 (s, 3H); ES-LCMS m/z 332.0 [M+H]⁺.

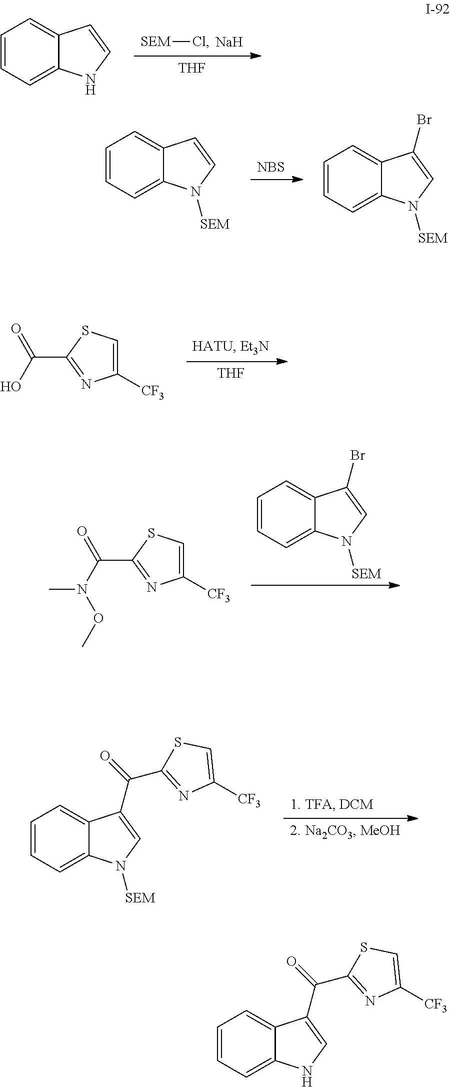

### Step 1: 2-(Indol-1-ylmethoxy)ethyl-trimethyl-silane

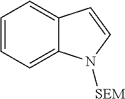 [SMILES: C[Si](C)(C)CCOCn1ccc2ccccc21]

To a solution of indole (10 g, 85.36 mmol, 1 eq) in THF (150 mL) was added NaH (5.12 g, 128.04 mmol, 60%, 1.5 eq) at 0° C. under N₂. After being stirred for 0.5 h, SEM-Cl (17.08 g, 102.43 mmol, 18.13 mL, 1.2 eq) was added dropwise. The mixture was stirred at 0° C. for 2 h. The reaction mixture was quenched by addition of water (200 mL) under 0° C., extracted with EtOAc (150 mL×3). The combined organic layers were washed with brine (50 mL), dried over Na₂SO₄, filtered and concentrated under reduced pressure to yield a residue which was purified by flash silica gel chromatography (from PE/EtOAc=1/0 to 1/0, TLC:PE/EtOAc=1/0, R_{f}=0.19) to yield 2-(indol-1-ylmethoxy)ethyl-trimethyl-silane (8 g, 29.10 mmol, 34.1% yield, 90.0% purity) as yellow oil. ¹H NMR (400 MHz, CDCl₃) δ ppm 7.74 (d, J=7.0 Hz, 1H), 7.63-7.55 (m, 1H), 7.38-7.31 (m, 1H), 7.27-7.22 (m, 2H), 6.63 (d, J=2.7 Hz, 1H), 5.53 (s, 2H), 3.56 (t, J=8.0 Hz, 2H), 0.99 (d, J=8.6 Hz, 2H), 0.09-0.00 (m, 9H); ES-LCMS m/z 248.1 [M+H]⁺.

### Step 2: 2-[(3-Bromoindol-1-yl)methoxy]ethyl-trimethyl-silane

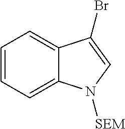 [SMILES: C[Si](C)(C)CCOCn1cc(Br)c2ccccc21]

To a solution of 2-(indol-1-ylmethoxy)ethyl-trimethyl-silane (1.08 g, 3.92 mmol, 90%, 1 eq) in THF (20 mL) was added NBS (697.81 mg, 3.92 mmol, 1 eq). The mixture was stirred at 0° C. for 15 min. The reaction mixture was quenched by addition of water (100 mL), extracted with EtOAc (50 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 3/1, TLC:PE/EtOAc=10/1, R_{f}=0.70) to yield 2-[(3-bromoindol-1-yl)methoxy]ethyl-trimethyl-silane (1.18 g, 3.25 mmol, 83.0% yield, 90.0% purity) as red oil. ¹H NMR (400 MHz, CDCl₃) δ ppm 7.63 (d, J=7.8 Hz, 1H), 7.54 (d, J=8.1 Hz, 1H), 7.38-7.33 (m, 1H), 7.33-7.31 (m, 1H), 7.29 (s, 1H), 5.51 (s, 2H), 3.56-3.51 (m, 2H), 0.96-0.92 (m, 2H), 0.00 (s, 9H); ES-LCMS no desired m/z was detected on LCMS.

### Step 3: N-Methoxy-N-methyl-4-(trifluoromethyl)thiazole-2-carboxamide

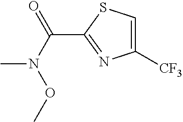

To a solution of N-methoxymethanamine (1.24 g, 12.68 mmol, 5 eq, HCl) in DMF (10 mL) was added HATU (1.74 g, 4.57 mmol, 1.8 eq), 4-(trifluoromethyl)thiazole-2-carboxylic acid (500 mg, 2.54 mmol, 1 eq) and TEA (769.95 mg, 7.61 mmol, 1.06 mL, 3 eq). The mixture was stirred at 20° C. for 1 h. The reaction mixture was quenched by addition of water (100 mL), extracted with EtOAc (50 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 5/1, TLC:PE/EtOAc=5/1, R_{f}=0.7) to yield N-methoxy-N-methyl-4-(trifluoromethyl)thiazole-2-carboxamide (617 mg, 2.52 mmol, 99.3% yield, 98.0% purity) as a white solid. ¹H NMR (400 MHz, CDCl₃) δ ppm 7.99 (s, 1H), 3.90 (s, 3H), 3.80-3.23 (m, 3H); ES-LCMS m/z 241.2 [M+H]⁺.

### Step 4: [4-(Trifluoromethyl)thiazol-2-yl]-[1-(2-trimethylsilylethoxymethyl)indol-3-yl]methanone

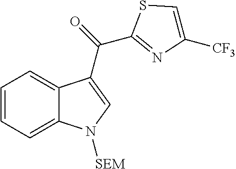 [SMILES: C[Si](C)(C)CCOCn1cc(C(=O)c2nc(C(F)(F)F)cs2)c2ccccc21]

To a solution of 2-[(3-bromoindol-1-yl)methoxy]ethyl-trimethyl-silane (916.67 mg, 2.53 mmol, 90%, 1 eq) in THF (3 mL) was added t-BuLi (1.3 M, 3.89 mL, 2 eq). The mixture was stirred at −78° C. for 10 min. N-methoxy-N-methyl-4-(trifluoromethyl)thiazole-2-carboxamide (613.51 mg, 2.50 mmol, 98%, 0.99 eq) in THF (3 mL) was added at −78° C. The mixture was stirred at −60° C. for 1 h. The reaction mixture was quenched by addition of water (100 mL), extracted with EtOAc (50 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 5/1, TLC:PE/EtOAc=5/1, R_{f}=0.7) to yield [4-(trifluoromethyl)thiazol-2-yl]-[1-(2-trimethylsilylethoxymethyl)indol-3-yl]methanone (97 mg, 190.12 μmol, 7.5% yield, 83.6% purity) as a yellow solid. ¹H NMR (400 MHz, CDCl₃) δ ppm 9.07 (s, 1H), 8.55-8.50 (m, 1H), 8.04 (s, 1H), 7.62-7.58 (m, 1H), 7.43-7.38 (m, 2H), 5.63 (s, 2H), 3.61-3.56 (m, 2H), 0.97-0.92 (m, 2H), −0.04 (s, 9H); ES-LCMS m/z 427.0 [M+H]⁺.

### Step 5: 1H-Indol-3-yl-[4-(trifluoromethyl)thiazol-2-yl]methanone

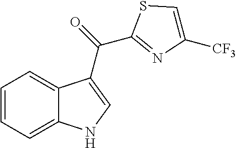 [SMILES: O=C(c1nc(C(F)(F)F)cs1)c1c[nH]c2ccccc12]

To a stirred solution of [4-(trifluoromethyl)thiazol-2-yl]-[1-(2-trimethylsilylethoxymethyl)indol-3-yl]methanone (97 mg, 188.76 μmol, 83% purity, 1 eq) in DCM (4 mL) was added TFA (3.08 g, 27.01 mmol, 2 mL, 143.11 eq). The reaction mixture was stirred at 20° C. for 1.5 h. TLC (PE/EtOAc=3/1, R_{f}=0.20) showed the starting material was consumed completely and one new spot was detected. The reaction mixture was concentrated at 20° C. to yield a residue which was dissolved in DCM (25 mL). The mixture was concentrated to yield a residue which was dissolved in MeOH (2 mL) and THF (2 mL). The mixture was adjusted pH to 9 by saturated Na₂HCO₃ solution then stirred at 20° C. for 2 h. The reaction mixture was quenched by addition of water (50 mL), extracted with EtOAc (30 mL×3). The combined organic layers were washed with brine (10 mL), dried over Na₂SO₄, filtered and concentrated under reduced pressure to yield a residue which was purified by preparative HPLC (column: Agela DuraShell C18 150*25 mm*5 μm; mobile phase: [water (0.05% NH₃.H₂O+10 mM NH₄HCO₃)-ACN]; B %: 46%-76%, 10 min) and lyophilized to yield 1H-indol-3-yl-[4-(trifluoromethyl)thiazol-2-yl]methanone (14.76 mg, 49.27 μmol, 26.1% yield, 98.9% purity) as a gray solid. ¹H NMR (400 MHz, CDCl₃) δ ppm 9.18 (d, J=3.2 Hz, 1H), 8.83 (s, 1H), 8.57-8.50 (m, 1H), 8.04 (s, 1H), 7.53-7.47 (m, 1H), 7.42-7.34 (m, 2H); ES-LCMS m/z 296.7 [M+H]⁺.

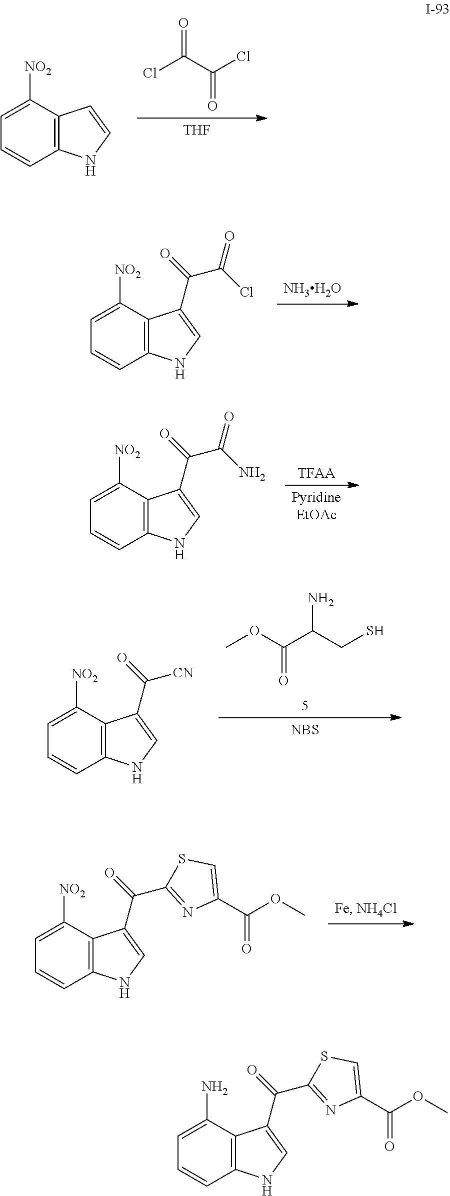

### Step 1: 2-(4-Nitro-1H-indol-3-yl)-2-oxoacetyl chloride

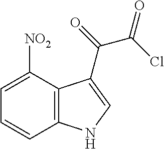 [SMILES: O=C(Cl)C(=O)c1c[nH]c2cccc([N+](=O)[O-])c12]

To a solution of 4-nitro-1H-indole (1 g, 6.17 μmol, 1 eq) in THF (10 mL) was added oxalyl dichloride (2.35 g, 18.50 μmol, 1.62 mL, 3 eq) under 0° C. The mixture was stirred at 25° C. for 12 h. The reaction mixture was concentrated to yield 2-(4-nitro-1H-indol-3-yl)-2-oxo-acetyl chloride (1.1 g, crude) as a brown solid which was used in the next step without further purification; ES-LCMS no desired m/z was detected on LCMS.

### Step 2: 2-(4-Nitro-1H-indol-3-yl)-2-oxoacetamide

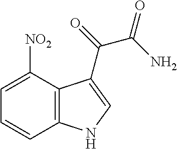 [SMILES: NC(=O)C(=O)c1c[nH]c2cccc([N+](=O)[O-])c12]

To a solution of 2-(4-nitro-1H-indol-3-yl)-2-oxo-acetyl chloride (1.1 g, 3.53 μmol, 81.0%, 1 eq) in NH₃.H₂O (10 mL, 28.0%) was added EtOH (5 mL). The mixture was stirred at 0° C. for 1 h. The reaction mixture was quenched by addition of water (30 mL), extracted with EtOAc (20 mL×3). The combined organic layers were washed with brine (10 mL), dried over Na₂SO₄, filtered and concentrated under reduced pressure to give a residue which was purified by flash silica gel chromatography (from pure PE to 3/5, TLC:PE/EtOAc=1/1, R_{f}=0.26) to yield 2-(4-nitro-1H-indol-3-yl)-2-oxo-acetamide (886 mg, 3.23 μmol, 91.5% yield, 85.0% purity) as a yellow solid. ¹H NMR (400 MHz, DMSO-d₆) δ ppm 12.76 (br s, 1H), 8.68 (s, 1H), 8.10 (br s, 1H), 7.86 (dd, J=0.8, 8.2 Hz, 1H), 7.78-7.68 (m, 2H), 7.42 (t, J=8.0 Hz, 1H); ES-LCMS m/z 256.1 [M+Na]⁺.

### Step 3: 4-Nitro-1H-indole-3-carbonyl cyanide

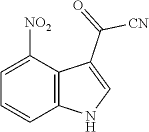 [SMILES: N#CC(=O)c1c[nH]c2cccc([N+](=O)[O-])c12]

To a solution of 2-(4-nitro-1H-indol-3-yl)-2-oxo-acetamide (100 mg, 364.53 μmol, 85%, 1 eq) in EtOAc (5 mL) was added Pyridine (288.34 mg, 3.65 μmol, 294.22 L, 10 eq) and TFAA (382.81 mg, 1.82 μmol, 253.51 μL, 5 eq). The mixture was stirred at 25 C for 3 h. The reaction mixture was quenched by addition of water (30 mL), extracted with EtOAc (30 mL×3). The combined organic layers were washed with brine (10 mL), dried over Na₂SO₄, filtered and concentrated under reduced pressure to give a residue which was purified by flash silica gel chromatography (from pure PE to 1/1, TLC:PE/EtOAc=1/1, R_{f}=0.44) to yield 4-nitro-1H-indole-3-carbonyl cyanide (63 mg, 225.46 μmol, 61.8% yield, 77.0% purity) as a yellow solid. ¹H NMR (400 MHz, DMSO-d₆) δ ppm 13.53 (br s, 1H), 8.91 (s, 1H), 7.92 (dd, J=0.8, 8.2 Hz, 1H), 7.80 (dd, J=0.8, 7.8 Hz, 1H), 7.58-7.48 (m, 1H); ES-LCMS no desired m/z was detected on LCMS.

### Step 4: Methyl 2-(4-nitro-1H-indole-3-carbonyl)thiazole-4-carboxylate

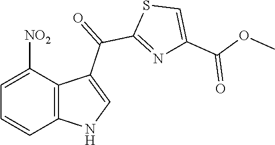 [SMILES: COC(=O)c1csc(C(=O)c2c[nH]c3cccc([N+](=O)[O-])c23)n1]

To a solution of 4-nitro-1H-indole-3-carbonyl cyanide (200 mg, 883.04 μmol, 95%, 1 eq) in pyridine (6 mL) was added DBU (13.44 mg, 88.30 umol, 13.31 μL, 0.1 eq) and methyl 2-amino-3-mercaptopropanoate hydrochloride (151.57 mg, 883.04 μmol, 1 eq). After being stirred at 40° C. for 2 h, the reaction mixture was diluted with DCM (50 mL), cooled to 0° C., added DBU (268.86 mg, 1.77 μmol, 266.20 μL, 2 eq), followed by NBS (172.88 mg, 971.35 μmol, 1.1 eq) portion-wise. The mixture was stirred at 0° C. for 1 h. The reaction mixture was quenched by addition of water (30 mL), extracted with EtOAc (50 mL×3). The combined organic layers were washed with brine (20 mL), dried over Na₂SO₄, filtered and concentrated under reduced pressure to give a residue which was purified by flash silica gel chromatography (from pure PE to 2/3, TLC:PE/EtOAc=1/1, R_{f}=0.43) to yield methyl 2-(4-nitro-1H-indole-3-carbonyl)thiazole-4-carboxylate (160 mg, 410.50 μmol, 46.4% yield, 85.0% purity) as a yellow solid. ¹H NMR (400 MHz, DMSO-d₆) δ ppm 12.93 (br s, 1H), 11.06 (br s, 1H), 8.93 (s, 1H), 7.99-7.94 (m, 1H), 7.79 (dd, J=0.8, 7.8 Hz, 1H), 7.52-7.48 (m, 1H), 3.90 (s, 3H); ES-LCMS m/z 354.0 [M+Na]⁺.

### Step 5: Methyl 2-(4-amino-1H-indole-3-carbonyl)thiazole-4-carboxylate

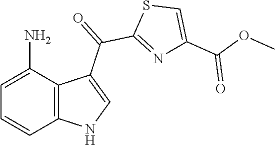 [SMILES: COC(=O)c1csc(C(=O)c2c[nH]c3cccc(N)c23)n1]

To a solution of methyl 2-(4-nitro-1H-indole-3-carbonyl)thiazole-4-carboxylate (110 mg, 282.22 μmol, 85%, 1 eq) in EtOH (10 mL) and H₂O (10 mL) was added Fe (78.80 mg, 1.41 μmol, 5 eq) and NH₄Cl (150.96 mg, 2.82 μmol, 10 eq). The mixture was stirred at 80° C. for 1 h. The reaction mixture was quenched by addition of water (30 mL), extracted with EtOAc (40 mL×3). The combined organic layers were washed with brine (20 mL), dried over Na₂SO₄, filtered and concentrated under reduced pressure to give a residue which was purified by preparative HPLC (column: Agela DuraShell C18 150*25 mm*5 μm; mobile phase: [water (0.05% NH₃.H₂O+10 mM NH₄HCO₃)-ACN]; B %: 28%-58%, 10 min), followed by lyophilization to yield methyl 2-(4-amino-1H-indole-3-carbonyl)thiazole-4-carboxylate (35.05 mg, 113.41 μmol, 40.1% yield, 97.5% purity) as a yellow solid. ¹H NMR (400 MHz, DMSO-d₆) δ ppm 12.26 (br s, 1H), 9.07 (d, J=3.1 Hz, 1H), 8.88 (s, 1H), 6.98 (t, J=7.8 Hz, 1H), 6.70-6.65 (m, 1H), 6.43-6.35 (m, 3H), 3.91 (s, 3H); ES-LCMS m/z 302.1[M+H]⁺.

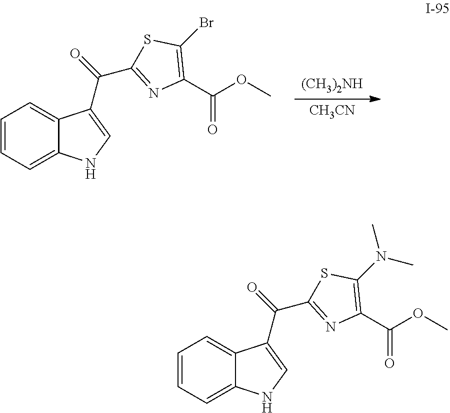

### Step 1: Methyl 5-(dimethylamino)-2-(1H-indole-3-carbonyl)thiazole-4-carboxylate

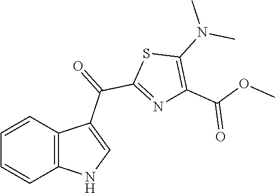 [SMILES: COC(=O)c1nc(C(=O)c2c[nH]c3ccccc23)sc1N(C)C]

To a stirred solution of methyl 5-bromo-2-(1H-indole-3-carbonyl)thiazole-4-carboxylate (60 mg, 164.29 μmol, 100.0%, 1 eq) in CH₃CN (15 mL) was added (CH₃)₂NH (4.01 g, 35.54 mmol, 74.04 mL, 40.0%, 216.30 eq). The reaction mixture was stirred at 25° C. for 12 h. The reaction mixture was partitioned between water (30 mL) and EtOAc (50×3 mL). The organic phase was separated, washed with saturated brine (30 mL), dried over Na₂SO₄, filtered and concentrated under reduced pressure. To the crude product was added MeCN (30 mL) and the mixture was stirred at 25° C. for 2 h. The slurry was filtered and the cake was rinsed with PE (2×30 mL). The solid was collected and dried in vacuo to yield methyl 5-(dimethylamino)-2-(1H-indole-3-carbonyl)thiazole-4-carboxylate (47.27 mg, 143.52 μmol, 87.4% yield, 100.0% purity) as a yellow solid. ¹H NMR (500 MHz, CDCl₃) δ ppm 9.09 (d, J=2.0 Hz, 1H), 8.89 (s, 1H), 8.51 (d, J=7.0 Hz, 1H), 7.44 (d, J=8.0 Hz, 1H), 7.32 (q, J=7.0 Hz, 2H), 3.95 (s, 3H), 3.20 (s, 6H); ES-LCMS m/z 330.2 [M+H]⁺.

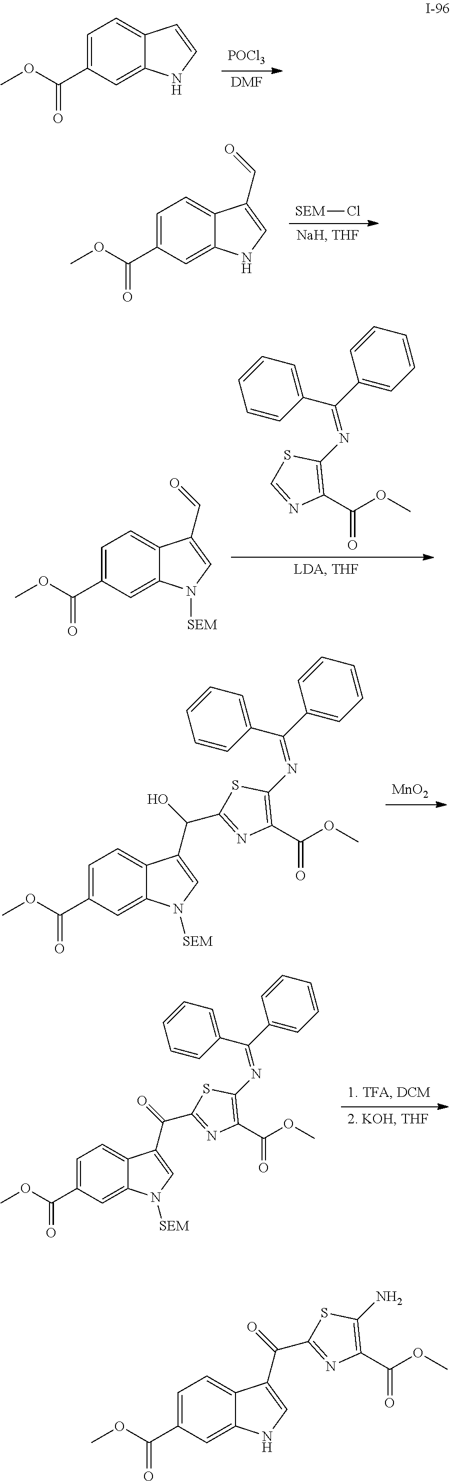

### Step 1: Methyl 3-formyl-1H-indole-6-carboxylate

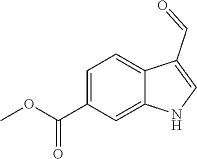

POCl₃ (5.25 g, 34.25 mmol, 3.18 mL, 1.5 eq) was added to DMF (15 mL) dropwise and stirred at 0° C. for 1 h. Then a solution of methyl 1H-indole-6-carboxylate (4 g, 22.83 mmol, 1 eq) in DMF (25 mL) was added to the mixture. The mixture was stirred at 80° C. for 16 h. The reaction mixture was quenched by addition H₂O (100 mL) at 0° C. and stirred for 30 min at 25° C. The reaction mixture was filtered and the filter cake was concentrated to yield a residue which was added MeOH (35 mL) and stirred at 25° C. for 1 h. The slurry was filtered and the filter cake was washed with MeOH (20 mL×2). The filter cake was concerned to yield methyl 3-formyl-1H-indole-6-carboxylate (3.15 g, 13.95 mmol, 61.1% yield, 90.0% purity) as a light yellow solid. ¹H NMR (500 MHz, DMSO-d₆) δ ppm 12.44 (s, 1H), 10.10-9.85 (m, 1H), 8.50 (s, 1H), 8.23-8.07 (m, 2H), 7.94-7.68 (m, 1H), 3.90-3.84 (m, 3H).

### Step 2: Methyl 3-formyl-1-(2-trimethylsilylethoxymethyl)indole-6-carboxylate

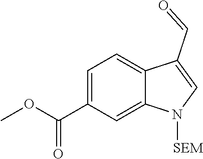 [SMILES: COC(=O)c1ccc2c(C=O)cn(COCC[Si](C)(C)C)c2c1]

To a solution of methyl 3-formyl-1H-indole-6-carboxylate (3.1 g, 13.73 mmol, 1 eq) in THF (30 mL) was added NaH (823.84 mg, 20.60 mmol, 60.0% purity, 1.5 eq) at 0° C. The mixture was stirred for 30 min at 0° C. under N₂ atmosphere. Then SEM-Cl (2.75 g, 16.48 mmol, 2.92 mL, 1.2 eq) was added to the mixture dropwise. The mixture was stirred at 0° C. for 2 h. TLC (PE/EtOAc=1/1, R_{f}=0.90) indicated the starting material was consumed completely and two new spot formed. To the mixture was added water (80 mL) and extracted with EtOAc (50 mL×3). The combined organic layers were washed with brine (50 mL), dried over Na₂SO₄, filtered and concentrated under reduced pressure to give a residue which was purified by flash silica gel chromatography (from PE/EtOAc=1/0 to 2/1, TLC:PE/EtOAc=1/1, R_{f}=0.90) to yield 3-formyl-1-(2-trimethylsilylethoxymethyl)indole-6-carboxylate (1.89 g, 5.50 mmol, 40.0% yield, 97.0% purity) as a light yellow oil. ¹H NMR (500 MHz, CDCl₃) δ ppm 10.07 (s, 1H), 8.34 (d, J=8.2 Hz, 1H), 8.27 (s, 1H), 8.02 (dd, J=1.1, 8.4 Hz, 1H), 7.93 (s, 1H), 5.59 (s, 2H), 3.96 (s, 3H), 3.56-3.52 (m, 2H), 0.94-0.90 (m, 2H), −0.05 (s, 9H); ES-LCMS m/z 334.7 [M+H]⁺.

### Step 3: Methyl 5-(benzhydrylideneamino)-2-[hydroxy-[6-methoxycarbonyl-1-(2-trimethylsilylethoxymethyl)indol-3-yl]methyl]thiazole-4-carboxylate

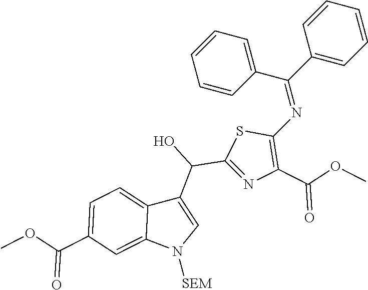 [SMILES: COC(=O)c1ccc2c(C(O)c3nc(C(=O)OC)c(N=C(c4ccccc4)c4ccccc4)s3)cn(COCC[Si](C)(C)C)c2c1]

To a stirred solution of DIPA (470.97 mg, 4.65 mmol, 657.78 μL, 2 eq) in THF (5 mL) was cooled to −75° C. then added n-BuLi (2.5 M, 1.86 mL, 2 eq) dropwise under N₂ atmosphere. The reaction mixture was stirred at −75° C. for 30 min under N₂. To a solution of methyl 3-formyl-1-(2-trimethylsilylethoxymethyl)indole-6-carboxylate (800 mg, 2.33 mmol, 1 eq) in THF (15 mL) was added methyl 5-(benzhydrylideneamino)thiazole-4-carboxylate (773.43 mg, 2.33 mmol, 1 eq). The LDA reaction mixture was added to the above mixture, then stirred at −75° C. for 30 min under N₂. The reaction mixture was quenched with aq. NH₄Cl (60 mL), extracted with EtOAc (80 mL×3). The combined organic layers were washed with brine (10 mL), dried over Na₂SO₄, filtered and concentrated under reduced pressure to give a residue which was purified by flash silica gel chromatography (from PE/EtOAc=1/0 to 3/1, TLC:PE/EtOAc=3/1, R_{f}=0.37) to yield methyl 5-(benzhydrylideneamino)-2-[hydroxy-[6-methoxycarbonyl-1-(2-trimethylsilylethoxymethyl)indol-3-yl]methyl]thiazole-4-carboxylate (490 mg, 597.71 μmol, 25.6% yield, 80.0% purity) as yellow oil. ¹H NMR (400 MHz, DMSO-d₆) δ ppm 8.16 (s, 1H), 7.70 (s, 2H), 7.64 (dd, J=1.3, 8.4 Hz, 1H), 7.59 (s, 1H), 7.48 (s, 5H), 7.41 (d, J=8.3 Hz, 2H), 7.34-7.14 (m, 2H), 6.68 (d, J=4.4 Hz, 1H), 5.98 (d, J=4.4 Hz, 1H), 5.65-5.56 (m, 2H), 3.87 (s, 3H), 3.66 (s, 3H), 3.45 (t, J=8.1 Hz, 2H), 0.82 (t, J=8.1 Hz, 2H), −0.09 (s, 9H); ES-LCMS m/z 656.2 [M+H]⁺.

### Step 4: Methyl 5-(benzhydrylideneamino)-2-[6-methoxycarbonyl-1-(2-trimethylsilylethoxymethyl)indole-3-carbonyl]thiazole-4-carboxylate

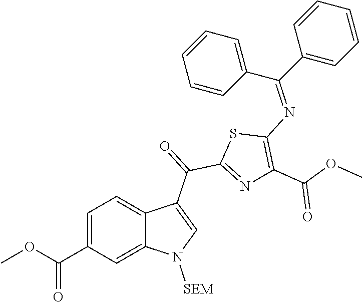

To a solution of methyl 5-(benzhydrylideneamino)-2-[hydroxy-[6-methoxycarbonyl-1-(2-trimethylsilylethoxymethyl)indol-3-yl]methyl]thiazole-4-carboxylate (390 mg, 475.73 μmol, 1 eq) in DCM (10 mL) was added MnO₂ (827.17 mg, 9.51 mmol, 20 eq). The mixture was stirred at 25° C. for 2 h. The mixture was filtered through celite and the cake was rinsed with DCM (30 mL×2). The filtrated was concentrated under reduced pressure to yield a residue which was purified by flash silica gel chromatography (from PE/EtOAc=1/0 to 5/1, TLC:PE/EtOAc=3/1, R_{f}=0.64) to yield methyl 5-(benzhydrylideneamino)-2-[6-methoxycarbonyl-1-(2-trimethylsilylethoxymethyl)indole-3-carbonyl]thiazole-4-carboxylate (280 mg, 398.28 μmol, 83.7% yield, 93.0% purity) as a yellow solid. ¹H NMR (400 MHz, DMSO-d₆) δ ppm 9.14 (s, 1H), 8.40-8.29 (m, 2H), 7.93 (d, J=8.3 Hz, 1H), 7.67-7.38 (m, 10H), 5.83 (s, 2H), 3.89 (s, 3H), 3.81 (s, 3H), 3.54 (t, J=7.9 Hz, 2H), 0.86 (t, J=7.9 Hz, 2H), −0.11 (s, 9H); ES-LCMS m/z 654.2 [M+H]⁺.

### Step 5: Methyl 5-amino-2-(6-methoxycarbonyl-1H-indole-3-carbonyl)thiazole-4-carboxylate

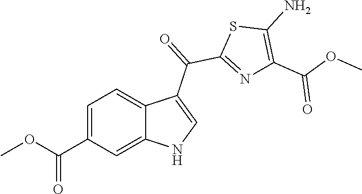

To a solution of methyl 5-(benzhydrylideneamino)-2-[6-methoxycarbonyl-1-(2-trimethylsilylethoxymethyl)indole-3-carbonyl]thiazole-4-carboxylate (280 mg, 398.28 μmol, 1 eq) in DCM (3 mL) was added TFA (5.39 g, 47.27 mmol, 3.50 mL, 118.69 eq) The mixture was stirred at 25° C. for 2 h. The mixture was concentrated under reduced pressure to yield a residue which was dissolved in MeOH (10 mL) added sat.aq. Na₂CO₃ until pH 9. The resulting mixture was stirred at 25° C. for 0.5 h. The reaction mixture was diluted with water (20 mL) and extracted with EtOAc (40 mL×3). The combined organic phases were washed with brine (20 mL), dried over anhydrous Na₂SO₄, filtered and concentrated under reduced pressure to give a residue which was added MeOH (10 mL), and stirred at 25° C. for 2 h. The slurry was filtered, and the cake was rinsed with MeOH (2×10 mL). The solid was collected and dried in vacuo to yield methyl 5-amino-2-(6-methoxycarbonyl-1H-indole-3-carbonyl)thiazole-4-carboxylate (127.07 mg, 353.60 μmol, 88.8% yield, 100.0% purity) as a yellow solid. ¹H NMR (400 MHz, DMSO-d₆) δ ppm 12.44 (s, 1H), 9.07 (s, 1H), 8.34 (d, J=8.6 Hz, 1H), 8.18 (d, J=0.7 Hz, 1H), 8.05 (s, 2H), 7.84 (dd, J=1.3, 8.4 Hz, 1H), 3.88 (s, 3H), 3.84 (s, 3H); ES-LCMS m/z 360.1 [M+H]⁺.

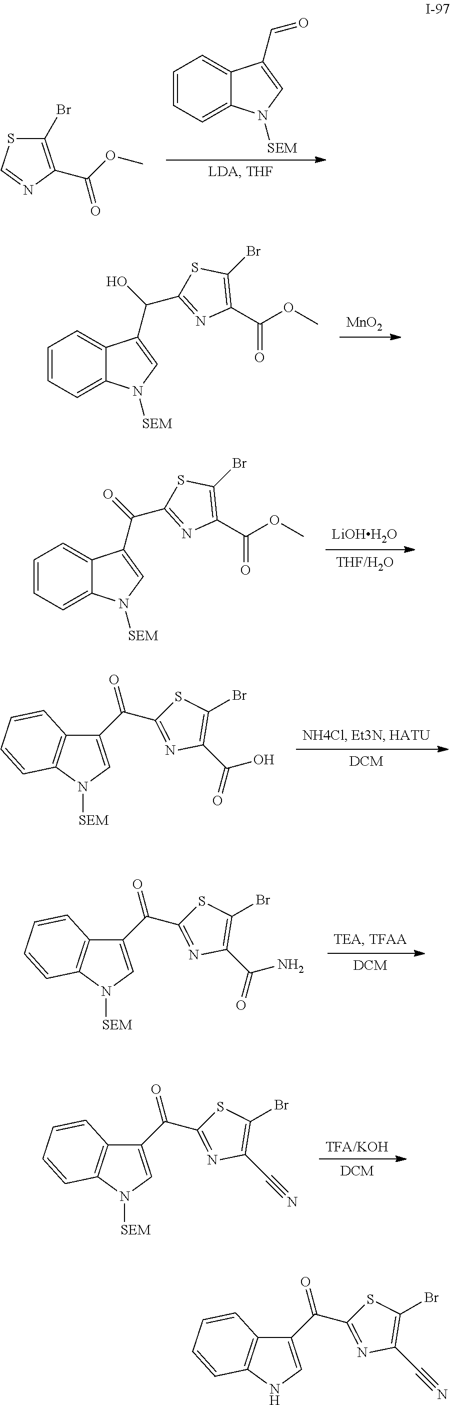

### Step 1: Methyl 5-bromo-2-[hydroxy-[1-(2-trimethylsilylethoxymethyl)indol-3-yl]methyl]thiazole-4-carboxylate

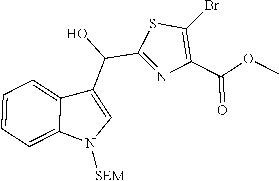 [SMILES: COC(=O)c1nc(C(O)c2cn(COCC[Si](C)(C)C)c3ccccc23)sc1Br]

To a solution of methyl 5-bromothiazole-4-carboxylate (7 g, 30.26 mmol, 96% purity, 1 eq) and 1-(2-trimethylsilylethoxymethyl)indole-3-carbaldehyde (9.02 g, 31.78 mmol, 97% purity, 1.05 eq) in THF (60 mL) was added LDA (2.0 M, 30.26 mL, 2 eq) dropwise below −60° C. under N₂ atmosphere. The mixture was stirred at −60° C. for 0.5 h. The reaction mixture was quenched by addition of sat. aq. NH₄Cl (50 mL) and extracted with EtOAc (80 mL×3). The combined organic layers were washed with brine (20 mL), dried over Na₂SO₄, filtered and concentrated under reduced pressure to yield a residue which was purified by flash silica gel chromatography (from PE/EtOAc=1/0 to 2/1, TLC:PE/EtOAc=3/1, R_{f}=0.15) to yield methyl 5-bromo-2-[hydroxy-[1-(2-trimethylsilylethoxymethyl)indol-3-yl]methyl]thiazole-4-carboxylate (1.9 g, 1.95 mmol, 6.4% yield, 51.0% purity) as a yellow solid. ES-LCMS m/z 479.0, 481.0 [M-OH]⁺.

### Step 2: Methyl 5-bromo-2-[1-(2-trimethylsilylethoxymethyl)indole-3-carbonyl]thiazole-4-carboxylate

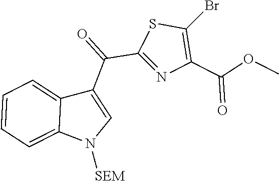 [SMILES: COC(=O)c1nc(C(=O)c2cn(COCC[Si](C)(C)C)c3ccccc23)sc1Br]

To a solution of methyl 5-bromo-2-[hydroxy-[1-(2-trimethylsilylethoxymethyl)indol-3-yl]methyl]thiazole-4-carboxylate (1.6 g, 1.64 mmol, 51% purity, 1 eq) in CHCl₃ (20 mL) was added MnO₂ (2.85 g, 32.81 mmol, 20 eq). The mixture was stirred at 80° C. for 1 h. The mixture was filtered through celite and the cake was rinsed with DCM (30 mL×2). The filtrate was concentrated under reduced pressure to yield a residue which was purified by flash silica gel chromatography (from PE/EtOAc=1/0 to 3/1, TLC:PE/EtOAc=3/1, R_{f}=0.43) to yield methyl 5-bromo-2-[1-(2-trimethylsilylethoxymethyl)indole-3-carbonyl]thiazole-4-carboxylate (500 mg, 807.33 μmol, 49.2% yield, 80.0% purity) as a yellow solid. ¹H NMR (400 MHz, CDCl₃) δ ppm 8.40-8.33 (m, 1H), 7.59-7.52 (m, 2H), 7.44-7.36 (m, 2H), 5.52-5.46 (m, 1H), 5.49 (s, 1H), 3.77 (s, 3H), 3.54-3.47 (m, 2H), 0.95-0.87 (m, 2H), −0.04 (s, 9H); ES-LCMS m/z 495.0, 497.1 [M+H]⁺.

### Step 3: 5-Bromo-2-[1-(2-trimethylsilylethoxymethyl)indole-3-carbonyl]thiazole-4-carboxylic acid

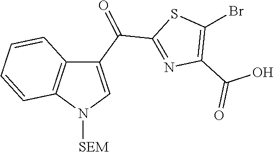 [SMILES: C[Si](C)(C)CCOCn1cc(C(=O)c2nc(C(=O)O)c(Br)s2)c2ccccc21]

To a solution of methyl 5-bromo-2-[1-(2-trimethylsilylethoxymethyl)indole-3-carbonyl]thiazole-4-carboxylate (400 mg, 645.86 μmol, 80.0% purity, 1 eq) in THF (3 mL) was added the mixture of LiOH.H₂O (1 M, 12.00 mL, 18.58 eq) in H₂O (3 mL). The mixture was stirred at 25° C. for 2 h. The mixture was acidified with aqueous HCl (1 M) to pH=5 and extracted with EtOAc (30 mL×3). The combined organic layers were washed with brine (30 mL), dried over Na₂SO₄, filtered and concentrated under reduced pressure to yield 5-bromo-2-[1-(2-trimethylsilylethoxymethyl)indole-3-carbonyl]thiazole-4-carboxylic acid (380 mg, 631.45 μmol, 97.8% yield, 80.0% purity) as a white solid, which was used in the next step without further purification. ¹H NMR (500 MHz, CDCl₃) δ ppm 8.38-8.34 (m, 1H), 7.89 (s, 1H), 7.60-7.56 (m, 1H), 7.46-7.41 (m, 2H), 5.58-5.55 (m, 2H), 3.55 (t, J=8.0 Hz, 2H), 0.93 (t, J=8.0 Hz, 2H), −0.03-−0.04 (m, 9H); ES-LCMS m/z 483.1 [M+H]⁺.

### Step 4: 5-Bromo-2-[1-(2-trimethylsilylethoxymethyl)indole-3-carbonyl]thiazole-4-carboxamide

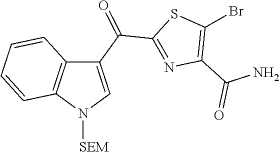 [SMILES: C[Si](C)(C)CCOCn1cc(C(=O)c2nc(C(N)=O)c(Br)s2)c2ccccc21]

To a solution of 5-bromo-2-[1-(2-trimethylsilylethoxymethyl)indole-3-carbonyl]thiazole-4-carboxylic acid (380 mg, 631.45 μmol, 80% purity, 1 eq) in DCM (5 mL) was added NH₄Cl (67.55 mg, 1.26 mmol, 2 eq), Et₃N (191.69 mg, 1.89 mmol, 263.67 μL, 3 eq) and HATU (480.19 mg, 1.26 mmol, 2 eq). The mixture was stirred at 25° C. for 2 h. TLC (PE/EtOAc=1/1, R_{f}=0.45) showed that new point was formed and start material was consumed completely. The reaction mixture was quenched by addition H₂O (20 mL) and extracted with EtOAc (30 mL×3). The combined organic layers were washed with brine (30 mL), dried over Na₂SO₄, filtered and concentrated under reduced pressure to yield a residue which was purified by preparative HPLC (column: Welch Xtimate C18 150*25 mm*5 μm; mobile phase: [water (10 mM NH₄HCO₃)-ACN]; B %: 51%-81%, 10 min) to yield 5-bromo-2-[1-(2-trimethylsilylethoxymethyl)indole-3-carbonyl]thiazole-4-carboxamide (600 mg, 624.41 μmol, 98.9% yield, 50.0% purity) as a white solid. ES-LCMS m/z 482.0 [M+H]⁺.

### Step 5: 5-Bromo-2-[1-(2-trimethylsilylethoxymethyl)indole-3-carbonyl]thiazole-4-carbonitrile

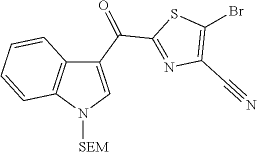 [SMILES: C[Si](C)(C)CCOCn1cc(C(=O)c2nc(C#N)c(Br)s2)c2ccccc21]

To a solution of 5-bromo-2-[1-(2-trimethylsilylethoxymethyl)indole-3-carbonyl]thiazole-4-carboxamide (580 mg, 603.60 μmol, 50.0% purity, 1 eq) in DCM (15 mL) was added TEA (727.00 mg, 1 mL) and TFAA (1.51 g, 1 mL) at 0° C. under N₂ atmosphere. The mixture was stirred at 0° C. for 1 h. The mixture was allowed to warm to room temperature (25° C.) with stirred under N₂ atmosphere for 8 h. TLC (PE/EtOAc=3/1, R_{f}=0.5) showed that new point was formed and start material was consumed completely. The reaction mixture was quenched by addition saturated aqueous NaHCO₃ (20 mL) and extracted with EtOAc (30 mL×3). The combined organic layers were washed with brine (30 mL), dried over Na₂SO₄, filtered and concentrated under reduced pressure to give a residue which was purified by flash silica gel chromatography (from PE/EtOAc=100/1 to 10/1, TLC:PE/EtOAc=10/1, R_{f}=0.40) to yield 5-bromo-2-[1-(2-trimethylsilylethoxymethyl)indole-3-carbonyl]thiazole-4-carbonitrile (380 mg, 575.22 μmol, 95.3% yield, 70.0% purity) as a white solid. ES-LCMS m/z 464.1 [M+H]⁺.

### Step 6: 5-Bromo-2-(1H-indole-3-carbonyl)thiazole-4-carbonitrile

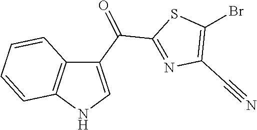 [SMILES: N#Cc1nc(C(=O)c2c[nH]c3ccccc23)sc1Br]

To a solution of 5-bromo-2-[1-(2-trimethylsilylethoxymethyl)indole-3-carbonyl]thiazole-4-carbonitrile (200 mg, 302.75 μmol, 70.0% purity, 1 eq) in DCM (10 mL) was added TFA (8.14 g, 71.35 mmol, 5.28 mL, 235.69 eq). The mixture was stirred at 25° C. for 2 h. The mixture was basified by NaOH (1M) to pH 10. The mixture was stirred at 25° C. for 1 h. The reaction mixture was quenched by addition H₂O (20 mL) and extracted with EtOAc (30 mL×3). The combined organic layers were washed with brine (30 mL), dried over Na₂SO₄, filtered and concentrated under reduced pressure to give a residue which was purified by preparative HPLC (column: Boston Prime C18 150*30 mm*5 μm; mobile phase: [water (0.05% NH₃.H₂O+10 mM NH₄HCO₃)-ACN]; B %: 44%-74%, 10 min) to yield 5-bromo-2-(1H-indole-3-carbonyl)thiazole-4-carbonitrile (6.5 mg, 19.57 μmol, 6.5% yield, 100.0% purity) as a white solid. ¹H NMR (400 MHz, DMSO-d₆) δ ppm 12.55 (s, 1H), 8.47 (d, J=2.8 Hz, 1H), 8.20 (d, J=6.8 Hz, 1H), 7.56 (d, J=7.2 Hz, 1H), 7.37-7.28 (m, 2H); ES-LCMS m/z 334.1 [M+H]⁺.

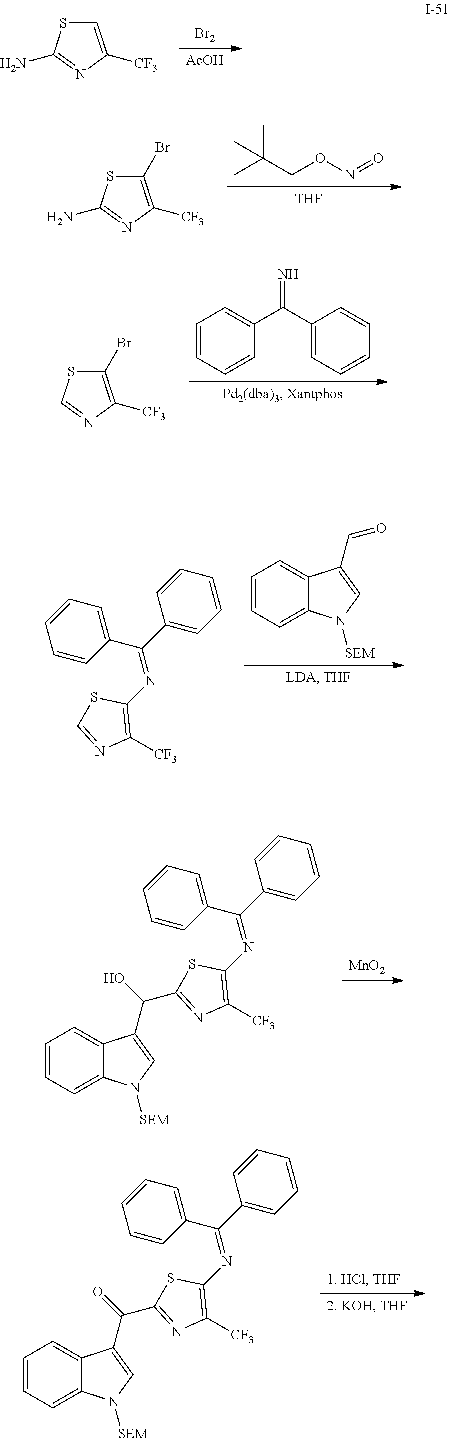

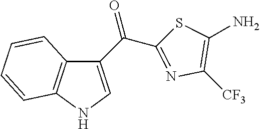 [SMILES: Nc1sc(C(=O)c2c[nH]c3ccccc23)nc1C(F)(F)F]

### Step 1: 5-Bromo-4-(trifluoromethyl)thiazol-2-amine

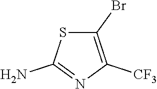 [SMILES: Nc1nc(C(F)(F)F)c(Br)s1]

A solution of 4-(trifluoromethyl)thiazol-2-amine (1.8 g, 10.71 mmol, 1 eq) in AcOH (20 mL) was cooled to 0° C. Br₂ (2.57 g, 16.06 mmol, 827.81 μL, 1.5 eq) was added dropwise at 0° C. The mixture was stirred at 25° C. for 2 h. TLC (PE/EtOAc=3/1, R_{f}=0.55) indicated starting material was consumed completely and one new spot formed. The mixture was filtered and washed with aq. NaHCO₃. The solid was concentrated under reduced pressure to yield 5-bromo-4-(trifluoromethyl)thiazol-2-amine (2.3 g, 9.31 mmol, 87.0% yield, 100.0% purity) as a yellow solid, which was used in the next step without further purification. ¹H NMR (400 MHz, DMSO-d₆) δ ppm 7.69 (s, 2H); ES-LCMS m/z 249.0 [M+H]⁺.

### Step 2: 5-Bromo-4-(trifluoromethyl)thiazole

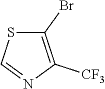

To a solution of 5-bromo-4-(trifluoromethyl)thiazol-2-amine (2.3 g, 9.31 mmol, 100% purity, 1 eq) in THF (25 mL) was added tert-butyl nitrite (1.44 g, 13.97 mmol, 1.66 mL, 1.5 eq). The mixture was stirred at 60° C. for 1 h. The mixture was diluted with water (30 mL) and extracted with EtOAc (30 mL×3). The combined organic layers were dried over Na₂SO₄, filtered and concentrated under reduced pressure to yield 5-bromo-4-(trifluoromethyl)thiazole (2.4 g, 9.31 mmol, 100.0% yield, 90.0% purity) as a yellow oil, which was used in the next step without further purification. ¹H NMR (400 MHz, DMSO-d₆) δ ppm 9.28 (s, 1H).

### Step 3: 1,1-Diphenyl-N-[4-(trifluoromethyl)thiazol-5-yl]methanimine

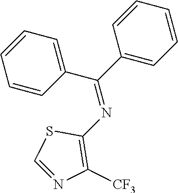 [SMILES: FC(F)(F)c1ncsc1N=C(c1ccccc1)c1ccccc1]

A mixture of 5-bromo-4-(trifluoromethyl)thiazole (1.5 g, 5.82 mmol, 90% purity, 1 eq), diphenylmethanimine (1.27 g, 6.98 mmol, 1.17 mL, 1.2 eq), Cs₂CO₃ (4.17 g, 12.80 mmol, 2.2 eq), xantphos (673.33 mg, 1.16 mmol, 0.2 eq) and Pd₂(dba)₃ (532.80 mg, 581.84 μmol, 0.1 eq) in toluene (15 mL) was degassed and purged with N₂ for 3 times and the mixture was stirred under N₂ atmosphere at 80° C. for 16 h. TLC (PE/EtOAc=3/1, R_{f}=0.65) indicated starting material was consumed completely and many new spots formed. The mixture was diluted with water (20 mL) and extracted with EtOAc (20 mL×3). The combined organic layers were dried over Na₂SO₄, filtered and concentrated under reduced pressure to yield a residue which was purified by flash silica gel chromatography (from PE/EtOAc=100/1 to 10/1, TLC:PE/EtOAc=3/1, R_{f}=0.65) to yield 1,1-diphenyl-N-[4-(trifluoromethyl)thiazol-5-yl]methanimine (1.5 g, 2.26 mmol, 38.8% yield, 50.0% purity) as a yellow solid. ¹H NMR (400 MHz, DMSO-d₆) δ ppm 8.81 (s, 1H), 7.74-7.73 (m, 2H), 7.63-7.59 (m, 4H), 7.55-7.52 (m, 2H), 7.33 (dd, J=1.6, 7.8 Hz, 2H); ES-LCMS m/z 333.6 [M+H]⁺.

### Step 4: [5-(Benzhydrylideneamino)-4-(trifluoromethyl)thiazol-2-yl]-[1-(2-trimethylsilylethoxymethyl)indol-3-yl]methanol

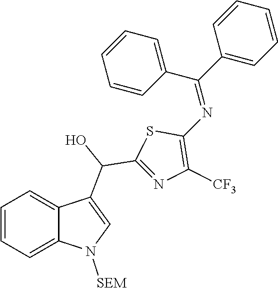 [SMILES: C[Si](C)(C)CCOCn1cc(C(O)c2nc(C(F)(F)F)c(N=C(c3ccccc3)c3ccccc3)s2)c2ccccc21]

A mixture of 1,1-diphenyl-N-[4-(trifluoromethyl)thiazol-5-yl]methanimine (1.5 g, 2.26 mmol, 50% purity, 1 eq) and 1-(2-trimethylsilylethoxymethyl)indole-3-carbaldehyde (512.61 mg, 1.81 mmol, 97% purity, 0.8 eq) in THF (20 mL) was cooled to −75° C. LDA (2 M, 2.26 mL, 2 eq) was added dropwise into the mixture under N₂ atmosphere. The reaction mixture was stirred under N₂ atmosphere at −75° C. for 3 h. The reaction mixture was quenched by addition aq. NH₄Cl (20 mL) at −75° C. The mixture was extracted with EtOAc (20 mL×3). The combined organic layers were dried over Na₂SO₄, filtered and concentrated under reduced pressure to yield a residue which was purified by flash silica gel chromatography (from PE/EtOAc=100/1 to 4/1, TLC:PE/EtOAc=3/1, R_{f}=0.50) to yield [5-(benzhydrylideneamino)-4-(trifluoromethyl)thiazol-2-yl]-[1-(2-trimethylsilylethoxymethyl)indol-3-yl]methanol (720 mg, 947.74 μmol, 42.0% yield, 80.0% purity) as a yellow solid. ¹H NMR (400 MHz, CDCl₃) δ ppm 7.88-7.75 (m, 2H), 7.57-7.35 (m, 8H), 7.29-7.26 (m, 2H), 7.23-7.08 (m, 4H), 6.14 (d, J=4.3 Hz, 1H), 5.47-5.41 (m, 2H), 3.50-3.42 (m, 2H), 0.91-0.86 (m, 2H), −0.03-−0.06 (m, 9H); ES-LCMS m/z 608.3 [M+H]⁺.

### Step 5: [5-(Benzhydrylideneamino)-4-(trifluoromethyl)thiazol-2-yl]-[1-(2-trimethylsilylethoxymethyl)indol-3-yl]methanone

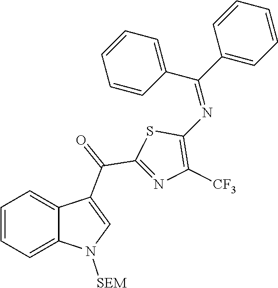 [SMILES: C[Si](C)(C)CCOCn1cc(C(=O)c2nc(C(F)(F)F)c(N=C(c3ccccc3)c3ccccc3)s2)c2ccccc21]

To a solution of [5-(benzhydrylideneamino)-4-(trifluoromethyl)thiazol-2-yl]-[1-(2-trimethylsilylethoxymethyl)indol-3-yl]methanol (650 mg, 855.60 μmol, 80% purity, 1 eq) in CHCl₃ (10 mL) was added MnO₂ (743.86 mg, 8.56 mmol, 10 eq). The mixture was stirred at 76° C. for 5 h. TLC (PE/EtOAc=3/1, R_{f}=0.80) indicated starting material was consumed completely and two new spots formed. The mixture was filtered and concentrated under reduced pressure to yield a residue which was purified by flash silica gel chromatography (from PE/EtOAc=100/1 to 20/1, TLC:PE/EtOAc=3/1, R_{f}=0.80) to yield [5-(benzhydrylideneamino)-4-(trifluoromethyl)thiazol-2-yl]-[1-(2-trimethylsilylethoxymethyl)indol-3-yl]methanone (320 mg, 422.62 μmol, 49.4% yield, 80.0% purity) as a yellow solid. ¹H NMR (400 MHz, DMSO-d₆) δ ppm 8.97 (s, 1H), 8.23 (d, J=7.1 Hz, 1H), 7.78 (d, J=4.9 Hz, 2H), 7.72 (d, J=7.8 Hz, 1H), 7.68-7.62 (m, 4H), 7.57 (d, J=5.1 Hz, 2H), 7.43-7.28 (m, 4H), 5.75 (s, 2H), 3.53 (t, J=8.1 Hz, 2H), 0.85 (t, J=8.1 Hz, 2H), −0.10 (s, 9H); ES-LCMS m/z 606.1 [M+H]⁺.

### Step 6: [5-Amino-4-(trifluoromethyl)thiazol-2-yl]-[1-(hydroxymethyl)indol-3-yl]methanone

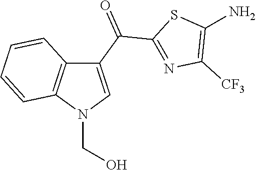 [SMILES: Nc1sc(C(=O)c2cn(CO)c3ccccc23)nc1C(F)(F)F]

To a solution of [5-(benzhydrylideneamino)-4-(trifluoromethyl)thiazol-2-yl]-[1-(2-trimethylsilylethoxymethyl)indol-3-yl]methanone (130 mg, 171.69 μmol, 80% purity, 1 eq) in THF (1.5 mL) was added HCl (12 M, 1.5 mL, 104.84 eq). The mixture was stirred at 25° C. for 12 h. The mixture was concentrated under reduced pressure to yield [5-amino-4-(trifluoromethyl)thiazol-2-yl]-[1-(hydroxymethyl)indol-3-yl]methanone (130 mg, 79.99 μmol, 46.6% yield, 21.0% purity) as a brown solid, which was used in the next step without further purification. ES-LCMS m/z 342.1 [M+H]⁺.

### Step 7: [5-Amino-4-(trifluoromethyl)thiazol-2-yl]-(1H-indol-3-yl)methanone

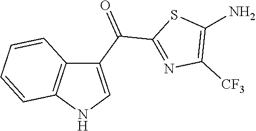 [SMILES: Nc1sc(C(=O)c2c[nH]c3ccccc23)nc1C(F)(F)F]

To a solution of [5-amino-4-(trifluoromethyl)thiazol-2-yl]-[1-(hydroxymethyl)indol-3-yl]methanone (130 mg, 79.99 μmol, 21% purity, 1 eq) in THF (2 mL) was added KOH (440 mg, 7.84 mmol, 98.05 eq) in H₂O (4 mL). The mixture was stirred at 25° C. for 10 min. The mixture was diluted with water (10 mL) and extracted with EtOAc (10 mL×3). The combined organic layers were dried over Na₂SO₄, filtered and concentrated under reduced pressure to yield a residue which was purified by preparative TLC (PE/EtOAc=3/1, R_{f}=0.40). The residue was lyophilized to yield [5-amino-4-(trifluoromethyl)thiazol-2-yl]-(1H-indol-3-yl)methanone (15 mg, 48.19 μmol, 60.2% yield, 100.0% purity) as a yellow solid. ¹H NMR (400 MHz, CDCl₃) δ ppm 9.02 (d, J=3.2 Hz, 1H), 8.79 (br s, 1H), 8.51-8.47 (m, 1H), 7.48-7.45 (m, 1H), 7.35-7.32 (m, 2H), 4.93 (br s, 2H); ES-LCMS m/z 312.2 [M+H]⁺.

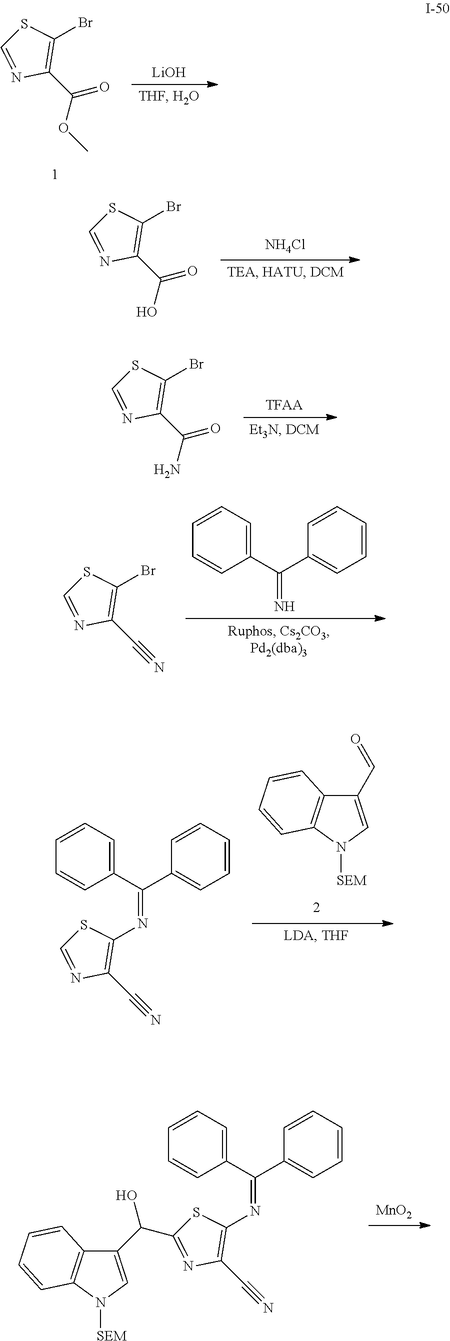

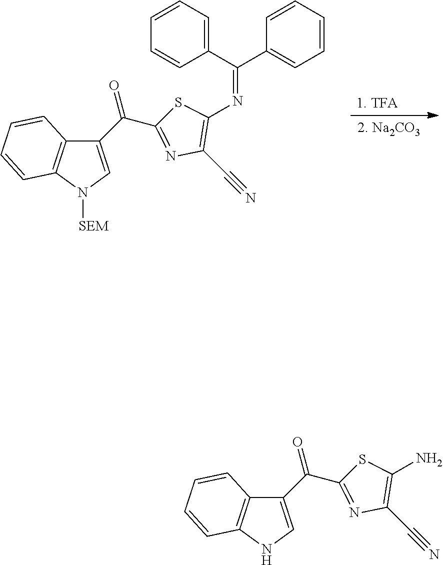 [SMILES: C[Si](C)(C)CCOCn1cc(C(=O)c2nc(C#N)c(N=C(c3ccccc3)c3ccccc3)s2)c2ccccc21 ; "1. TFA" ; "2. Na2CO3" ; N#Cc1nc(C(=O)c2c[nH]c3ccccc23)sc1N]

### Step 1: 5-Bromothiazole-4-carboxylic acid

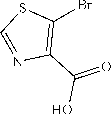

To a solution of methyl 5-bromothiazole-4-carboxylate (3.16 g, 13.51 mmol, 95.0% purity, 1 eq) in THF (15 mL) was added LiOH.H₂O (1 M, 15 mL, 1.1 eq). The mixture was stirred at 25° C. for 2 h. TLC (PE/EtOAc=3/1, R_{f}=0.1) showed that new point was formed and start material was consumed completely. The reaction mixture was quenched by addition H₂O (100 mL) and extracted with EtOAc (100 mL×3). The combined organic layers were washed with brine (100 mL), dried over Na₂SO₄, filtered and concentrated under reduced pressure to yield 5-bromothiazole-4-carboxylic acid (2.5 g, 11.42 mmol, 84.5% yield, 95.0% purity) as a white solid, which was used in the next step without further purification. ¹H NMR (400 MHz, DMSO-d₆) δ=13.35 (s, 1H), 9.14 (s, 1H).

### Step 2: 5-Bromothiazole-4-carboxamide

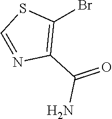

To a solution of 5-bromothiazole-4-carboxylic acid (2.5 g, 10.82 mmol, 90.0% purity, 1.0 eq) and NH₄Cl (867.79 mg, 16.22 mmol, 1.5 eq) in DCM (20 mL) was added HATU (4.93 g, 12.98 mmol, 1.2 eq) and Et₃N (3.28 g, 32.45 mmol, 4.52 mL, 3.0 eq). The mixture was stirred at 25° C. for 3 h. TLC (PE/EtOAc=1/1, R_{f}=0.4) showed that new point was formed and start material was consumed completely. The reaction mixture was quenched by addition of H₂O (100 mL) and extracted with EtOAc (80 mL×3). The combined organic layers were washed with brine (80 mL), dried over Na₂SO₄, filtered and concentrated under reduced pressure to yield 5-bromothiazole-4-carboxamide (2.5 g, 9.66 mmol, 89.3% yield, 80.0% purity) as a white solid, which was used in the next step without further purification. ES-LCMS: no desired MS found.

### Step 3: 5-Bromothiazole-4-carbonitrile

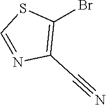

To a solution of 5-bromothiazole-4-carboxamide (2.2 g, 8.50 mmol, 80.0% purity, 1 eq) in DCM (10 mL) was added Et₃N (4.27 g, 42.15 mmol, 5.87 mL, 4.96 eq) and TFAA (8.86 g, 42.18 mmol, 5.87 mL, 4.96 eq) at 0° C. The mixture was stirred at 0° C. for 2 h. TLC (PE/EtOAc=1/1, R_{f}=0.6) showed that new point was formed and start material was consumed completely. The reaction mixture was quenched by addition of H₂O (80 mL) and extracted with EtOAc (80 mL×3). The combined organic layers were washed with brine (80 mL), dried over Na₂SO₄, filtered and concentrated under reduced pressure to yield a residue which was purified by flash silica gel chromatography (from PE/EtOAc=100/1 to 8/1, TLC:PE/EtOAc=10/1, R_{f}=0.60) to yield 5-bromothiazole-4-carbonitrile (1.28 g, 6.09 mmol, 71.7% yield, 90.0% purity) as a yellow solid. ¹H NMR (400 MHz, DMSO-d₆) δ ppm 9.28 (s, 1H).

### Step 4: 5-(Benzhydrylideneamino)thiazole-4-carbonitrile

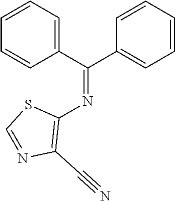 [SMILES: N#Cc1ncsc1N=C(c1ccccc1)c1ccccc1]

To a solution of 5-bromothiazole-4-carbonitrile (1.28 g, 6.09 mmol, 90% purity, 1 eq) and diphenylmethanimine (1.10 g, 6.09 mmol, 1.02 mL, 1 eq) in toluene (3 mL) was added Xantphos (634.72 mg, 1.10 mmol, 0.18 eq), Cs₂CO₃ (4.37 g, 13.41 mmol, 2.2 eq) and Pd₂(dba)₃ (334.83 mg, 365.65 μmol, 0.06 eq) under N₂ atmosphere. The mixture was stirred under N₂ atmosphere at 90° C. for 3 h. TLC (PE/EtOAc=3/1, R_{f}=0.5) showed that new point was formed and start material was consumed completely. The reaction mixture was quenched by addition of H₂O (80 mL) and extracted with EtOAc (80 mL×3). The combined organic layers were washed with brine (80 mL), dried over Na₂SO₄, filtered and concentrated under reduced pressure to yield a residue which was purified by flash silica gel chromatography (from PE/EtOAc=100/1 to 85/15, TLC:PE/EtOAc=3/1, R_{f}=0.50) to yield 5-(benzhydrylideneamino)thiazole-4-carbonitrile (900 mg, 3.11 mmol, 51.0% yield, 100.0% purity) as a white solid. ¹H NMR (400 MHz, CDCl₃) δ ppm 8.39 (s, 1H), 7.91 (d, J=7.6 Hz, 2H), 7.65-7.53 (m, 4H), 7.48-7.42 (m, 2H), 7.24 (d, J=6.6 Hz, 2H); ES-LCMS m/z 290.2 [M+H]⁺.

### Step 5: 5-(Benzhydrylideneamino)-2-[hydroxy-[1-(2-trimethylsilylethoxymethyl)indol-3-yl]methyl]thiazole-4-carbonitrile

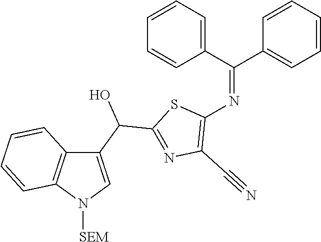 [SMILES: C[Si](C)(C)CCOCn1cc(C(O)c2nc(C#N)c(N=C(c3ccccc3)c3ccccc3)s2)c2ccccc21]

To a solution of 1-(2-trimethylsilylethoxymethyl)indole-3-carbaldehyde (300 mg, 1.03 mmol, 95.0% purity, 1 eq) and 5-(benzhydrylideneamino)thiazole-4-carbonitrile (299.42 mg, 1.03 mmol, 100.0% purity, 1 eq) in THF (8 mL) was added LDA (2 M, 1.29 mL, 2.5 eq) at −70° C. under N₂ atmosphere. The mixture was stirred under N₂ atmosphere at −70° C. for 3 h. TLC (PE/EtOAc=3/1, R_{f}=0.45) showed that new point was formed and start material was consumed completely. The reaction mixture was quenched by saturated NH₄Cl (40 mL) and extracted with EtOAc (30 mL×3). The combined organic layers were washed with brine (30 mL), dried over Na₂SO₄, filtered and concentrated under reduced pressure to yield a residue which was purified by flash silica gel chromatography (from PE/EtOAc=100/1 to 2/1, TLC:PE/EtOAc=3/1, R_{f}=0.45) to yield 5-(benzhydrylideneamino)-2-[hydroxy-[1-(2-trimethylsilylethoxymethyl)indol-3-yl]methyl]thiazole-4-carbonitrile (285 mg, 479.40 μmol, 46.3% yield, 95.0% purity) as a white solid. ¹H NMR (400 MHz, DMSO-d₆) δ ppm 7.79-7.68 (m, 3H), 7.67-7.59 (m, 3H), 7.52 (dd, J=7.6, 14.8 Hz, 3H), 7.41-7.34 (m, 4H), 7.16 (t, J=7.6 Hz, 1H), 7.08-6.99 (m, 1H), 6.64 (d, J=4.4 Hz, 1H), 5.98 (d, J=4.4 Hz, 1H), 5.49 (s, 2H), 3.42 (t, J=8.0 Hz, 2H), 0.83-0.75 (m, 2H), −0.09 (s, 9H); ES-LCMS m/z 565.2 [M+H]⁺.

### Step 6: 5-(Benzhydrylideneamino)-2-[1-(2-trimethylsilylethoxymethyl)indole-3-carbonyl]thiazole-4-carbonitrile

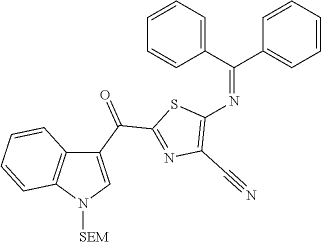 [SMILES: C[Si](C)(C)CCOCn1cc(C(=O)c2nc(C#N)c(N=C(c3ccccc3)c3ccccc3)s2)c2ccccc21]

To a solution of methyl 5-(benzhydrylideneamino)-2-[hydroxy-[7-(2-trimethylsilylethoxymethyl)pyrrolo[2,3-d]pyrimidin-5-yl]methyl]thiazole-4-carboxylate (60 mg, 75.03 μmol, 75% purity, 1 eq) in CHCl₃ (5 mL) was added MnO₂ (195.69 mg, 2.25 mmol, 30 eq). The mixture was stirred at 70° C. for 3 h. The reaction mixture was filtered and concentrated under reduced pressure to yield methyl 5-(benzhydrylideneamino)-2-[7-(2-trimethylsilylethoxymethyl)pyrrolo[2,3-d]pyrimidine-5-carbonyl]thiazole-4-carboxylate (30 mg, 40.15 μmol, 53.5% yield, 80.0% purity) as a white solid, which was used in the next step without further purification. ES-LCMS m/z 563.2 [M+H]⁺.

### Step 7: 5-Amino-2-(1H-indole-3-carbonyl)thiazole-4-carbonitrile

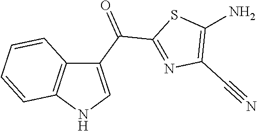 [SMILES: N#Cc1nc(C(=O)c2c[nH]c3ccccc23)sc1N]

To a solution of 5-(benzhydrylideneamino)-2-[1-(2-trimethylsilylethoxymethyl)indole-3-carbonyl]thiazole-4-carbonitrile (240 mg, 341.18 μmol, 80% purity, 1 eq) in DCM (20 mL) was added TFA (7.10 g, 62.24 mmol, 4.61 mL, 182.42 eq). The mixture was stirred at 25° C. for 2 h. Saturated aqueous NaHCO₃ (20 mL) was added into the mixture with stirred at 25° C. for 2 h. The reaction mixture was quenched by addition H₂O of (20 mL) and extracted with EtOAc (30 mL×3). The combined organic layers were washed with brine (30 mL), dried over Na₂SO₄, filtered and concentrated under reduced pressure to yield a residue which was purified by preparative HPLC (column: Welch Xtimate C18 150*25 mm*5 μm; mobile phase: [water (10 mM NH₄HCO₃)-ACN]; B %: 30%-60%, 10 min) to yield 5-amino-2-(1H-indole-3-carbonyl)thiazole-4-carbonitrile (18.99 mg, 69.65 μmol, 20.4% yield, 98.4% purity) as a white solid. ¹H NMR (400 MHz, DMSO-d₆) δ ppm 12.17 (s, 1H), 8.84 (s, 1H), 8.24 (dd, J=2.0, 6.2 Hz, 1H), 8.17 (s, 2H), 7.58-7.53 (m, 1H), 7.29-7.21 (m, 2H); ES-LCMS m/z 269.2 [M+H]⁺.

### Example 2. In Vitro Assay

### DRE-Luciferase Reporter Assay

AHR binds to Dioxin Responsive Elements (DRE) upstream of genes that it activates. One measure of AHR activity is activation of a reporter gene, such as luciferase, downstream of one or multiple DRE elements. Luciferase activity will reflect activation and inhibition of AHR in the cells expressing this reporter.

Murine Hepa1-6 or Hepa-1c1c7 or other murine cell line with a DRE-luciferase reporter either stably or transiently transfected are plated in media in plates (96-well, 384-well or other plates) and incubated overnight at 37 C in a CO2 incubator. Likewise, human HepG2 or other human cell line with a DRE-luciferase reporter either stably or transiently transfected are plated in media in plates (96-well, 384-well or other plates) and incubated overnight at 37 C in a CO₂ incubator.

The next day, an AHR agonist compound is added. Cells are incubated for 6, 16 or 24 hours or another time point and then lysed for determination of luciferase activity as a read-out of the AHR activation. Luciferase can be measured with a commercial kit such as the Promega Luciferase kit or any kit or reagents that provide the luciferin substrate for measuring luciferase activity. The level of luciferase with only activating ligand (e.g. such as TCDD, kynurenine, ITE (2-(1H-indole-3-ylcarbonyl)-4-thiazolecarboxylic methyl ester), VAF347, BNF (beta-naphthoflavone), ICZ (6-formylindolo(3,2-b) carbazole or other AHR ligands) added is the maximum signal while the luciferase with no ligand is the minimum signal. EC50s can be determined as the concentration which activates half of the maximum luciferase activity.

In some embodiments, compounds have an EC50>1 μM. In some embodiments, compounds have an EC50<1 μM. In some embodiments, compounds have an EC50<0.1 μM. In some embodiments, compounds have an EC50<0.01 μM.

### P450 CYP1A1 Luciferase Assay

AHR binds to Dioxin Responsive Elements (DRE) upstream of genes that it activates. One measure of AHR activity is P450 CYP1A1 protein levels determined by measuring CYP1A1 enzyme activity using a luminogenic CYP1A1 luciferin-based substrate. Luciferase activity will reflect CYP1A1 activity resulting from activation of AHR in the cells.

Murine Hepa1-6 or Hepa-1c1c7 or other murine cell line, human HepG2 or other human cell line are plated in media (96-well, 384-well or other plates) and incubated overnight at 37 C in a CO2 incubator.

The next day, an AHR agonist compound is added. Cells are incubated for 6, 16 or 24 hours or another time point and then lysed and incubated with a CYP1A1 luciferase-based substrate (e.g., Luciferin-CEE) for 3, 6, or 12 hours of another time point. Determination of luciferase activity as a read-out of CYP1A1 enzyme activity can be measured with a commercial kit such as the Promega P450 Glo CYP1A1 detection reagent or any kit or reagents that provide for measuring luciferase activity. The level of luciferase with only activating ligand (e.g., such as TCDD, kynurenine, ITE (2-(1H-indole-3-ylcarbonyl)-4-thiazolecarboxylic methyl ester), VAF347, BNF (beta-naphthoflavone), ICZ (6-formylindolo(3,2-b) carbazole or other AHR ligands) added is the maximum signal while the luciferase with no ligand is the minimum signal. EC50s can be determined as the concentration which activates half of the maximum luciferase activity.

Certain compounds were tested in the assays. The data are listed in Table 2 below. A: EC50≤0.010 μM; B: 0.010 μM<EC50≤0.1 μM; C: 0.1 μM<EC50≤1.0 μM; and D: EC50>1.0 μM.

TABLE 2
In vitro Data of Certain Exemplary Compounds.

| | DRE-Luc | Cyp1a1 | | DRE-Luc | Cyp1a1 |
|---|---|---|---|---|---|
| | HepG2 - | Hepa1.6 - | | HepG2 - | Hepa1.6 - |
| | Agonist: | Agonist: | | Agonist: | Agonist: |
| Com- | Average | Average | Com- | Average | Average |
| pound | EC50 | EC50 | pound | EC50 | EC50 |
| # | (μM) | (μM) | # | (μM) | (μM) |
| I-1 | A | | P-1 | A | |
| I-2 | A | D | P-2 | A | D |
| I-3 | B | | P-3 | B | D |
| I-4 | C | D | P-4 | B | D |
| I-5 | C | D | P-5 | B | |
| I-6 | C | | P-6 | C | |
| I-7 | C | D | P-7 | C | |
| I-8 | C | D | P-8 | C | |
| I-9 | C | D | P-9 | C | |
| I-10 | C | D | P-10 | C | |
| I-11 | C | D | P-11 | D | |
| I-12 | C | | P-12 | D | |
| I-13 | C | | P-13 | D | |
| I-14 | C | | P-14 | D | |
| I-15 | C | | P-15 | D | |
| I-16 | C | | P-16 | D | |
| I-17 | C | | P-17 | D | |
| I-18 | D | D | P-18 | D | |
| I-19 | D | D | P-19 | D | |
| I-20 | D | D | | | |
| I-21 | D | | | | |
| I-22 | D | | | | |
| I-23 | D | D | | | |
| I-24 | D | | | | |
| I-25 | D | | | | |
| I-26 | D | | | | |
| I-27 | D | | | | |
| I-28 | D | | | | |
| I-29 | D | | | | |
| I-30 | D | | | | |
| I-31 | D | | | | |
| I-32 | D | | | | |
| I-33 | D | | | | |
| I-34 | D | | | | |
| I-35 | D | | | | |
| I-36 | D | | | | |
| I-37 | D | | | | |
| I-38 | D | | | | |
| I-39 | D | | | | |
| I-40 | D | | | | |
| I-41 | D | | | | |
| I-42 | D | | | | |
| I-43 | D | | | | |
| I-44 | D | | | | |
| I-45 | D | | | | |
| I-46 | D | | | | |
| I-47 | D | D | | | |

Certain compounds were tested in the assays. The data are listed in Table 3 below. A: EC50≤0.010 μM; B: 0.010 μM<EC50≤0.1 μM; C: 0.1 μM<EC50≤1.0 μM; and D: EC50>1.0 μM.

TABLE 3
In vitro Data of Certain Exemplary Compounds.

| | DRE-Luc | DRE-Luc |
|---|---|---|
| | HepG2 - | Hepa1.6 - |
| | Agonist: | Agonist: |
| | Average | Average |
| Compound # | EC50 (μM) | EC50 (μM) |
| I-50 | A | A |
| I-51 | A | A |
| I-52 | B | D |
| I-53 | A | B |
| I-54 | A | D |
| I-55 | A | B |
| I-56 | A | A |
| I-57 | A | A |
| I-58 | B | B |
| I-59 | C | C |
| I-60 | A | A |
| I-61 | A | A |
| I-62 | D | D |
| I-63 | B | D |
| I-64 | A | B |
| I-65 | B | B |
| I-66 | D | D |
| I-67 | C | D |
| I-68 | A | A |
| I-69 | A | B |
| I-70 | D | D |
| I-71 | C | B |
| I-72 | D | B |
| I-73 | B | A |
| I-74 | A | A |
| I-75 | D | D |
| I-76 | D | D |
| I-77 | A | A |
| I-78 | C | D |
| I-79 | A | C |
| I-80 | B | C |
| I-81 | C | D |
| I-82 | C | A |
| I-83 | D | D |
| I-84 | A | A |
| I-85 | D | C |
| I-86 | D | D |
| I-87 | A | A |
| I-88 | D | D |
| I-89 | D | D |
| I-90 | B | B |
| I-91 | D | D |
| I-92 | A | A |
| I-93 | C | B |
| I-94 | D | D |
| I-95 | B | D |
| I-96 | A | A |
| I-97 | C | D |

### Example 3. Liver and Colon Pharmacodynamics (PD) Assays and Methods

C57BL/6N mice are weighed and randomized into treatment groups with group size of 3-5 mice. On study Day 1, treatment is initiated and necropsies follow on day 1 at 4 and 12 hours post-dose and on Day 2, 24 hours post-dose.

On Day 1, mice are dosed orally with one dose of the AHR agonist compound(s) that are in a suspension and mixed well before dosing. At the designated time, animals are euthanized and plasma and tissue taken for compound levels (PK) and compound effect (PD) on gene expression. Liver samples and proximal colon are weighed and then frozen for subsequent RNA extraction and RT-PCR analysis. AHR activation is determined by measuring Cyp1a1 gene expression relative to a housekeeping gene, such as GAPDH or HPRT. Cyp1a1 expression levels in the liver are compared to Cyp1a1 levels in the colon to determine a colon:liver ratio, in order to assess the level of “GI-preferred” AHR activation.

### Example 4: DSS IBD Study Method

On study day −1, C57Bl/6 mice are weighed and randomized into treatment groups based on body weight. On study day 0, treatment groups are given 2.5% DSS in drinking water and treatment is initiated on the same day, with either vehicle or AHR agonist compound(s).

On study day 7, DSS drinking water is replaced with normal drinking water for the remainder of the study. Body weight is measured daily during the entire study.

On study day 10, animals are anesthetized with Isoflurane and bled to exsanguination followed by cervical dislocation. The entire colon is removed and measured for length, weight, and weight per length. Overall efficacy of test AR agonist compounds is based on body weight, colon length, and colon histopathology.

Histopathology data is assessed for appropriate parameters, as determined by a pathologist and the parameters for these DSS studies can include inflammation, erosion, gland loss, edema, hyperplasia, neutrophil count, mucosal thickening, lymphoid aggregate count and lymphoid aggregate size. The different parameter scores can be added for a summed score for the study histopathology.

### Example 5: Th17 Assay

On Day 1, naive CD62L+ human T-Cells are plated in a 96 well plate (25,000 cells in 200 uL media). Cells are activated with human CD3/CD28 tetramer (12.5 μL/1×10⁶ cells) and differentiated with human Th17 cytokines (50 ng/mL IL-6, 20 ng/mL IL-1 (3, 10 ng/mL IL-23, 1 ng/mL TGF-β, 12 μg/mL anti-human IFN-γ antibody and 10 μg/mL anti-human IL-4 antibody) for 10 days. Media containing cytokine cocktail and CD3/CD28 is refreshed every 2-3 days.

On Day 10, cell supernatant is collected and frozen for cytokine analysis. Cells are stimulated with 1× Cell Stimulation Cocktail (PMA and Ionomycin) for 5 hours. After 5 hours of stimulation, cells are stained for intracellular cytokines (human CD4, IL-17A, IL-22). Samples are run on BD LSR FORTESSA and analyzed in FLOWJO software.

### Example 6: Treg Assay

On day 0, nave T cells from cryopreserved human derived PBMCs are isolated. These cells are plated in 48 well plate at 500,000 cells/mL concentration with human CD3/CD28 activation tetramer (12.5 μL/1×10⁶ cells) and differentiated into regulatory T cells (Tregs) with 1 ng/mL TGF-β and 5 ng/mL human recombinant IL-2 in the presence of DMSO or different concentrations of AHR agonist compounds.

On day 5, the Tregs are counted and washed. CD25-Effector T cells (Teffs) are isolated from the same human donor and labeled with Cell Trace Violet. The Tregs and Teffs are cocultured for 4 days in 96 well plate at 1:2 or 1:1 ratio with human CD3/CD28 tetramer (12.5 μL/1×10⁶ cells).

At the end of a 4 day co-culture, the cells are washed and stained with LiveDead stain. The cells are run on a flow cytometer and analyzed using FLOWJO software.

### Example 7: T Cell Transfer IBD Model

On study day 0, donor Balb/C mice are terminated, and spleens obtained for CD4⁺CD45RB^{high} cell isolation (Using a SCID IBD Cell Separation Protocol). After cells have been sorted and obtained, each recipient SCID animal receives an IP injection of, at a minimum, 4×10⁵ cells (200 μl/mouse injections).

Also on study day 0, SCID mice are weighed and randomized into treatment groups based on body weight. On study day 14, AHR agonist compound treatments are initiated and dosed orally daily; the control group receiving anti-IL12 (0.5 mg/mouse) is dosed IP once a week.

On study day 49, animals are anesthetized with Isoflurane and bled to exsanguination followed by cervical dislocation. The entire colon is removed, measured, and weighed. Overall efficacy of AHR agonist compounds are based on a ratio of colon weight to length, and colon histopathology and colon cytokines (Th17 panel).

### Example 8: IBD Ex Vivo Treat Methods

The studies described herein are to assess the effect of various AHR agonist compounds in human Crohn's and ulcerative colitis tissue cultures ex vivo. Following this culture, the resulting culture supernatant samples are collected for analysis of cytokine release. Briefly, Crohn's Disease or ulcerative colitis donor samples are obtained with full ethical consent from patients undergoing therapeutic resection for Crohn's disease or ulcerative colitis. A minimum of 18×5 mm² mucosal biopsies are taken using a scalpel. Three baseline biopsy samples are collected at time 0, and a minimum of 9 biopsies are incubated in 12 well culture plates. Tissues are placed apical (mucosal) side facing upwards on a Netwell filter. The biopsies are then cultured in either control media or media fortified with the appropriate AHR agonist compound in an incubator at 37° C. and high 02 atmospheric conditions (95% 02/5% CO₂). To minimize variation, the biopsies are cultured in the presence of the inflammatory stimulant Staphylococcal Enterotoxin B (SEB) to normalize cytokine levels. The positive control BIRB796 (Selleck Chemicals catalogue No: S1574) is purchased as a powder. A 1 mM stock solution is prepared in DMSO and used at 1 μM. At approximately 18 hours post-culture start, media samples are collected, protease inhibitor is added and samples are stored at −80° C. Supernatant is collected at the 18-hour timepoint and divided into aliquots for cytokine analysis: analysis of cytokines, such as TNF-α, IFN-γ, IL-1β, IL17-α, IL-22, and IL-10) are performed in duplicate after completion of each set of 3 donors.

While we have described a number of embodiments of this invention, it is apparent that our basic examples may be altered to provide other embodiments that utilize the compounds and methods of this invention. Therefore, it will be appreciated that the scope of this invention is to be defined by the application and appended claims rather than by the specific embodiments that have been represented by way of example.

1. A compound of Formula (I-a): 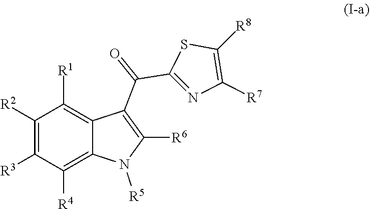
or a pharmaceutically acceptable salt thereof, wherein
each of R¹, R², R³, R⁴, R⁶ and R⁸ is independently halogen, —CN, —NO₂, R^{W}, —C(O)—R^{W}, —C(═NR^{W})—R^{W}, —N(R^{W})—C(O)—R^{W}, —N(R^{W})—C(═NR^{W})—R^{W}, —OC(O)—R^{W}, —OC(═NR^{W})—R^{W}, —S(O)₂—R^{W}, —N(R^{W})—S(O)₂—R^{W}, —OS(O)₂—R^{W}, —S(O)—R^{W}, —N(R^{W})—S(O)—R^{W}, or —OS(O)—R^{W};
R⁵ is —R, —C(O)—R^{W}, —C(═NR^{W})—R^{W}, —S(O)₂—R^{W}, or —S(O)—R^{W};
R⁷ is halogen, —CN, —NO₂, R^{W}, —C(O)R^{W}, —C(═NR^{W})—R^{W}, —N(R^{W})—C(O)—R^{W}, —N(R^{W})—C(═NR^{W})—R^{W}, —OC(O)—R^{W}, —OC(═NR^{W})R^{W}, —S(O)₂—R^{W}, —N(R^{W})—S(O)₂—R^{W}, —OS(O)₂—R^{W}, —S(O)—R^{W}, —N(R^{W})S(O)R^{W}, or —OS(O)—R^{W};
R^{W} is —R, —N(R)₂, —NR—OR, —N(R)—N(R)₂, —N(OR)—N(R)₂, —N(R)—N(OR)R, —OR, —O—N(R)₂, or —SR; and
R is hydrogen, optionally substituted C₁₋₆ aliphatic, an optionally substituted 3-7 membered carbocyclic ring, or an optionally substituted 3-7 membered heterocyclic ring having 1-3 heteroatoms independently selected from N, O, or S, or two R's together with the nitrogen to which they attach form an optionally substituted 5-7 membered heterocyclic ring having 0-2 heteroatoms independently selected from N, O, or S in addition to the nitrogen to which the two R's attach,
provided that the compound is not

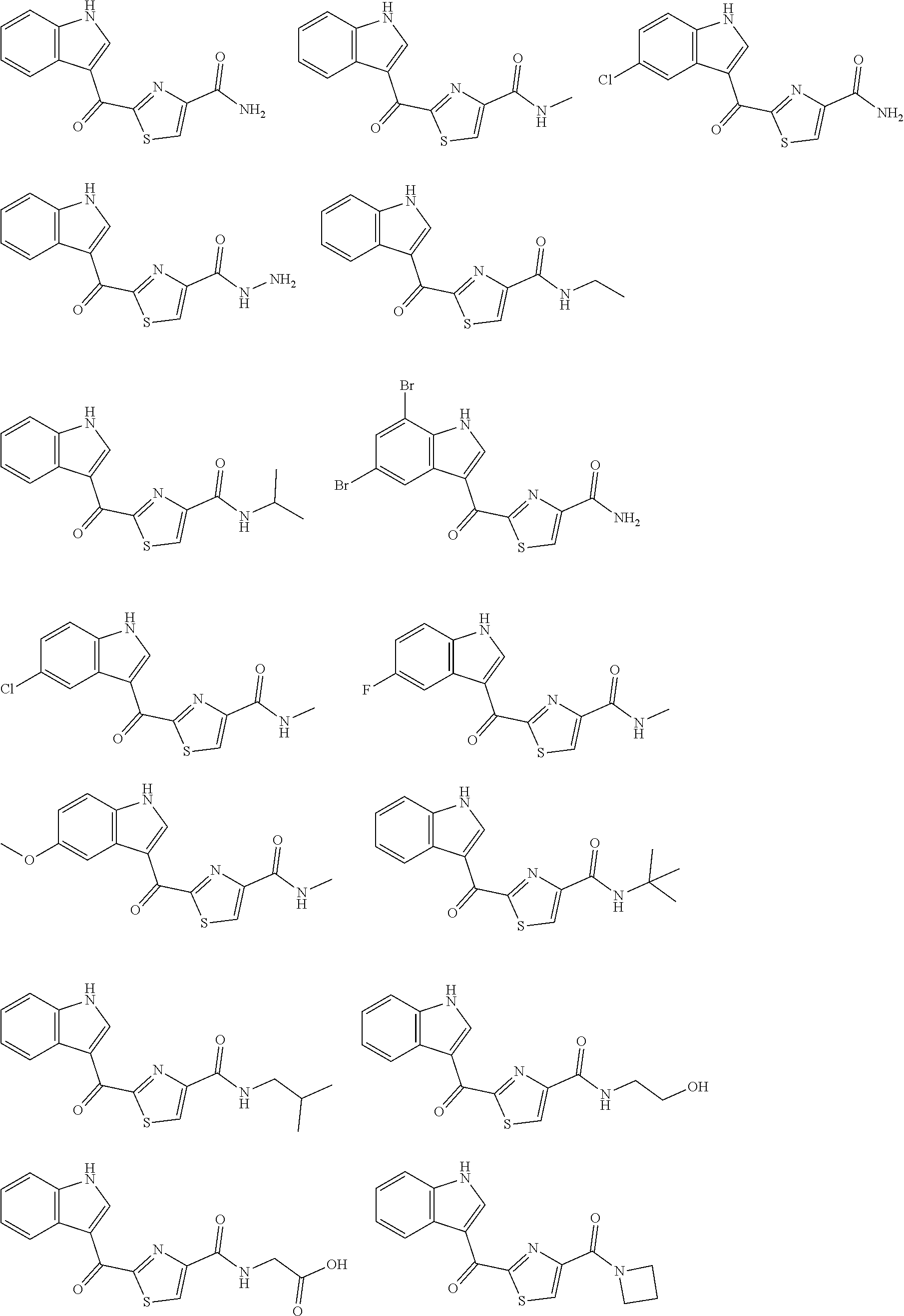

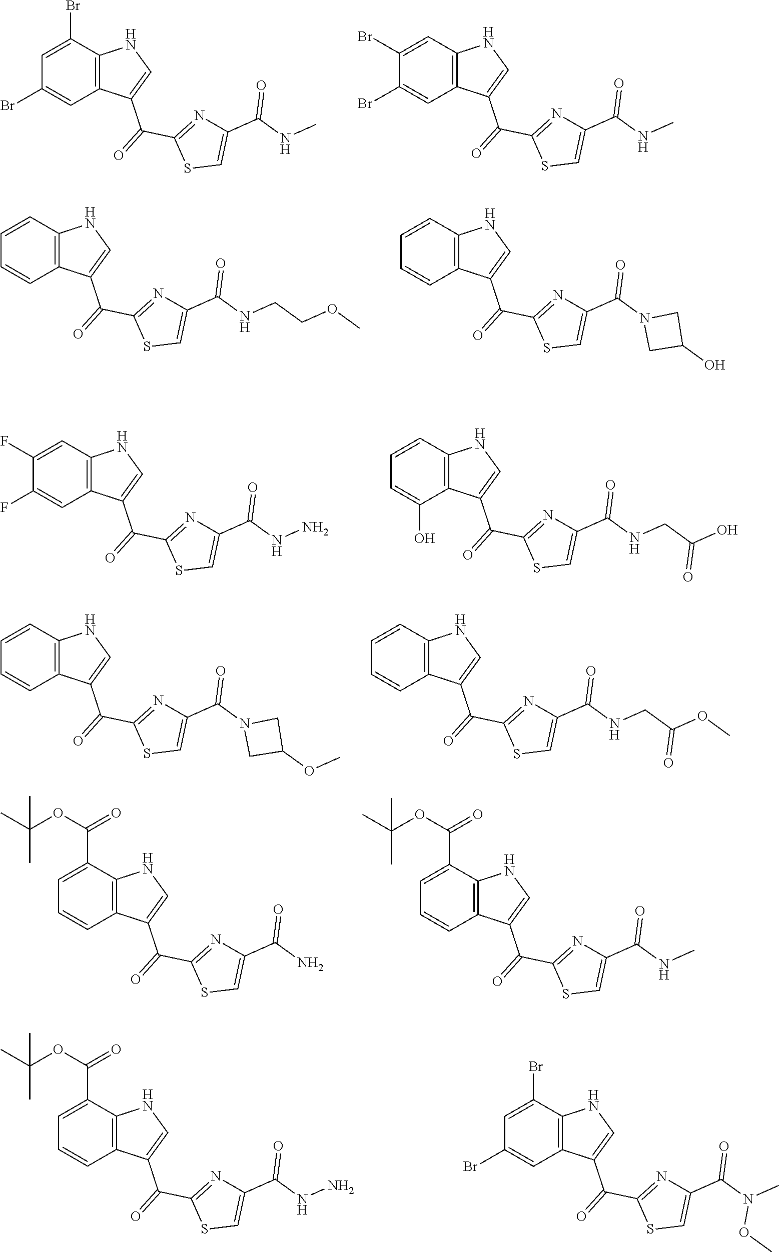

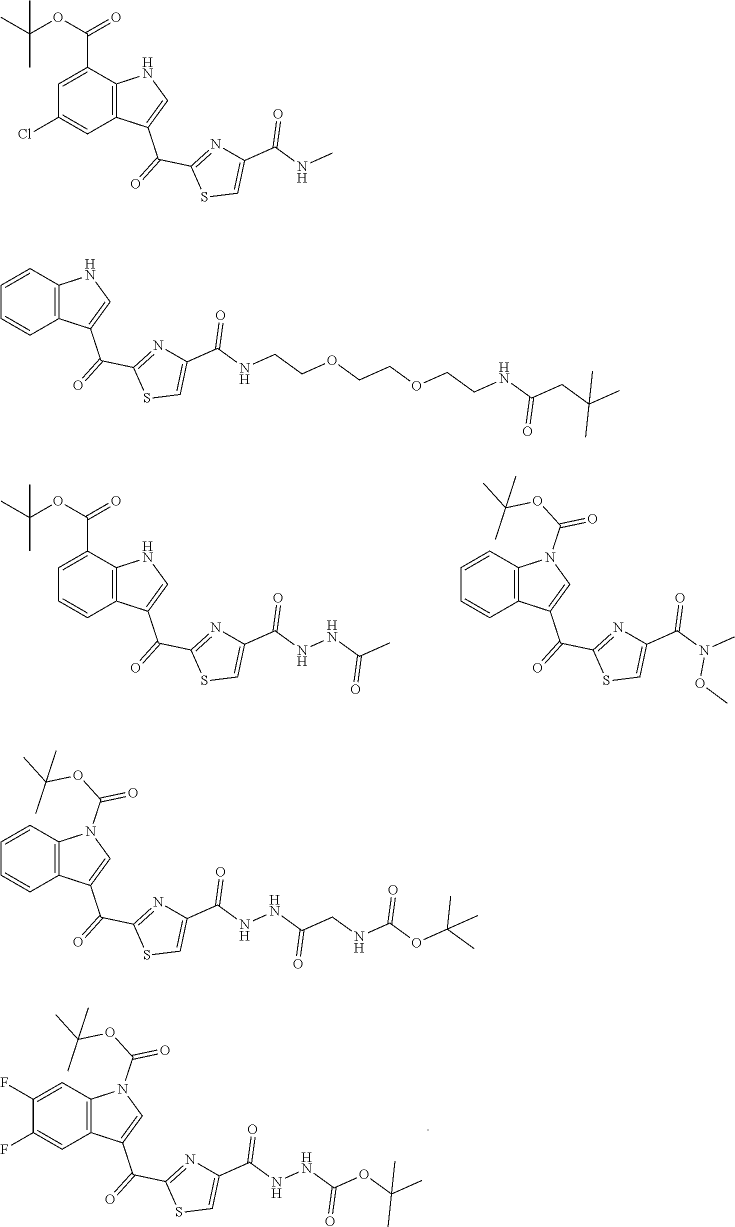

2. The compound of claim 1, or a pharmaceutically acceptable salt thereof, wherein R⁵ is H.

3. The compound of claim 1, or a pharmaceutically acceptable salt thereof, wherein R⁸ is R^{W}.

4. The compound of claim 1, or a pharmaceutically acceptable salt thereof, wherein R⁷ is —C(O)—R^{W}.

5. The compound of claim 1, or a pharmaceutically acceptable salt thereof, wherein R¹ is H, or optionally substituted C₁₋₆ aliphatic or —OC₁₋₆ aliphatic.

6. The compound of claim 1, or a pharmaceutically acceptable salt thereof, wherein R² is H, or optionally substituted C₁₋₆ aliphatic or —OC₁₋₆ aliphatic.

7. The compound of claim 1, or a pharmaceutically acceptable salt thereof, wherein R³ is H, or optionally substituted C₁₋₆ aliphatic or —OC₁₋₆ aliphatic.

8. The compound of claim 1, or a pharmaceutically acceptable salt thereof, wherein R⁴ is H, or optionally substituted C₁₋₆ aliphatic or —OC₁₋₆ aliphatic.

9. A compound selected from 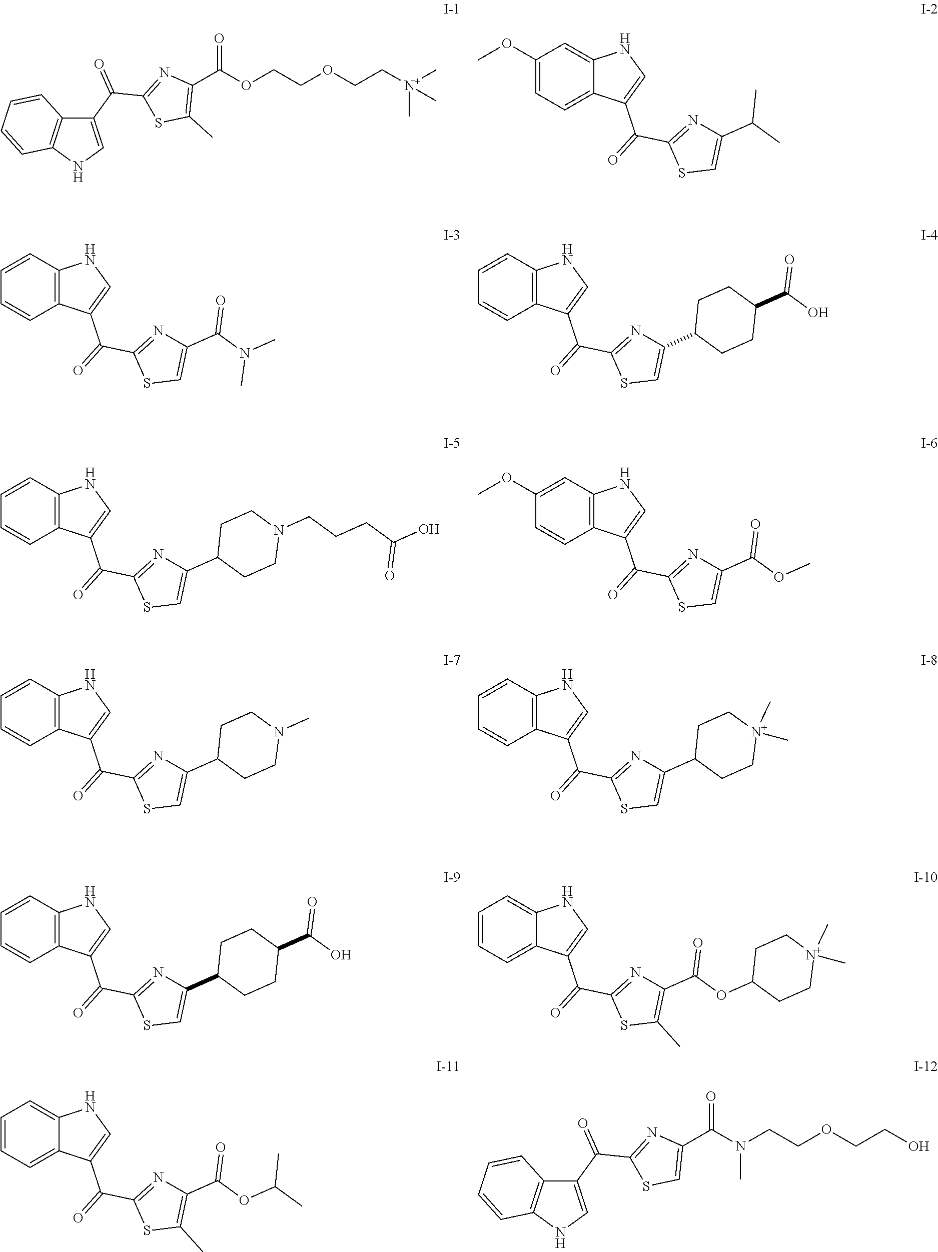 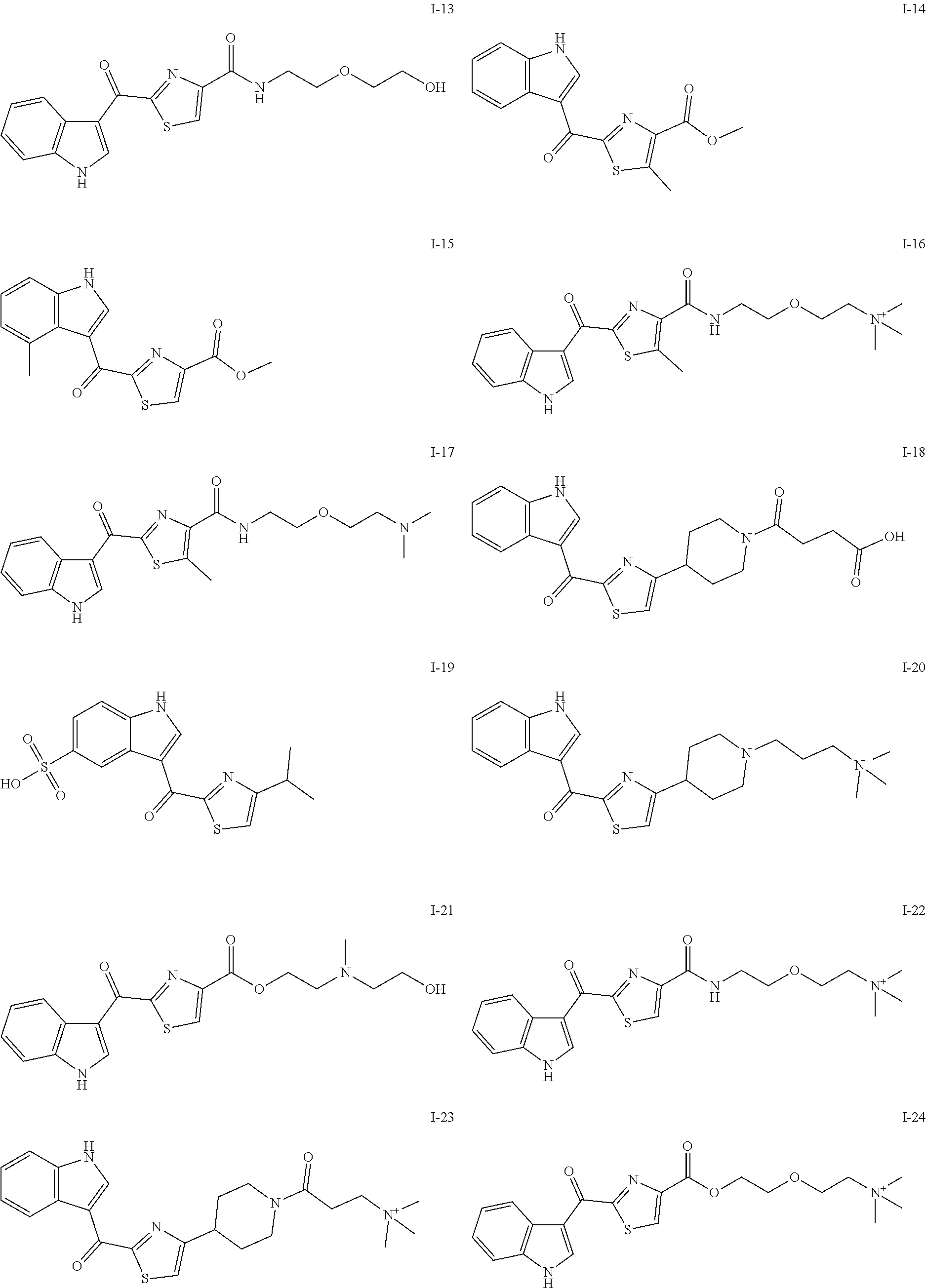 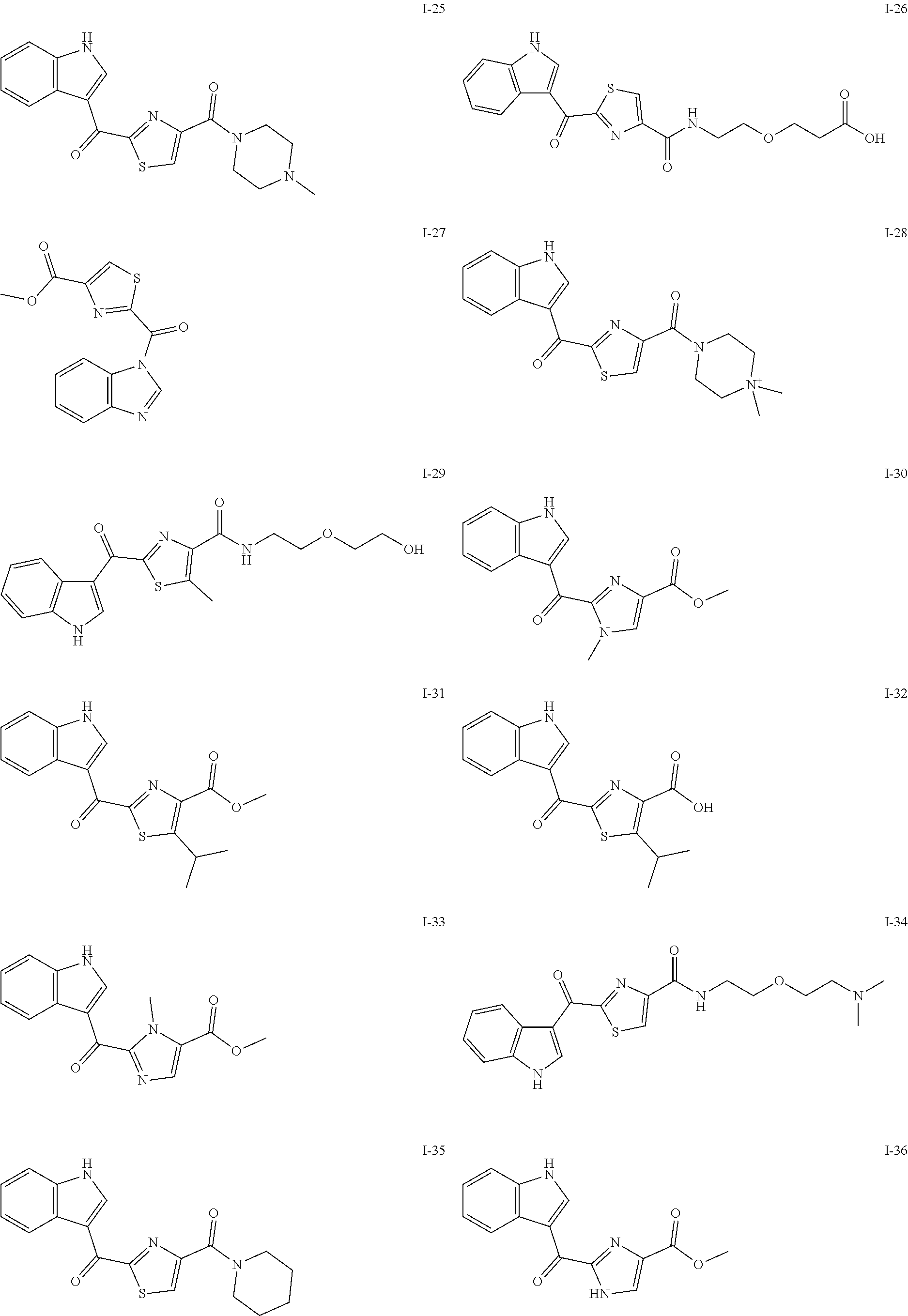 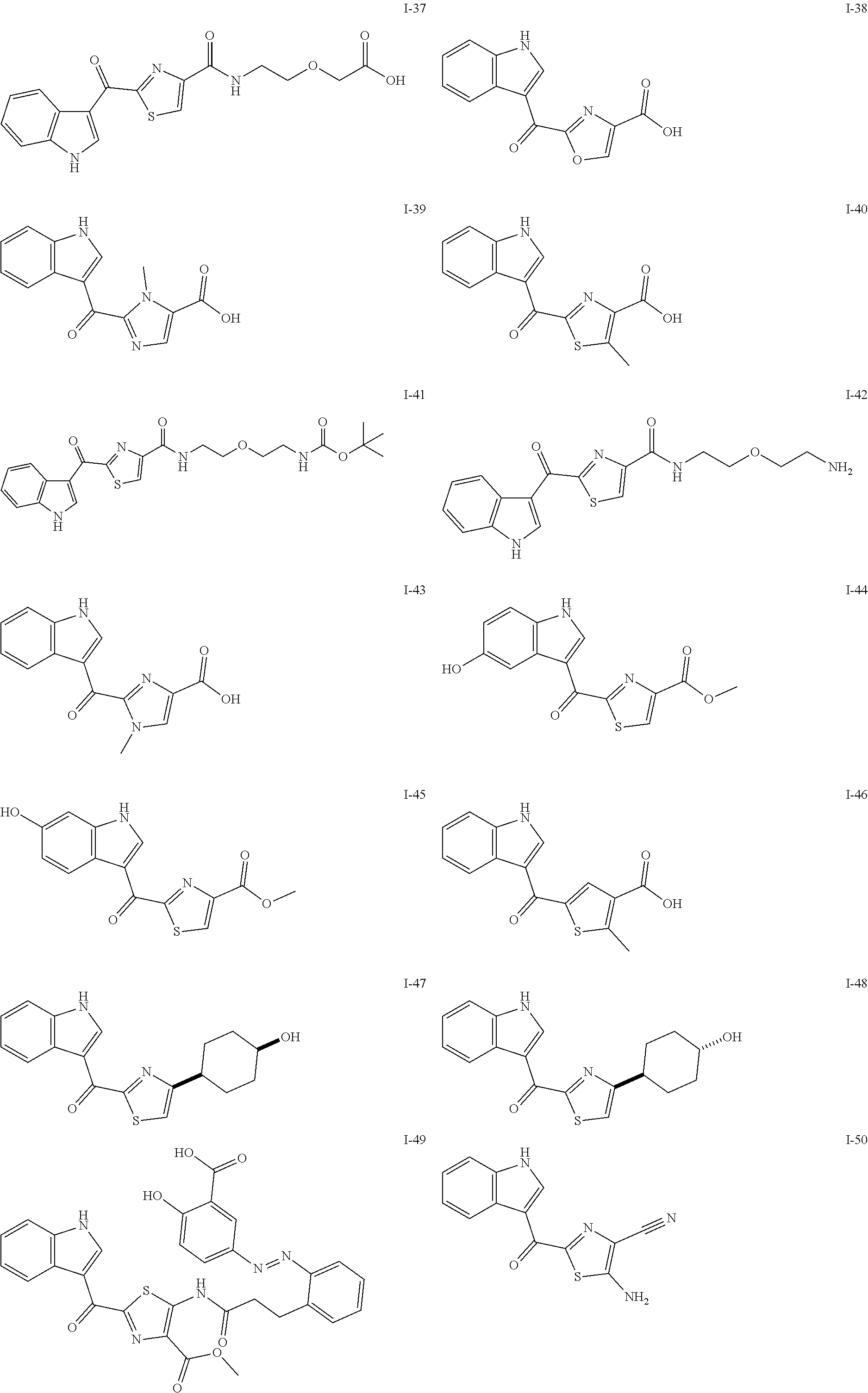 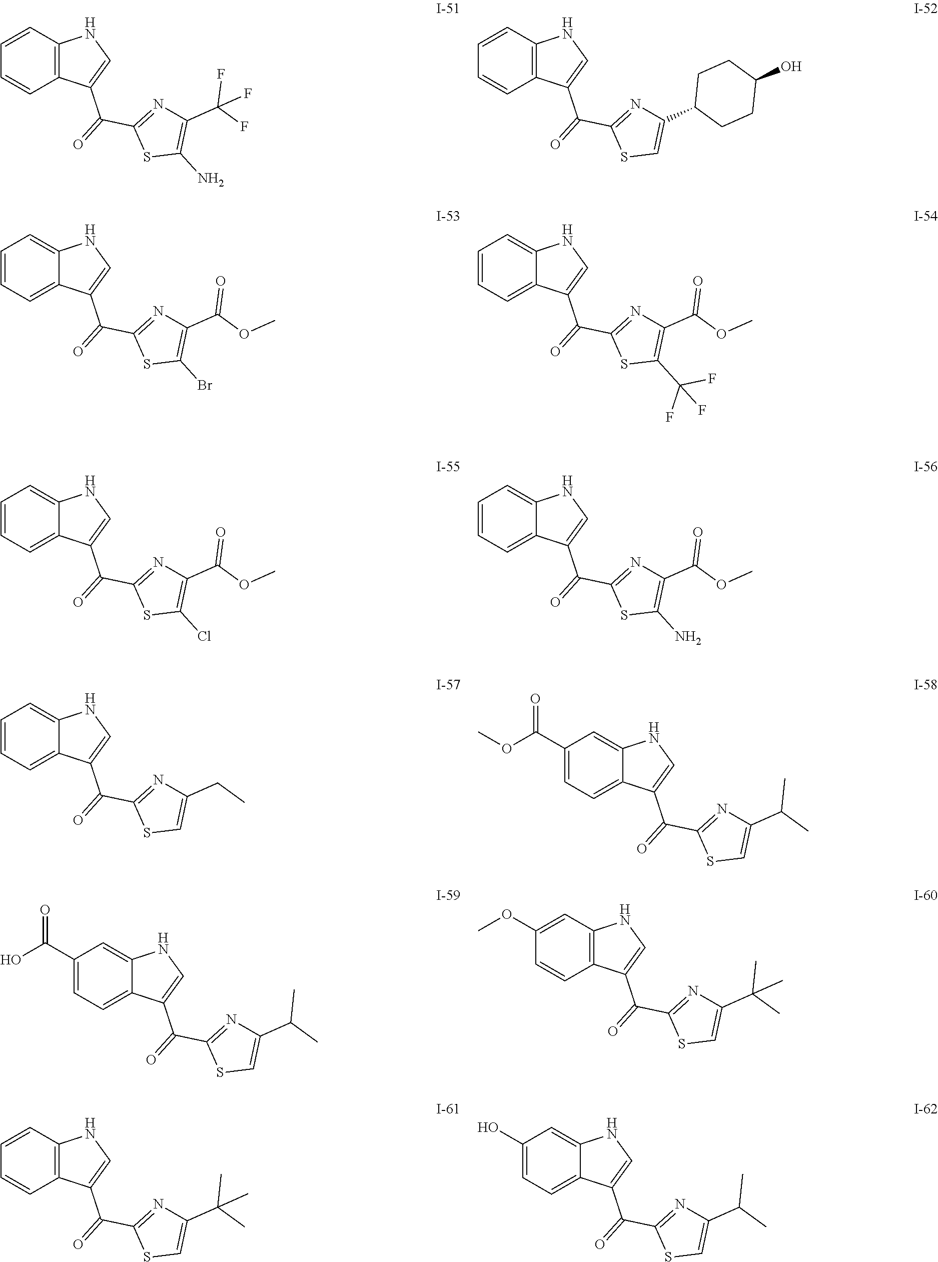 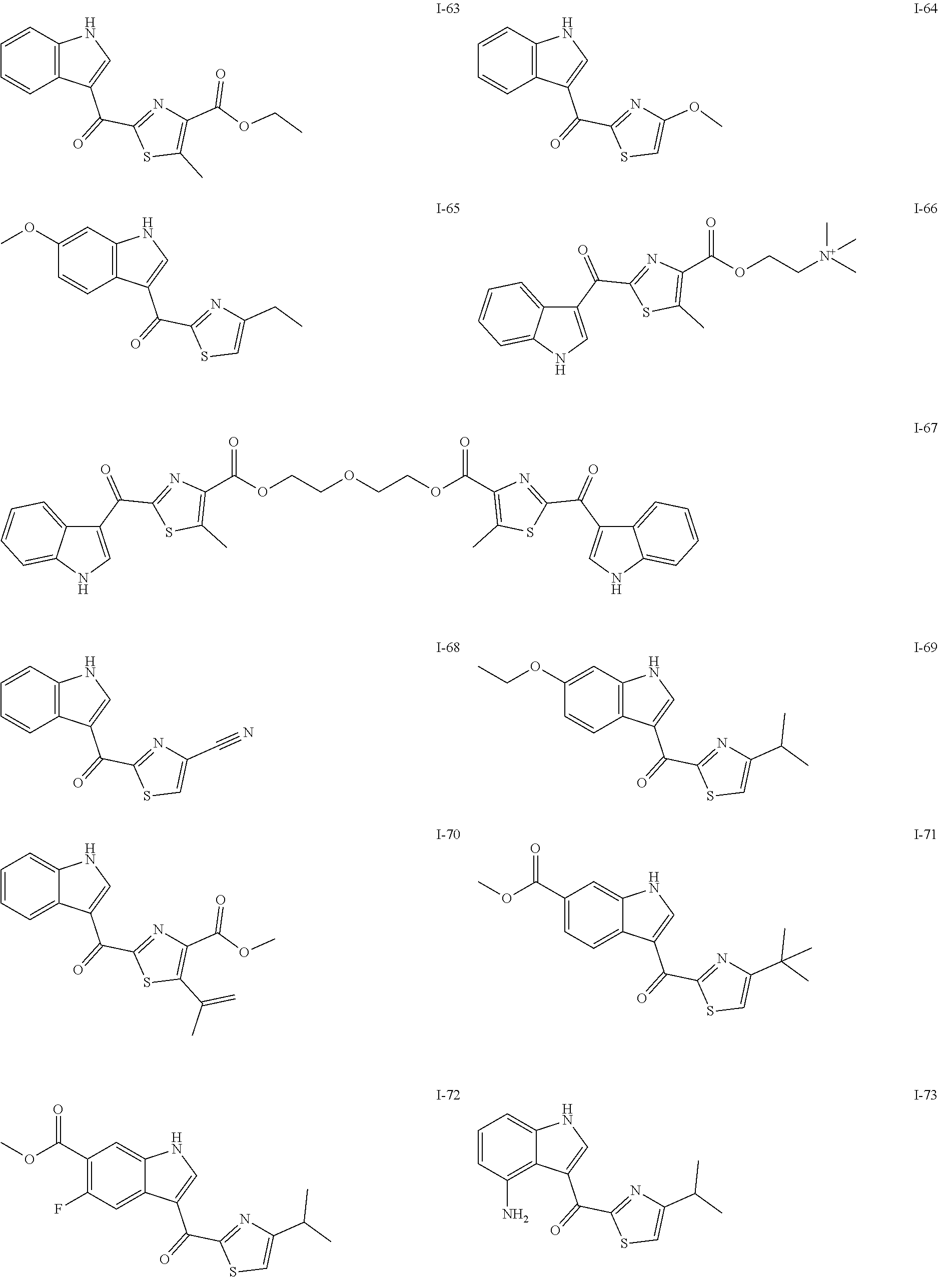 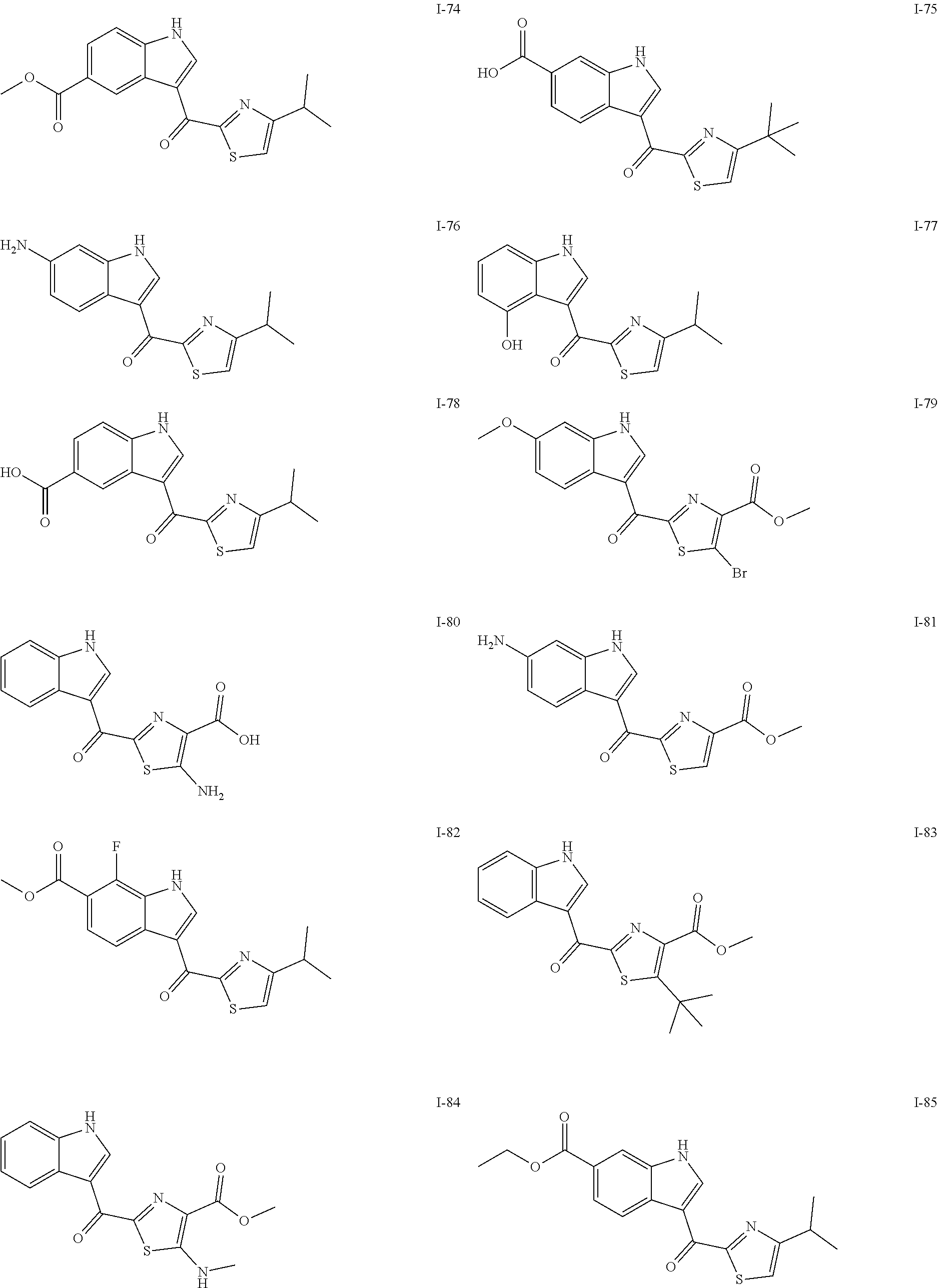 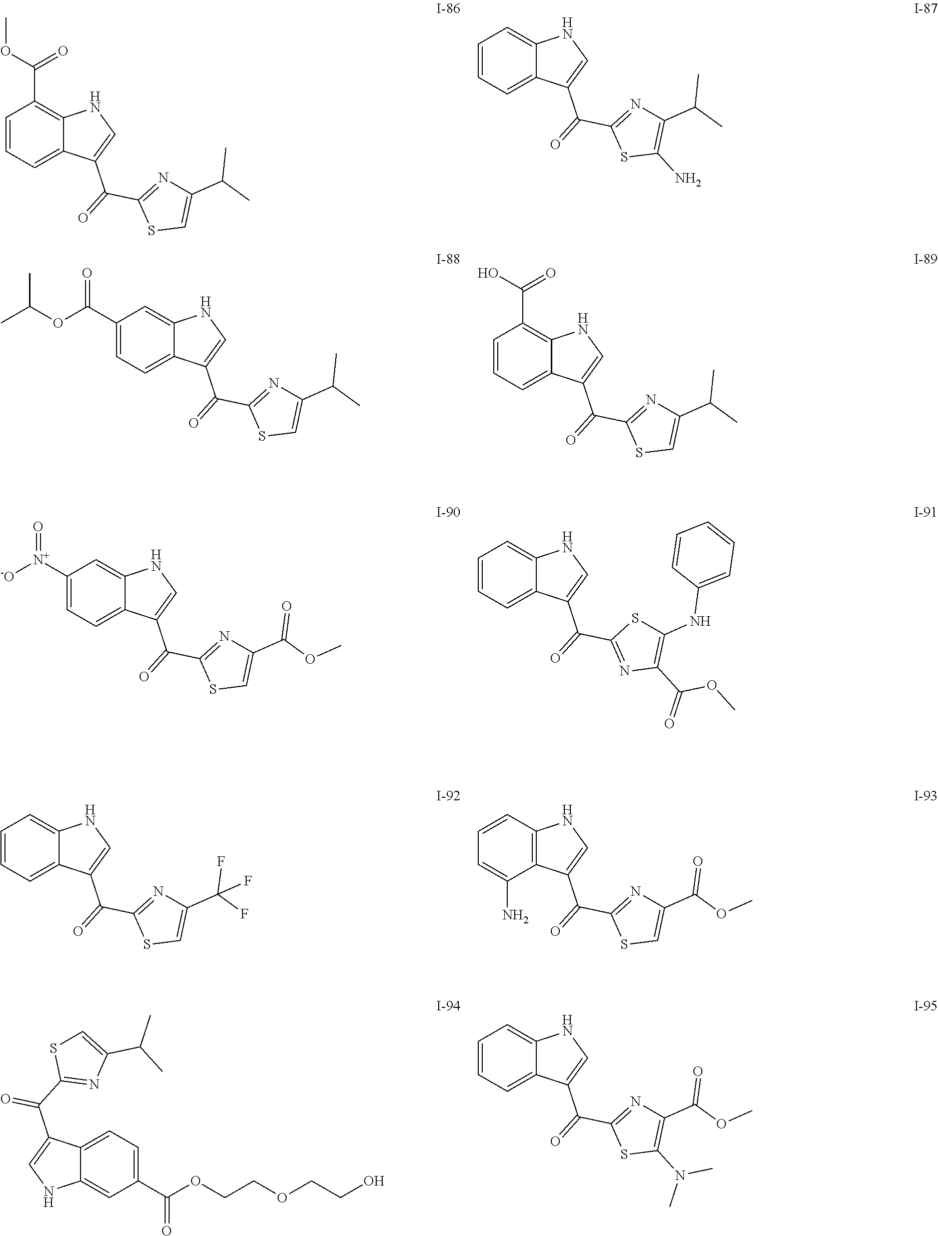 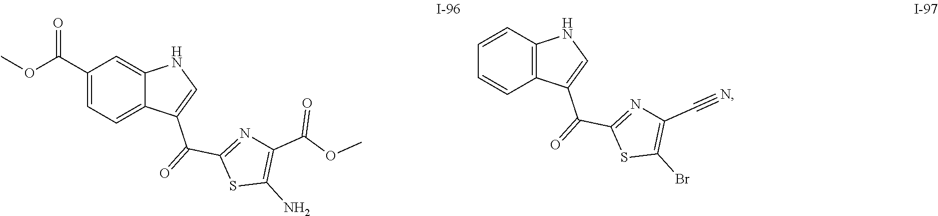
or a pharmaceutically acceptable salt thereof.

10. A pharmaceutical composition comprising the compound of claim 1, or a pharmaceutically acceptable salt thereof, and a pharmaceutically acceptable carrier, adjuvant, or vehicle.

11. A method for treating or preventing or reducing the risk of an angiogenesis implicated disorder in a patient comprising administering to the patient the compound of any claim 1, or a pharmaceutically acceptable salt thereof.

12. (canceled)

13. A method for treating or preventing or reducing the risk of an inflammatory disorder in a patient comprising administering to the patient the compound of claim 1, or a pharmaceutically acceptable salt thereof.

14. The compound of claim 1, or a pharmaceutically acceptable salt thereof, wherein R⁶ is H.

15. The compound of claim 1, or a pharmaceutically acceptable salt thereof, wherein R is hydrogen, or optionally substituted C₁₋₆ aliphatic.

16. The compound of claim 3, or a pharmaceutically acceptable salt thereof, wherein R⁸ is —N(R)₂.

17. The compound of claim 4, or a pharmaceutically acceptable salt thereof, wherein R⁷ is —C(O)—OR.

18. The compound of claim 1, which is a compound selected from Formulas (I-b) to (I-h): 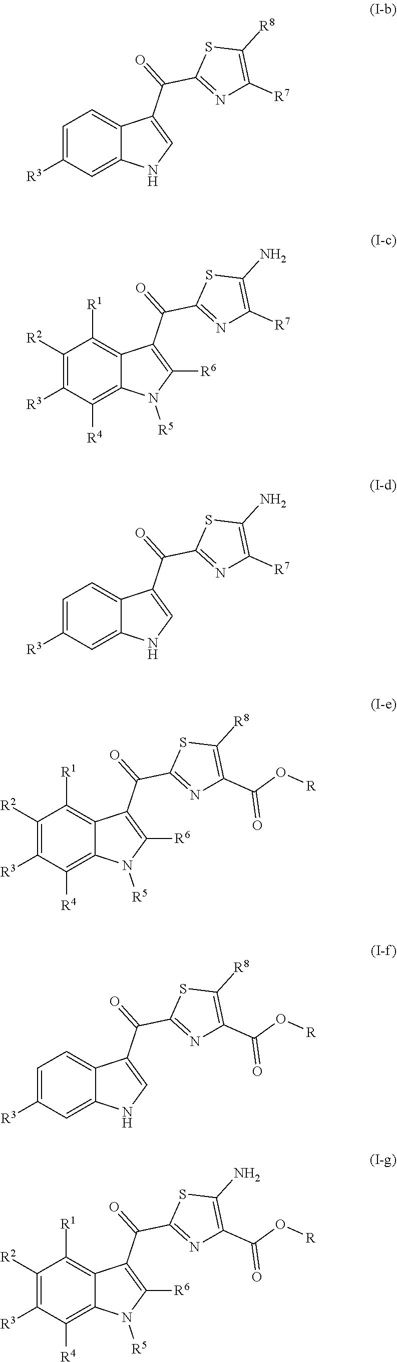 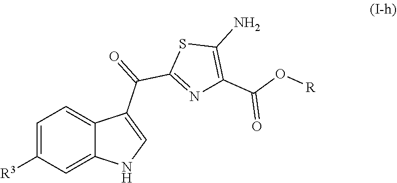
or a pharmaceutically acceptable salt thereof.

19. A pharmaceutical composition comprising the compound of claim 9, or a pharmaceutically acceptable salt thereof, and a pharmaceutically acceptable carrier, adjuvant, or vehicle.

20. A method for treating or preventing or reducing the risk of an angiogenesis implicated disorder in a patient comprising administering to the patient the compound of claim 9, or a pharmaceutically acceptable salt thereof.

21. A method for treating or preventing or reducing the risk of an inflammatory disorder in a patient comprising administering to the patient the compound of claim 9, or a pharmaceutically acceptable salt thereof.
